SEQUENCE LISTING (59 sequences)

SEQ ID NO: 1 - DNA, 3471 bp, Artificial Sequence
  Description of Artificial Sequence Synthetic polynucleotide
  pT7/VP1SA11
  modified_base (365)..(365): a, c, t, or g
aagctttaatacgactcactataggctattaaagctgtacaatggggaagtacaatctaatcttgtcagaatatctatcatttatatataattcacaatctgcagttcaaattccaatatattactcttccaacagtgaattagaaaatagatgtattgaatttcattccaagtgtttagagaactcaaagaatgggttatcgttaagaaagttgtttgttgaatataatgatgtcatagaaaatgccacattactgtcaatactatcatattcttacgacaagtataacgctgttgaaagaaaattggtgaagtatgcgaaaggcaaaccattggaggcagacttaacagtgaatgaattggantatgagaacaataaaataacatctgaattatttccaacagcggaggaatatacggactcactaatggatccagcaattttaacttcgctatcatcaaatttaaatgcagtcatgttctggttggaaaaacatgaaaatgatgtcgctgaaaaacttaaagtttataaaaggagattagacctattcaccatagtagcctcaacgataaataaatatggcgtaccaaggcataacgcaaagtacagatatgaatacgacgtaatgaaagataaaccgtactacttagtgacatgggcaaattcttcaattgaaatgttaatgtcagttttctctcatgacgactatttgatagcaaaagagttaatagtgttatcatattctaatagatctactctagcaaagttagtgtcatcaccaatgtcgattttggtagccttggtggatattaatggaacatttattacaaatgaagaattagaattggaattttcaaataaatatgtacgagcaatagttccggatcaaacatttgacgaattaaatcaaatgcttgacaatatgaggaaagctggattagttgacatacctaagatgatacaggactggttagttgatcgttctatcgaaaaatttccattaatggctaagatatattcatggtcgtttcatgttggatttagaaagcaaaaaatgctagatgctgcgctggatcaattgaaaactgagtatacagaaaatgtggacgatgaaatgtatcgggaatatacaatgttaataagagatgaagtagttaaaatgcttgaagaaccagttaaacatgatgatcacttgctacgagattctgagttagctggtttactatcaatgtcgtcagcatcgaatggtgagtcaaggcagctaaagtttggtaggaaaacaattttttcaactaaaaagaatatgcatgtcatggatgatatggctaacgaaagatacacgcctggtataataccaccagtgaatgttgataaaccaataccattaggaagaagagatgttccaggaagaaggactagaataatattcattctgccatacgaatatttcatagcacagcacgctgtagttgaaaaaatgttgatttacgcaaaacatacgagagaatacgctgaattttattcacaatcaaaccaattattgtcatacggcgatgtaacgcgttttttgtctaataacacaatggtcttgtatacggatgtatctcagtgggattcgtctcagcataatacacagccatttaggaaaggaataataatgggactggacatattagctaacatgactaatgatgctaaagttcttcagacattaaacttatacaaacaaacacaaatcaatctcatggattcatacgttcaaataccagatggcaacgtcattaagaaaatacaatacggggcagtagcatcaggagagaaacaaacgaaagcagcaaattcaatagcaaatttggcactgattaaaacggttttgtcacgtatttctaacaaacattcattcgcaacaaaaataataagagttgatggagatgataactatgcggtgctacaatttaatacagaggtgactaagcagatgatccaagacgtatcgaacgatgtaagagaaacttatgcacgcatgaatgctaaagttaaagctctggtatccacagtaggaatagaaattgctaaaaggtacattgcaggtggaaaaatattttttcgagctggaataaatctacttaataatgaaaagagagggcagagtacgcagtgggatcaagcagcaattttatattcaaattatattgtaaatagacttagaggatttgaaactgatagggagtttattttaactaagataatgcagatgacgtcagtcgcaattactggatcattaagactatttccttctgaacgcgtattaactacgaattcaacatttaaagtatttgactcggaggattttattatagagtacggaacgactgatgacgaagtatatatacaaagagcgttcatgtctttatcaagtcagaaatcaggaatagccgatgagatagcggcatcatcaacatttaaaaattacgtcacgagactatctgaacagttattattttcaaagaataatatagtgtccagaggaatagctttgactgaaaaagcgaaattgaattcatacgctccaatatcgcttgagaaaagacgtgcacagatatcagctttattgactatgttgcagaaaccggtcaccttcaaatcaagtaaaataacaataaatgacatactcagagatataaaaccattttttacagtaagtgatgcacacttacctatacaataccaaaaatttatgccaactttgccagataacgtacagtatataattcaatgtataggatccagaacttatcaaattgaagatgacggttcgaagtcagccatatctagactaatatcaaagtattcagtttataagccatcaattgaagaattgtataaagtgatttcattgcatgaaaacgaaatacaattatatctgatttcattaggaataccgaaaatagacgctgacacgtatgttggatcaaagatttattctcaagataagtatagaatactagaatcatacgtgtacaatttattgtccattaattatggatgctatcaattatttgatttcaattcaccggacttggagaagctgataagaataccatttaagggaaaaataccagctgttacattcatattacacttatatgcaaagctagaagttataaactacgctataaaaaatggttcatggataagcctattttgcaattaccctaaatcagaaatgataaaattatggaagaagatgtggaacatcacgtcattacgttcgccgtacactaacgcgaacttctttcaagattagaacgcttagatgtgaccgggtcggcatggcatctccacctcctcgcggtccgacctgggcatccgaaggaggacgcacgtccactcggatggctaagggagagcctgcagtagcataaccccttggggcctctaaacgggtcttgaggggttttttgggtacc

SEQ ID NO: 2 - protein, 1088 aa, Rotavirus A
  MOD_RES (108)..(108): Any amino acid
MGKYNLILSEYLSFIYNSQSAVQIPIYYSSNSELENRCIEFHSKCLENSKNGLSLRKLFVEYNDVIENATLLSILSYSYDKYNAVERKLVKYAKGKPLEADLTVNELXYENNKITSELFPTAEEYTDSLMDPAILTSLSSNLNAVMFWLEKHENDVAEKLKVYKRRLDLFTIVASTINKYGVPRHNAKYRYEYDVMKDKPYYLVTWANSSIEMLMSVFSHDDYLIAKELIVLSYSNRSTLAKLVSSPMSILVALVDINGTFITNEELELEFSNKYVRAIVPDQTFDELNQMLDNMRKAGLVDIPKMIQDWLVDRSIEKFPLMAKIYSWSFHVGFRKQKMLDAALDQLKTEYTENVDDEMYREYTMLIRDEVVKMLEEPVKHDDHLLRDSELAGLLSMSSASNGESRQLKFGRKTIFSTKKNMHVMDDMANERYTPGIIPPVNVDKPIPLGRRDVPGRRTRIIFILPYEYFIAQHAVVEKMLIYAKHTREYAEFYSQSNQLLSYGDVTRFLSNNTMVLYTDVSQWDSSQHNTQPFRKGIIMGLDILANMTNDAKVLQTLNLYKQTQINLMDSYVQIPDGNVIKKIQYGAVASGEKQTKAANSIANLALIKTVLSRISNKHSFATKIIRVDGDDNYAVLQFNTEVTKQMIQDVSNDVRETYARMNAKVKALVSTVGIEIAKRYIAGGKIFFRAGINLLNNEKRGQSTQWDQAAILYSNYIVNRLRGFETDREFILTKIMQMTSVAITGSLRLFPSERVLTTNSTFKVFDSEDFIIEYGTTDDEVYIQRAFMSLSSQKSGIADEIAASSTFKNYVTRLSEQLLFSKNNIVSRGIALTEKAKLNSYAPISLEKRRAQISALLTMLQKPVTFKSSKITINDILRDIKPFFTVSDAHLPIQYQKFMPTLPDNVQYIIQCIGSRTYQIEDDGSKSAISRLISKYSVYKPSIEELYKVISLHENEIQLYLISLGIPKIDADTYVGSKIYSQDKYRILESYVYNLLSINYGCYQLFDFNSPDLEKLIRIPFKGKIPAVTFILHLYAKLEVINYAIKNGSWISLFCNYPKSEMIKLWKKMWNITSLRSPYTNANFFQD

SEQ ID NO: 3 - DNA, 2862 bp, Artificial Sequence
  Description of Artificial Sequence Synthetic polynucleotide
  pT7/VP2SA11
actagttaatacgactcactataggctattaaaggctcaatggcgtatcgaaaacgtggagcgcgtcgtgagacgaatctaaaacaagatgaacgaatgcaagaaaaagaagatagcaagaacattaataatgacagtcctaaatcacaattatcagaaaaagtattatctaagaaagaagagataattacagataatcaagaagaagttaagatatctgatgaggtaaaaaaatctaataaagaagaatcgaaacagttgttagaagtacttaaaacaaaagaggaacatcaaaaagaagttcagtatgaaatattacaaaaaactatccctacatttgaaccaaaagagtcaatactcaaaaaattagaagacataaaaccagaacaagcaaagaaacaaactaaactgtttcgaatatttgaaccgaaacaattgcctatttatagagctaatggagaaagagagcttcgtaatagatggtattggaaattgaaacgagatactcttcctgatggagattatgatgttagagagtattttttaaatttatatgatcaagtattaatggaaatgccggattatctattacttaaagatatggctgtagagaataaaaattcaagggatgctggcaaagtagttgattctgaaacagccgcaatatgcgatgctatttttcaagatgaagaaaccgaaggtgcagtaagaagattcatagctgagatgagacaacgagttcaagctgatcgaaatgtagtcaattatccatctatattgcatccaattgaccatgcgtttaacgaatacttcttacaacatcagttggtagaaccattaaataatgatatcattttcaattacataccagagagaataagaaatgatgtcaactatatattaaatatggacaggaatttaccgtctactgctagatatatcagaccaaacttgctacaagataggttaaatttacatgataattttgagtcactctgggatactataactacatctaattatattttagcaagatctgtggtgccagacctaaaagaattagtatctactgaggcacaaatccagaaaatgtcacaagatttgcaattggaagctttgacaatacaatcagagactcagtttttaacaggtataaactcacaagccgctaatgattgttttaaaactttgattgctgctatgttgagtcagagaaccatgtcattagatttcgtaacgacaaattacatgtcacttatttcaggcatgtggttactcactgtgattccaaatgatatgtttataagagaatcattagtagcatgtcaactagccataataaataccattgtttatccggcattcggaatgcaaagaatgcattataggaatggtgatccacagactccctttcaaattgcagagcaacagattcaaaattttcaggtagctaattggttacattttgttaattataatcagtttagacaagtagtgattgatggagtgttaaatcaagtcttgaatgataatataagaaatggtcatgtagtcaaccaattaatggaagctctgatgcaattatctagacaacagtttcccacaatgccagttgattataaaagatctatacagagaggaattttgctgctttctaacagacttggtcagcttgtcgatttaacaagattgttatcatacaattatgagacattaatggcatgcataacaatgaatatgcagcatgttcaaacattaacaactgaaaaattgcaattaacatcagtaacatcattatgtatgctaattggaaatgctacggttataccgagtccgcaaacattgttccattactataatgtgaatgtcaattttcattcaaattataatgaaagaattaatgacgcagttgcaattataactgcggcaaatagattaaatttatatcaaaagaaaatgaaatcaatagttgaggactttctgaaaagattacagatatttgatgttgcgagagtaccagatgaccaaatgtatagattgagagatagattaagactattaccagttgaaataagaagattagatatttttaatttgatagcaatgaatatggaacagattgaacgtgcatcagataaaattgcacaaggagttataatagcataccgagatatgcagttagaacgagatgagatgtatggttacgtcaatattgccagaaacttggacggatttcaacaaataaatcttgaagaattgatgagatcaggagattatgctcaaattactaacatgctacttaataatcaaccagtagctttagttggagcgctaccatttataacggattcatcagtgatttcgttaatagctaaactagatgcaaccgtttttgcacagattgtcaaacttagaaaggtcgacacgttaaaacccatcctatataagataaattcagattctaatgacttttatttggtggctaattatgattggattcctacatctactacaaaagtgtataaacaagttccacaacaatttgattttagagcgtcaatgcatatgttaacgtctaacctaacatttaccgtatattcagatttgcttgcgttcgtttcagctgatactgttgaaccaattaatgctgttgcttttgataatatgcgcatcatgaacgaactgtaaacgccaaccccattgtggagatatgaccgggtcggcatggcatctccacctcctcgcggtccgacctgggcatccgaaggaggacgcacgtccactcggatggctaagggagagcctgcagtagcataaccccttggggcctctaaacgggtcttgaggggttttttgggtacc

SEQ ID NO: 4 - protein, 882 aa, Rotavirus A
MAYRKRGARRETNLKQDERMQEKEDSKNINNDSPKSQLSEKVLSKKEEIITDNQEEVKISDEVKKSNKEESKQLLEVLKTKEEHQKEVQYEILQKTIPTFEPKESILKKLEDIKPEQAKKQTKLFRIFEPKQLPIYRANGERELRNRWYWKLKRDTLPDGDYDVREYFLNLYDQVLMEMPDYLLLKDMAVENKNSRDAGKVVDSETAAICDAIFQDEETEGAVRRFIAEMRQRVQADRNVVNYPSILHPIDHAFNEYFLQHQLVEPLNNDIIFNYIPERIRNDVNYILNMDRNLPSTARYIRPNLLQDRLNLHDNFESLWDTITTSNYILARSVVPDLKELVSTEAQIQKMSQDLQLEALTIQSETQFLTGINSQAANDCFKTLIAAMLSQRTMSLDFVTTNYMSLISGMWLLTVIPNDMFIRESLVACQLAIINTIVYPAFGMQRMHYRNGDPQTPFQIAEQQIQNFQVANWLHFVNYNQFRQVVIDGVLNQVLNDNIRNGHVVNQLMEALMQLSRQQFPTMPVDYKRSIQRGILLLSNRLGQLVDLTRLLSYNYETLMACITMNMQHVQTLTTEKLQLTSVTSLCMLIGNATVIPSPQTLFHYYNVNVNFHSNYNERINDAVAIITAANRLNLYQKKMKSIVEDFLKRLQIFDVARVPDDQMYRLRDRLRLLPVEIRRLDIFNLIAMNMEQIERASDKIAQGVIIAYRDMQLERDEMYGYVNIARNLDGFQQINLEELMRSGDYAQITNMLLNNQPVALVGALPFITDSSVISLIAKLDATVFAQIVKLRKVDTLKPILYKINSDSNDFYLVANYDWIPTSTTKVYKQVPQQFDFRASMHMLTSNLTFTVYSDLLAFVSADTVEPINAVAFDNMRIMNEL

SEQ ID NO: 5 - DNA, 2760 bp, Artificial Sequence
  Description of Artificial Sequence Synthetic polynucleotide
  pT7/VP3SA11
tctagataatacgactcactataggctattaaagcagtaccagtagtgtgttttacctctgatggtgtaaacatgaaagtactagctttaagacacagtgtggctcaagtgtatgcagacactcaagtctacgttcatgatgatacaaaagatagttatgaaaacgcttttttaatctctaatcttacgacccataatattttatacttaaattatagcattaaaacattagaaatattaaataagtcaggaatagctgcaattgctttacaatcacttgaagaattattcacattaataaggtgtaatttcacttatgattatgaacttgatataatatatttacatgattattcatattataccaataatgaaattagaacagaccaacattggataacaaaaacaaatattgaagaatatttactacctggatggaaattaacatatgttggttataatggaagtgaaactagaggacattataacttttcatttaaatgtcaaaacgctgcaacagatgatgatctaataattgaatacatttattcagaagcgttggacttccaaaattttatgttaaaaaagataaaggaaagaatgactacatcgttgcctatagctagattatctaacagagtatttagggataagttattcccatcattattgaaagaacataagaatgtagtgaacgttggtccgcgtaatgaatctatgtttacatttttaaattatccaactataaaacaattttcaaatggtgcgtatttagtaaaagatactataaaattaaaacaagaacgatggttaggtaaaaggatatctcagtttgatattggtcagtataaaaatatgctgaatgttcttacagcaatttattattactataatttatataaaagtaaaccaattatatatatgatcggatctgctccatcttattggatatatgacgttaggcattattccgattttttctttgaaacttgggatccattggacacaccatattcatcaatccatcacaaagaattattttttataaatgatgtgaagaaactgaaggataactcaatattgtatattgatataagaaccgataggggcaatgctgattggaaaaaatggagaaagacagtagaagaacaaactattaataatttggacatagcttatgaatatttacgaacgggtaaagcgaaggtgtgttgtgttaagatgacagctatggatttggaactgccaatttcagctaaattactgcaccacccaactacggaaataagatcagaattttatttattactagatacttgggatttaactaacattaggaggttcattcctaaaggcgtgttatattcatttataaacaatataataactgaaaatgtgtttattcaacaaccatttaaagtaaaagtactgaatgatagttatattgtagcgttatatgcattatcaaatgattttaataatagatcagaagtaattaaattaattaataatcagaaacaatctctaataactgttagaataaataatacgtttaaggatgaaccaaaagttgggttcaaaaatatctatgattggacctttcttccaaccgactttgataccaaagaagctataattacttcatacgacggttgtttaggactctttggtttgtctatatcgttagcatcaaaaccaacagggaataatcatttattcattttaagtggtacagataagtattataaattggatcaatttgctaatcacaccagtatatcgagaagatcacaccaaattaggttttcggaatctgctacttcatattcaggttatatatttagagatttgtccaataataattttaatctaattggtactaatatagagaattcagtatcaggtcatgtatataatgctttaatttattatagatataattattcatttgatcttaaacgctggatttatttacattctatagataaagttgatatagaaggaggaaagtattatgaacacgcaccaatagaattaatttatgcatgtagatcagcaaaagaatttgctacattgcaggatgacttaactgtattgagatattcaaacgaaatagagaattatattaatacagtatatagtataacatacgctgatgatccgaattactttatcggaatacaatttagaaatataccatataaatatgatgttaaaataccgcatttaaccttcggagtattacatatttctgataacatggtgccagacgtgattgacatactaaagataatgaagaatgaattatttaaaatggatattacgaccagttatacatatatgttatcagatggaatctacgtagcaaatgttagtggagtattatctacatactttaaaatctataacgtattttataaaaatcaaataacttttggccaatccagaatgtttattccgcacataacattaagcttcaataacatgagaacagtaaggatagagactactaaattacaaattaaatccatttatttaagaaagattaagggtgatacagtgtttgatatggttgagtgagctaaaaacttaacacactagtcatgatgtgaccgggtcggcatggcatctccacctcctcgcggtccgacctgggcatccgaaggaggacgcacgtccactcggatggctaagggagagcctgcagtagcataaccccttggggcctctaaacgggtcttgaggggttttttgggtacc

SEQ ID NO: 6 - protein, 835 aa, Rotavirus A
MKVLALRHSVAQVYADTQVYVHDDTKDSYENAFLISNLTTHNILYLNYSIKTLEILNKSGIAAIALQSLEELFTLIRCNFTYDYELDIIYLHDYSYYTNNEIRTDQHWITKTNIEEYLLPGWKLTYVGYNGSETRGHYNFSFKCQNAATDDDLIIEYIYSEALDFQNFMLKKIKERMTTSLPIARLSNRVFRDKLFPSLLKEHKNVVNVGPRNESMFTFLNYPTIKQFSNGAYLVKDTIKLKQERWLGKRISQFDIGQYKNMLNVLTAIYYYYNLYKSKPIIYMIGSAPSYWIYDVRHYSDFFFETWDPLDTPYSSIHHKELFFINDVKKLKDNSILYIDIRTDRGNADWKKWRKTVEEQTINNLDIAYEYLRTGKAKVCCVKMTAMDLELPISAKLLHHPTTEIRSEFYLLLDTWDLTNIRRFIPKGVLYSFINNIITENVFIQQPFKVKVLNDSYIVALYALSNDFNNRSEVIKLINNQKQSLITVRINNTFKDEPKVGFKNIYDWTFLPTDFDTKEAIITSYDGCLGLFGLSISLASKPTGNNHLFILSGTDKYYKLDQFANHTSISRRSHQIRFSESATSYSGYIFRDLSNNNFNLIGTNIENSVSGHVYNALIYYRYNYSFDLKRWIYLHSIDKVDIEGGKYYEHAPIELIYACRSAKEFATLQDDLTVLRYSNEIENYINTVYSITYADDPNYFIGIQFRNIPYKYDVKIPHLTFGVLHISDNMVPDVIDILKIMKNELFKMDITTSYTYMLSDGIYVANVSGVLSTYFKIYNVFYKNQITFGQSRMFIPHITLSFNNMRTVRIETTKLQIKSIYLRKIKGDTVFDMVE

SEQ ID NO: 7 - DNA, 2531 bp, Artificial Sequence
  Description of Artificial Sequence Synthetic polynucleotide
  pT7/VP4SA11
actagttaatacgactcactataggctataaaatggcttcgctcatttatagacaattgctcacgaattcttatacagtagatttatccgatgagatacaagagattggatcaactaaatcacaaaatgtcacaattaatcctggaccatttgcgcaaacaggttatgctccagttaactggggacctggagaaattaatgattctacgacagttggaccattgctggatgggccttatcaaccaacgacattcaatccaccagtcgattattggatgttactggctccaacgacacctggcgtaattgttgaaggtacaaataatacagatagatggttagccacaattttaatcgagccaaatgttcagtctgaaaatagaacttacactatatttggtattcaagaacaattaacggtatccaatacttcacaagaccagtggaaatttattgatgtcgtaaaaacaactgcaaatggaagtataggacaatatggaccattactatccagtccgaaattatatgcagttatgaagcataatgaaaaattatatacatatgaaggacagacacctaacgctaggacagcacattattcaacaacgaattatgattctgttaacatgactgctttttgtgacttttatataattcctagatctgaagagtctaaatgtacggaatacattaataatggattaccaccaatacaaaatactagaaatgttgtaccattatcgttgactgctagagatgtaatacactatagagctcaagctaatgaagatattgtgatatccaagacatcattatggaaagaaatgcaatataatagagatataactattagatttaaatttgcaaatacaattataaaatcaggagggctgggatataagtggtcagaaatatcatttaagccagcgaattatcaatacacatatactcgtgatggtgaagaagttaccgcacatactacttgttcagtgaatggcgttaatgacttcagttttaatggaggatatttaccaactgattttgttgtatctaaatttgaagtaattaaagagaattcatacgtctatatcgattactgggatgattcacaagcatttcgtaacgtggtgtatgtccgatcgttagcagcaaacttgaattcagttatgtgtactggaggcagctataattttagtctaccagttggacaatggcctgttttaactgggggagcagtttctttacattcagctggtgtaacactatctactcaatttacagatttcgtatcattaaattcattaagatttagatttagactagctgtcgaagaaccacactttaaactgactagaactagattagatagattgtatggtctgcctgctgcagatccaaataatggtaaagaatattatgaaattgctggacgattttcacttatatcattagtgccatcaaatgatgactatcagactcctatagcaaactcagttactgtacgacaagatttagaaaggcagttaggagaactaagagaagagtttaacgctttgtctcaagaaattgcaatgtcgcagttaatcgatttagcgcttctaccattagatatgttctcaatgttttctggcattaaaagtactattgatgctgcaaaatcaatggctactaatgttatgaaaaaattcaaaaagtcaggattagcgaattcagtttcaacactgacagattctttatcagacgcagcatcatcaatatcaagaggttcatctatacgttcgattggatcttcagcatcagcatggacggatgtatcaacacaaataactgatatatcgtcatcagtaagttcagtttcgacacaaacgtcaactatcagtagaagattgagactaaaggaaatggcaacacaaactgagggtatgaattttgatgatatatcagcggctgttttgaagactaagatagataaatcgactcaaatatcaccaaacacaatacctgacattgttactgaagcatcggaaaaattcataccaaatagggcttaccgcgttataaacaacgatgatgtgtttgaagctggaattgatgggaaattttttgcttataaagtggatacatttgaggaaataccatttgatgtacaaaaattcgctgacttagttacagattctccagtaatatccgctataattgattttaaaacacttaaaaatttgaacgataattacggcattactaagcaacaagcatttaatcttttaagatctgacccaagagttttacgtgaattcattaatcaggacaatcctataattagaaatagaattgaacaactgattatgcaatgcaggttgtgagtaatttctagaggatgtgaccgggtcggcatggcatctccacctcctcgcggtccgacctgggcatccgaaggaggacgcacgtccactcggatggctaagggagagcctgcagtagcataaccccttggggcctctaaacgggtcttgaggggttttttgggtacc

SEQ ID NO: 8 - protein, 776 aa, Rotavirus A
MASLIYRQLLTNSYTVDLSDEIQEIGSTKSQNVTINPGPFAQTGYAPVNWGPGEINDSTTVGPLLDGPYQPTTFNPPVDYWMLLAPTTPGVIVEGTNNTDRWLATILIEPNVQSENRTYTIFGIQEQLTVSNTSQDQWKFIDVVKTTANGSIGQYGPLLSSPKLYAVMKHNEKLYTYEGQTPNARTAHYSTTNYDSVNMTAFCDFYIIPRSEESKCTEYINNGLPPIQNTRNVVPLSLTARDVIHYRAQANEDIVISKTSLWKEMQYNRDITIRFKFANTIIKSGGLGYKWSEISFKPANYQYTYTRDGEEVTAHTTCSVNGVNDFSFNGGYLPTDFVVSKFEVIKENSYVYIDYWDDSQAFRNVVYVRSLAANLNSVMCTGGSYNFSLPVGQWPVLTGGAVSLHSAGVTLSTQFTDFVSLNSLRFRFRLAVEEPHFKLTRTRLDRLYGLPAADPNNGKEYYEIAGRFSLISLVPSNDDYQTPIANSVTVRQDLERQLGELREEFNALSQEIAMSQLIDLALLPLDMFSMFSGIKSTIDAAKSMATNVMKKFKKSGLANSVSTLTDSLSDAASSISRGSSIRSIGSSASAWTDVSTQITDISSSVSSVSTQTSTISRRLRLKEMATQTEGMNFDDISAAVLKTKIDKSTQISPNTIPDIVTEASEKFIPNRAYRVINNDDVFEAGIDGKFFAYKVDTFEEIPFDVQKFADLVTDSPVISAIIDFKTLKNLNDNYGITKQQAFNLLRSDPRVLREFINQDNPIIRNRIEQLIMQCRL

SEQ ID NO: 9 - DNA, 1525 bp, Artificial Sequence
  Description of Artificial Sequence Synthetic polynucleotide
  pT7/VP6SA11
aagctttaatacgactcactataggcttttaaacgaagtcttcaacatggatgtcctatactctttgtcaaagactcttaaagacgctagagacaaaattgtcgaaggcacattgtattctaacgtgagtgatctaattcaacaatttaatcaaatgataattactatgaatggaaatgaatttcaaactggaggaatcggtaatttgccaattagaaactggaattttaatttcgggttacttggaacaactttgctgaacttagacgctaattatgttgaaacggcaagaaatacaattgattatttcgtggattttgtagacaatgtatgcatggatgagatggttagagaatcacaaaggaacggaattgcacctcaatcagactcgctaagaaagctgtcagccattaaattcaaaagaataaattttgataattcgtcggaatacatagaaaactggaatttgcaaaatagaagacagaggacaggtttcacttttcataaaccaaacatttttccttattcagcatcatttacactaaatagatcacaacccgctcatgataatttgatgggcacaatgtggttaaacgcaggatcggaaattcaagtcgctggatttgactactcatgtgctattaacgcaccagccaatatacaacaatttgagcatattgtgccactccgaagagtgttaactacagctacgataactcttctaccagacgcggaaaggtttagttttccaagagtgatcaattcagctgacggggcaactacatggtttttcaacccagtgattctcaggccgaataacgttgaagtggagtttctattgaatggacagataataaacacttatcaagcaagatttggaactatcgtagctagaaattttgatactattagactatcattccagttaatgagaccaccaaacatgacaccagcagtagcagtactattcccgaatgcacagccattcgaacatcatgcaacagtgggattgacacttagaattgagtctgcagtttgtgagtctgtactcgccgatgcaagtgaaactctattagcaaatgtaacatccgttaggcaagagtacgcaataccagttggaccagtctttccaccaggtatgaactggactgatttaatcaccaattattcaccgtctagggaggacaatttgcaacgcgtatttacagtggcttccattagaagcatgctcattaaatgaggaccaagctaacaacttggtatccaactttggtgagtatgtagctatatcaagctgtttgaactctgtaagtaaggatgcgtatacgcattcgctacacagagtaatcactcagatggtatagtgagaggatgtgaccgggtcggcatggcatctccacctcctcgcggtccgacctgggcatccgaaggaggacgcacgtccactcggatggctaagggagagcctgcagtagcataaccccttggggcctctaaacgggtcttgaggggttttttgggtacc

SEQ ID NO: 10 - protein, 397 aa, Rotavirus A
MDVLYSLSKTLKDARDKIVEGTLYSNVSDLIQQFNQMIITMNGNEFQTGGIGNLPIRNWNFNFGLLGTTLLNLDANYVETARNTIDYFVDFVDNVCMDEMVRESQRNGIAPQSDSLRKLSAIKFKRINFDNSSEYIENWNLQNRRQRTGFTFHKPNIFPYSASFTLNRSQPAHDNLMGTMWLNAGSEIQVAGFDYSCAINAPANIQQFEHIVPLRRVLTTATITLLPDAERFSFPRVINSADGATTWFFNPVILRPNNVEVEFLLNGQIINTYQARFGTIVARNFDTIRLSFQLMRPPNMTPAVAVLFPNAQPFEHHATVGLTLRIESAVCESVLADASETLLANVTSVRQEYAIPVGPVFPPGMNWTDLITNYSPSREDNLQRVFTVASIRSMLIK

SEQ ID NO: 11 - DNA, 1232 bp, Artificial Sequence
  Description of Artificial Sequence Synthetic polynucleotide
  pT7/VP7SA11
aagctttaatacgactcactataggctttaaaaagagagaatttccgtttggctagcggttagctccttttaatgtatggtattgaatataccacagttctaacctttctgatatcgattattctactaaattacatacttaaatcattaactagaataatggactttataatttatagatttctttttataattgtgatattgtcaccatttctcagagcacaaaattatggtattaatcttccaatcacaggctccatggacattgcatacgctaattcaacgcaagaagaaccattcctcacttctacactttgcctatattatccgactgaggctgcgactgaaataaacgataattcatggaaagacacactgtcacaactatttcttacgaaagggtggccaactggatccgtatattttaaagaatatactaacattgcatcgttttctgttgatccgcagttgtattgtgattataacgtagtactaatgaaatatgacgcgacgttgcaattggatatgtcagaacttgcggatctaatattaaacgaatggttgtgtaatccaatggatattactctgtattattatcagcaaactgacgaagcgaataaatggatatcaatgggctcatcatgtacaattaaagtatgtccacttaatacacaaactcttggaattggatgcttgacaactgatgctacaacttttgaagaagttgcgacagctgaaaagttggtaattactgacgtggttgatggcgttaatcataagctggatgtcacaacagcaacgtgtactattagaaactgtaagaaattgggaccaagagaaaacgtagccgttatacaagttggtggttctgacatcctcgatataactgctgatccaactactgcaccacagacagaacggatgatgcgaattaactggaaaaaatggtggcaagttttttatactgtagtagactatgtagatcagataatacaagttatgtccaaaagatcaagatcactaaattcagcagcattttattacagagtgtaggtataacttaggttagaattgtatgatgtgaccgggtcggcatggcatctccacctcctcgcggtccgacctgggcatccgaaggaggacgcacgtccactcggatggctaagggagagcctgcagtagcataaccccttggggcctctaaacgggtcttgaggggttttttgggtacc

SEQ ID NO: 12 - protein, 326 aa, Rotavirus A
MYGIEYTTVLTFLISIILLNYILKSLTRIMDFIIYRFLFIIVILSPFLRAQNYGINLPITGSMDIAYANSTQEEPFLTSTLCLYYPTEAATEINDNSWKDTLSQLFLTKGWPTGSVYFKEYTNIASFSVDPQLYCDYNVVLMKYDATLQLDMSELADLILNEWLCNPMDITLYYYQQTDEANKWISMGSSCTIKVCPLNTQTLGIGCLTTDATTFEEVATAEKLVITDVVDGVNHKLDVTTATCTIRNCKKLGPRENVAVIQVGGSDILDITADPTTAPQTERMMRINWKKWWQVFYTVVDYVDQIIQVMSKRSRSLNSAAFYYRV

SEQ ID NO: 13 - DNA, 1779 bp, Artificial Sequence
  Description of Artificial Sequence Synthetic polynucleotide
  pT7/NSP1SA11
actagttaatacgactcactataggcttttttttgaaaagtcttgtgttagccatggctacttttaaagatgcatgctttcattatcgtagattaactgctttaaatcggagattatgcaacattggtgcaaattctatttggatgccagttcctgatgcgaaaattaaggggtggtgtttagaatgttgtcaaatagctgatttaacccattgttatggttgctcattgccgcatgtttgcaaatggtgtgttcagaacagaagatgcttccttgacaatgaacctcatttgcttaagcttagaactgtgaaacatccaattaccaaagacaaattacagtgtatcatagacttgtacaatataatatttccaattaatgataaagtaattagaaaatttgaaagaatgataaagcaaagagaatgtaggaatcaatataaaattgaatggtataatcatttgctgctcccaattacattaaatgctgctgcatttaagtttgatgaaaataatctttattatgtttttgggttatatgagaaatcagtcagtgatatatatgctccatatagaattgttaactttataaatgaatttgataaattattgcttgatcatattaactttacaagaatgtccaatctaccaatagagttgagaaaccattacgcaaagaaatacttccaattatcaagactgccatcatcaaaactaaagcaaatttacttttcagattttactaaagaaactgtgatttttaatacttatacaaaaacgccaggaagatcaatatacagaaatgtaactgaatttaattggagagatgaattggagctttattctgatttaaaaaatgataagaataaattaattgctgcaatgatgacgagtaagtatactcggttctatgctcatgataataattttggaaggttgaaaatgacaatatttgagttgggacatcattgtcagcctaactacgtggcatctaatcacccaggcaatgcttccgatatccagtactgtaaatggtgtaatataaaatattttcttagtaaaattgattggcggattcgtgatatgtataatttattgatggaatttattaaggattgttataaaagtaatgttaacgttggacattgtagttctgttgaaaacatatatcctttaattaaaagattaatttggagtttgtttactaatcacatggatcaaacaattgaagaagtgtttaatcacatgtcgccagtgtcagttgaaggtacgaatgtcatcatgttgattcttggattgaatattagtttgtataatgaaattaagcgcactttgaatgtagatagcataccaatggtacttaatttaaatgaattcagtagtatagttaaatcaattagcagtaaatggtataatgttgatgaattggataaattgccaatgtcaataaaatcaacggaggaactgattgaaatgaagaattctggaactttaactgaagaatttgagctactgatctccaactcagaagatgacaatgagtgaaattatgtcactatctaattatacagtatttagccatcacaagaccgtccagactagagtagcgcctagctggcaaaatactgtgaaccgggtcggcatggcatctccacctcctcgcggtccgacctgggcatccgaaggaggacgcacgtccactcggatggctaagggagagcctgcagtagcataaccccttggggcctctaaacgggtcttgaggggttttttgggtacc

SEQ ID NO: 14 - protein, 496 aa, Rotavirus A
MATFKDACFHYRRLTALNRRLCNIGANSIWMPVPDAKIKGWCLECCQIADLTHCYGCSLPHVCKWCVQNRRCFLDNEPHLLKLRTVKHPITKDKLQCIIDLYNIIFPINDKVIRKFERMIKQRECRNQYKIEWYNHLLLPITLNAAAFKFDENNLYYVFGLYEKSVSDIYAPYRIVNFINEFDKLLLDHINFTRMSNLPIELRNHYAKKYFQLSRLPSSKLKQIYFSDFTKETVIFNTYTKTPGRSIYRNVTEFNWRDELELYSDLKNDKNKLIAAMMTSKYTRFYAHDNNFGRLKMTIFELGHHCQPNYVASNHPGNASDIQYCKWCNIKYFLSKIDWRIRDMYNLLMEFIKDCYKSNVNVGHCSSVENIYPLIKRLIWSLFTNHMDQTIEEVFNHMSPVSVEGTNVIMLILGLNISLYNEIKRTLNVDSIPMVLNLNEFSSIVKSISSKWYNVDELDKLPMSIKSTEELIEMKNSGTLTEEFELLISNSEDDNE

SEQ ID NO: 15 - DNA, 1228 bp, Artificial Sequence
  Description of Artificial Sequence Synthetic polynucleotide
  pT7/NSP2SA11
aagctttaatacgactcactataggcttttaaagcgtctcagtcgccgtttgagccttgcggtgtagccatggctgagctagcttgcttttgctatcctcatttggagaatgatagctataaatttattccttttaataatttagctattaaagctatgctgacagctaaagtagacaaaaaggacatggataagttttatgattcaattatttatggaatagcaccgcctcctcaatttaagaaacggtataatactaatgataattcaagaggcatgaattttgaaacaattatgtttactaaggtggctatgttgatatgtgaagctctaaattcattgaaagtgacgcaagcaaacgtctctaatgtattatcacgagtagtatcaataaggcatttagaaaatttggtgatacgtaaagaaaatccacaggatattctatttcattcaaaagatttacttttgaaatcaacactgattgctattggacagtctaaagaaattgaaactacaataactgcagaaggaggagaaattgtatttcaaaacgctgccttcaccatgtggaaactaacttatttagaacatcaattgatgccaattctggatcagaattttattgaatataaagttacattgaacgaagataaaccaatttcagatgttcatgttaaagaattagtcgctgaacttcgatggcaatataacaagtttgctgtaatcacacatggtaagggtcattatagaattgtaaagtattcatcagttgctaatcacgctgacagagtatatgcaactttcaagagtaatgttaaaactggagttaataatgattttaacctacttgatcaaagaattatttggcaaaactggtatgcatttacatcatcaatgaaacagggtaatacacttgacgtgtgtaaaaggttgcttttccaaaaaatgaaaccagaaaaaaatccatttaaagggctgtcaacggatagaaaaatggacgaagtttctcaagttggcgtttaattcgctatcaatttgaggatgatgatggcttagcaagaatagaaagcgcttatgtgaccgggtcggcatggcatctccacctcctcgcggtccgacctgggcatccgaaggaggacgcacgtccactcggatggctaagggagagcctgcagtagcataaccccttggggcctctaaacgggtcttgaggggttttttgggtacc

SEQ ID NO: 16 - protein, 317 aa, Rotavirus A
MAELACFCYPHLENDSYKFIPFNNLAIKAMLTAKVDKKDMDKFYDSIIYGIAPPPQFKKRYNTNDNSRGMNFETIMFTKVAMLICEALNSLKVTQANVSNVLSRVVSIRHLENLVIRKENPQDILFHSKDLLLKSTLIAIGQSKEIETTITAEGGEIVFQNAAFTMWKLTYLEHQLMPILDQNFIEYKVTLNEDKPISDVHVKELVAELRWQYNKFAVITHGKGHYRIVKYSSVANHADRVYATFKSNVKTGVNNDFNLLDQRIIWQNWYAFTSSMKQGNTLDVCKRLLFQKMKPEKNPFKGLSTDRKMDEVSQVGV

SEQ ID NO: 17 - DNA, 1274 bp, Artificial Sequence
  Description of Artificial Sequence Synthetic polynucleotide
  pT7/NSP3SA11
actagttaatacgactcactataggcatttaatgcttttcagtggttgatgctcaagatggagtctacgcaacagatggccgtctcaattattaactcttcttttgaagctgcagttgtagctgcaacctcagctcttgagaatatgggaatagaatatgattatcaggatatatattctagagtaaagaataaatttgattttgtgatggacgattctggtgttaaaaataatctgattggtaaagcaataactattgatcaagctttgaataataaatttggatctgctataagaaatagaaactggcttgctgatacttctagagcagctaaattagatgaggatgtaaacaaactaagaatgatgttatcatcaaaaggaattgatcaaaaaatgagagttttaaacgcatgcttcagtgtaaaaagaatacctggaaaatcatcatctattattaaatgcacaaaattgatgcgtgataaattggaacgtggtgaagttgaagtggatgattcatttgtggatgaaaaaatggaagtggataccattgactggaaatcgcgctatgagcaattggagcaaaggtttgaatcattgaaatccagggtaaatgaaaaatataataattgggtgttgaaagcaagaaaaatgaatgaaaatatgcattctcttcaaaatgtcatctctcaacagcaagcacatatagctgagcttcaagtgtacaataataaactagaacgtgatttgcaaaataaaattggatcccttacttcttcgattgaatggtatttaagatcaatggaattagaccctgaaataaaggcagacattgaacagcaaattaactcaattgatgcgataaatccattgcacgcttttgatgacttagaatcagtaatacgtaatttgatatctgattatgacaaattattccttatgttcaaaggattaatacagagatgtaattatcaatattcatttggttgcgaataaccattttgatacatgttgaacaatcaaatacagtgttagtatgttgtcatctatgcataaccctctatgagcacaatagttaaaagctaacactgtcaaaaacctaaatggctataggggcgttatgtggccgggtcggcatggcatctccacctcctcgcggtccgacctgggcatccgaaggaggacgcacgtccactcggatggctaagggagagcctgcagtagcataaccccttggggcctctaaacgggtcttgaggggttttttgggtacc

SEQ ID NO: 18 - protein, 315 aa, Rotavirus A
MLKMESTQQMAVSIINSSFEAAVVAATSALENMGIEYDYQDIYSRVKNKFDFVMDDSGVKNNLIGKAITIDQALNNKFGSAIRNRNWLADTSRAAKLDEDVNKLRMMLSSKGIDQKMRVLNACFSVKRIPGKSSSIIKCTKLMRDKLERGEVEVDDSFVDEKMEVDTIDWKSRYEQLEQRFESLKSRVNEKYNNWVLKARKMNENMHSLQNVISQQQAHIAELQVYNNKLERDLQNKIGSLTSSIEWYLRSMELDPEIKADIEQQINSIDAINPLHAFDDLESVIRNLISDYDKLFLMFKGLIQRCNYQYSFGCE

SEQ ID NO: 19 - DNA, 920 bp, Artificial Sequence
  Description of Artificial Sequence Synthetic polynucleotide
  pT7/NSP4SA11
actagttaatacgactcactataggcttttaaaagttctgttccgagagagcgcgtgcggaaagatggaaaagcttaccgacctcaattatacattgagtgtaatcactctaatgaacaatacattgcacacaatacttgaggatccaggaatggcgtattttccttatatagcatctgtcttaacagttttgtttgcgctacataaagcatccattccaacaatgaaaattgcattgaaaacgtcaaaatgttcatataaagtggtgaaatattgtattgtaacaatttttaatacgttgttaaaattggcaggttataaagagcagataactactaaagatgagatagaaaagcaaatggacagagtagtcaaagaaatgagacgccagctagaaatgattgacaaattgactacacgtgaaattgaacaagtagagttgcttaaacgcatttacgataaattgacggtgcaaacgacaggtgaaatagatatgacaaaagagatcaatcaaaaaaacgtgagaacgctagaagaatgggaaagtggaaaaaatccttatgaaccaagagaagtgactgcagcaatgtaagaggttgagctgccgtcgactgtcctcggaagcggcggagttctttacagtaagcaccatcggacctgatggctgactgagaagccacagtcagccatatcgcgtgtggctcaagccttaatcccgtttaaccaatccggtcagcaccggacgttaatggaaggaacggtcttaatgtgaccgggtcggcatggcatctccacctcctcgcggtccgacctgggcatccgaaggaggacgcacgtccactcggatggctaagggagagcctgcagtagcataaccccttggggcctctaaacgggtcttgaggggttttttgggtacc

SEQ ID NO: 20 - protein, 175 aa, Rotavirus A
MEKLTDLNYTLSVITLMNNTLHTILEDPGMAYFPYIASVLTVLFALHKASIPTMKIALKTSKCSYKVVKYCIVTIFNTLLKLAGYKEQITTKDEIEKQMDRVVKEMRRQLEMIDKLTTREIEQVELLKRIYDKLTVQTTGEIDMTKEINQKNVRTLEEWESGKNPYEPREVTAAM

SEQ ID NO: 21 - DNA, 836 bp, Artificial Sequence
  Description of Artificial Sequence Synthetic polynucleotide
  pT7/NSP5SA11
aagctttaatacgactcactataggcttttaaagcgctacagtgatgtctctcagtattgacgtgacgagtcttccttctattccttcaactatatataagaatgaatcgtcttcaacaacgtcaactctttctggaaaatctattggtaggagtgaacagtacatttcaccagatgcagaagcattcaataaatacatgctgtcgaagtctccagaggatattggaccatctgattctgcttcaaacgatccactcaccagtttttcgattagatcgaatgcagttaagacaaacgcagacgctggcgtgtctatggattcatcagcacaatcacgaccttcaagtaatgtcggatgcgatcaagtggatttctccttaaataaaggcttaaaagtaaaagctaatttggactcatcaatatcaatatctacggatactaaaaaggagaaatcaaaacaaaaccataaaagtaggaagcactacccaagaattgaagcagagtctgattcagatgattatgtactggatgattcagatagtgatgatggtaaatgtaagaactgtaaatataagaagaaatacttcgcattaagaatgagaatgaaacaagtcgcaatgcaattgattgaagatttgtaagtctgacctgggaacacactagggagctccccactcccgttttgtgaccgggtcggcatggcatctccacctcctcgcggtccgacctgggcatccgaaggaggacgcacgtccactcggatggctaagggagagcctgcagtagcataaccccttggggcctctaaacgggtcttgaggggttttttgggtacc

SEQ ID NO: 22 - protein, 198 aa, Rotavirus A
MSLSIDVTSLPSIPSTIYKNESSSTTSTLSGKSIGRSEQYISPDAEAFNKYMLSKSPEDIGPSDSASNDPLTSFSIRSNAVKTNADAGVSMDSSAQSRPSSNVGCDQVDFSLNKGLKVKANLDSSISISTDTKKEKSKQNHKSRKHYPRIEAESDSDDYVLDDSDSDDGKCKNCKYKKKYFALRMRMKQVAMQLIEDL

SEQ ID NO: 23 - DNA, 4867 bp, Artificial Sequence
  Description of Artificial Sequence Synthetic polynucleotide
  pCMV vector (V1Jns)
agatctgctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggccgctgcggccaggtgctgaagaattgacccggttcctcctgggccagaaagaagcaggcacatccccttctctgtgacacaccctgtccacgcccctggttcttagttccagccccactcataggacactcatagctcaggagggctccgccttcaatcccacccgctaaagtacttggagcggtctctccctccctcatcagcccaccaaaccaaacctagcctccaagagtgggaagaaattaaagcaagataggctattaagtgcagagggagagaaaatgcctccaacatgtgaggaagtaatgagagaaatcatagaatttcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactcggggggggggggcgctgaggtctgcctcgtgaagaaggtgttgctgactcataccaggcctgaatcgccccatcatccagccagaaagtgagggagccacggttgatgagagctttgttgtaggtggaccagttggtgattttgaacttttgctttgccacggaacggtctgcgttgtcgggaagatgcgtgatctgatccttcaactcagcaaaagttcgatttattcaacaaagccgccgtcccgtcaagtcagcgtaatgctctgccagtgttacaaccaattaaccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctcgagcaagacgtttcccgttgaatatggctcataacaccccttgtattactgtttatgtaagcagacagttttattgttcatgatgatatatttttatcttgtgcaatgtaacatcagagattttgagacacaacgtggctttccccccccccccattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcagattggctattggccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccgcggccgggaacggtgcattggaacgcggattccccgtgccaagagtgacgtaagtaccgcctatagactctataggcacacccctttggctcttatgcatgctatactgtttttggcttggggcctatacacccccgcttccttatgctataggtgatggtatagcttagcctataggtgtgggttattgaccattattgaccactcccctattggtgacgatactttccattactaatccataacatggctctttgccacaactatctctattggctatatgccaatactctgtccttcagagactgacacggactctgtatttttacaggatggggtcccatttattatttacaaattcacatatacaacaacgccgtcccccgtgcccgcagtttttattaaacatagcgtgggatctccacgcgaatctcgggtacgtgttccggacatgggctcttctccggtagcggcggagcttccacatccgagccctggtcccatgcctccagcggctcatggtcgctcggcagctccttgctcctaacagtggaggccagacttaggcacagcacaatgcccaccaccaccagtgtgccgcacaaggccgtggcggtagggtatgtgtctgaaaatgagcgtggagattgggctcgcacggctgacgcagatggaagacttaaggcagcggcagaagaagatgcaggcagctgagttgttgtattctgataagagtcagaggtaactcccgttgcggtgctgttaacggtggagggcagtgtagtctgagcagtactcgttgctgccgcgcgcgccaccagacataatagctgacagactaacagactgttcctttccatgggtcttttctgcagtcaccgtcctt

SEQ ID NO: 24 - DNA, 972 bp, Artificial Sequence
  Description of Artificial Sequence Synthetic polynucleotide
  CMV/NSP2
agatctgccaccatggctgagctagcttgcttttgctatcctcatttggagaatgatagctataaatttattccttttaataatttagctattaaagctatgctgacagctaaagtagacaaaaaggacatggataagttttatgattcaattatttatggaatagcaccgcctcctcaatttaagaaacggtataatactaatgataattcaagaggcatgaattttgaaacaattatgtttactaaggtggctatgttgatatgtgaagctctaaattcattgaaagtgacgcaagcaaacgtctctaatgtattatcacgagtagtatcaataaggcatttagaaaatttggtgatacgtaaagaaaatccacaggatattctatttcattcaaaagatttacttttgaaatcaacactgattgctattggacagtctaaagaaattgaaactacaataactgcagaaggaggagaaattgtatttcaaaacgctgccttcaccatgtggaaactaacttatttagaacatcaattgatgccaattctggatcagaattttattgaatataaagttacattgaacgaagataaaccaatttcagatgttcatgttaaagaattagtcgctgaacttcgatggcaatataacaagtttgctgtaatcacacatggtaagggtcattatagaattgtaaagtattcatcagttgctaatcacgctgacagagtatatgcaactttcaagagtaatgttaaaactggagttaataatgattttaacctacttgatcaaagaattatttggcaaaactggtatgcatttacatcatcaatgaaacagggtaatacacttgacgtgtgtaaaaggttgcttttccaaaaaatgaaaccagaaaaaaatccatttaaagggctgtcaacggatagaaaaatggacgaagtttctcaagttggcgtttaaagatct

SEQ ID NO: 25 - DNA, 615 bp, Artificial Sequence
  Description of Artificial Sequence Synthetic polynucleotide
  CMV/NSP5
agatctgccaccatgtctctcagtattgacgtgacgagtcttccttctattccttcaactatatataagaatgaatcgtcttcaacaacgtcaactctttctggaaaatctattggtaggagtgaacagtacatttcaccagatgcagaagcattcaataaatacatgctgtcgaagtctccagaggatattggaccatctgattctgcttcaaacgatccactcaccagtttttcgattagatcgaatgcagttaagacaaacgcagacgctggcgtgtctatggattcatcagcacaatcacgaccttcaagtaatgtcggatgcgatcaagtggatttctccttaaataaaggcttaaaagtaaaagctaatttggactcatcaatatcaatatctacggatactaaaaaggagaaatcaaaacaaaaccataaaagtaggaagcactacccaagaattgaagcagagtctgattcagatgattatgtactggatgattcagatagtgatgatggtaaatgtaagaactgtaaatataagaagaaatacttcgcattaagaatgagaatgaaacaagtcgcaatgcaattgattgaagatttgtaaagatct

SEQ ID NO: 26 - DNA, 306 bp, Artificial Sequence
  Description of Artificial Sequence Synthetic polynucleotide
  CMV/ NBVFAST
agatctgccaccatgagcggagattgtgccggcctggtgtctgtgtttggcagcgtgcactgtcagagcagcaagaacaaagctggcggcgatctgcaggccaccagcgtgttgacaacatactggcctcacctggccatcggcggcagcatcatcctgatcattctgctgctgggcctgttctactgctgctacctgaagtggaaaaccagccacatccggcggacctaccacaaagaactggtggccctgaccagaggctacgtgcgacctattcctgccgatgtgacctccgtgtgaagatct

SEQ ID NO: 27 - protein, 95 aa, Unknown
  Description of Unknown Kampar orthoreovirus sequence
MSGDCAGLVSVFGSVHCQSSKNKAGGDLQATSVLTTYWPHLAIGGSIILIILLLGLFYCCYLKWKTSHIRRTYHKELVALTRGYVRPIPADVTSV

SEQ ID NO: 28 - DNA, 882 bp, Artificial Sequence
  Description of Artificial Sequence Synthetic polynucleotide
  CMV/D12L
agatctgccaccatggacgagatcgtgaagaacatccgcgagggcacacacgtgctgctgcctttctatgagacactgcccgagctgaacctgagcctgggaaagtctcctctgcctagcctggaatacggcgccaactacttcctgcagatcagcagagtgaacgacctgaacagaatgcccaccgacatgctgaagctgttcacccacgacatcatgctgcccgagagcgacctggacaaggtgtacgagattctgaagatcaacagcgtgaagtactacggcagaagcaccaaggccgacgccgttgtggctgatctgagcgccagaaacaagctgtttaagagagagcgggacgccatcaagagcaacaaccacctgaccgagaacaacctgtacatcagcgactacaagatgctgaccttcgacgtgttcagacccctgttcgacttcgtgaacgagaagtactgcatcatcaagctgcccacactgttcggcagaggcgtgatcgacaccatgcggatctactgcagcctgttcaagaacgtgcggctgctgaagtgcgtgtccgacagctggctgaaggacagcgccattatggtggccagcgacgtgtgcaagaagaacctggacctgttcatgagccacgtgaagtccgtgaccaagagcagcagctggaaggacgtgaacagcgtgcagttcagcatcctgaacaaccccgtggacaccgagttcatcaacaagttcctcgagttcagcaaccgcgtgtacgaggccctgtactacgtgcacagcctgctgtactccagcatgaccagcgacagcaagagcatcgagaacaagcaccagcggcggctggtcaaactgctgctgtaaagatct

SEQ ID NO: 29 - protein, 287 aa, Vaccinia virus
MDEIVKNIREGTHVLLPFYETLPELNLSLGKSPLPSLEYGANYFLQISRVNDLNRMPTDMLKLFTHDIMLPESDLDKVYEILKINSVKYYGRSTKADAVVADLSARNKLFKRERDAIKSNNHLTENNLYISDYKMLTFDVFRPLFDFVNEKYCIIKLPTLFGRGVIDTMRIYCSLFKNVRLLKCVSDSWLKDSAIMVASDVCKKNLDLFMSHVKSVTKSSSWKDVNSVQFSILNNPVDTEFINKFLEFSNRVYEALYYVHSLLYSSMTSDSKSIENKHQRRLVKLLL

SEQ ID NO: 30 - DNA, 2553 bp, Artificial Sequence
  Description of Artificial Sequence Synthetic polynucleotide
  CMV/D1R
agatctgccaccatggacgccaatgtggtgtccagctctacaatcgccacctacatcgacgccctggccaagaatgccagcgagctggaacagagaagcaccgcctacgagatcaacaacgaactggaactggtgttcatcaagcctccactgatcaccctgaccaacgtggtcaacatcagcaccatccaagagagcttcatccggtttaccgtgaccaacaaagaaggcgtgaagatccggaccaagattcccctgtctaaggtgcacggcctggacgtgaagaacgtgcagctggtggacgccatcgacaacatcgtgtgggagaagaagtccctggtcaccgagaaccggctgcacaaagagtgcctgctgagactgagcaccgaggaacggcacatctttctggactacaagaagtacggcagcagcatccggctggaactcgtgaatctgatccaggccaagaccaagaacttcaccatcgacttcaagctcaagtacttcctcggcagcggagcccagagcaagtctagtctgctgcacgccatcaatcaccccaagagcagacccaacaccagcctggaaatcgagttcacccctcgggacaacgagacagtgccctacgacgagctgatcaaagagctgaccacactgagccgccacatcttcatggctagccccgagaatgtgatcctgtctcctcctatcaacgcccctatcaagaccttcatgctgcccaagcaggacatcgtgggcctcgacctggaaaacctgtacgccgtgacaaagaccgacggcatccccatcaccatcagagtgaccagcaacggcctgtactgctacttcacccacctgggctacatcatcagataccccgtgaagcggatcatcgacagcgaggtggtggtgtttggagaggccgtgaaggacaagaactggaccgtgtacctgatcaagctgatcgagcccgtgaatgccatcaacgacagactggaagagagcaaatacgtcgagagcaaactggtggacatctgcgaccggatcgtgttcaagagcaagaagtatgagggcccctttaccaccaccagtgaagtggtggatatgctgagcacctacctgcctaagcagcccgagggcgtcatcctgttttacagcaagggccccaagtccaacatcgatttcaagattaagaaagagaacaccatcgatcagaccgccaacgtcgtgttccggtacatgagcagcgagcccatcatcttcggcgagagcagcatcttcgtcgagtataagaagttcagcaacgacaagggcttccccaaagagtacggctccggcaagatcgtgctgtacaacggcgtgaactacctgaacaacatctactgcctcgagtacatcaacacccacaacgaagtgggcatcaagagcgtggtggtgcccatcaagtttatcgccgagttcctggtcaacggcgagatcctgaagcctcggatcgacaagaccatgaagtatatcaacagcgaggactactacggcaaccagcacaacatcatcgtggaacacctgagggaccagagcatcaagatcggcgacatcttcaacgaggacaagctgagcgacgtgggccaccagtacgccaacaacgataagttccggctgaaccccgaggtgtcctactttaccaacaagcggaccagaggacccctgggcatcctgagcaactacgtgaaaaccctgctgatctccatgtactgcagcaagacattcctggacgacagcaacaagagaaaggtgctggccattgacttcggcaacggcgccgatctggaaaagtacttctatggcgagatcgccctgctggtggccacagatcctgatgccgatgccattgccagaggcaacgagcggtacaacaagctgaacagcggcattaagaccaagtactacaagttcgactacatccaagaaaccattcggagcgacaccttcgtgtccagcgtgcgcgaggtgttctatttcggcaagttcaatatcatcgactggcagttcgccatccactacagctttcaccccagacactacgccaccgtgatgaacaacctgagcgagctgacagcctctggcggcaaggtgctgatcaccacaatggacggcgacaagctgtccaagctgaccgacaagaaaaccttcatcatccacaagaatctgcccagcagcgagaactacatgagcgtggaaaagatcgccgacgacagaatcgtggtgtataaccccagcaccatgagcacccctatgaccgagtatatcatcaagaagaacgacatcgtccgggtgttcaacgagtatggcttcgtgctggtggataacgtggacttcgccaccatcatcgagcggtccaagaagtttatcaacggggccagcacaatggaagatcggccctccaccagaaactttttcgagctgaatagaggcgccatcaagtgcgaaggcctggatgtcgaggacctgctgtcctactacgtggtgtacgtgttcagcaagcgctgaagatct

SEQ ID NO: 31 - protein, 844 aa, Vaccinia virus
MDANVVSSSTIATYIDALAKNASELEQRSTAYEINNELELVFIKPPLITLTNVVNISTIQESFIRFTVTNKEGVKIRTKIPLSKVHGLDVKNVQLVDAIDNIVWEKKSLVTENRLHKECLLRLSTEERHIFLDYKKYGSSIRLELVNLIQAKTKNFTIDFKLKYFLGSGAQSKSSLLHAINHPKSRPNTSLEIEFTPRDNETVPYDELIKELTTLSRHIFMASPENVILSPPINAPIKTFMLPKQDIVGLDLENLYAVTKTDGIPITIRVTSNGLYCYFTHLGYIIRYPVKRIIDSEVVVFGEAVKDKNWTVYLIKLIEPVNAINDRLEESKYVESKLVDICDRIVFKSKKYEGPFTTTSEVVDMLSTYLPKQPEGVILFYSKGPKSNIDFKIKKENTIDQTANVVFRYMSSEPIIFGESSIFVEYKKFSNDKGFPKEYGSGKIVLYNGVNYLNNIYCLEYINTHNEVGIKSVVVPIKFIAEFLVNGEILKPRIDKTMKYINSEDYYGNQHNIIVEHLRDQSIKIGDIFNEDKLSDVGHQYANNDKFRLNPEVSYFTNKRTRGPLGILSNYVKTLLISMYCSKTFLDDSNKRKVLAIDFGNGADLEKYFYGEIALLVATDPDADAIARGNERYNKLNSGIKTKYYKFDYIQETIRSDTFVSSVREVFYFGKFNIIDWQFAIHYSFHPRHYATVMNNLSELTASGGKVLITTMDGDKLSKLTDKKTFIIHKNLPSSENYMSVEKIADDRIVVYNPSTMSTPMTEYIIKKNDIVRVFNEYGFVLVDNVDFATIIERSKKFINGASTMEDRPSTRNFFELNRGAIKCEGLDVEDLLSYYVVYVFSKR

SEQ ID NO: 32 - protein, 21 aa, Artificial Sequence
  Description of Artificial Sequence Synthetic peptide
  T2A peptide
GSGEGRGSLLTCGDVEENPGP

SEQ ID NO: 33 - protein, 22 aa, Artificial Sequence
  Description of Artificial Sequence Synthetic peptide
  P2A peptide
GSGATNFSLLKQAGDVEENPGP

SEQ ID NO: 34 - protein, 23 aa, Artificial Sequence
  Description of Artificial Sequence Synthetic peptide
  E2A peptide
GSGQCTNYALLKLAGDVESNPGP

SEQ ID NO: 35 - protein, 25 aa, Artificial Sequence
  Description of Artificial Sequence Synthetic peptide
  F2A peptide
GSGVKQTLNFDLLKLAGDVESNPGP

SEQ ID NO: 36 - protein, 681 aa, Severe acute respiratory syndrome coronavirus 2
  S1 domain of SARS-CoV-2 Spike protein
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSP

SEQ ID NO: 37 - protein, 288 aa, Severe acute respiratory syndrome coronavirus 2
  RBD of SARS-CoV-2 Spike protein
MFVFLVLLPLVSSQCRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCS

SEQ ID NO: 38 - RNA, 29903 nt, Severe acute respiratory syndrome coronavirus 2
auuaaagguuuauaccuucccagguaacaaaccaaccaacuuucgaucucuuguagaucuguucucuaaacgaacuuuaaaaucuguguggcugucacucggcugcaugcuuagugcacucacgcaguauaauuaauaacuaauuacugucguugacaggacacgaguaacucgucuaucuucugcaggcugcuuacgguuucguccguguugcagccgaucaucagcacaucuagguuucguccgggugugaccgaaagguaagauggagagccuugucccugguuucaacgagaaaacacacguccaacucaguuugccuguuuuacagguucgcgacgugcucguacguggcuuuggagacuccguggaggaggucuuaucagaggcacgucaacaucuuaaagauggcacuuguggcuuaguagaaguugaaaaaggcguuuugccucaacuugaacagcccuauguguucaucaaacguucggaugcucgaacugcaccucauggucauguuaugguugagcugguagcagaacucgaaggcauucaguacggucguaguggugagacacuugguguccuugucccucaugugggcgaaauaccaguggcuuaccgcaagguucuucuucguaagaacgguaauaaaggagcugguggccauaguuacggcgccgaucuaaagucauuugacuuaggcgacgagcuuggcacugauccuuaugaagauuuucaagaaaacuggaacacuaaacauagcagugguguuacccgugaacucaugcgugagcuuaacggaggggcauacacucgcuaugucgauaacaacuucuguggcccugauggcuacccucuugagugcauuaaagaccuucuagcacgugcugguaaagcuucaugcacuuuguccgaacaacuggacuuuauugacacuaagagggguguauacugcugccgugaacaugagcaugaaauugcuugguacacggaacguucugaaaagagcuaugaauugcagacaccuuuugaaauuaaauuggcaaagaaauuugacaccuucaauggggaauguccaaauuuuguauuucccuuaaauuccauaaucaagacuauucaaccaaggguugaaaagaaaaagcuugauggcuuuauggguagaauucgaucugucuauccaguugcgucaccaaaugaaugcaaccaaaugugccuuucaacucucaugaagugugaucauuguggugaaacuucauggcagacgggcgauuuuguuaaagccacuugcgaauuuuguggcacugagaauuugacuaaagaaggugccacuacuugugguuacuuaccccaaaaugcuguuguuaaaauuuauuguccagcaugucacaauucagaaguaggaccugagcauagucuugccgaauaccauaaugaaucuggcuugaaaaccauucuucguaaggguggucgcacuauugccuuuggaggcuguguguucucuuauguugguugccauaacaagugugccuauuggguuccacgugcuagcgcuaacauagguuguaaccauacagguguuguuggagaagguuccgaaggucuuaaugacaaccuucuugaaauacuccaaaaagagaaagucaacaucaauauuguuggugacuuuaaacuuaaugaagagaucgccauuauuuuggcaucuuuuucugcuuccacaagugcuuuuguggaaacugugaaagguuuggauuauaaagcauucaaacaaauuguugaauccugugguaauuuuaaaguuacaaaaggaaaagcuaaaaaaggugccuggaauauuggugaacagaaaucaauacugaguccucuuuaugcauuugcaucagaggcugcucguguuguacgaucaauuuucucccgcacucuugaaacugcucaaaauucugugcguguuuuacagaaggccgcuauaacaauacuagauggaauuucacaguauucacugagacucauugaugcuaugauguucacaucugauuuggcuacuaacaaucuaguuguaauggccuacauuacaggugguguuguucaguugacuucgcaguggcuaacuaacaucuuuggcacuguuuaugaaaaacucaaacccguccuugauuggcuugaagagaaguuuaaggaagguguagaguuucuuagagacgguugggaaauuguuaaauuuaucucaaccugugcuugugaaauugucgguggacaaauugucaccugugcaaaggaaauuaaggagaguguucagacauucuuuaagcuuguaaauaaauuuuuggcuuugugugcugacucuaucauuauugguggagcuaaacuuaaagccuugaauuuaggugaaacauuugucacgcacucaaagggauuguacagaaaguguguuaaauccagagaagaaacuggccuacucaugccucuaaaagccccaaaagaaauuaucuucuuagagggagaaacacuucccacagaaguguuaacagaggaaguugucuugaaaacuggugauuuacaaccauuagaacaaccuacuagugaagcuguugaagcuccauugguugguacaccaguuuguauuaacgggcuuauguugcucgaaaucaaagacacagaaaaguacugugcccuugcaccuaauaugaugguaacaaacaauaccuucacacucaaaggcggugcaccaacaaagguuacuuuuggugaugacacugugauagaagugcaagguuacaagagugugaauaucacuuuugaacuugaugaaaggauugauaaaguacuuaaugagaagugcucugccuauacaguugaacucgguacagaaguaaaugaguucgccuguguuguggcagaugcugucauaaaaacuuugcaaccaguaucugaauuacuuacaccacugggcauugauuuagaugaguggaguauggcuacauacuacuuauuugaugagucuggugaguuuaaauuggcuucacauauguauuguucuuucuacccuccagaugaggaugaagaagaaggugauugugaagaagaagaguuugagccaucaacucaauaugaguaugguacugaagaugauuaccaagguaaaccuuuggaauuuggugccacuucugcugcucuucaaccugaagaagagcaagaagaagauugguuagaugaugauagucaacaaacuguuggucaacaagacggcagugaggacaaucagacaacuacuauucaaacaauuguugagguucaaccucaauuagagauggaacuuacaccaguuguucagacuauugaagugaauaguuuuagugguuauuuaaaacuuacugacaauguauacauuaaaaaugcagacauuguggaagaagcuaaaaagguaaaaccaacagugguuguuaaugcagccaauguuuaccuuaaacauggaggagguguugcaggagccuuaaauaaggcuacuaacaaugccaugcaaguugaaucugaugauuacauagcuacuaauggaccacuuaaagugggugguaguuguguuuuaagcggacacaaucuugcuaaacacugucuucauguugucggcccaaauguuaacaaaggugaagacauucaacuucuuaagagugcuuaugaaaauuuuaaucagcacgaaguucuacuugcaccauuauuaucagcugguauuuuuggugcugacccuauacauucuuuaagaguuuguguagauacuguucgcacaaaugucuacuuagcugucuuugauaaaaaucucuaugacaaacuuguuucaagcuuuuuggaaaugaagagugaaaagcaaguugaacaaaagaucgcugagauuccuaaagaggaaguuaagccauuuauaacugaaaguaaaccuucaguugaacagagaaaacaagaugauaagaaaaucaaagcuuguguugaagaaguuacaacaacucuggaagaaacuaaguuccucacagaaaacuuguuacuuuauauugacauuaauggcaaucuucauccagauucugccacucuuguuagugacauugacaucacuuucuuaaagaaagaugcuccauauauagugggugauguuguucaagaggguguuuuaacugcugugguuauaccuacuaaaaaggcugguggcacuacugaaaugcuagcgaaagcuuugagaaaagugccaacagacaauuauauaaccacuuacccgggucaggguuuaaaugguuacacuguagaggaggcaaagacagugcuuaaaaaguguaaaagugccuuuuacauucuaccaucuauuaucucuaaugagaagcaagaaauucuuggaacuguuucuuggaauuugcgagaaaugcuugcacaugcagaagaaacacgcaaauuaaugccugucuguguggaaacuaaagccauaguuucaacuauacagcguaaauauaaggguauuaaaauacaagagggugugguugauuauggugcuagauuuuacuuuuacaccaguaaaacaacuguagcgucacuuaucaacacacuuaacgaucuaaaugaaacucuuguuacaaugccacuuggcuauguaacacauggcuuaaauuuggaagaagcugcucgguauaugagaucucucaaagugccagcuacaguuucuguuucuucaccugaugcuguuacagcguauaaugguuaucuuacuucuucuucuaaaacaccugaagaacauuuuauugaaaccaucucacuugcugguuccuauaaagauugguccuauucuggacaaucuacacaacuagguauagaauuucuuaagagaggugauaaaaguguauauuacacuaguaauccuaccacauuccaccuagauggugaaguuaucaccuuugacaaucuuaagacacuucuuucuuugagagaagugaggacuauuaagguguuuacaacaguagacaacauuaaccuccacacgcaaguuguggacaugucaaugacauauggacaacaguuugguccaacuuauuuggauggagcugauguuacuaaaauaaaaccucauaauucacaugaagguaaaacauuuuauguuuuaccuaaugaugacacucuacguguugaggcuuuugaguacuaccacacaacugauccuaguuuucuggguagguacaugucagcauuaaaucacacuaaaaaguggaaauacccacaaguuaaugguuuaacuucuauuaaaugggcagauaacaacuguuaucuugccacugcauuguuaacacuccaacaaauagaguugaaguuuaauccaccugcucuacaagaugcuuauuacagagcaagggcuggugaagcugcuaacuuuugugcacuuaucuuagccuacuguaauaagacaguaggugaguuaggugauguuagagaaacaaugaguuacuuguuucaacaugccaauuuagauucuugcaaaagagucuugaacgugguguguaaaacuuguggacaacagcagacaacccuuaaggguguagaagcuguuauguacaugggcacacuuucuuaugaacaauuuaagaaagguguucagauaccuuguacgugugguaaacaagcuacaaaauaucuaguacaacaggagucaccuuuuguuaugaugucagcaccaccugcucaguaugaacuuaagcaugguacauuuacuugugcuagugaguacacugguaauuaccaguguggucacuauaaacauauaacuucuaaagaaacuuuguauugcauagacggugcuuuacuuacaaaguccucagaauacaaagguccuauuacggauguuuucuacaaagaaaacaguuacacaacaaccauaaaaccaguuacuuauaaauuggaugguguuguuuguacagaaauugacccuaaguuggacaauuauuauaagaaagacaauucuuauuucacagagcaaccaauugaucuuguaccaaaccaaccauauccaaacgcaagcuucgauaauuuuaaguuuguaugugauaauaucaaauuugcugaugauuuaaaccaguuaacugguuauaagaaaccugcuucaagagagcuuaaaguuacauuuuucccugacuuaaauggugaugugguggcuauugauuauaaacacuacacacccucuuuuaagaaaggagcuaaauuguuacauaaaccuauuguuuggcauguuaacaaugcaacuaauaaagccacguauaaaccaaauaccugguguauacguugucuuuggagcacaaaaccaguugaaacaucaaauucguuugauguacugaagucagaggacgcgcagggaauggauaaucuugccugcgaagaucuaaaaccagucucugaagaaguaguggaaaauccuaccauacagaaagacguucuugaguguaaugugaaaacuaccgaaguuguaggagacauuauacuuaaaccagcaaauaauaguuuaaaaauuacagaagagguuggccacacagaucuaauggcugcuuauguagacaauucuagucuuacuauuaagaaaccuaaugaauuaucuagaguauuagguuugaaaacccuugcuacucaugguuuagcugcuguuaauagugucccuugggauacuauagcuaauuaugcuaagccuuuucuuaacaaaguuguuaguacaacuacuaacauaguuacacgguguuuaaaccguguuuguacuaauuauaugccuuauuucuuuacuuuauugcuacaauuguguacuuuuacuagaaguacaaauucuagaauuaaagcaucuaugccgacuacuauagcaaagaauacuguuaagagugucgguaaauuuugucuagaggcuucauuuaauuauuugaagucaccuaauuuuucuaaacugauaaauauuauaauuugguuuuuacuauuaaguguuugccuagguucuuuaaucuacucaaccgcugcuuuagguguuuuaaugucuaauuuaggcaugccuucuuacuguacugguuacagagaaggcuauuugaacucuacuaaugucacuauugcaaccuacuguacugguucuauaccuuguaguguuugucuuagugguuuagauucuuuagacaccuauccuucuuuagaaacuauacaaauuaccauuucaucuuuuaaaugggauuuaacugcuuuuggcuuaguugcagagugguuuuuggcauauauucuuuucacuagguuuuucuauguacuuggauuggcugcaaucaugcaauuguuuuucagcuauuuugcaguacauuuuauuaguaauucuuggcuuaugugguuaauaauuaaucuuguacaaauggccccgauuucagcuaugguuagaauguacaucuucuuugcaucauuuuauuauguauggaaaaguuaugugcauguuguagacgguuguaauucaucaacuuguaugauguguuacaaacguaauagagcaacaagagucgaauguacaacuauuguuaaugguguuagaagguccuuuuaugucuaugcuaauggagguaaaggcuuuugcaaacuacacaauuggaauuguguuaauugugauacauucugugcugguaguacauuuauuagugaugaaguugcgagagacuugucacuacaguuuaaaagaccaauaaauccuacugaccagucuucuuacaucguugauaguguuacagugaagaaugguuccauccaucuuuacuuugauaaagcuggucaaaagacuuaugaaagacauucucucucucauuuuguuaacuuagacaaccugagagcuaauaacacuaaagguucauugccuauuaauguuauaguuuuugaugguaaaucaaaaugugaagaaucaucugcaaaaucagcgucuguuuacuacagucagcuuaugugucaaccuauacuguuacuagaucaggcauuagugucugauguuggugauagugcggaaguugcaguuaaaauguuugaugcuuacguuaauacguuuucaucaacuuuuaacguaccaauggaaaaacucaaaacacuaguugcaacugcagaagcugaacuugcaaagaauguguccuuagacaaugucuuaucuacuuuuauuucagcagcucggcaaggguuuguugauucagauguagaaacuaaagauguuguugaaugucuuaaauugucacaucaaucugacauagaaguuacuggcgauaguuguaauaacuauaugcucaccuauaacaaaguugaaaacaugacaccccgugaccuuggugcuuguauugacuguagugcgcgucauauuaaugcgcagguagcaaaaagucacaacauugcuuugauauggaacguuaaagauuucaugucauugucugaacaacuacgaaaacaaauacguagugcugcuaaaaagaauaacuuaccuuuuaaguugacaugugcaacuacuagacaaguuguuaauguuguaacaacaaagauagcacuuaagggugguaaaauuguuaauaauugguugaagcaguuaauuaaaguuacacuuguguuccuuuuuguugcugcuauuuucuauuuaauaacaccuguucaugucaugucuaaacauacugacuuuucaagugaaaucauaggauacaaggcuauugaugguggugucacucgugacauagcaucuacagauacuuguuuugcuaacaaacaugcugauuuugacacaugguuuagccagcguggugguaguuauacuaaugacaaagcuugcccauugauugcugcagucauaacaagagaaguggguuuugucgugccugguuugccuggcacgauauuacgcacaacuaauggugacuuuuugcauuucuuaccuagaguuuuuagugcaguugguaacaucuguuacacaccaucaaaacuuauagaguacacugacuuugcaacaucagcuuguguuuuggcugcugaauguacaauuuuuaaagaugcuucugguaagccaguaccauauuguuaugauaccaauguacuagaagguucuguugcuuaugaaaguuuacgcccugacacacguuaugugcucauggauggcucuauuauucaauuuccuaacaccuaccuugaagguucuguuagagugguaacaacuuuugauucugaguacuguaggcacggcacuugugaaagaucagaagcugguguuuguguaucuacuagugguagauggguacuuaacaaugauuauuacagaucuuuaccaggaguuuucugugguguagaugcuguaaauuuacuuacuaauauguuuacaccacuaauucaaccuauuggugcuuuggacauaucagcaucuauaguagcuggugguauuguagcuaucguaguaacaugccuugccuacuauuuuaugagguuuagaagagcuuuuggugaauacagucauguaguugccuuuaauacuuuacuauuccuuaugucauucacuguacucuguuuaacaccaguuuacucauucuuaccugguguuuauucuguuauuuacuuguacuugacauuuuaucuuacuaaugauguuucuuuuuuagcacauauucaguggaugguuauguucacaccuuuaguaccuuucuggauaacaauugcuuauaucauuuguauuuccacaaagcauuucuauugguucuuuaguaauuaccuaaagagacguguagucuuuaaugguguuuccuuuaguacuuuugaagaagcugcgcugugcaccuuuuuguuaaauaaagaaauguaucuaaaguugcguagugaugugcuauuaccucuuacgcaauauaauagauacuuagcucuuuauaauaaguacaaguauuuuaguggagcaauggauacaacuagcuacagagaagcugcuuguugucaucucgcaaaggcucucaaugacuucaguaacucagguucugauguucuuuaccaaccaccacaaaccucuaucaccucagcuguuuugcagagugguuuuagaaaaauggcauucccaucugguaaaguugaggguuguaugguacaaguaacuugugguacaacuacacuuaacggucuuuggcuugaugacguaguuuacuguccaagacaugugaucugcaccucugaagacaugcuuaacccuaauuaugaagauuuacucauucguaagucuaaucauaauuucuugguacaggcugguaauguucaacucaggguuauuggacauucuaugcaaaauuguguacuuaagcuuaagguugauacagccaauccuaagacaccuaaguauaaguuuguucgcauucaaccaggacagacuuuuucaguguuagcuuguuacaaugguucaccaucugguguuuaccaaugugcuaugaggcccaauuucacuauuaaggguucauuccuuaaugguucaugugguaguguugguuuuaacauagauuaugacugugucucuuuuuguuacaugcaccauauggaauuaccaacuggaguucaugcuggcacagacuuagaagguaacuuuuauggaccuuuuguugacaggcaaacagcacaagcagcugguacggacacaacuauuacaguuaauguuuuagcuugguuguacgcugcuguuauaaauggagacaggugguuucucaaucgauuuaccacaacucuuaaugacuuuaaccuuguggcuaugaaguacaauuaugaaccucuaacacaagaccauguugacauacuaggaccucuuucugcucaaacuggaauugccguuuuagauaugugugcuucauuaaaagaauuacugcaaaaugguaugaauggacguaccauauuggguagugcuuuauuagaagaugaauuuacaccuuuugauguuguuagacaaugcucagguguuacuuuccaaagugcagugaaaagaacaaucaaggguacacaccacugguuguuacucacaauuuugacuucacuuuuaguuuuaguccagaguacucaauggucuuuguucuuuuuuuuguaugaaaaugccuuuuuaccuuuugcuauggguauuauugcuaugucugcuuuugcaaugauguuugucaaacauaagcaugcauuucucuguuuguuuuuguuaccuucucuugccacuguagcuuauuuuaauauggucuauaugccugcuaguugggugaugcguauuaugacaugguuggauaugguugauacuaguuugucugguuuuaagcuaaaagacuguguuauguaugcaucagcuguaguguuacuaauccuuaugacagcaagaacuguguaugaugauggugcuaggagaguguggacacuuaugaaugucuugacacucguuuauaaaguuuauuaugguaaugcuuuagaucaagccauuuccaugugggcucuuauaaucucuguuacuucuaacuacucagguguaguuacaacugucauguuuuuggccagagguauuguuuuuauguguguugaguauugcccuauuuucuucauaacugguaauacacuucaguguauaaugcuaguuuauuguuucuuaggcuauuuuuguacuuguuacuuuggccucuuuuguuuacucaaccgcuacuuuagacugacucuugguguuuaugauuacuuaguuucuacacaggaguuuagauauaugaauucacagggacuacucccacccaagaauagcauagaugccuucaaacucaacauuaaauuguuggguguugguggcaaaccuuguaucaaaguagccacuguacagucuaaaaugucagauguaaagugcacaucaguagucuuacucucaguuuugcaacaacucagaguagaaucaucaucuaaauugugggcucaauguguccaguuacacaaugacauucucuuagcuaaagauacuacugaagccuuugaaaaaaugguuucacuacuuucuguuuugcuuuccaugcagggugcuguagacauaaacaagcuuugugaagaaaugcuggacaacagggcaaccuuacaagcuauagccucagaguuuaguucccuuccaucauaugcagcuuuugcuacugcucaagaagcuuaugagcaggcuguugcuaauggugauucugaaguuguucuuaaaaaguugaagaagucuuugaauguggcuaaaucugaauuugaccgugaugcagccaugcaacguaaguuggaaaagauggcugaucaagcuaugacccaaauguauaaacaggcuagaucugaggacaagagggcaaaaguuacuagugcuaugcagacaaugcuuuucacuaugcuuagaaaguuggauaaugaugcacucaacaacauuaucaacaaugcaagagaugguuguguucccuugaacauaauaccucuuacaacagcagccaaacuaaugguugucauaccagacuauaacacauauaaaaauacgugugaugguacaacauuuacuuaugcaucagcauugugggaaauccaacagguuguagaugcagauaguaaaauuguucaacuuagugaaauuaguauggacaauucaccuaauuuagcauggccucuuauuguaacagcuuuaagggccaauucugcugucaaauuacagaauaaugagcuuaguccuguugcacuacgacagaugucuugugcugccgguacuacacaaacugcuugcacugaugacaaugcguuagcuuacuacaacacaacaaagggagguagguuuguacuugcacuguuauccgauuuacaggauuugaaaugggcuagauucccuaagagugauggaacugguacuaucuauacagaacuggaaccaccuuguagguuuguuacagacacaccuaaagguccuaaagugaaguauuuauacuuuauuaaaggauuaaacaaccuaaauagagguaugguacuugguaguuuagcugccacaguacgucuacaagcugguaaugcaacagaagugccugccaauucaacuguauuaucuuucugugcuuuugcuguagaugcugcuaaagcuuacaaagauuaucuagcuagugggggacaaccaaucacuaauuguguuaagauguuguguacacacacugguacuggucaggcaauaacaguuacaccggaagccaauauggaucaagaauccuuugguggugcaucguguugucuguacugccguugccacauagaucauccaaauccuaaaggauuuugugacuuaaaagguaaguauguacaaauaccuacaacuugugcuaaugacccuguggguuuuacacuuaaaaacacagucuguaccgucugcgguauguggaaagguuauggcuguaguugugaucaacuccgcgaacccaugcuucagucagcugaugcacaaucguuuuuaaacggguuugcgguguaagugcagcccgucuuacaccgugcggcacaggcacuaguacugaugucguauacagggcuuuugacaucuacaaugauaaaguagcugguuuugcuaaauuccuaaaaacuaauuguugucgcuuccaagaaaaggacgaagaugacaauuuaauugauucuuacuuuguaguuaagagacacacuuucucuaacuaccaacaugaagaaacaauuuauaauuuacuuaaggauuguccagcuguugcuaaacaugacuucuuuaaguuuagaauagacggugacaugguaccacauauaucacgucaacgucuuacuaaauacacaauggcagaccucgucuaugcuuuaaggcauuuugaugaagguaauugugacacauuaaaagaaauacuugucacauacaauuguugugaugaugauuauuucaauaaaaaggacugguaugauuuuguagaaaacccagauauauuacgcguauacgccaacuuaggugaacguguacgccaagcuuuguuaaaaacaguacaauucugugaugccaugcgaaaugcugguauuguugguguacugacauuagauaaucaagaucucaaugguaacugguaugauuucggugauuucauacaaaccacgccagguaguggaguuccuguuguagauucuuauuauucauuguuaaugccuauauuaaccuugaccagggcuuuaacugcagagucacauguugacacugacuuaacaaagccuuacauuaagugggauuuguuaaaauaugacuucacggaagagagguuaaaacucuuugaccguuauuuuaaauauugggaucagacauaccacccaaauuguguuaacuguuuggaugacagaugcauucugcauugugcaaacuuuaauguuuuauucucuacaguguucccaccuacaaguuuuggaccacuagugagaaaaauauuuguugaugguguuccauuuguaguuucaacuggauaccacuucagagagcuagguguuguacauaaucaggauguaaacuuacauagcucuagacuuaguuuuaaggaauuacuuguguaugcugcugacccugcuaugcacgcugcuucugguaaucuauuacuagauaaacgcacuacgugcuuuucaguagcugcacuuacuaacaauguugcuuuucaaacugucaaacccgguaauuuuaacaaagacuucuaugacuuugcugugucuaaggguuucuuuaaggaaggaaguucuguugaauuaaaacacuucuucuuugcucaggaugguaaugcugcuaucagcgauuaugacuacuaucguuauaaucuaccaacaaugugugauaucagacaacuacuauuuguaguugaaguuguugauaaguacuuugauuguuacgaugguggcuguauuaaugcuaaccaagucaucgucaacaaccuagacaaaucagcugguuuuccauuuaauaaaugggguaaggcuagacuuuauuaugauucaaugaguuaugaggaucaagaugcacuuuucgcauauacaaaacguaaugucaucccuacuauaacucaaaugaaucuuaaguaugccauuagugcaaagaauagagcucgcaccguagcuggugucucuaucuguaguacuaugaccaauagacaguuucaucaaaaauuauugaaaucaauagccgccacuagaggagcuacuguaguaauuggaacaagcaaauucuauggugguuggcacaacauguuaaaaacuguuuauagugauguagaaaacccucaccuuauggguugggauuauccuaaaugugauagagccaugccuaacaugcuuagaauuauggccucacuuguucuugcucgcaaacauacaacguguuguagcuugucacaccguuucuauagauuagcuaaugagugugcucaaguauugagugaaauggucauguguggcgguucacuauauguuaaaccagguggaaccucaucaggagaugccacaacugcuuaugcuaauaguguuuuuaacauuugucaagcugucacggccaauguuaaugcacuuuuaucuacugaugguaacaaaauugccgauaaguauguccgcaauuuacaacacagacuuuaugagugucucuauagaaauagagauguugacacagacuuugugaaugaguuuuacgcauauuugcguaaacauuucucaaugaugauacucucugacgaugcuguuguguguuucaauagcacuuaugcaucucaaggucuaguggcuagcauaaagaacuuuaagucaguucuuuauuaucaaaacaauguuuuuaugucugaagcaaaauguuggacugagacugaccuuacuaaaggaccucaugaauuuugcucucaacauacaaugcuaguuaaacagggugaugauuauguguaccuuccuuacccagauccaucaagaauccuaggggccggcuguuuuguagaugauaucguaaaaacagaugguacacuuaugauugaacgguucgugucuuuagcuauagaugcuuacccacuuacuaaacauccuaaucaggaguaugcugaugucuuucauuuguacuuacaauacauaagaaagcuacaugaugaguuaacaggacacauguuagacauguauucuguuaugcuuacuaaugauaacacuucaagguauugggaaccugaguuuuaugaggcuauguacacaccgcauacagucuuacaggcuguuggggcuuguguucuuugcaauucacagacuucauuaagauguggugcuugcauacguagaccauucuuauguuguaaaugcuguuacgaccaugucauaucaacaucacauaaauuagucuugucuguuaauccguauguuugcaaugcuccagguugugaugucacagaugugacucaacuuuacuuaggagguaugagcuauuauuguaaaucacauaaaccacccauuaguuuuccauugugugcuaauggacaaguuuuugguuuauauaaaaauacauguguugguagcgauaauguuacugacuuuaaugcaauugcaacaugugacuggacaaaugcuggugauuacauuuuagcuaacaccuguacugaaagacucaagcuuuuugcagcagaaacgcucaaagcuacugaggagacauuuaaacugucuuaugguauugcuacuguacgugaagugcugucugacagagaauuacaucuuucaugggaaguugguaaaccuagaccaccacuuaaccgaaauuaugucuuuacugguuaucguguaacuaaaaacaguaaaguacaaauaggagaguacaccuuugaaaaaggugacuauggugaugcuguuguuuaccgagguacaacaacuuacaaauuaaauguuggugauuauuuugugcugacaucacauacaguaaugccauuaagugcaccuacacuagugccacaagagcacuauguuagaauuacuggcuuauacccaacacucaauaucucagaugaguuuucuagcaauguugcaaauuaucaaaagguugguaugcaaaaguauucuacacuccagggaccaccugguacugguaagagucauuuugcuauuggccuagcucucuacuacccuucugcucgcauaguguauacagcuugcucucaugccgcuguugaugcacuaugugagaaggcauuaaaauauuugccuauagauaaauguaguagaauuauaccugcacgugcucguguagaguguuuugauaaauucaaagugaauucaacauuagaacaguaugucuuuuguacuguaaaugcauugccugagacgacagcagauauaguugucuuugaugaaauuucaauggccacaaauuaugauuugaguguugucaaugccagauuacgugcuaagcacuauguguacauuggcgacccugcucaauuaccugcaccacgcacauugcuaacuaagggcacacuagaaccagaauauuucaauucaguguguagacuuaugaaaacuauagguccagacauguuccucggaacuugucggcguuguccugcugaaauuguugacacugugagugcuuugguuuaugauaauaagcuuaaagcacauaaagacaaaucagcucaaugcuuuaaaauguuuuauaaggguguuaucacgcaugauguuucaucugcaauuaacaggccacaaauaggcgugguaagagaauuccuuacacguaacccugcuuggagaaaagcugucuuuauuucaccuuauaauucacagaaugcuguagccucaaagauuuugggacuaccaacucaaacuguugauucaucacagggcucagaauaugacuaugucauauucacucaaaccacugaaacagcucacucuuguaauguaaacagauuuaauguugcuauuaccagagcaaaaguaggcauacuuugcauaaugucugauagagaccuuuaugacaaguugcaauuuacaagucuugaaauuccacguaggaauguggcaacuuuacaagcugaaaauguaacaggacucuuuaaagauuguaguaagguaaucacuggguuacauccuacacaggcaccuacacaccucaguguugacacuaaauucaaaacugaagguuuauguguugacauaccuggcauaccuaaggacaugaccuauagaagacucaucucuaugauggguuuuaaaaugaauuaucaaguuaaugguuacccuaacauguuuaucacccgcgaagaagcuauaagacauguacgugcauggauuggcuucgaugucgaggggugucaugcuacuagagaagcuguugguaccaauuuaccuuuacagcuagguuuuucuacagguguuaaccuaguugcuguaccuacagguuauguugauacaccuaauaauacagauuuuuccagaguuagugcuaaaccaccgccuggagaucaauuuaaacaccucauaccacuuauguacaaaggacuuccuuggaauguagugcguauaaagauuguacaaauguuaagugacacacuuaaaaaucucucugacagagucguauuugucuuaugggcacauggcuuugaguugacaucuaugaaguauuuugugaaaauaggaccugagcgcaccuguugucuaugugauagacgugccacaugcuuuuccacugcuucagacacuuaugccuguuggcaucauucuauuggauuugauuacgucuauaauccguuuaugauugauguucaacaaugggguuuuacagguaaccuacaaagcaaccaugaucuguauugucaaguccaugguaaugcacauguagcuaguugugaugcaaucaugacuaggugucuagcuguccacgagugcuuuguuaagcguguugacuggacuauugaauauccuauaauuggugaugaacugaagauuaaugcggcuuguagaaagguucaacacaugguuguuaaagcugcauuauuagcagacaaauucccaguucuucacgacauugguaacccuaaagcuauuaaguguguaccucaagcugauguagaauggaaguucuaugaugcacagccuuguagugacaaagcuuauaaaauagaagaauuauucuauucuuaugccacacauucugacaaauucacagaugguguaugccuauuuuggaauugcaaugucgauagauauccugcuaauuccauuguuuguagauuugacacuagagugcuaucuaaccuuaacuugccugguugugaugguggcaguuuguauguaaauaaacaugcauuccacacaccagcuuuugauaaaagugcuuuuguuaauuuaaaacaauuaccauuuuucuauuacucugacaguccaugugagucucauggaaaacaaguagugucagauauagauuauguaccacuaaagucugcuacguguauaacacguugcaauuuagguggugcugucuguagacaucaugcuaaugaguacagauuguaucucgaugcuuauaacaugaugaucucagcuggcuuuagcuuguggguuuacaaacaauuugauacuuauaaccucuggaacacuuuuacaagacuucagaguuuagaaaauguggcuuuuaauguuguaaauaagggacacuuugauggacaacagggugaaguaccaguuucuaucauuaauaacacuguuuacacaaaaguugaugguguugauguagaauuguuugaaaauaaaacaacauuaccuguuaauguagcauuugagcuuugggcuaagcgcaacauuaaaccaguaccagaggugaaaauacucaauaauuuggguguggacauugcugcuaauacugugaucugggacuacaaaagagaugcuccagcacauauaucuacuauugguguuuguucuaugacugacauagccaagaaaccaacugaaacgauuugugcaccacucacugucuuuuuugaugguagaguugauggucaaguagacuuauuuagaaaugcccguaaugguguucuuauuacagaagguaguguuaaagguuuacaaccaucuguaggucccaaacaagcuagucuuaauggagucacauuaauuggagaagccguaaaaacacaguucaauuauuauaagaaaguugaugguguuguccaacaauuaccugaaacuuacuuuacucagaguagaaauuuacaagaauuuaaacccaggagucaaauggaaauugauuucuuagaauuagcuauggaugaauucauugaacgguauaaauuagaaggcuaugccuucgaacauaucguuuauggagauuuuagucauagucaguuaggugguuuacaucuacugauuggacuagcuaaacguuuuaaggaaucaccuuuugaauuagaagauuuuauuccuauggacaguacaguuaaaaacuauuucauaacagaugcgcaaacagguucaucuaaguguguguguucuguuauugauuuauuacuugaugauuuuguugaaauaauaaaaucccaagauuuaucuguaguuucuaagguugucaaagugacuauugacuauacagaaauuucauuuaugcuuugguguaaagauggccauguagaaacauuuuacccaaaauuacaaucuagucaagcguggcaaccggguguugcuaugccuaaucuuuacaaaaugcaaagaaugcuauuagaaaagugugaccuucaaaauuauggugauagugcaacauuaccuaaaggcauaaugaugaaugucgcaaaauauacucaacugugucaauauuuaaacacauuaacauuagcuguacccuauaauaugagaguuauacauuuuggugcugguucugauaaaggaguugcaccagguacagcuguuuuaagacagugguugccuacggguacgcugcuugucgauucagaucuuaaugacuuugucucugaugcagauucaacuuugauuggugauugugcaacuguacauacagcuaauaaaugggaucucauuauuagugauauguacgacccuaagacuaaaaauguuacaaaagaaaaugacucuaaagaggguuuuuucacuuacauuuguggguuuauacaacaaaagcuagcucuuggagguuccguggcuauaaagauaacagaacauucuuggaaugcugaucuuuauaagcucaugggacacuucgcaugguggacagccuuuguuacuaaugugaaugcgucaucaucugaagcauuuuuaauuggauguaauuaucuuggcaaaccacgcgaacaaauagaugguuaugucaugcaugcaaauuacauauuuuggaggaauacaaauccaauucaguugucuuccuauucuuuauuugacaugaguaaauuuccccuuaaauuaagggguacugcuguuaugucuuuaaaagaaggucaaaucaaugauaugauuuuaucucuucuuaguaaagguagacuuauaauuagagaaaacaacagaguuguuauuucuagugauguucuuguuaacaacuaaacgaacaauguuuguuuuucuuguuuuauugccacuagucucuagucaguguguuaaucuuacaaccagaacucaauuacccccugcauacacuaauucuuucacacgugguguuuauuacccugacaaaguuuucagauccucaguuuuacauucaacucaggacuuguucuuaccuuucuuuuccaauguuacuugguuccaugcuauacaugucucugggaccaaugguacuaagagguuugauaacccuguccuaccauuuaaugaugguguuuauuuugcuuccacugagaagucuaacauaauaagaggcuggauuuuugguacuacuuuagauucgaagacccagucccuacuuauuguuaauaacgcuacuaauguuguuauuaaagucugugaauuucaauuuuguaaugauccauuuuuggguguuuauuaccacaaaaacaacaaaaguuggauggaaagugaguucagaguuuauucuagugcgaauaauugcacuuuugaauaugucucucagccuuuucuuauggaccuugaaggaaaacaggguaauuucaaaaaucuuagggaauuuguguuuaagaauauugaugguuauuuuaaaauauauucuaagcacacgccuauuaauuuagugcgugaucucccucaggguuuuucggcuuuagaaccauugguagauuugccaauagguauuaacaucacuagguuucaaacuuuacuugcuuuacauagaaguuauuugacuccuggugauucuucuucagguuggacagcuggugcugcagcuuauuauguggguuaucuucaaccuaggacuuuucuauuaaaauauaaugaaaauggaaccauuacagaugcuguagacugugcacuugacccucucucagaaacaaaguguacguugaaauccuucacuguagaaaaaggaaucuaucaaacuucuaacuuuagaguccaaccaacagaaucuauuguuagauuuccuaauauuacaaacuugugcccuuuuggugaaguuuuuaacgccaccagauuugcaucuguuuaugcuuggaacaggaagagaaucagcaacuguguugcugauuauucuguccuauauaauuccgcaucauuuuccacuuuuaaguguuauggagugucuccuacuaaauuaaaugaucucugcuuuacuaaugucuaugcagauucauuuguaauuagaggugaugaagucagacaaaucgcuccagggcaaacuggaaagauugcugauuauaauuauaaauuaccagaugauuuuacaggcugcguuauagcuuggaauucuaacaaucuugauucuaagguuggugguaauuauaauuaccuguauagauuguuuaggaagucuaaucucaaaccuuuugagagagauauuucaacugaaaucuaucaggccgguagcacaccuuguaaugguguugaagguuuuaauuguuacuuuccuuuacaaucauaugguuuccaacccacuaaugguguugguuaccaaccauacagaguaguaguacuuucuuuugaacuucuacaugcaccagcaacuguuuguggaccuaaaaagucuacuaauuugguuaaaaacaaaugugucaauuucaacuucaaugguuuaacaggcacagguguucuuacugagucuaacaaaaaguuucugccuuuccaacaauuuggcagagacauugcugacacuacugaugcuguccgugauccacagacacuugagauucuugacauuacaccauguucuuuugguggugucaguguuauaacaccaggaacaaauacuucuaaccagguugcuguucuuuaucaggauguuaacugcacagaagucccuguugcuauucaugcagaucaacuuacuccuacuuggcguguuuauucuacagguucuaauguuuuucaaacacgugcaggcuguuuaauaggggcugaacaugucaacaacucauaugagugugacauacccauuggugcagguauaugcgcuaguuaucagacucagacuaauucuccucggcgggcacguaguguagcuagucaauccaucauugccuacacuaugucacuuggugcagaaaauucaguugcuuacucuaauaacucuauugccauacccacaaauuuuacuauuaguguuaccacagaaauucuaccagugucuaugaccaagacaucaguagauuguacaauguacauuuguggugauucaacugaaugcagcaaucuuuuguugcaauauggcaguuuuuguacacaauuaaaccgugcuuuaacuggaauagcuguugaacaagacaaaaacacccaagaaguuuuugcacaagucaaacaaauuuacaaaacaccaccaauuaaagauuuuggugguuuuaauuuuucacaaauauuaccagauccaucaaaaccaagcaagaggucauuuauugaagaucuacuuuucaacaaagugacacuugcagaugcuggcuucaucaaacaauauggugauugccuuggugauauugcugcuagagaccucauuugugcacaaaaguuuaacggccuuacuguuuugccaccuuugcucacagaugaaaugauugcucaauacacuucugcacuguuagcggguacaaucacuucugguuggaccuuuggugcaggugcugcauuacaaauaccauuugcuaugcaaauggcuuauagguuuaaugguauuggaguuacacagaauguucucuaugagaaccaaaaauugauugccaaccaauuuaauagugcuauuggcaaaauucaagacucacuuucuuccacagcaagugcacuuggaaaacuucaagauguggucaaccaaaaugcacaagcuuuaaacacgcuuguuaaacaacuuagcuccaauuuuggugcaauuucaaguguuuuaaaugauauccuuucacgucuugacaaaguugaggcugaagugcaaauugauagguugaucacaggcagacuucaaaguuugcagacauaugugacucaacaauuaauuagagcugcagaaaucagagcuucugcuaaucuugcugcuacuaaaaugucagaguguguacuuggacaaucaaaaagaguugauuuuuguggaaagggcuaucaucuuauguccuucccucagucagcaccucaugguguagucuucuugcaugugacuuaugucccugcacaagaaaagaacuucacaacugcuccugccauuugucaugauggaaaagcacacuuuccucgugaaggugucuuuguuucaaauggcacacacugguuuguaacacaaaggaauuuuuaugaaccacaaaucauuacuacagacaacacauuugugucugguaacugugauguuguaauaggaauugucaacaacacaguuuaugauccuuugcaaccugaauuagacucauucaaggaggaguuagauaaauauuuuaagaaucauacaucaccagauguugauuuaggugacaucucuggcauuaaugcuucaguuguaaacauucaaaaagaaauugaccgccucaaugagguugccaagaauuuaaaugaaucucucaucgaucuccaagaacuuggaaaguaugagcaguauauaaaauggccaugguacauuuggcuagguuuuauagcuggcuugauugccauaguaauggugacaauuaugcuuugcuguaugaccaguugcuguaguugucucaagggcuguuguucuuguggauccugcugcaaauuugaugaagacgacucugagccagugcucaaaggagucaaauuacauuacacauaaacgaacuuauggauuuguuuaugagaaucuucacaauuggaacuguaacuuugaagcaaggugaaaucaaggaugcuacuccuucagauuuuguucgcgcuacugcaacgauaccgauacaagccucacucccuuucggauggcuuauuguuggcguugcacuucuugcuguuuuucagagcgcuuccaaaaucauaacccucaaaaagagauggcaacuagcacucuccaaggguguucacuuuguuugcaacuugcuguuguuguuuguaacaguuuacucacaccuuuugcucguugcugcuggccuugaagccccuuuucucuaucuuuaugcuuuagucuacuucuugcagaguauaaacuuuguaagaauaauaaugaggcuuuggcuuugcuggaaaugccguuccaaaaacccauuacuuuaugaugccaacuauuuucuuugcuggcauacuaauuguuacgacuauuguauaccuuacaauaguguaacuucuucaauugucauuacuucaggugauggcacaacaaguccuauuucugaacaugacuaccagauuggugguuauacugaaaaaugggaaucuggaguaaaagacuguguuguauuacacaguuacuucacuucagacuauuaccagcuguacucaacucaauugaguacagacacugguguugaacauguuaccuucuucaucuacaauaaaauuguugaugagccugaagaacauguccaaauucacacaaucgacgguucauccggaguuguuaauccaguaauggaaccaauuuaugaugaaccgacgacgacuacuagcgugccuuuguaagcacaagcugaugaguacgaacuuauguacucauucguuucggaagagacagguacguuaauaguuaauagcguacuucuuuuucuugcuuucgugguauucuugcuaguuacacuagccauccuuacugcgcuucgauugugugcguacugcugcaauauuguuaacgugagucuuguaaaaccuucuuuuuacguuuacucucguguuaaaaaucugaauucuucuagaguuccugaucuucuggucuaaacgaacuaaauauuauauuaguuuuucuguuuggaacuuuaauuuuagccauggcagauuccaacgguacuauuaccguugaagagcuuaaaaagcuccuugaacaauggaaccuaguaauagguuuccuauuccuuacauggauuugucuucuacaauuugccuaugccaacaggaauagguuuuuguauauaauuaaguuaauuuuccucuggcuguuauggccaguaacuuuagcuuguuuugugcuugcugcuguuuacagaauaaauuggaucaccgguggaauugcuaucgcaauggcuugucuuguaggcuugauguggcucagcuacuucauugcuucuuucagacuguuugcgcguacgcguuccauguggucauucaauccagaaacuaacauucuucucaacgugccacuccauggcacuauucugaccagaccgcuucuagaaagugaacucguaaucggagcugugauccuucguggacaucuucguauugcuggacaccaucuaggacgcugugacaucaaggaccugccuaaagaaaucacuguugcuacaucacgaacgcuuucuuauuacaaauugggagcuucgcagcguguagcaggugacucagguuuugcugcauacagucgcuacaggauuggcaacuauaaauuaaacacagaccauuccaguagcagugacaauauugcuuugcuuguacaguaagugacaacagauguuucaucucguugacuuucagguuacuauagcagagauauuacuaauuauuaugaggacuuuuaaaguuuccauuuggaaucuugauuacaucauaaaccucauaauuaaaaauuuaucuaagucacuaacugagaauaaauauucucaauuagaugaagagcaaccaauggagauugauuaaacgaacaugaaaauuauucuuuucuuggcacugauaacacucgcuacuugugagcuuuaucacuaccaagaguguguuagagguacaacaguacuuuuaaaagaaccuugcucuucuggaacauacgagggcaauucaccauuucauccucuagcugauaacaaauuugcacugacuugcuuuagcacucaauuugcuuuugcuuguccugacggcguaaaacacgucuaucaguuacgugccagaucaguuucaccuaaacuguucaucagacaagaggaaguucaagaacuuuacucuccaauuuuucuuauuguugcggcaauaguguuuauaacacuuugcuucacacucaaaagaaagacagaaugauugaacuuucauuaauugacuucuauuugugcuuuuuagccuuucugcuauuccuuguuuuaauuaugcuuauuaucuuuugguucucacuugaacugcaagaucauaaugaaacuugucacgccuaaacgaacaugaaauuucuuguuuucuuaggaaucaucacaacuguagcugcauuucaccaagaauguaguuuacagucauguacucaacaucaaccauauguaguugaugacccguguccuauucacuucuauucuaaaugguauauuagaguaggagcuagaaaaucagcaccuuuaauugaauugugcguggaugaggcugguucuaaaucacccauucaguacaucgauaucgguaauuauacaguuuccuguuuaccuuuuacaauuaauugccaggaaccuaaauuggguagucuuguagugcguuguucguucuaugaagacuuuuuagaguaucaugacguucguguuguuuuagauuucaucuaaacgaacaaacuaaaaugucugauaauggaccccaaaaucagcgaaaugcaccccgcauuacguuugguggacccucagauucaacuggcaguaaccagaauggagaacgcaguggggcgcgaucaaaacaacgucggccccaagguuuacccaauaauacugcgucuugguucaccgcucucacucaacauggcaaggaagaccuuaaauucccucgaggacaaggcguuccaauuaacaccaauagcaguccagaugaccaaauuggcuacuaccgaagagcuaccagacgaauucgugguggugacgguaaaaugaaagaucucaguccaagaugguauuucuacuaccuaggaacugggccagaagcuggacuucccuauggugcuaacaaagacggcaucauauggguugcaacugagggagccuugaauacaccaaaagaucacauuggcacccgcaauccugcuaacaaugcugcaaucgugcuacaacuuccucaaggaacaacauugccaaaaggcuucuacgcagaagggagcagaggcggcagucaagccucuucucguuccucaucacguagucgcaacaguucaagaaauucaacuccaggcagcaguaggggaacuucuccugcuagaauggcuggcaauggcggugaugcugcucuugcuuugcugcugcuugacagauugaaccagcuugagagcaaaaugucugguaaaggccaacaacaacaaggccaaacugucacuaagaaaucugcugcugaggcuucuaagaagccucggcaaaaacguacugccacuaaagcauacaauguaacacaagcuuucggcagacgugguccagaacaaacccaaggaaauuuuggggaccaggaacuaaucagacaaggaacugauuacaaacauuggccgcaaauugcacaauuugcccccagcgcuucagcguucuucggaaugucgcgcauuggcauggaagucacaccuucgggaacgugguugaccuacacaggugccaucaaauuggaugacaaagauccaaauuucaaagaucaagucauuuugcugaauaagcauauugacgcauacaaaacauucccaccaacagagccuaaaaaggacaaaaagaagaaggcugaugaaacucaagccuuaccgcagagacagaagaaacagcaaacugugacucuucuuccugcugcagauuuggaugauuucuccaaacaauugcaacaauccaugagcagugcugacucaacucaggccuaaacucaugcagaccacacaaggcagaugggcuauauaaacguuuucgcuuuuccguuuacgauauauagucuacucuugugcagaaugaauucucguaacuacauagcacaaguagauguaguuaacuuuaaucucacauagcaaucuuuaaucaguguguaacauuagggaggacuugaaagagccaccacauuuucaccgaggccacgcggaguacgaucgaguguacagugaacaaugcuagggagagcugccuauauggaagagcccuaauguguaaaauuaauuuuaguagugcuauccccaugugauuuuaauagcuucuuaggagaaugacaaaaaaaaaaaaaaaaaaaaaaaaaaaaaaaaa

SEQ ID NO: 39 - protein, 1273 aa, Severe acute respiratory syndrome coronavirus 2
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 40 - DNA, 2559 bp, Artificial Sequence
  Description of Artificial Sequence Synthetic polynucleotide
  pT7/NSP1SA11-2A-GFP
actagttaatacgactcactataggcttttttttgaaaagtcttgtgttagccatggctacttttaaagatgcatgctttcattatcgtagattaactgctttaaatcggagattatgcaacattggtgcaaattctatttggatgccagttcctgatgcgaaaattaaggggtggtgtttagaatgttgtcaaatagctgatttaacccattgttatggttgctcattgccgcatgtttgcaaatggtgtgttcagaacagaagatgcttccttgacaatgaacctcatttgcttaagcttagaactgtgaaacatccaattaccaaagacaaattacagtgtatcatagacttgtacaatataatatttccaattaatgataaagtaattagaaaatttgaaagaatgataaagcaaagagaatgtaggaatcaatataaaattgaatggtataatcatttgctgctcccaattacattaaatgctgctgcatttaagtttgatgaaaataatctttattatgtttttgggttatatgagaaatcagtcagtgatatatatgctccatatagaattgttaactttataaatgaatttgataaattattgcttgatcatattaactttacaagaatgtccaatctaccaatagagttgagaaaccattacgcaaagaaatacttccaattatcaagactgccatcatcaaaactaaagcaaatttacttttcagattttactaaagaaactgtgatttttaatacttatacaaaaacgccaggaagatcaatatacagaaatgtaactgaatttaattggagagatgaattggagctttattctgatttaaaaaatgataagaataaattaattgctgcaatgatgacgagtaagtatactcggttctatgctcatgataataattttggaaggttgaaaatgacaatatttgagttgggacatcattgtcagcctaactacgtggcatctaatcacccaggcaatgcttccgatatccagtactgtaaatggtgtaatataaaatattttcttagtaaaattgattggcggattcgtgatatgtataatttattgatggaatttattaaggattgttataaaagtaatgttaacgttggacattgtagttctgttgaaaacatatatcctttaattaaaagattaatttggagtttgtttactaatcacatggatcaaacaattgaagaagtgtttaatcacatgtcgccagtgtcagttgaaggtacgaatgtcatcatgttgattcttggattgaatattagtttgtataatgaaattaagcgcactttgaatgtagatagcataccaatggtacttaatttaaatgaattcagtagtatagttaaatcaattagcagtaaatggtataatgttgatgaattggataaattgccaatgtcaataaaatcaacggaggaactgattgaaatgaagaattctggaactttaactgaagaatttgagctactgatctccaactcagaagatgacaatgagggctccggcgagggcaggggaagtcttctaacatgcggggacgtggaggaaaatcccggcccagtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaatgaaattatgtcactatctaattatacagtatttagccatcacaagaccgtccagactagagtagcgcctagctggcaaaatactgtgaaccgggtcggcatggcatctccacctcctcgcggtccgacctgggcatccgaaggaggacgcacgtccactcggatggctaagggagagcctgcagtagcataaccccttggggcctctaaacgggtcttgaggggttttttgggtacc

SEQ ID NO: 41 - DNA, 836 bp, Artificial Sequence; identical to SEQ ID NO: 21
  Description of Artificial Sequence Synthetic polynucleotide
  pT7/NSP5SA11-2A-GFP

SEQ ID NO: 42 - protein, 574 aa, Human orthopneumovirus
MELLIHRSSAIFLTLAINALYLTSSQNITEEFYQSTCSAVSRGYFSALRTGWYTSVITIELSNIKETKCNGTDTKVKLIKQELDKYKNAVTELQLLTQNTPAANNRARREAPQYMNYTINTTKNLNVSISKKRKRRFLGFLLGVGSAIASGIAVSKVLHLEGEVNKIKNALLSTNKAVVSLSNGVSVLTSKVLDLKSYINNQLLPIVNQQSCRISNIETVIEFQQKNSRLLEITREFSVNAGVTTPLSTYMLTNSELLSLINDMPITNDQKKLMSSNVQIVRQQSYSIMSIIKEEVLAYVVQLPIYGVIDTPCWKLHTSPLCTTNIKEGSNICLTRTDRGWYCDNAGSVSFFPQADTCKVQSNRVFCDTMNSLTLPSEVSLCNTDIFNSKYDCKIMTSKTDISSSVITSLGAIVSCYGKTKCTASNKNRGIIKTFSNGCDYVSNKGVDTVSVGNTLYYVNKLEGKNLYVKGEPIINYYDPLVFPSDEFDASISQVNEKINQSLAFIRRSDELLHNVNTGKSTTNIMITTIIIVIIVVLLSLIAIGLLLYCKAKNTPVTLSKDQLSGINNIAFSK

SEQ ID NO: 43 - DNA, 3549 bp, Artificial Sequence
  Description of Artificial Sequence Synthetic polynucleotide
  pT7/RSV-T4PreF
actagttaatacgactcactataggcttttttttgaaaagtcttgtgttagccatggctacttttaaagatgcatgctttcattatcgtagattaactgctttaaatcggagattatgcaacattggtgcaaattctatttggatgccagttcctgatgcgaaaattaaggggtggtgtttagaatgttgtcaaatagctgatttaacccattgttatggttgctcattgccgcatgtttgcaaatggtgtgttcagaacagaagatgcttccttgacaatgaacctcatttgcttaagcttagaactgtgaaacatccaattaccaaagacaaattacagtgtatcatagacttgtacaatataatatttccaattaatgataaagtaattagaaaatttgaaagaatgataaagcaaagagaatgtaggaatcaatataaaattgaatggtataatcatttgctgctcccaattacattaaatgctgctgcatttaagtttgatgaaaataatctttattatgtttttgggttatatgagaaatcagtcagtgatatatatgctccatatagaattgttaactttataaatgaatttgataaattattgcttgatcatattaactttacaagaatgtccaatctaccaatagagttgagaaaccattacgcaaagaaatacttccaattatcaagactgccatcatcaaaactaaagcaaatttacttttcagattttactaaagaaactgtgatttttaatacttatacaaaaacgccaggaagatcaatatacagaaatgtaactgaatttaattggagagatgaattggagctttattctgatttaaaaaatgataagaataaattaattgctgcaatgatgacgagtaagtatactcggttctatgctcatgataataattttggaaggttgaaaatgacaatatttgagttgggacatcattgtcagcctaactacgtggcatctaatcacccaggcaatgcttccgatatccagtactgtaaatggtgtaatataaaatattttcttagtaaaattgattggcggattcgtgatatgtataatttattgatggaatttattaaggattgttataaaagtaatgttaacgttggacattgtagttctgttgaaaacatatatcctttaattaaaagattaatttggagtttgtttactaatcacatggatcaaacaattgaagaagtgtttaatcacatgtcgccagtgtcagttgaaggtacgaatgtcatcatgttgattcttggattgaatattagtttgtataatgaaattaagcgcactttgaatgtagatagcataccaatggtacttaatttaaatgaattcagtagtatagttaaatcaattagcagtaaatggtataatgttgatgaattggataaattgccaatgtcaataaaatcaacggaggaactgattgaaatgaagaattctggaactttaactgaagaatttgagctactgatctccaactcagaagatgacaatgagggctccggcgagggcaggggaagtcttctaacatgcggggacgtggaggaaaatcccggcccaatggaactccttatccttaaagcaaatgctattaccaccatacttactgcagtcacattctgctttgcatctgggcagaacataactgaagagttctaccagtcaacctgcagtgccgtgtctaaaggctacctcagcgcactgcgaaccggatggtacacgtcagtcataacgatcgaactttcaaatataaaggagaataaatgcaatgggactgacgccaaagttaaactcatcaaacaggaacttgataaatataagaatgcggtcaccgagttgcagctccttatgcagagcacgcctgccacgaacaatcgcgcccgacgagaacttccacggtttatgaattatacactgaacaacgccaaaaaaactaacgtaacacttagtaagaagaggaagaggcggtttcttggttttttgctgggggtagggtcagcaatcgcatctggagtggccgtttgtaaggtgctgcatctcgaaggtgaggtgaataagatcaagtccgcccttctttctacaaataaggcagtggtcagtttgtcaaatggagttagtgtgttgactttcaaggtactggacctgaaaaactatatagacaagcagcttctcccaattttgaataaacaatcttgctcaataagtaacatcgaaacagtgattgagtttcagcaaaaaaataacaggctgcttgaaatcacacgggagttttccgttaacgccggcgtaacgactccggtctctacttatatgctcacaaattcagaattgctttctttgataaatgatatgccaataaccaacgaccaaaagaaactgatgagtaacaatgtacagatagttagacagcagtcatattctatcatgtgtattataaaagaagaggtcttggcctacgtagtacaactcccgctctatggagtgatcgacacaccgtgctggaagttgcacaccagccccctgtgtactactaacacaaaggagggttctaatatttgtctcacccgcacggaccgaggctggtattgcgacaacgctggatccgtaagtttcttcccccaggcggaaacatgtaaagtgcaaagcaaccgcgtattttgcgacacaatgaatagtctgacgcttccatcagaggtcaatctttgtaacgtggacatcttcaatccaaagtacgactgtaaaattatgacatctaagacagacgtctcatccagcgtcatcacctccctcggcgcgatcgtaagctgttatggcaagactaagtgtacagctagcaataagaacagggggatcatcaaaaccttttctaacggctgtgattacgtgtccaacaaaggagtagatactgtatcagtcggcaatacgctctattacgtgaacaagcaagaaggtaagagcctttacgtcaaaggggaacccattattaacttttacgacccattggtctttcctagtgatgaattcgacgcttctataagtcaagtaaacgagaagatcaatcagagtctcgccttcatcaggaaatccgatgaacttctttctgcgattggaggatatattcccgaagcacccagggatgggcaagcatacgtaagaaaagatggagaatgggttctgctttctacatttcttggggggcttgtcccgaggggtagccatcatcatcatcaccacagtgcatggtcacacccccaatttgaaaagtaatgaaattatgtcactatctaattatacagtatttagccatcacaagaccgtccagactagagtagcgcctagctggcaaaatactgtgaaccgggtcggcatggcatctccacctcctcgcggtccgacctgggcatccgaaggaggacgcacgtccactcggatggctaagggagagcctgcagtagcataaccccttggggcctctaaacgggtcttgaggggttttttgggtacc

SEQ ID NO: 44 - protein, 568 aa, Artificial Sequence
  Description of Artificial Sequence Synthetic polypeptide
  RSV-T4PreF
MELLILKANAITTILTAVTFCFASGQNITEEFYQSTCSAVSKGYLSALRTGWYTSVITIELSNIKENKCNGTDAKVKLIKQELDKYKNAVTELQLLMQSTPATNNRARRELPRFMNYTLNNAKKTNVTLSKKRKRRFLGFLLGVGSAIASGVAVCKVLHLEGEVNKIKSALLSTNKAVVSLSNGVSVLTFKVLDLKNYIDKQLLPILNKQSCSISNIETVIEFQQKNNRLLEITREFSVNAGVTTPVSTYMLTNSELLSLINDMPITNDQKKLMSNNVQIVRQQSYSIMCIIKEEVLAYVVQLPLYGVIDTPCWKLHTSPLCTTNTKEGSNICLTRTDRGWYCDNAGSVSFFPQAETCKVQSNRVFCDTMNSLTLPSEVNLCNVDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSDEFDASISQVNEKINQSLAFIRKSDELLSAIGGYIPEAPRDGQAYVRKDGEWVLLSTFLGGLVPRGSHHHHHHSAWSHPQFEK

SEQ ID NO: 45 - DNA, 3444 bp, Artificial Sequence
  Description of Artificial Sequence Synthetic polynucleotide
  pT7/RSV-T4scPreF
actagttaatacgactcactataggcttttttttgaaaagtcttgtgttagccatggctacttttaaagatgcatgctttcattatcgtagattaactgctttaaatcggagattatgcaacattggtgcaaattctatttggatgccagttcctgatgcgaaaattaaggggtggtgtttagaatgttgtcaaatagctgatttaacccattgttatggttgctcattgccgcatgtttgcaaatggtgtgttcagaacagaagatgcttccttgacaatgaacctcatttgcttaagcttagaactgtgaaacatccaattaccaaagacaaattacagtgtatcatagacttgtacaatataatatttccaattaatgataaagtaattagaaaatttgaaagaatgataaagcaaagagaatgtaggaatcaatataaaattgaatggtataatcatttgctgctcccaattacattaaatgctgctgcatttaagtttgatgaaaataatctttattatgtttttgggttatatgagaaatcagtcagtgatatatatgctccatatagaattgttaactttataaatgaatttgataaattattgcttgatcatattaactttacaagaatgtccaatctaccaatagagttgagaaaccattacgcaaagaaatacttccaattatcaagactgccatcatcaaaactaaagcaaatttacttttcagattttactaaagaaactgtgatttttaatacttatacaaaaacgccaggaagatcaatatacagaaatgtaactgaatttaattggagagatgaattggagctttattctgatttaaaaaatgataagaataaattaattgctgcaatgatgacgagtaagtatactcggttctatgctcatgataataattttggaaggttgaaaatgacaatatttgagttgggacatcattgtcagcctaactacgtggcatctaatcacccaggcaatgcttccgatatccagtactgtaaatggtgtaatataaaatattttcttagtaaaattgattggcggattcgtgatatgtataatttattgatggaatttattaaggattgttataaaagtaatgttaacgttggacattgtagttctgttgaaaacatatatcctttaattaaaagattaatttggagtttgtttactaatcacatggatcaaacaattgaagaagtgtttaatcacatgtcgccagtgtcagttgaaggtacgaatgtcatcatgttgattcttggattgaatattagtttgtataatgaaattaagcgcactttgaatgtagatagcataccaatggtacttaatttaaatgaattcagtagtatagttaaatcaattagcagtaaatggtataatgttgatgaattggataaattgccaatgtcaataaaatcaacggaggaactgattgaaatgaagaattctggaactttaactgaagaatttgagctactgatctccaactcagaagatgacaatgagggctccggcgagggcaggggaagtcttctaacatgcggggacgtggaggaaaatcccggcccaatggaacttttgatactgaaagccaatgcaattacaacaatcctcacggccgtcactttctgtttcgcgtctgggcaaaatatcacagaagagttctaccaatctacatgttcagctgtgtctaaaggttaccttagcgccctccgcactggctggtatacatccgtcataactatcgagctctctaatataaaagaaaataagtgtaacggaacagacgctaaagtaaagcttattaaacaagagttggataagtataaaaacgcggtgacagaattgcagttgctgatgggtgggggttccgggggtgggtccgggggcggatctggaagcgccatagcttccggagtagcagtttgtaaggttttgcacttggagggtgaagttaataaaattaagtcagctctgctcagtactaataaagctgttgtcagcctcagcaacggtgtcagcgtcctcacgtttaaagtactcgatcttaaaaactatatagataaacaactgcttcccatacttaacaaacagtcatgcagcatctctaacattgaaacagttatcgagttccagcaaaagaataaccgccttctcgaaataacgcgggagttttctgtaaatgccggagttacgacgccagtaagcacatatatgctcacaaactccgaattgctgagtcttataaatgacatgccaataacaaacgatcagaagaagctgatgtctaataacgtccagattgtccgacaacagtcttactctattatgtgtataataaaagaggaagtgcttgcatacgtggtgcaactccctctgtacggagttatagacaccccctgctggaagctgcacacatctccactctgtactacgaacactaaagaaggttccaatatatgccttacccgaacagaccgcggctggtattgtgacaatgcagggagtgtatcattttttccccaagcggagacgtgtaaggttcaatctaatcgcgtcttctgtgacacaatgaatagcttgacacttccatccgaggtgaatctttgtaacgtcgacatctttaatcccaaatacgattgcaagatcatgaccagtaaaacagatgtttcatctagcgtaataacttcactcggcgcgattgtctcatgctacggaaagaccaaatgcacggcatccaataagaacagaggaattatcaagactttctccaatgggtgcgactacgtgagtaataaaggcgtcgatacagtgagtgtagggaatacgctgtattacgtgaacaaacaggagggaaaaagtttgtatgtgaagggagaacccataataaacttttacgatcccctcgtctttccctcttgtgaattctgtgcctcaatatctcaggtaaatgaaaaaataaatcaatctttggcctttatacgcaaaagcgacgaactcctgagtgcgattggaggatatattcccgaggccccccgcgacggacaggcctatgtaagaaaggacggggaatgggtactcttgagtacgttcctcggaggccttgtccccaggggatcccatcatcaccaccaccattccgcttggtcacatcctcaatttgagaaataatgaaattatgtcactatctaattatacagtatttagccatcacaagaccgtccagactagagtagcgcctagctggcaaaatactgtgaaccgggtcggcatggcatctccacctcctcgcggtccgacctgggcatccgaaggaggacgcacgtccactcggatggctaagggagagcctgcagtagcataaccccttggggcctctaaacgggtcttgaggggttttttgggtacc

SEQ ID NO: 46 - protein, 533 aa, Artificial Sequence
  Description of Artificial Sequence Synthetic polypeptide
  RSV-T4scPreF
MELLILKANAITTILTAVTFCFASGQNITEEFYQSTCSAVSKGYLSALRTGWYTSVITIELSNIKENKCNGTDAKVKLIKQELDKYKNAVTELQLLMGGGSGGGSGGGSGSAIASGVAVCKVLHLEGEVNKIKSALLSTNKAVVSLSNGVSVLTFKVLDLKNYIDKQLLPILNKQSCSISNIETVIEFQQKNNRLLEITREFSVNAGVTTPVSTYMLTNSELLSLINDMPITNDQKKLMSNNVQIVRQQSYSIMCIIKEEVLAYVVQLPLYGVIDTPCWKLHTSPLCTTNTKEGSNICLTRTDRGWYCDNAGSVSFFPQAETCKVQSNRVFCDTMNSLTLPSEVNLCNVDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSCEFCASISQVNEKINQSLAFIRKSDELLSAIGGYIPEAPRDGQAYVRKDGEWVLLSTFLGGLVPRGSHHHHHHSAWSHPQFEK

SEQ ID NO: 47 - DNA, 3567 bp, Artificial Sequence
  Description of Artificial Sequence Synthetic polynucleotide
  pT7/RSV-A2PreF
actagttaatacgactcactataggcttttttttgaaaagtcttgtgttagccatggctacttttaaagatgcatgctttcattatcgtagattaactgctttaaatcggagattatgcaacattggtgcaaattctatttggatgccagttcctgatgcgaaaattaaggggtggtgtttagaatgttgtcaaatagctgatttaacccattgttatggttgctcattgccgcatgtttgcaaatggtgtgttcagaacagaagatgcttccttgacaatgaacctcatttgcttaagcttagaactgtgaaacatccaattaccaaagacaaattacagtgtatcatagacttgtacaatataatatttccaattaatgataaagtaattagaaaatttgaaagaatgataaagcaaagagaatgtaggaatcaatataaaattgaatggtataatcatttgctgctcccaattacattaaatgctgctgcatttaagtttgatgaaaataatctttattatgtttttgggttatatgagaaatcagtcagtgatatatatgctccatatagaattgttaactttataaatgaatttgataaattattgcttgatcatattaactttacaagaatgtccaatctaccaatagagttgagaaaccattacgcaaagaaatacttccaattatcaagactgccatcatcaaaactaaagcaaatttacttttcagattttactaaagaaactgtgatttttaatacttatacaaaaacgccaggaagatcaatatacagaaatgtaactgaatttaattggagagatgaattggagctttattctgatttaaaaaatgataagaataaattaattgctgcaatgatgacgagtaagtatactcggttctatgctcatgataataattttggaaggttgaaaatgacaatatttgagttgggacatcattgtcagcctaactacgtggcatctaatcacccaggcaatgcttccgatatccagtactgtaaatggtgtaatataaaatattttcttagtaaaattgattggcggattcgtgatatgtataatttattgatggaatttattaaggattgttataaaagtaatgttaacgttggacattgtagttctgttgaaaacatatatcctttaattaaaagattaatttggagtttgtttactaatcacatggatcaaacaattgaagaagtgtttaatcacatgtcgccagtgtcagttgaaggtacgaatgtcatcatgttgattcttggattgaatattagtttgtataatgaaattaagcgcactttgaatgtagatagcataccaatggtacttaatttaaatgaattcagtagtatagttaaatcaattagcagtaaatggtataatgttgatgaattggataaattgccaatgtcaataaaatcaacggaggaactgattgaaatgaagaattctggaactttaactgaagaatttgagctactgatctccaactcagaagatgacaatgagggctccggcgagggcaggggaagtcttctaacatgcggggacgtggaggaaaatcccggcccaatggagttgctaatcctcaaagcaaatgcaattaccacaatcctcactgcagtcacattttgttttgcttctggtcaaaacatcactgaagaattttatcaatcaacatgcagtgcagttagcaaaggctatcttagtgctctgagaactggttggtataccagtgttataactatagaattaagtaatatcaaggaaaataagtgtaatggaacagatgctaaggtaaaattgataaaacaagaattagataaatataaaaatgctgtaacagaattgcagttgctcatgcaaagcacaccaccaacaaacaatcgagccagaagagaactaccaaggtttatgaattatacactcaacaatgccaaaaaaaccaatgtaacattaagcaagaaaaggaaaagaagatttcttgtttttttgttaggtgttggatctgcaatcgccagtggcgttgctgtatgtaaggtcctgcacctagaaggggaagtgaacaagatcaaaagtgctctactatccacaaacaaggctctagtcagcttatcaaatggagttagtgtcttaaccttcaaagtgttagacctcaaaaactatatagataaacaattgttacctattctgaacaagcaaagctgcagcatatcaaatatagaaactgtgatagagttccaacaaaagaacaacagactactagagattaccagggaatttagtgttaatgcaggtgtaactacacctgtaagcacttacatgttaactaatagtgaattattgtcattaatcaatgatatgcctataacaaatgatcagaaaaagttaatgtccaacaatgttcaaatagttagacagcaaagttactctatcatgtgcataataaaagaggaagtcttagcatatgtagtacaattaccactatatggtgttatagatacaccctgttggaaactacacacatcccctctatgtacaaccaacacaaaagaagggtccaacatctgtttaacaagaactgacagaggatggtactgtgacaatgcaggatcagtatctttcttcccacaagctgaaacatgtaaagttcaatcaaatcgagtattttgtgacacaatgaacagtttaacattaccaagtgaaataaatctctgcaatgttgacatattcaaccccaaatatgattgtaaaattatgacttcaaaaacagatgtaagcagctccgttatcacatctctaggagccattgtgtcatgctatggcaaaactaaatgtacagcatccaataaaaatcgtggaatcataaagacattttctaacgggtgcgattatgtatcaaataaagggatggacactgtgtctgtaggtaacacattatattatgtaaataagcaagaaggtaaaagtctctatgtaaaaggtgaaccaataataaatttctatgacccattagtattcccctctgatgaatttgatgcatcaatatctcaagtcaacgagaagattaaccagagcctagcatttattcgtaaatccgatgaattattacataatgtaaatgctggtaaatccaccacaaatatcatgataactactataattatagtgattatagtaatattgttatcattaattgctgttggactgctcttatactgtaaggccagaagcacaccagtcacactaagcaaagatcaactgagtggtataaataatattgcatttagtaactaatgaaattatgtcactatctaattatacagtatttagccatcacaagaccgtccagactagagtagcgcctagctggcaaaatactgtgaaccgggtcggcatggcatctccacctcctcgcggtccgacctgggcatccgaaggaggacgcacgtccactcggatggctaagggagagcctgcagtagcataaccccttggggcctctaaacgggtcttgaggggttttttgggtacc

SEQ ID NO: 48 - protein, 574 aa, Artificial Sequence
  Description of Artificial Sequence Synthetic polypeptide
  RSV-A2PreF
MELLILKANAITTILTAVTFCFASGQNITEEFYQSTCSAVSKGYLSALRTGWYTSVITIELSNIKENKCNGTDAKVKLIKQELDKYKNAVTELQLLMQSTPPTNNRARRELPRFMNYTLNNAKKTNVTLSKKRKRRFLVFLLGVGSAIASGVAVCKVLHLEGEVNKIKSALLSTNKALVSLSNGVSVLTFKVLDLKNYIDKQLLPILNKQSCSISNIETVIEFQQKNNRLLEITREFSVNAGVTTPVSTYMLTNSELLSLINDMPITNDQKKLMSNNVQIVRQQSYSIMCIIKEEVLAYVVQLPLYGVIDTPCWKLHTSPLCTTNTKEGSNICLTRTDRGWYCDNAGSVSFFPQAETCKVQSNRVFCDTMNSLTLPSEINLCNVDIFNPKYDCKIMTSKTDVSSSVITSLGAIVSCYGKTKCTASNKNRGIIKTFSNGCDYVSNKGMDTVSVGNTLYYVNKQEGKSLYVKGEPIINFYDPLVFPSDEFDASISQVNEKINQSLAFIRKSDELLHNVNAGKSTTNIMITTIIIVIIVILLSLIAVGLLLYCKARSTPVTLSKDQLSGINNIAFSN

SEQ ID NO: 49 - DNA, 3459 bp, Artificial Sequence
  Description of Artificial Sequence Synthetic polynucleotide
  pT7/RSV-A2scPreF
actagttaatacgactcactataggcttttttttgaaaagtcttgtgttagccatggctacttttaaagatgcatgctttcattatcgtagattaactgctttaaatcggagattatgcaacattggtgcaaattctatttggatgccagttcctgatgcgaaaattaaggggtggtgtttagaatgttgtcaaatagctgatttaacccattgttatggttgctcattgccgcatgtttgcaaatggtgtgttcagaacagaagatgcttccttgacaatgaacctcatttgcttaagcttagaactgtgaaacatccaattaccaaagacaaattacagtgtatcatagacttgtacaatataatatttccaattaatgataaagtaattagaaaatttgaaagaatgataaagcaaagagaatgtaggaatcaatataaaattgaatggtataatcatttgctgctcccaattacattaaatgctgctgcatttaagtttgatgaaaataatctttattatgtttttgggttatatgagaaatcagtcagtgatatatatgctccatatagaattgttaactttataaatgaatttgataaattattgcttgatcatattaactttacaagaatgtccaatctaccaatagagttgagaaaccattacgcaaagaaatacttccaattatcaagactgccatcatcaaaactaaagcaaatttacttttcagattttactaaagaaactgtgatttttaatacttatacaaaaacgccaggaagatcaatatacagaaatgtaactgaatttaattggagagatgaattggagctttattctgatttaaaaaatgataagaataaattaattgctgcaatgatgacgagtaagtatactcggttctatgctcatgataataattttggaaggttgaaaatgacaatatttgagttgggacatcattgtcagcctaactacgtggcatctaatcacccaggcaatgcttccgatatccagtactgtaaatggtgtaatataaaatattttcttagtaaaattgattggcggattcgtgatatgtataatttattgatggaatttattaaggattgttataaaagtaatgttaacgttggacattgtagttctgttgaaaacatatatcctttaattaaaagattaatttggagtttgtttactaatcacatggatcaaacaattgaagaagtgtttaatcacatgtcgccagtgtcagttgaaggtacgaatgtcatcatgttgattcttggattgaatattagtttgtataatgaaattaagcgcactttgaatgtagatagcataccaatggtacttaatttaaatgaattcagtagtatagttaaatcaattagcagtaaatggtataatgttgatgaattggataaattgccaatgtcaataaaatcaacggaggaactgattgaaatgaagaattctggaactttaactgaagaatttgagctactgatctccaactcagaagatgacaatgagggctccggcgagggcaggggaagtcttctaacatgcggggacgtggaggaaaatcccggcccaatggagctgctcatcctcaaggccaacgccatcaccaccattctgaccgctgtgaccttctgtttcgcttccggccagaacatcacagaagagttctaccagtccacatgcagcgccgtctccaagggatatctctccgctctgagaaccggctggtatacaagcgtgatcacaatcgaactgagcaatattaaggagaataagtgcaacggcaccgacgctaaggtcaagctgatcaagcaagagctcgacaaatacaagaacgctgtgaccgaactccagctgctgatgggcggcggcagcggcggcggcagcggcggcggcagcagcgctattgctagcggcgtggccgtgtgcaaggtcctccatctggagggagaggtcaacaagatcaagagcgctctgctgtccaccaacaaggctctggtgtccctcagcaacggagtgagcgtgctcaccttcaaagtgctcgatctgaaaaactacattgataagcagctgctgcccattctgaacaagcaaagctgcagcatcagcaatatcgagaccgtgatcgaatttcaacagaaaaacaatagactgctcgagatcacaagagaattttccgtgaatgccggagtgacaacccccgtgagcacctacatgctgaccaattccgagctgctgtccctcatcaacgacatgcccatcaccaacgaccagaagaagctcatgtccaacaacgtccagattgtgaggcagcagagctatagcattatgtgtattattaaggaggaggtgctggcctacgtggtccaactgcctctgtatggcgtcatcgacaccccttgctggaagctccatacaagccctctctgtaccacaaacaccaaagagggctccaacatttgtctgaccagaacagatagaggctggtattgtgataacgccggaagcgtcagcttctttccccaagccgagacatgcaaggtgcaatccaatagagtgttctgcgacaccatgaactctctgacactgcccagcgaaatcaatctgtgcaacgtcgacatcttcaaccccaagtacgactgcaagatcatgacctccaaaaccgacgtctccagcagcgtcatcacatctctgggcgccatcgtgagctgctatggcaagaccaaatgcaccgctagcaacaagaatagaggaatcatcaaaacctttagcaacggctgtgactacgtctccaacaagggaatggacacagtgtccgtgggcaacacactgtactatgtgaacaagcaagagggcaagtctctgtacgtcaaaggcgagcccatcatcaacttctatgaccccctcgtgttcccttcctgcgagttttgcgcttccatcagccaagtgaacgagaaaatcaaccagtctctggccttcattaggaagagcgacgagctgctccacaacgtgaacgccggcaagagcaccaccaacatcatgatcaccacaattatcatcgtgattattgtcattctgctgtctctgattgccgtgggactgctgctctattgcaaggctagatccacacccgtgacactgtccaaggatcagctgagcggcatcaacaacattgccttcagcaactaatgaaattatgtcactatctaattatacagtatttagccatcacaagaccgtccagactagagtagcgcctagctggcaaaatactgtgaaccgggtcggcatggcatctccacctcctcgcggtccgacctgggcatccgaaggaggacgcacgtccactcggatggctaagggagagcctgcagtagcataaccccttggggcctctaaacgggtcttgaggggttttttgggtacc

SEQ ID NO: 50 - protein, 538 aa, Artificial Sequence
  Description of Artificial Sequence Synthetic polypeptide
  RSV-A2scPreF
MELLILKANAITTILTAVTFCFASGQNITEEFYQSTCSAVSKGYLSALRTGWYTSVITIELSNIKENKCNGTDAKVKLIKQELDKYKNAVTELQLLMGGGSGGGSGGGSSAIASGVAVCKVLHLEGEVNKIKSALLSTNKALVSLSNGVSVLTFKVLDLKNYIDKQLLPILNKQSCSISNIETVIEFQQKNNRLLEITREFSVNAGVTTPVSTYMLTNSELLSLINDMPITNDQKKLMSNNVQIVRQQSYSIMCIIKEEVLAYVVQLPLYGVIDTPCWKLHTSPLCTTNTKEGSNICLTRTDRGWYCDNAGSVSFFPQAETCKVQSNRVFCDTMNSLTLPSEINLCNVDIFNPKYDCKIMTSKTDVSSSVITSLGAIVSCYGKTKCTASNKNRGIIKTFSNGCDYVSNKGMDTVSVGNTLYYVNKQEGKSLYVKGEPIINFYDPLVFPSCEFCASISQVNEKINQSLAFIRKSDELLHNVNAGKSTTNIMITTIIIVIIVILLSLIAVGLLLYCKARSTPVTLSKDQLSGINNIAFSN

SEQ ID NO: 51 - DNA, 28 bp, Artificial Sequence
  Description of Artificial Sequence Synthetic primer
  RA-PCR forward primer
caacggaggaactgattgaaatgaagaa

SEQ ID NO: 52 - DNA, 19 bp, Artificial Sequence
  Description of Artificial Sequence Synthetic primer
  RA-PCR reverse primer
ttgccagctaggcgctact

SEQ ID NO: 53 - DNA, 26 bp, Artificial Sequence
  Description of Artificial Sequence Synthetic primer
  Sequencing forward primer
gctactgatctccaactcagaagatg

SEQ ID NO: 54 - DNA, 20 bp, Artificial Sequence
  Description of Artificial Sequence Synthetic primer
  Sequencing reverse primer
tagtctggacggtcttgtga

SEQ ID NO: 55 - DNA, 3009 bp, Artificial Sequence
  Description of Artificial Sequence Synthetic polynucleotide
  pT7/NSP1-GFP-NSP1repeat
actagttaatacgactcactataggcttttttttgaaaagtcttgtgttagccatggctacttttaaagatgcatgctttcattatcgtagattaactgctttaaatcggagattatgcaacattggtgcaaattctatttggatgccagttcctgatgcgaaaattaaggggtggtgtttagaatgttgtcaaatagctgatttaacccattgttatggttgctcattgccgcatgtttgcaaatggtgtgttcagaacagaagatgcttccttgacaatgaacctcatttgcttaagcttagaactgtgaaacatccaattaccaaagacaaattacagtgtatcatagacttgtacaatataatatttccaattaatgataaagtaattagaaaatttgaaagaatgataaagcaaagagaatgtaggaatcaatataaaattgaatggtataatcatttgctgctcccaattacattaaatgctgctgcatttaagtttgatgaaaataatctttattatgtttttgggttatatgagaaatcagtcagtgatatatatgctccatatagaattgttaactttataaatgaatttgataaattattgcttgatcatattaactttacaagaatgtccaatctaccaatagagttgagaaaccattacgcaaagaaatacttccaattatcaagactgccatcatcaaaactaaagcaaatttacttttcagattttactaaagaaactgtgatttttaatacttatacaaaaacgccaggaagatcaatatacagaaatgtaactgaatttaattggagagatgaattggagctttattctgatttaaaaaatgataagaataaattaattgctgcaatgatgacgagtaagtatactcggttctatgctcatgataataattttggaaggttgaaaatgacaatatttgagttgggacatcattgtcagcctaactacgtggcatctaatcacccaggcaatgcttccgatatccagtactgtaaatggtgtaatataaaatattttcttagtaaaattgattggcggattcgtgatatgtataatttattgatggaatttattaaggattgttataaaagtaatgttaacgttggacattgtagttctgttgaaaacatatatcctttaattaaaagattaatttggagtttgtttactaatcacatggatcaaacaattgaagaagtgtttaatcacatgtcgccagtgtcagttgaaggtacgaatgtcatcatgttgattcttggattgaatattagtttgtataatgaaattaagcgcactttgaatgtagatagcataccaatggtacttaatttaaatgaattcagtagtatagttaaatcaattagcagtaaatggtataatgttgatgaattggataaattgccaatgtcaataaaatcaacggaggaactgattgaaatgaagaattctggaactttaactgaagaatttgagctactgatctccaactcagaagatgacaatgagggctccggcgagggcaggggaagtcttctaacatgcggggacgtggaggaaaatcccggcccagtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaattattgatggaatttattaaggattgttataaaagtaatgttaacgttggacattgtagttctgttgaaaacatatatcctttaattaaaagattaatttggagtttgtttactaatcacatggatcaaacaattgaagaagtgtttaatcacatgtcgccagtgtcagttgaaggtacgaatgtcatcatgttgattcttggattgaatattagtttgtataatgaaattaagcgcactttgaatgtagatagcataccaatggtacttaatttaaatgaattcagtagtatagttaaatcaattagcagtaaatggtataatgttgatgaattggataaattgccaatgtcaataaaatcaacggaggaactgattgaaatgaagaattctggaactttaactgaagaatttgagctactgatctccaactcagaagatgacaatgagtgaaattatgtcactatctaattatacagtatttagccatcacaagaccgtccagactagagtagcgcctagctggcaaaatactgtgaaccgggtcggcatggcatctccacctcctcgcggtccgacctgggcatccgaaggaggacgcacgtccactcggatggctaagggagagcctgcagtagcataaccccttggggcctctaaacgggtcttgaggggttttttgggtacc

SEQ ID NO: 56 - DNA, 2176 bp, Artificial Sequence
  Description of Artificial Sequence Synthetic polynucleotide
  pT7/NSP2-GFP-NSP2repeat
aagctttaatacgactcactataggcttttaaagcgtctcagtcgccgtttgagccttgcggtgtagccatggctgagctagcttgcttttgctatcctcatttggagaatgatagctataaatttattccttttaataatttagctattaaagctatgctgacagctaaagtagacaaaaaggacatggataagttttatgattcaattatttatggaatagcaccgcctcctcaatttaagaaacggtataatactaatgataattcaagaggcatgaattttgaaacaattatgtttactaaggtggctatgttgatatgtgaagctctaaattcattgaaagtgacgcaagcaaacgtctctaatgtattatcacgagtagtatcaataaggcatttagaaaatttggtgatacgtaaagaaaatccacaggatattctatttcattcaaaagatttacttttgaaatcaacactgattgctattggacagtctaaagaaattgaaactacaataactgcagaaggaggagaaattgtatttcaaaacgctgccttcaccatgtggaaactaacttatttagaacatcaattgatgccaattctggatcagaattttattgaatataaagttacattgaacgaagataaaccaatttcagatgttcatgttaaagaattagtcgctgaacttcgatggcaatataacaagtttgctgtaatcacacatggtaagggtcattatagaattgtaaagtattcatcagttgctaatcacgctgacagagtatatgcaactttcaagagtaatgttaaaactggagttaataatgattttaacctacttgatcaaagaattatttggcaaaactggtatgcatttacatcatcaatgaaacagggtaatacacttgacgtgtgtaaaaggttgcttttccaaaaaatgaaaccagaaaaaaatccatttaaagggctgtcaacggatagaaaaatggacgaagtttctcaagttggcgttggctccggcgagggcaggggaagtcttctaacatgcggggacgtggaggaaaatcccggcccagtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaacaaagaattatttggcaaaactggtatgcatttacatcatcaatgaaacagggtaatacacttgacgtgtgtaaaaggttgcttttccaaaaaatgaaaccagaaaaaaatccatttaaagggctgtcaacggatagaaaaatggacgaagtttctcaagttggcgtttaattcgctatcaatttgaggatgatgatggcttagcaagaatagaaagcgcttatgtgaccgggtcggcatggcatctccacctcctcgcggtccgacctgggcatccgaaggaggacgcacgtccactcggatggctaagggagagcctgcagtagcataaccccttggggcctctaaacgggtcttgaggggttttttgggtacc

SEQ ID NO: 57 - DNA, 2219 bp, Artificial Sequence
  Description of Artificial Sequence Synthetic polynucleotide
  pT7/NSP3-GFP-NSP3repeat
actagttaatacgactcactataggcatttaatgcttttcagtggttgatgctcaagatggagtctacgcaacagatggccgtctcaattattaactcttcttttgaagctgcagttgtagctgcaacctcagctcttgagaatatgggaatagaatatgattatcaggatatatattctagagtaaagaataaatttgattttgtgatggacgattctggtgttaaaaataatctgattggtaaagcaataactattgatcaagctttgaataataaatttggatctgctataagaaatagaaactggcttgctgatacttctagagcagctaaattagatgaggatgtaaacaaactaagaatgatgttatcatcaaaaggaattgatcaaaaaatgagagttttaaacgcatgcttcagtgtaaaaagaatacctggaaaatcatcatctattattaaatgcacaaaattgatgcgtgataaattggaacgtggtgaagttgaagtggatgattcatttgtggatgaaaaaatggaagtggataccattgactggaaatcgcgctatgagcaattggagcaaaggtttgaatcattgaaatccagggtaaatgaaaaatataataattgggtgttgaaagcaagaaaaatgaatgaaaatatgcattctcttcaaaatgtcatctctcaacagcaagcacatatagctgagcttcaagtgtacaataataaactagaacgtgatttgcaaaataaaattggatcccttacttcttcgattgaatggtatttaagatcaatggaattagaccctgaaataaaggcagacattgaacagcaaattaactcaattgatgcgataaatccattgcacgcttttgatgacttagaatcagtaatacgtaatttgatatctgattatgacaaattattccttatgttcaaaggattaatacagagatgtaattatcaatattcatttggttgcgaaggctccggcgagggcaggggaagtcttctaacatgcggggacgtggaggaaaatcccggcccagtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaagacattgaacagcaaattaactcaattgatgcgataaatccattgcacgcttttgatgacttagaatcagtaatacgtaatttgatatctgattatgacaaattattccttatgttcaaaggattaatacagagatgtaattatcaatattcatttggttgcgaataaccattttgatacatgttgaacaatcaaatacagtgttagtatgttgtcatctatgcataaccctctatgagcacaatagttaaaagctaacactgtcaaaaacctaaatggctataggggcgttatgtggccgggtcggcatggcatctccacctcctcgcggtccgacctgggcatccgaaggaggacgcacgtccactcggatggctaagggagagcctgcagtagcataaccccttggggcctctaaacgggtcttgaggggttttttgggtacc

SEQ ID NO: 58 - DNA, 1865 bp, Artificial Sequence
  Description of Artificial Sequence Synthetic polynucleotide
  pT7/NSP4-GFP-NSP4repeat
actagttaatacgactcactataggcttttaaaagttctgttccgagagagcgcgtgcggaaagatggaaaagcttaccgacctcaattatacattgagtgtaatcactctaatgaacaatacattgcacacaatacttgaggatccaggaatggcgtattttccttatatagcatctgtcttaacagttttgtttgcgctacataaagcatccattccaacaatgaaaattgcattgaaaacgtcaaaatgttcatataaagtggtgaaatattgtattgtaacaatttttaatacgttgttaaaattggcaggttataaagagcagataactactaaagatgagatagaaaagcaaatggacagagtagtcaaagaaatgagacgccagctagaaatgattgacaaattgactacacgtgaaattgaacaagtagagttgcttaaacgcatttacgataaattgacggtgcaaacgacaggtgaaatagatatgacaaaagagatcaatcaaaaaaacgtgagaacgctagaagaatgggaaagtggaaaaaatccttatgaaccaagagaagtgactgcagcaatgggctccggcgagggcaggggaagtcttctaacatgcggggacgtggaggaaaatcccggcccagtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaattgaacaagtagagttgcttaaacgcatttacgataaattgacggtgcaaacgacaggtgaaatagatatgacaaaagagatcaatcaaaaaaacgtgagaacgctagaagaatgggaaagtggaaaaaatccttatgaaccaagagaagtgactgcagcaatgtaagaggttgagctgccgtcgactgtcctcggaagcggcggagttctttacagtaagcaccatcggacctgatggctgactgagaagccacagtcagccatatcgcgtgtggctcaagccttaatcccgtttaaccaatccggtcagcaccggacgttaatggaaggaacggtcttaatgtgaccgggtcggcatggcatctccacctcctcgcggtccgacctgggcatccgaaggaggacgcacgtccactcggatggctaagggagagcctgcagtagcataaccccttggggcctctaaacgggtcttgaggggttttttgggtacc

SEQ ID NO: 59 - DNA, 1781 bp, Artificial Sequence
  Description of Artificial Sequence Synthetic polynucleotide
  pT7/NSP4-GFP-NSP4repeat
aagctttaatacgactcactataggcttttaaagcgctacagtgatgtctctcagtattgacgtgacgagtcttccttctattccttcaactatatataagaatgaatcgtcttcaacaacgtcaactctttctggaaaatctattggtaggagtgaacagtacatttcaccagatgcagaagcattcaataaatacatgctgtcgaagtctccagaggatattggaccatctgattctgcttcaaacgatccactcaccagtttttcgattagatcgaatgcagttaagacaaacgcagacgctggcgtgtctatggattcatcagcacaatcacgaccttcaagtaatgtcggatgcgatcaagtggatttctccttaaataaaggcttaaaagtaaaagctaatttggactcatcaatatcaatatctacggatactaaaaaggagaaatcaaaacaaaaccataaaagtaggaagcactacccaagaattgaagcagagtctgattcagatgattatgtactggatgattcagatagtgatgatggtaaatgtaagaactgtaaatataagaagaaatacttcgcattaagaatgagaatgaaacaagtcgcaatgcaattgattgaagatttgggctccggcgagggcaggggaagtcttctaacatgcggggacgtggaggaaaatcccggcccagtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaaggaagcactacccaagaattgaagcagagtctgattcagatgattatgtactggatgattcagatagtgatgatggtaaatgtaagaactgtaaatataagaagaaatacttcgcattaagaatgagaatgaaacaagtcgcaatgcaattgattgaagatttgtaagtctgacctgggaacacactagggagctccccactcccgttttgtgaccgggtcggcatggcatctccacctcctcgcggtccgacctgggcatccgaaggaggacgcacgtccactcggatggctaagggagagcctgcagtagcataaccccttggggcctctaaacgggtcttgaggggttttttgggtacc

(19) United States
(12) Patent Application Publication
(10) Pub. No.: US 2024/0200099 A1
(43) Pub. Date: Jun. 20, 2024

(54) ROTAVIRUS VECTORS FOR HETEROLOGOUS GENE DELIVERY

(71) Applicant: Merck Sharp & Dohme LLC, Rahway, NJ (US)

(72) Inventors: Dai Wang, Blue Bell, PA (US); Jiajie Wei, Devon, PA (US)

(73) Assignee: Merck Sharp & Dohme LLC, Rahway, NJ (US)

(21) Appl. No.: 18/555,035

(22) PCT Filed: Apr. 11, 2022
(86) PCT No.: PCT/US2022/024189

§ 371 Date: Oct. 12, 2023

Related U.S. Application Data
(60) Provisional application No. 63/274,615, filed on Nov. 2, 2021.
(60) Provisional application No. 63/175,437, filed on Apr. 15, 2021.

(51) Int. Cl. C12N 15/86

(52) U.S. Cl. CPC C12N 15/86; C12N 2720/12322; C12N 2720/12343; C12N 2720/12352

(57) ABSTRACT

Rotavirus vectors encoding in their genome a heterologous gene, and nucleic acid constructs encoding such rotavirus vectors. The rotavirus vector genome may include a rotavirus non-structural protein, a 2A peptide downstream of the rotavirus non-structural protein, and a heterologous protein downstream of the 2A peptide. The heterologous gene may be, for example, a SARS-CoV-2 spike protein or a fragment thereof, or an RSV F protein or a fragment thereof.

### SEQUENCE LISTING

The instant application contains a Sequence Listing which has been submitted electronically in ASCII format and is hereby incorporated by reference in its entirety. Said ASCII copy, created on Mar. 31, 2022, is named 25217-WO-PCT_SL.txt and is 230,113 bytes in size.

### FIELD

This disclosure relates generally to rotavirus vectors useful for delivering immunogenic proteins.

### BACKGROUND

Live attenuated rotavirus is used as a vaccine in infants to reduce the likelihood of rotaviral infection, including ROTATEQR and ROTARIXR. Such live attenuated rotavirus may be given orally, a convenient route of administration relative to intramuscular or intradermal injection (see, e.g., U.S. Ser. No. 10/874,732B2 and U.S. Pat. No. 8,192,747B2). Recently, a plasmid-based reverse genetics system for rotavirus was developed that expresses a full set of viral proteins (Kanai et al., Proc Natl Acad Sci USA. 2017 Feb. 28:114(9):2349-2354). Kanai et al. used a split GFP system to fuse the NSP1 protein with the GFP 11 fragment of green fluorescent protein (GFP) and detect the NSP1-GFP11 fusion protein with the detector fragment GFP1-10. However, it is unclear if rotavirus can be harnessed to express longer full-length proteins.

It would be useful to be able to express heterologous antigenic proteins from a rotavirus vector.

### SUMMARY OF THE INVENTION

In one aspect, the present disclosure provides isolated nucleic acid molecules comprising: a promoter sequence; and a nucleic acid encoding: a rotavirus non-structural protein, a 2A peptide downstream of the rotavirus non-structural protein, and a heterologous protein downstream of the 2A peptide. In some embodiments, the nucleic acid encoding the rotavirus non-structural protein, the 2A peptide downstream of the rotavirus non-structural protein, and the heterologous protein downstream of the 2A peptide is a cDNA.

In some embodiments of the isolated nucleic acid molecule, the rotavirus non-structural protein is NSP1, NSP3, or NSP5. In some embodiments, the rotavirus non-structural protein is NSP1. In some embodiments, the rotavirus non-structural protein is NSP3. In some embodiments, the rotavirus non-structural protein is NSP5. In some embodiments, the encoded NSP1 protein has the amino acid sequence of SEQ ID NO: 14. In some embodiments, the encoded NSP3 protein has the amino acid sequence of SEQ ID NO: 18. In some embodiments, the encoded NSP5 protein has the amino acid sequence of SEQ ID NO: 22.

In some embodiments, the 2A peptide is T2A peptide (SEQ ID NO: 32). In some embodiments, the 2A peptide is P2A peptide (SEQ ID NO: 33). In some embodiments, the 2A peptide is E2A peptide (SEQ ID NO: 34). In some embodiments, the 2A peptide is F2A peptide (SEQ ID NO: 35).

In some embodiments of any one of the above aspect and embodiments, the isolated nucleic acid molecule comprises a nucleic acid encoding an antigenomic hepatitis delta ribozyme. In some embodiments of any one of the above aspect and embodiments, the promoter is a T7 promoter.

In some embodiments of any one of the above aspect and embodiments, the heterologous protein is a viral protein or fragment thereof. In some embodiments, the viral protein or fragment thereof is a SARS-COV-2 spike protein or a fragment thereof. In some embodiments, the viral protein or fragment thereof is the S1 domain of SARS-COV-2 spike protein (SEQ ID NO: 36) or the receptor binding domain of SARS-COV-2 spike protein (SEQ ID NO: 37). In some embodiments, the viral protein or fragment thereof is an RSV F protein or fragment thereof. In some embodiments, the viral protein or fragment thereof is RSV-T4PreF (SEQ ID NO: 44), RSV-T4scPreF (SEQ ID NO: 46), RSV-A2PreF (SEQ ID NO: 48), or RSV-A2scPreF (SEQ ID NO: 50).

In some embodiments of the above aspect, the heterologous protein is a reporter protein. In some embodiments of the above aspect, the heterologous protein is a fluorescent protein. In some embodiments, the fluorescent protein is: green fluorescent protein (GFP); enhanced GFP (eGFP); superfolder GFP; AcGFPl; ZsGreenl; enhanced blue fluorescent protein (EBFP), EBFP2, Azurite, mKalama; cyan fluorescent protein (CFP); enhanced CFP (ECFP); Cerulean; mHoneydew; CyPet; yellow fluorescent protein (YFP); Citrine; Venus; mBanana; ZsYellow1; Ypet; mOrange; tdTomato; LSSmOrange, PSmOrange PSmOrange2; DsRed; DsRed-monomer; DsRed-Express2; mRFPi; mCherry; mStrawberry; mRaspberry; niPluni; E2-Crimson; iRFP670; iRFP682; iRFP702; or iRFP720. In some embodiments, the fluorescent protein is GFP.

In a second aspect, the disclosure provides a recombinant rotavirus comprising in its genome a nucleic acid sequence encoding a 2A peptide downstream of NSP1, NSP3, or NSP5, and a heterologous gene downstream of the 2A peptide. In some embodiments, the nucleic acid sequence is a cDNA. In some embodiments, the heterologous gene is downstream of NSP1. In some embodiments, the heterologous gene is downstream of NSP3. In some embodiments, the heterologous gene is downstream of NSP5. In some embodiments, the encoded NSP1 protein has the amino acid sequence of SEQ ID NO: 14. In some embodiments, the encoded NSP3 has the amino acid sequence of SEQ ID NO: 18. In some embodiments, the encoded NSP5 protein has the amino acid sequence of SEQ ID NO: 22.

In some embodiments of the second aspect, the 2A peptide is T2A peptide (SEQ ID NO: 32). In some embodiments, the 2A peptide is P2A peptide (SEQ ID NO: 33). In some embodiments, the 2A peptide is E2A peptide (SEQ ID NO: 34). In some embodiments, the 2A peptide is F2A peptide (SEQ ID NO: 35).

In some embodiments of the second aspect, the heterologous gene encodes a viral protein or fragment thereof. In some embodiments, the viral protein or fragment thereof is a SARS-CoV-2 spike protein or a variant or fragment thereof. In some embodiments, the viral protein or fragment thereof is the S1 domain of SARS-COV-2 spike protein (SEQ ID NO: 36) or the receptor binding domain of SARS-COV-2 spike protein (SEQ ID NO: 37). In some embodiments, the viral protein or fragment thereof is an RSV F protein or a variant or fragment thereof. In one embodiment, the RSV F protein or a variant or fragment thereof is SEQ ID NO: 28. In some embodiments, the RSV F protein or a variant or fragment thereof is RSV-T4PreF (SEQ ID NO: 44), RSV-T4scPreF (SEQ ID NO: 46), RSV-A2PreF (SEQ ID NO: 48), or RSV-A2scPreF (SEQ ID NO: 50). In some embodiments, the viral protein or fragment thereof is RSV-T4PreF (SEQ ID NO: 44), RSV-T4scPreF (SEQ ID NO: 46), RSV-A2PreF (SEQ ID NO: 48), or RSV-A2scPreF (SEQ ID NO: 50).

In some embodiments of the second aspect, the heterologous gene encodes a reporter gene. In some embodiments of the second aspect, the heterologous gene encodes a fluorescent protein. In some embodiments, the fluorescent protein is: green fluorescent protein (GFP); enhanced GFP (eGFP); superfolder GFP; AcGFPl; ZsGreenl; enhanced blue fluorescent protein (EBFP), EBFP2, Azurite, mKalama; cyan fluorescent protein (CFP); enhanced CFP (ECFP); Cerulean; mHoneydew; CyPet; yellow fluorescent protein (YFP); Citrine; Venus; mBanana; ZsYellow1; Ypet; mOrange; tdTomato; LSSmOrange, PSmOrange PSmOrange2; DsRed; DsRed-monomer; DsRed-Express2; mRFPi; mCherry; mStrawberry; mRaspberry; niPluni; E2-Crimson; iRFP670; iRFP682; iRFP702; or iRFP720. In some embodiments, the fluorescent protein is GFP.

In one embodiment, the disclosure provides methods for measuring antibody neutralizing activity against rotavirus, comprising: a) combining the i) the recombinant rotavirus of the second aspect or the embodiments of the second aspect, and ii) one or more epithelial cells, and iii) one or more antibodies; and b) detecting expression of the reporter gene in the epithelial cells.

In some embodiments, the epithelial cells are Vero cells or CV-1 cells. In some embodiments, the epithelial cells are CV-1 cells. In some embodiments, the epithelial cells are Vero cells.

In some embodiments, the reporter gene is an enzyme, a fluorescent protein, or a protein detectable by an antibody binding interaction. In some embodiments, the reporter gene encodes a luciferase. In some embodiments, the reporter gene is a fluorescent protein. In some embodiments, the fluorescent protein is: green fluorescent protein (GFP); enhanced GFP (eGFP); superfolder GFP; AcGFPl; ZsGreenl; enhanced blue fluorescent protein (EBFP), EBFP2, Azurite, mKalama; cyan fluorescent protein (CFP); enhanced CFP (ECFP); Cerulean; mHoneydew; CyPet; yellow fluorescent protein (YFP); Citrine; Venus; mBanana; ZsYellow1; Ypet; mOrange; tdTomato; LSSmOrange, PSmOrange PSmOrange2; DsRed; DsRed-monomer; DsRed-Express2; mRFPi; mCherry; mStrawberry; mRaspberry; niPluni; E2-Crimson; iRFP670; iRFP682; iRFP702; or iRFP720. In some embodiments, the fluorescent protein is GFP.

In some embodiments, the disclosure provides an immunogenic composition comprising (i) an effective amount of the recombinant rotavirus of any one of the first and second aspect and their related embodiments, and (ii) a pharmaceutically acceptable carrier.

In some embodiments, the disclosure provides a method for treating or preventing an infection in a subject, comprising administering an effective amount of the immunogenic composition to the subject.

In some embodiments, the disclosure provides a method for inducing a protective immune response in a subject, comprising administering an effective amount of the immunogenic composition to the subject. In some embodiments, the immunogenic composition is administered to a mucous membrane of the subject. In some embodiments, administration of the immunogenic composition is oral.

In some embodiments of the above methods, the method comprises a first administration of the immunogenic composition and a second administration of the immunogenic composition. In some embodiments, the protective immune response is a humoral immune response and/or a cellular immune response. In some embodiments, the second administration is performed from one month to two months after the first administration. In some embodiments, the subject is a human.

In some embodiments, the disclosure provides for use of the recombinant rotavirus of any one of the above aspects and related embodiments or the immunogenic composition and its related embodiments above for preventing or treating an infection.

In some embodiments, the disclosure provides for the recombinant rotavirus of any one of the aspects or embodiments above or the immunogenic composition and its related embodiments above, for use in preventing or treating an infection in a subject.

In some embodiments, the disclosure provides for in vitro use of the recombinant rotavirus of any one of the above aspects and related embodiments or the immunogenic composition and its related embodiments for expressing the heterologous protein in eukaryotic cells.

In a third aspect, the disclosure provides a method for rescuing recombinant rotavirus, the method comprising: a) transfecting cells with i) eleven individual rotavirus genomic segment plasmids (RGSP), each RGSP having a promoter and encoding one of a single rotavirus protein VP1, VP2, VP3, VP4, NSP1, VP6, NSP3, NSP2, VP7, NSP4, or NSP5, wherein one or more of the plasmids encoding NSP1, NSP3, and NSP5 protein include a sequence encoding a 2A protein that is downstream of the NSP protein and a sequence encoding a heterologous protein that is downstream of the sequence encoding the 2A protein, and ii) five individual helper plasmids (HPs), each HP having a promoter and encoding one of a fusogenic Fusion-Associated Small Transmembrane (FAST) protein, RNA capping enzyme DIR, RNA capping enzyme D12L, NSP2 protein, or NSP5 protein; b) maintaining the transfected cells in conditions suitable for the production of recombinant rotavirus; and c) harvesting the resulting recombinant rotavirus. In some embodiments, the encoded NSP1 protein has the amino acid sequence of SEQ ID NO: 14. In some embodiments, the encoded NSP3 protein has the amino acid sequence of SEQ ID NO: 18. In some embodiments, the encoded NSP5 protein has the amino acid sequence of SEQ ID NO: 22. In some embodiments, the encoded VP1 protein has the amino acid sequence of SEQ ID NO: 2. In some embodiments, the encoded VP2 protein has the amino acid sequence of SEQ ID NO: 4. In some embodiments, the encoded VP3 protein has the amino acid sequence of SEQ ID NO: 6. In some embodiments, the encoded VP4 protein has the amino acid sequence of SEQ ID NO: 8. In some embodiments, the encoded VP6 protein has the amino acid sequence of SEQ ID NO: 10. In some embodiments, the encoded VP7 protein has the amino acid sequence of SEQ ID NO: 12.

In some embodiments of the third aspect, the 2A peptide is T2A peptide (SEQ ID NO: 32). In some embodiments, the 2A peptide is P2A peptide (SEQ ID NO: 33). In some embodiments, the 2A peptide is E2A peptide (SEQ ID NO: 34). In some embodiments, the 2A peptide is F2A peptide (SEQ ID NO: 35).

In some embodiments of the third aspect, the RGSPs comprise a nucleic acid encoding an antigenomic hepatitis delta ribozyme. In some embodiments, the promoter of the RGSPs is a T7 promoter. In some embodiments, the transfected cells are Vero cells. In some embodiments, the method comprises co-culturing the transfected cells of step (b) with cells that amplify replication of the recombinant rotavirus from the transfected cells. In some embodiments, the cells that amplify replication of the recombinant rotavirus from the transfected cells are MA104 cells.

The summary of the technology described above is non-limiting and other features and advantages of the technology will be apparent from the following detailed description, and from the claims.

### BRIEF DESCRIPTION OF THE FIGURES

FIG. 1 is a schematic diagram of the components used to rescue recombinant rotavirus from cells.

FIG. 2 shows scatterplots of mock infected cells (left panel) or wild type rSA11 infected cells (right panel), stained with Rotavirus VP6 antibody, and detected by flow cytometry.

FIG. 3A is a schematic diagram of a 2A-GFP sequence inserted between a rotavirus NSP ORF and a 3′ portion of the same NSP ORF. FIGS. 3B-3H show representative flow cytometry scatterplots of GFP expression from rSA11 strains encoding GFP at indicated genome segments.

FIG. 4A is a schematic representation of plasmids used for the recovery of rSA11 virus encoding GFP downstream from the NSP1 or the NSP5 rotavirus genome segment. FIG. 4B shows representative flow cytometry scatterplots of GFP expression from rSA11 strains encoding GFP at indicated genome segments.

FIG. 5A is a schematic representation of plasmids used for the recovery of recombinant rSA11 viruses containing either a SARS-COV2 Spike domain (e.g. spike receptor binding domain or S1 domain) or an RSV fusion protein (e.g. RSV-T4PreF, RSVT4scPreF, RSVA2PreF, or RSV-A2scPreF). FIG. 5B shows representative intracellular flow cytometry scatterplots of the expression of SARS-COV2 spike protein domains from recombinant rotavirus rSA11 strains encoding SARS-COV2 S protein RBD (CoV2-S-RBD) and S1 domain (CoV2-S-S1). FIG. 5C shows representative surface flow cytometry scatterplots for expression of SARS-COV2 spike protein domains from recombinant rotavirus rSA11 strains encoding SARS-COV2 S protein RBD (CoV2-S-RBD) and S1 domain (CoV2-S-S1). FIG. 5D shows representative intracellular flow cytometry scatterplots of the intracellular staining of RSV fusion proteins from recombinant rotavirus rSA11 strains expressing RSV-T4PreF, RSVT4scPreF, RSVA2PreF, or RSV-A2scPreF. FIG. 5E shows representative surface flow cytometry scatterplots of the cell surface staining of RSV fusion proteins from recombinant rotavirus rSA11 strains expressing RSV-T4PreF, RSVT4scPreF, RSVA2PreF, or RSV-A2scPreF.

FIG. 6A shows photographs of a series of gels comparing RT-PCR amplification products from recombinant rotavirus containing CoV2-S-RBD, CoV2-S-S1 and RSV-A2scPreF compared to wild type rotavirus, using primers flanking the insertion site. FIG. 6B is a line chart of the growth kinetics of various recombinant rotaviruses compared to wild type rotavirus (CoV2-S-RBD, CoV2-S-S1, and RSV-A2scPreF). FIG. 6C shows photographs of plaque formation on MA104 cells by various recombinant rotaviruses (Cov2-S-RBD, CoV2-S-S1, and RSV-A2scPreF) and wild type SA11 rotavirus.

FIG. 7A shows photographs of a gel comparing expected RT-PCR fragments for rSA11-WT and rSA11-GFP serially passaged ten times on MA104 cells. Expected band sizes are indicated in parentheses. FIG. 7B shows a graph of growth kinetics for MA104 cells infected with rSA11-WT and rSA11-GFP (expressed as the mean and range of duplicates). FIG. 7C shows photographs of plaque formation on MA104 cells by rSA11 and rSA11-GFP (data is representative of three independent experiments).

FIG. 8A shows representative serum neutralization curves of four simians. FIG. 8B shows a representative review of a 384-well plate. Wells are colored based on the numbers of GFP positive cells. FIG. 8C shows a graph of the correlation of neutralization titers and ELISA titers of serum samples from 12 African green monkeys.

FIG. 9A shows a histogram of serum neutralization titers (NT50) in twenty donors exposed to rSA11. FIG. 9B shows a dot plot of serum neutralization titers (NT50) of animal samples from indicated species. The bars indicate the median.

### DETAILED DESCRIPTION OF THE DISCLOSURE

Rotavirus is a genus of double-stranded RNA viruses in the family Reoviridae and is a mucosal viral vector which naturally infects the gastrointestinal tract. The rotavirus genus has nine species (A, B, C, D, F, G, H, I and J), with rotavirus A causing more than 90% of rotavirus infections in humans. Rotavirus has 11 genomic segments encoding six non-structural proteins (NSP1, NSP2, NSP3, NSP4, NSP5, and NSP6) and six structural viral proteins (VP1, VP2, VP3, VP4, VP6, and VP7). Segment 11 of the rotaviral genome encodes NSP5 and NSP6 from overlapping reading frames (ORFs). Following translation, VP4 is cleaved into two proteins, VP5* and VP8*.

The inventors identified two genome locations, the C-termini of NSP1 on segment 5 and NSP5 on segment 11, that can tolerate the insertion of foreign genes. In a reverse genetics system, the inventors replaced NSP1 (or NSP5) open reading frame (ORF) with an ORF encoding NSP1 (or NSP5) fused to a 2A peptide and green fluorescent protein (GFP). The 2A peptide leads to the separation of NSP1 (or NSP5) and GFP, which can be detected using microscopy or flow cytometry. This recombination strategy generates rotavirus that expresses a full set of viral proteins and foreign proteins, allowing the use of rotavirus as a mucosal vector to deliver transgenes to induce an immunogenic response, including transgenes encoding antigens from pathogens such as viruses and bacteria.

### Definitions

Listed below are definitions of various terms used herein. These definitions apply to the terms as they are used throughout this specification and claims, unless otherwise limited in specific instances, either individually or as part of a larger group.

Unless defined otherwise, all technical and scientific terms used herein have the same meaning as commonly understood by one of ordinary skill in the art. Generally, the nomenclature used herein and the laboratory procedures in cell culture, molecular genetics, organic chemistry, and peptide chemistry are those well-known and commonly employed in the art.

As used herein, the articles “a” and “an” refer to one or to more than one (i.e., to at least one) of the grammatical object of the article. By way of example, “an element” means one element or more than one element. Furthermore, use of the term “including” as well as other forms, such as “include,” “includes,” and “included,” is not limiting.

All ranges disclosed herein are inclusive of the recited endpoints and independently combinable (for example, the range of “from 50 mg to 500 mg” is inclusive of the endpoints, 50 mg and 500 mg, and all the intermediate values). The endpoints of the ranges and any values disclosed herein are not limited to the precise range or value; they are sufficiently imprecise to include values within 10% of these ranges and/or values, rounded up.

As used herein, the term “comprising” may include the embodiments “consisting of” and “consisting essentially of.” The terms “comprise(s),” “include(s),” “having,” “has,” “may,” “contain(s),” and variants thereof, as used herein, are intended to be open-ended transitional phrases, terms, or words that require the presence of the named ingredients/steps and permit the presence of other ingredients/steps. However, such description should be construed as also describing compositions or processes as “consisting of” and “consisting essentially of” the enumerated components, which allows the presence of only the named components or compounds, along with any acceptable carriers or fluids, and excludes other components or compounds.

“Administration” and “treatment,” as the terms apply to an animal, human, experimental subject, cell, tissue, organ, or biological fluid, refer to contact of an exogenous pharmaceutical, therapeutic, diagnostic agent, or composition to the animal, human, subject, cell, tissue, organ, or biological fluid. Treatment of a cell encompasses contact of a reagent to the cell, as well as contact of a reagent to a fluid, where the fluid is in contact with the cell. “Administration” and “treatment” also means in vitro and ex vivo treatments, e.g., of a cell, by a reagent, diagnostic, binding compound, or by another cell.

“Prevent” or “preventing” means to administer a prophylactic agent, such as a composition containing any of the recombinant rotavirus vectors of the present invention, internally or externally to a subject or patient at risk of becoming infected by a pathogen, for which the agent has prophylactic activity. Preventing includes reducing the likelihood or severity of a subsequent pathogenic infection, ameliorating symptoms associated with pathogenic infection, and inducing immunity to protect against pathogenic infection. The amount of a prophylactic agent that is effective to ameliorate any particular disease symptom may vary according to factors such as the age, and weight of the patient, and the ability of the agent to elicit a desired response in the subject. Whether a disease symptom has been ameliorated can be assessed by any clinical measurement typically used by physicians or other skilled healthcare providers to assess the severity or progression status of that symptom or in certain instances will ameliorate the need for hospitalization.

A “subject” (alternatively referred to herein as a “patient”) refers to a mammal capable of being infected with an infectious agent, e.g., a virus. In preferred embodiments, the subject is a human. A subject can be treated prophylactically or therapeutically. Prophylactic treatment provides sufficient protective immunity to reduce the likelihood or severity of an infection or the effects thereof. Prophylactic treatment can be performed using a composition of the invention, as described herein. Therapeutic treatment can be performed to reduce the severity or prevent recurrence of an infection or the clinical effects thereof. The recombinant rotavirus of the invention can be administered to the general population or to those persons at an increased risk of infection.

As used herein, the terms “effective amount” and “effective dose” in reference to a dose or amount of a vaccine composition disclosed herein refers to a dose required to elicit a humoral and/or cellular immune response that significantly reduces the likelihood or severity of infectivity of an infectious agent, e.g., respiratory syncytial virus or SARS-COV-2 virus. In some embodiments, the effective dose is a dose listed in a package insert for the vaccine composition. When applied to an individual active ingredient administered alone, an effective dose refers to that ingredient alone. When applied to a combination, an effective dose refers to combined amounts of the active ingredients that result in the prophylactic effect, whether administered in combination, serially or simultaneously.

“Immunogenic protein” refers to a protein which is capable of inducing an immune response to the pathogen (e.g. virus) from which the protein is derived. The term “immunogenic protein or fragment thereof” refers to immunogenic protein and fragments of such proteins which are also immunogenic. Immunogenic proteins may include proteins from pathogens such as viruses, bacteria, fungi, protozoa, and worms. Immunogenic proteins may include the SARS-CoV-2 spike protein (SEQ ID NO: 25), the S1 domain of SARS-COV-2 spike protein (SEQ ID NO: 36), the receptor binding domain of SARS-COV-2 spike protein (SEQ ID NO: 37), RSV-T4PreF (SEQ ID NO: 44), RSV-T4scPreF (SEQ ID NO: 46), RSV-A2PreF (SEQ ID NO: 48), or RSV-A2scPreF (SEQ ID NO: 50).

The term “2A peptide” refers to viral oligopeptides that are 18-22 amino acids in length and mediate cleavage of different polypeptides encoded by polycistronic mRNA during translation in eukaryotic cells. Coding sequences (CDS) for 2A peptides can be inserted between coding sequences for two polypeptides, and ribozyme skipping of the formation of glycl-prolyl peptide bond at the C-terminus results in separation of the two polypeptides flanking the 2A peptide coding sequence (see Liu et al., Sci Rep. 2017 May 19:7(1):2193). A 2A peptide may be derived from various viruses, including but not limited to: T2A (Thosea asigna virus 2A; SEQ ID NO: 32, GSGEGRGSLLTCGDVEENPGP); P2A (porcine teschovirus-1 2A; SEQ ID NO: 33, GSGATNFSLLKQAGDVEENPGP); E2A (equine rhinitis A virus; SEQ ID NO: 34, GSGQCTNY ALLKLAGDVESNPGP); and foot-and-mouth disease virus (F2A; SEQ ID NO: 35, GSGVKQTLNFDLLKLAGDVESNPGP). In some embodiments, the GSG sequence at the N-terminal residues 1-3 can be removed, although this can decrease cleavage efficiency.

A “reporter gene” is a gene encoding a protein that is detectable by fluorescence, luminescence, color change, enzyme assay, or histochemistry. A “reporter protein” is a protein encoded by a reporter gene. For example, a reporter protein encoded by a reporter gene may be a fluorescent protein that fluoresces when exposed to a certain wavelength of light (e.g., GFP). A reporter protein may be an enzyme that catalyzes a reaction with a substrate to produce an observable change in that substrate. Enzymes such as luciferase (exemplary substrate luciferin) or β-lactamase (exemplary substrate CCF4) can cause luminescence or allow fluorescence on substrate cleavage, and enzymes such as β-galactosidase (exemplary substrate X-gal (5-bromo-4-chloro-3-indolyl-P-D-galactopyranoside)) and secreted alkaline phosphatase (exemplary substrate PNPP (p-Nitrophenyl Phosphate, Disodium Salt)) can result in a visualizable precipitate upon substrate cleavage. In some embodiments, a reporter protein is detectable by an antibody binding interaction.

The term “fluorescent protein” refers to a protein that emits light at some wavelength after excitation by light at another wavelength. Exemplary fluorescent proteins that emit in the green spectrum range include but are not limited to: green fluorescent protein (GFP); enhanced GFP (eGFP); superfolder GFP; AcGFPl; and ZsGreenl. Exemplary fluorescent proteins that emit light in the blue spectrum range include but are not limited to: enhanced blue fluorescent protein (EBFP), EBFP2, Azurite, and mKalama. Exemplary fluorescent proteins that emit light in the cyan spectrum range include but are not limited to: cyan fluorescent protein (CFP); enhanced CFP (ECFP); Cerulean; mHoneydew; and CyPet. Exemplary fluorescent proteins that emit light in the yellow spectrum range include but are not limited to: yellow fluorescent protein (YFP); Citrine; Venus; mBanana; ZsYellow 1; and Ypet. Exemplary fluorescent proteins that emit in the orange spectrum range include but are not limited to: mOrange; tdTomato; LSSmOrange, PSmOrange and PSmOrange2. Exemplary fluorescent proteins that emit light in the red and far-red spectrum range include but are not limited to: DsRed; DsRed-monomer; DsRed-Express2; mRFPi; mCherry; mStrawberry; mRaspberry; niPluni; E2-Crimson; iRFP670); iRFP682; iRFP702; iRFP720. Exemplary listings of fluorescent proteins and their characteristics may be found in Day and Davidson, Chem Soc Rev 2009 October; 38(10): 2887-2921, incorporated herein by reference.

As used herein the term “epithelial cells” refers to cells from an inner or outer membrane of an organ in the body. Epithelial cells may come from organ membranes including, but not limited to: skin, nose, mouth, lung, mammary gland, heart, trachea, esophagus, blood vessels, stomach, large intestine, small intestine, bladder, urinary tract, kidney, prostate, liver, pancreas, and gallbladder. Exemplary epithelial cell lines include, but are not limited to: human Primary Renal Cortical Epithelial Cells (HRCE; PCS-400-011™; American Tissue Culture Collection (ATCC), Manassas, VA); Primary Renal Proximal Tubule Epithelial Cells; Normal, Human (RPTEC; PCS-400-010™); MA-104 cells (CRL-2378.1™; ATCC); CV-1 cells (CCL-70™; ATCC); Vero cells (CCL-81™; ATCC); HIEC-6 cells (CRL-3266™; ATCC); intestine 407 cells (CCL-6™; ATCC); Hs1.Int cells (CRL-7820™; ATCC); and FHs 74 Int cells (CCL-241™; ATCC).

“Isolated nucleic acid molecule” or “isolated polynucleotide” means a DNA or RNA of genomic, mRNA, cDNA, or synthetic origin, or some combination thereof which is not associated with all or a portion of a polynucleotide in which the isolated polynucleotide is found in nature or is linked to a polynucleotide to which it is not linked in nature. For purposes of this disclosure, it should be understood that “a nucleic acid molecule comprising” a particular nucleotide sequence does not encompass intact chromosomes. Isolated nucleic acid molecules “comprising” specified nucleic acid sequences may include, in addition to the specified sequences, coding sequences for up to ten or even up to twenty or more other proteins or portions or fragments thereof or may include operably linked regulatory sequences that control expression of the coding region of the recited nucleic acid sequences, and/or may include vector sequences.

The phrase “control sequences” refers to DNA sequences necessary for the expression of an operably linked coding sequence in a particular host organism. The control sequences that are suitable for prokaryotes, for example, include a promoter, optionally an operator sequence, and a ribosome binding site. Eukaryotic cells are known to use promoters, polyadenylation signals, and enhancers.

A nucleic acid or polynucleotide is “operably linked” when it is placed into a functional relationship with another nucleic acid sequence. For example, DNA for a presequence or secretory leader is operably linked to DNA for a polypeptide if it is expressed as a preprotein that participates in the secretion of the polypeptide; a promoter or enhancer is operably linked to a coding sequence if it affects the transcription of the sequence; or a ribosome binding site is operably linked to a coding sequence if it is positioned so as to facilitate translation. Generally, but not always, “operably linked” means that the DNA sequences being linked are contiguous, and, in the case of a secretory leader, contiguous and in reading phase. However, enhancers do not have to be contiguous. Linking is accomplished by ligation at convenient restriction sites. If such sites do not exist, the synthetic oligonucleotide adaptors or linkers are used in accordance with conventional practice.

As used herein, the expressions “cell,” “cell line,” and “cell culture” are used interchangeably and all such designations include progeny. Thus, the words “transformants” and “transformed cells” include the primary subject cell and cultures derived therefrom without regard for the number of transfers. It is also understood that not all progeny will have precisely identical DNA content, due to deliberate or inadvertent mutations. Mutant progeny that have the same function or biological activity as screened for in the originally transformed cell are included. Where distinct designations are intended, it will be clear from the context.

As used herein, the term “variant” is a molecule that differs in its amino acid sequence or nucleic acid sequence relative to a native sequence or a reference sequence. Sequence variants may possess substitutions, deletions, insertions, or a combination of any two or three of the foregoing, at certain positions within the sequence, as compared to a native sequence or a reference sequence. Ordinarily, variants possess at least 50% identity to a native sequence or a reference sequence. In some embodiments, variants share at least 80% identity or at least 90%, 91%, 92%, 93%, 94%, 95%, 96%, 97%, 98% or 99% identity with a native sequence or a reference sequence.

The present disclosure provides several types of compositions that are polynucleotide or polypeptide based, including variants and derivatives. These include, for example, substitutional, insertional, deletion and covalent variants and derivatives. The term “derivative” is synonymous with the term “variant” and generally refers to a molecule that has been modified and/or changed in any way relative to a reference molecule or a starting molecule.

As such, polynucleotides encoding peptides or polypeptides containing substitutions, insertions and/or additions, deletions and covalent modifications with respect to reference sequences; in particular, the polypeptide sequences disclosed herein are included within the scope of this disclosure. For example, sequence tags or amino acids, such as one or more lysines, can be added to peptide sequences (e.g., at the N-terminal or C-terminal ends). Sequence tags can be used for peptide detection, purification or localization. Lysines can be used to increase peptide solubility or to allow for biotinylation. Alternatively, amino acid residues located at the carboxy and amino terminal regions of the amino acid sequence of a peptide or protein may optionally be deleted providing for truncated sequences. Certain amino acids (e.g., C-terminal residues or N-terminal residues) alternatively may be deleted depending on the use of the sequence, as for example, expression of the sequence as part of a larger sequence that is soluble or linked to a solid support.

“Substitutional variants” when referring to polypeptides, are those that have at least one amino acid residue in a native or starting sequence removed and a different amino acid inserted in its place at the same position. Substitutions may be single, where only one amino acid in the molecule has been substituted, or they may be multiple, where two or more (e.g., 3, 4 or 5) amino acids have been substituted in the same molecule.

As used herein the term “conservative amino acid substitution” refers to the substitution of an amino acid that is normally present in the sequence with a different amino acid of similar size, charge, or polarity. Examples of conservative substitutions include the substitution of a non-polar (hydrophobic) residue such as isoleucine, valine and leucine for another non-polar residue. Likewise, examples of conservative substitutions include the substitution of one polar (hydrophilic) residue for another such as between arginine and lysine, between glutamine and asparagine, and between glycine and serine. Additionally, the substitution of a basic residue such as lysine, arginine or histidine for another, or the substitution of one acidic residue such as aspartic acid or glutamic acid for another acidic residue are additional examples of conservative substitutions. Examples of non-conservative substitutions include the substitution of a non-polar (hydrophobic) amino acid residue such as isoleucine, valine, leucine, alanine, methionine for a polar (hydrophilic) residue such as cysteine, glutamine, glutamic acid or lysine and/or a polar residue for a non-polar residue. Those of skill in this art recognize that, in general, single amino acid substitutions in non-essential regions of a polypeptide do not substantially alter biological activity (see, e.g., Watson et al. (1987) Molecular Biology of the Gene, The Benjamin/Cummings Pub. Co., p. 224 (4th Ed.)). In addition, substitutions of structurally or functionally similar amino acids are less likely to disrupt biological activity. Exemplary conservative substitutions are set forth in Table 1 below.

TABLE 1
Exemplary Conservative Amino Acid Substitutions

| Original residue | Conservative substitution |
|---|---|
| Ala (A) | Gly; Ser |
| Arg (R) | Lys; His |
| Asn (N) | Gln; His |
| Asp (D) | Glu; Asn |
| Cys (C) | Ser; Ala |
| Gln (Q) | Asn |
| Glu (E) | Asp; Gln |
| Gly (G) | Ala |
| His (H) | Asn; Gln |
| Ile (I) | Leu; Val |
| Leu (L) | Ile; Val |
| Lys (K) | Arg; His |
| Met (M) | Leu; Ile; Tyr |
| Phe (F) | Tyr; Met; Leu |
| Pro (P) | Ala |
| Ser (S) | Thr |
| Thr (T) | Ser |
| Trp (W) | Tyr; Phe |
| Tyr (Y) | Trp; Phe |
| Val (V) | Ile; Leu |

As used herein when referring to polypeptides the term “domain” refers to a motif of a polypeptide having one or more identifiable structural or functional characteristics or properties (e.g., binding capacity, serving as a site for protein-protein interactions).

As used herein when referring to polypeptides the terms “site” as it pertains to amino acid-based embodiments is used synonymously with “amino acid residue” and “amino acid side chain.” As used herein when referring to polynucleotides the terms “site” as it pertains to nucleotide-based embodiments is used synonymously with “nucleotide.” A site represents a position within a peptide or polypeptide or polynucleotide that may be modified, manipulated, altered, derivatized, or varied within the polypeptide-based or polynucleotide-based molecules.

As used herein the terms “termini” or “terminus,” when referring to polypeptides or polynucleotides, refer to an extremity of a polypeptide or polynucleotide respectively. Such extremity is not limited only to the first or final site of the polypeptide or polynucleotide but may include additional amino acids or nucleotides in the terminal regions. Polypeptide-based molecules may be characterized as having both an N-terminus (terminated by an amino acid with a free amino group (NH2)) and a C-terminus (terminated by an amino acid with a free carboxyl group (COOH)). Proteins are in some cases made up of multiple polypeptide chains brought together by disulfide bonds or by non-covalent forces (multimers, oligomers). These proteins have multiple N- and C-termini. Alternatively, the termini of the polypeptides may be modified such that they begin or end with a non-polypeptide-based moiety such as an organic conjugate.

As used herein, the term “humoral immune response” refers to the generation of antibodies that exhibit one or more immune effector functions against a heterologous protein encoded in the genome of any of the recombinant rotavirus vectors described herein. Detection of a humoral immune response may be accomplished using a plaque reduction neutralization test (PRNT), in which serial dilutions of serum from subjects who have received a recombinant rotavirus of the invention are incubated with a pathogen the recombinant rotavirus is intended to generate an antibody response against. The mixture is then added to cultured cells susceptible to infection by the pathogen, and the plaques generated are counted. A PRNT50 value can be calculated as the dilution reducing the number of plaques to less than 50% of the control value (i.e. cells infected with virus only). An enzyme-linked immunospot (ELISpot) assay may also be used to measure the frequency of antibody-secreting cells at the single-cell level. In the ELISpot assay, peripheral blood mononuclear cells (PBMCs) collected from a subject's serum are cultured on a surface coated with the antigen of interest. The cultured PBMCs are removed and enzyme or fluorescent labeled secondary antibodies are added to visualize spots of antibody secretion and binding to plate-bound antigen where B cells secreted antibodies that bind the antigen of interest. Flow cytometry detection techniques may also be used (see e.g., Boonyaratanakornkit and Taylor, Front Immunol. 2019 Jul. 24:10; 1694).

As used herein, the term “cellular immune response” refers to the generation of antigen-specific T cells that exhibit one or more immune effector functions against a heterologous protein encoded in the genome of any of the recombinant rotavirus vectors described herein. Detection of a cellular immune response may be accomplished using an indirect or direct T cell assay to identify and measure a response in PBMCs collected from the blood of subjects that have received recombinant rotavirus vectors described herein. For example, a sandwich enzyme-linked immunosorbent assay (ELISA) may be used to detect T cell cytokines such as IFN-γ, IL-2, or IL-4 in subject serum. An ELISpot assay may also be used, which measures the frequency of cytokine-secreting cells in the PBMCs collected from subject serum. In the ELISpot assay, PBMCs are cultured on a surface coated with a capture antibody for a T cell cytokine (e.g. IFN-γ, IL-2, or IL-4), in the presence of molecules to stimulate T cells. The cultured PBMCs are then removed, and captured T cell cytokine is detected using enzyme or fluorescently labeled detection antibodies. Flow cytometry detection techniques may also be used (see Albert-Vega et al., Front Immunol. 2018 Oct. 16:9; 2367).

### Pharmaceutical Formulations

The invention also comprises pharmaceutical formulations comprising a recombinant rotavirus of the invention and a pharmaceutically acceptable carrier.

In one embodiment, the invention relates to a pharmaceutical composition comprising a recombinant rotavirus comprising in its genome a nucleic acid sequence (including but not limited to a cDNA sequence) encoding a 2A peptide downstream of NSP1, NSP3, or NSP5, and a heterologous gene downstream of the 2A peptide. In some embodiments of the recombinant rotavirus, the heterologous gene is downstream of NSP1. In some embodiments of the recombinant rotavirus, the heterologous gene is downstream of NSP3. In some embodiments of the recombinant rotavirus, the heterologous gene is downstream of NSP5. In some embodiments of the recombinant rotavirus, the heterologous gene encodes the S1 domain of SARS-COV-2 spike protein (SEQ ID NO: 36) or the receptor binding domain of SARS-COV-2 spike protein (SEQ ID NO: 37), RSV-T4PreF (SEQ ID NO: 44), RSV-T4scPreF (SEQ ID NO: 46), RSV-A2PreF (SEQ ID NO: 48), or RSV-A2scPreF (SEQ ID NO: 50).

As utilized herein, the term “pharmaceutically acceptable” means a non-toxic material that does not interfere with the effectiveness of the biological activity of the active ingredient(s), approved by a regulatory agency of the Federal or a state government or listed in the U.S.

Pharmacopoeia or other generally recognized pharmacopoeia for use in animals and, more particularly, in humans. The term “carrier” refers to a diluent, adjuvant, excipient, or vehicle with which the therapeutic is administered and includes but is not limited to such sterile liquids as water and oils. The characteristics of the carrier will depend on the route of administration.

Pharmaceutical formulations of therapeutic and diagnostic agents may be prepared by mixing with acceptable carriers, excipients, or stabilizers in the form of, e.g., lyophilized powders, slurries, aqueous solutions or suspensions (see, e.g., Hardman et al. (2001) Goodman and Gilman's The Pharmacological Basis of Therapeutics, McGraw-Hill, New York, N.Y.; Gennaro (2000) Remington: The Science and Practice of Pharmacy, Lippincott, Williams, and Wilkins, New York, N.Y.; Avis, et al. (eds.) (1993) Pharmaceutical Dosage Forms: Parenteral Medications, Marcel Dekker, NY; Lieberman, et al. (eds.) (1990) Pharmaceutical Dosage Forms: Tablets, Marcel Dekker, NY; Lieberman, et al. (eds.) (1990) Pharmaceutical Dosage Forms: Disperse Systems, Marcel Dekker, NY; Weiner and Kotkoskie (2000₎ Excipient Toxicity and Safety, Marcel Dekker, Inc., New York, N.Y.).

The mode of administration can vary. Suitable routes of administration include oral, rectal, transmucosal, intestinal, parenteral; intramuscular, subcutaneous, intradermal, intramedullary, intrathecal, direct intraventricular, intravenous, intraperitoneal, intranasal, intraocular, inhalation, insufflation, topical, cutaneous, transdermal, or intra-arterial.

In certain embodiments, the recombinant rotavirus of the invention can be administered by an invasive route such as by injection (see above). In some embodiments, the recombinant rotavirus of the invention, or pharmaceutical composition thereof, is administered intravenously, subcutaneously, intramuscularly, intraarterially, intra-articularly (e.g. in arthritis joints), intratumorally, or by inhalation, aerosol delivery. Administration by non-invasive routes (e.g., for example, as a liquid or aerosol, or in or capsule, or tablet) is also within the scope of the present invention. Doses of recombinant rotavirus may be administered, e.g., intravenously, subcutaneously, topically, orally, nasally, rectally, intramuscular, intracerebrally, intraspinally, or by inhalation. In some embodiments, recombinant rotavirus may be administered to a mucous membrane of a subject, e.g., orally or nasally.

Compositions can be administered with medical devices known in the art. For example, a pharmaceutical composition of the invention can be administered by injection with a hypodermic needle, including, e.g., a prefilled syringe or autoinjector.

The pharmaceutical compositions of the invention may also be administered with a needleless hypodermic injection device; such as the devices disclosed in U.S. Pat. Nos. 6,620,135; 6,096,002; 5,399,163; 5,383,851; 5,312,335; 5,064,413; 4,941,880; 4,790,824 or 4,596,556.

### EXAMPLES

The following examples are meant to be illustrative and should not be construed as further limiting. The contents of the figures and all references, patents, and published patent applications cited throughout this application are expressly incorporated herein by reference.

### Example 1: Overview of Reverse Genetics System

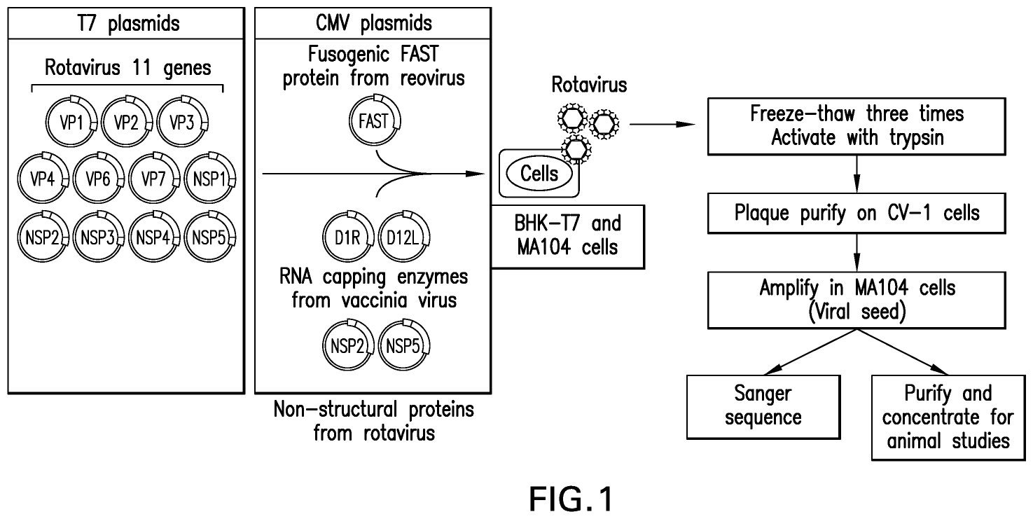

The reverse genetic system contains 11 rotavirus plasmids, each plasmid encoding one of six rotavirus genomic segments (SEQ ID NOs: 1, 3, 5, 7, 9, 11), and 5 helper plasmids that each encode a single helper protein: a fusogenic Fusion-Associated Small Transmembrane (FAST) protein from reovirus (SEQ ID NO: 26); two different RNA capping enzymes from vaccinia virus (SEQ ID NOs: 28, 30); and two additional rotavirus nonstructural proteins NSP2 and NSP5 (SEQ ID NOs: 24-25). The reverse genetics system was modified from Kanai et al. Proc Natl Acad Sci U.S.A. 2017 Feb. 28:114(9):2349-2354 to include additional helper plasmids encoding NSP2 and NSP5 to enhance the formation of viroplasm (see Kawagishi et al. J Virol. 2020 Jan. 6; 94(2): e00963-19). BHK-T7 cells (Buchholz et al., J Virol. 1999 January; 73(1):251-9.) were used for initial virus generation from plasmid transfection, and MA104 cells (Millipore Sigma Cat. #85102918) were co-cultured for virus amplification. After freeze-thaw cycles and trypsin activation, rotavirus was purified by plaque assay on CV-1 cells (CC-170™, American Type Culture Collection (ATCC), Manassas, VA), and further amplified in MA104 cells. Viral seed was confirmed by Sanger sequencing and purified. FIG. 1 is a schematic diagram of the components used to rescue recombinant rotavirus from cells.

### Example 2: Methods

### Cell Culture

CV1, MA104, and baby hamster kidney cells expressing T7 RNA polymerase (BHK-T7) were maintained in Dulbecco's Modified Eagle's Medium (DMEM) with 10% fetal bovine serum (FBS) and 1% penicillin-streptomycin. All cultures were grown at 37° ° C., in a 5% CO2 incubator.

### Plasmid Construction

Sequences of all 16 plasmids used for the generation of wild type SA11 strain were obtained from GenBank Acc. Nos. LC178564-LC178574. pUC19 is the backbone of 11 plasmids, each encoding one rotavirus genome segment insert; pT7/VPISA11 (SEQ ID NO: 1); pT7/VP2SA11 (SEQ ID NO: 3); pT7/VP3SA11 (SEQ ID NO: 5); pT7/VP4SA11 (SEQ ID NO: 7); pT7/VP6SA11 (SEQ ID NO: 9); pT7/VP7SA11 (SEQ ID NO: 11); pT7/NSPISA11 (SEQ ID NO: 13); pT7/NSP2SA11 (SEQ ID NO: 15); pT7/NSP3SA11 (SEQ ID NO: 17); pT7/NSP4SA11 (SEQ ID NO: 19); and pT7/NSP5SA11 (SEQ ID NO: 21). pV1Jns (SEQ ID NO: 23) is the backbone for each of the five helper plasmids, each helper plasmid containing one of the following inserts (inserted using the BglII restriction site): CMV/NSP2 (SEQ ID NO: 24); CMV/NSP5 (SEQ ID NO: 25); CMV/NBVFAST (SEQ ID NO: 26); CMV/D12L (SEQ ID NO: 28), and CMV/DIR (SEQ ID NO: 30). The 2A peptide sequence used was GSGEGRGSLLTCGDVEENPGP (SEQ ID NO: 32). The coding sequences for S1 domain of SARS-COV-2 spike protein (aa Met1-Pro681; SEQ ID NO: 36) and the receptor binding domain (RBD) of SARS-COV-2 spike protein (aa Met1-Cys15 and Arg319-Ser591; SEQ ID NO: 37) were designed based on GenBank: MN908947 (SEQ ID NO: 38), and the SARS-COV-2 spike protein (GenPept: QHD43416; SEQ ID NO: 39). RSV F protein plasmids used were T7/RSV-T4PreF (SEQ ID NO: 42; RSV F protein with DS-Cav1 mutations and T4 foldon at C terminus), T7/RSV-T4scPreF (SEQ ID NO: 45; RSV F protein with linker insertion to prevent furin cleavage, DS-Cav1 mutations, F111 mutations, T4 foldon at C terminus), T7/RSV-A2PreF (SEQ ID NO: 47; RSV F protein with DS-Cav1 mutations), and T7/RSV-A2scPreF (SEQ ID NO: 49; RSV F protein with linker insertion to prevent furin cleavage, DS-Cav1 mutations, F111 mutations); see Zhang et al., Vaccine. 2018 Dec. 18:36(52):8119-8130. All plasmids were synthesized by Genewiz.

### Recombinant Rotavirus Rescue

Recombinant SA1l (rSA11) strains were generated by reverse genetics as described previously with modifications (see Kanai et al., Proc Natl Acad Sci USA. 2017 Feb. 28:114(9):2349-2354). A monolayer of BHK-T7 cells in a 6-well plate (1×10⁶ cells/well) was used for transfection. Sixteen plasmids (0.75 μg/plasmid except 0.015 μg pCMV/NSVFAST) were mixed in 150 μl Opti-MEM and added to 150 μl Opti-MEM containing 12.5 μl Lipofectamine2000. Transfection complexes were incubated at room temperature for 20 minutes and then added drop-wise to BHK-T7 cells. 24 hours post transfection, culture medium was exchanged for serum free DMEM. 48 hours post transfection, 1.5×10⁵ MA104 cells were added to transfected cells and incubated for 3 days in serum free DMEM supplemented with 1 μg/ml trypsin. To generate recombinant viruses with foreign genes, pT7/NSPISA11, pT7/NSP2SA11, pT7/NSP3SA11, pT7/NSP4SA11, and pT7/NSP5SA11 were replaced with the corresponding plasmid with foreign gene insertion. Where indicated, 450 nucleotides at the 3′ end of the open reading frame were inserted after GFP. rSA11 strains were plaque purified for three rounds using MA104 cells.

### Virus Infection

Recombinant viruses were treated with 10 μg/ml trypsin at 37° C. for 1 hour. Cells were washed with serum free DMEM three times and infected with trypsin-treated viruses in serum free DMEM at 37° C. After 1 hour, the inoculums were removed.

### Plaque Assay

MA104 cells cultured in 6-well plates were infected with recombinant viruses and overlaid with 2 ml phenol-red free MEM containing 0.8% agarose and 0.5 μg/ml trypsin. After 4 days, plaques were visualized by adding 0.2 ml 5 mg/ml MTT in PBS or picked directly.

### Flow Cytometer and Data Analysis

CV1 cells in 12-well plate were infected with recombinant viruses and sub-cultured in DMEM containing 10% FBS. After overnight, cells were harvested, fixed with 4% paraformaldehyde, and stained with primary antibodies and then secondary antibodies. Antibodies used include anti-RotaVP6 (UK1, ThermoFisher), anti-RSVF (D25, Creative Biolabs), and anti-SARS-COV2-S-RBD (BS-R2B17, GenScript), Alexa Fluor 647 AffiniPure Goat Anti-Mouse IgG (H+L) (Jackson Immuno Research Labs, West Grove, PA), Alexa Fluor 488 AffiniPure Goat Anti-Human IgG (H+L) (Jackson Immuno Research Labs, West Grove, PA) and Alexa Fluor 488 AffiniPure Goat Anti-Rabbit IgG (H+L) (Jackson Immuno Research Labs, West Grove, PA). Staining and washing were performed in Perm/Wash buffer (BD Biosciences) or cell staining buffer (BioLegend) for intracellular staining or surface staining, respectively. Flow cytometric data were acquired using a BD LSRII flow cytometer (BD Biosciences) and gated on single cells. Data analysis was conducted using FlowJo version 10 software (FlowJo LLC).

### Growth Kinetics

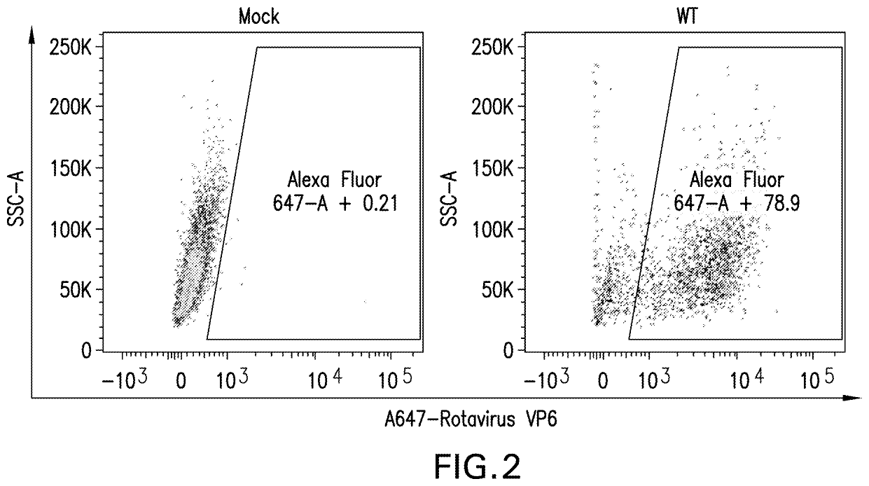

MA104 cells in 6-well plate were infected with recombinant viruses at a multiplicity of infection (MOI) of 0.01 infective units (IU)/cell, and sub-cultured in serum free DMEM supplemented with 1 μg/ml trypsin. Viruses were harvested at 6, 12, 24, 48, and 72 hours post-infection by freezing/thawing three times. Virus titer was determined by a flow-cytometry based infectivity assay as shown in the flow cytometry scatterplots of FIG. 2. Mock infected cells (FIG. 2, left scatterplot) and wild type (WT) rSA11 infected cells (FIG. 2, right scatterplot) were stained with Rotavirus VP6 antibody (Invitrogen; Rotavirus A VP6 Monoclonal Antibody (UK1) Catalog #MA5-16297). MOI was calculated using the percentage of VP6 positive cell population base on Poisson distribution. IU/ml was calculated using the formula below:

IU/ml=(# of cells at Infection)×[MOI/(ml of Viral Stock used at Infection)]

### RT-PCR

Viral RNA was extracted from 140 μl virus using QIAamp viral RNA kit, and 15 μl RNA was used in SuperScript IV one-step RA-PCR system with forward primer 5′-CAACGGAGGAACTGATTGAAATGAAGAA-3′ (SEQ ID NO: 51) and reverse primer 5′-TTGCCAGCTAGGCGCTACT-3′ (SEQ ID NO: 52) following manufacturers' instructions. PCR reactions were analyzed by 1.2% E-gel (ThermoFisher). E-Gel 1 Kb Plus Express DNA Ladder was used. Sanger sequence reactions were conducted by Genewiz using primers 5′-GCTACTGATCTCCAACTCAGAAGATG-3′ (SEQ ID NO: 53) and 5′-TAGTCTGGACGGTCTTGTGA-3′ (SEQ ID NO: 54).

### Example 2: Expressing Heterologous Genes from Recombinant Rotavirus

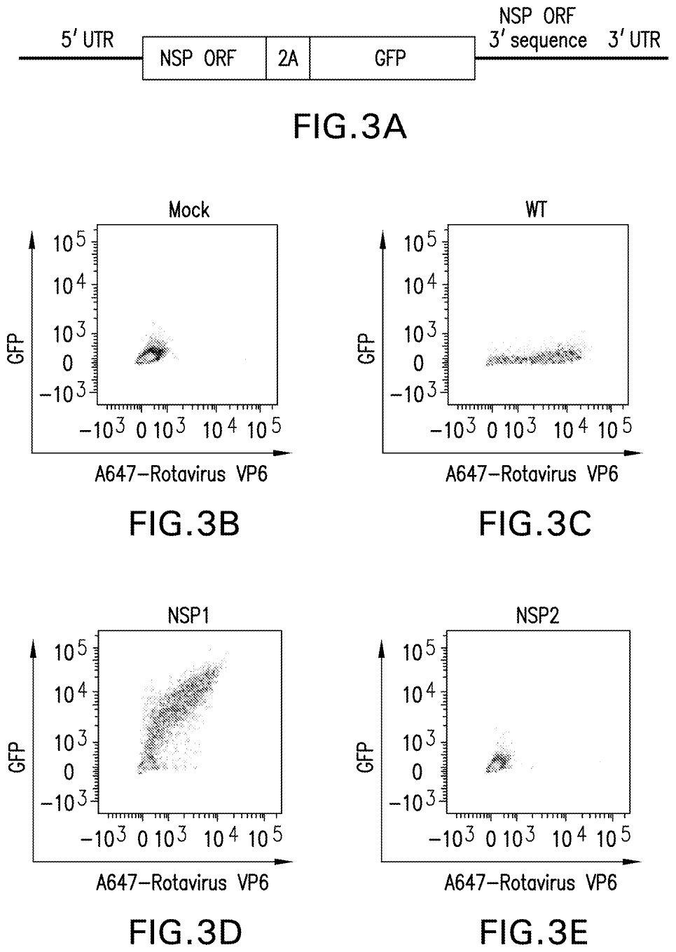

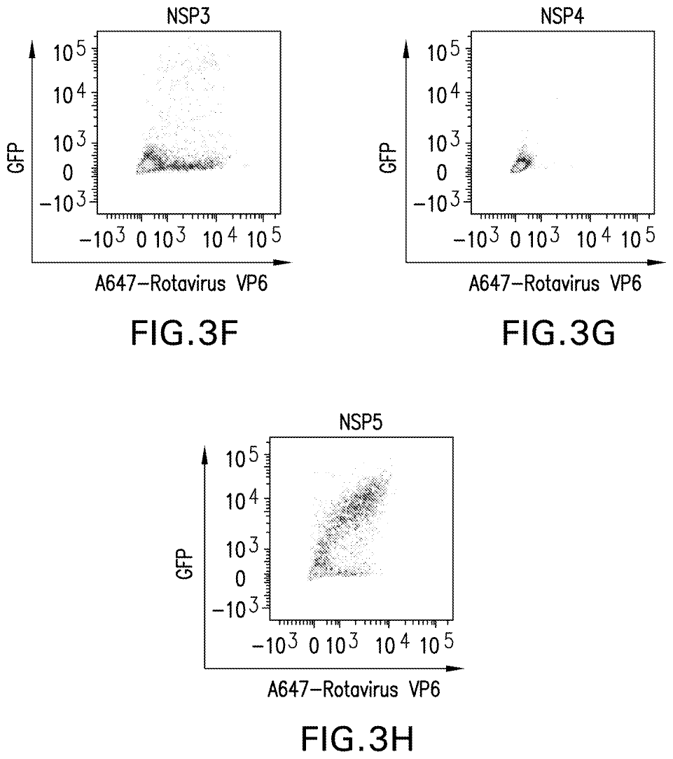

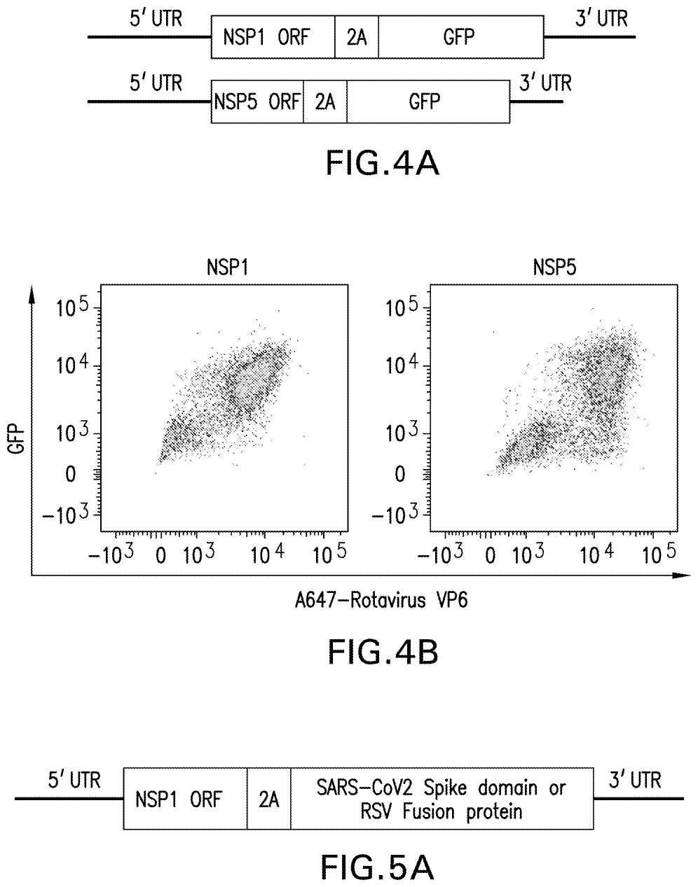

GFP coding sequence was inserted after a 2A self-cleavage sequence at the C termini of various rotavirus NSP ORFs; pT7/NSP1-GFP-NSP1repeat (SEQ ID NO: 55), pT7/NSP2-GFP-NSP2repeat (SEQ ID NO: 56), pT7/NSP3-GFP-NSP3repeat (SEQ ID NO: 57), pT7/NSP4-GFP-NSP4repeat (SEQ ID NO: 58), and pT7/NSP5-GFP-NSP5repeat (SEQ ID NO: 59). In each plasmid, partial NSP ORF sequence was repeated before the 3′ UTR. FIG. 3A is a schematic diagram of a 2A-GFP sequence inserted within portions of a rotavirus NSP ORF. For each recombinant NSP with inserted 2A-GFP sequence, rotavirus was then rescued using the techniques described herein. CV-1 cells were infected with each recombinant rotavirus, and viral protein VP6 and GFP expression was then determined by flow cytometry.

Cells infected with wild type (WT) virus show only VP6 signal (see FIG. 3B). Among all NSP proteins with inserted 2A-GFP sequence, NSP1. NSP3 and NSP5 showed GFP and VP6 double-positive CV1 cell populations, indicating that GFP gene insertion in these three locations was successful. Because the NSP1 and NSP5 recombinant rotavirus designs showed fewer VP6 single positive cells, these two genome positions were selected for further study.

Further testing showed that the NSP ORF 3′ sequence repeat can be deleted (data not shown). SEQ ID NO: 40 provides an example of pT7/NSPISA11-2A-GFP lacking the additional NSP1 ORF 3′ sequence repeat and SEQ ID NO: 41 provides an example of pT7/NSP5SA11-2A-GFP lacking the additional NSP1 ORF 3′ sequence repeat.

### Example 3: Expressing Heterologous Viral Antigens from Recombinant Rotavirus

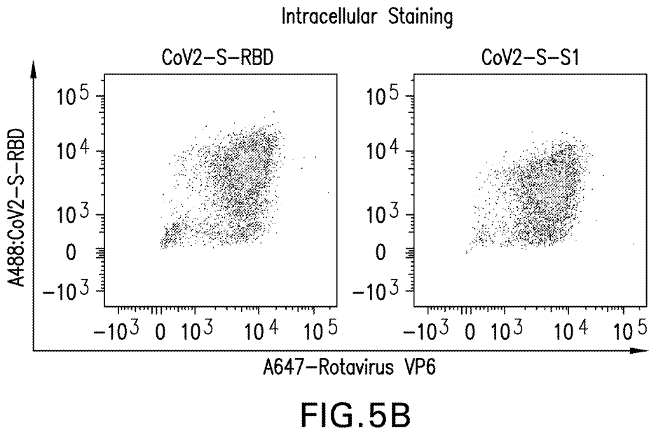

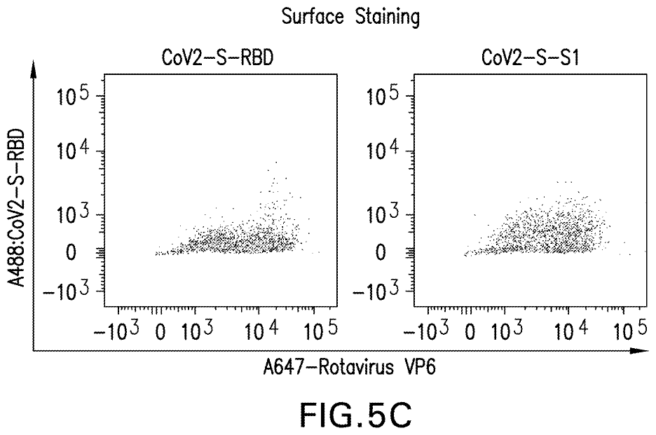

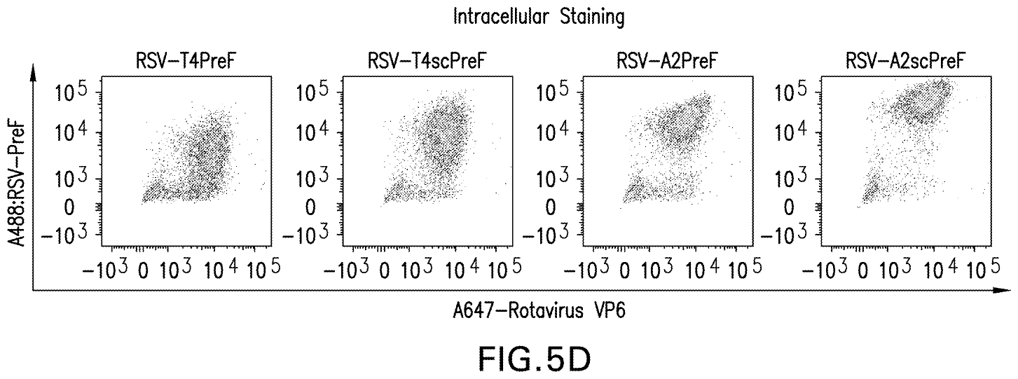

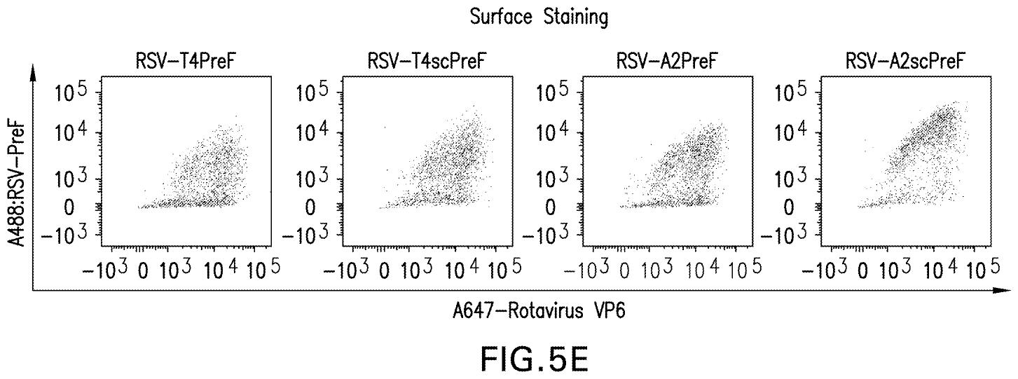

Viral polypeptides from SARS-COV-2 and from respiratory syncytial virus (RSV) were inserted after a 2A self-cleavage sequence at the C termini of rotavirus NSP1 ORF, and intracellular and cell surface expression of the viral polypeptides was measured by flow cytometry. FIG. 5A is a schematic diagram of a 2A-viral polypeptide sequence inserted within portions of a rotavirus NSP1 ORF. The SARS-COV-2 polypeptides were the receptor binding domain (RBD) of SARS-COV-2 spike protein (SEQ ID NO: 37) and the S1 domain of SARS-CoV-2 spike protein (SEQ ID NO: 36). RSV F protein plasmids used were T7/RSV-T4PreF (SEQ ID NO: 43; RSV F protein with DS-Cav1 mutations and T4 foldon at C terminus). T7/RSV-T4scPreF (SEQ ID NO: 45; RSV F protein with linker insertion to prevent furin cleavage. DS-Cav1 mutations. F111 mutations. T4 foldon at C terminus), T7/RSV-A2PreF (SEQ ID NO: 47; RSV F protein with DS-Cav1 mutations), and T7/RSV-A2scPreF (SEQ ID NO: 48; RSV F protein with linker insertion to prevent furin cleavage, DS-Cav1 mutations, F111 mutations).

With the signal peptide inserted upstream of SARS-COV2-RBD and S1, the inventors did not observe strong surface staining by flow cytometry, suggesting that most proteins are not membrane bound. In contrast, a significant portion of RSV-A2scPreF protein was observed by surface staining, especially for the constructs with a WT transmembrane domain. FIG. 5B shows representative intracellular flow cytometry scatterplots of the expression of recombinant rotavirus encoding SARS-COV2 S protein RBD (CoV2-S-RBD, left panel) and S1 domain (CoV2-S-S1, right panel). FIG. 5C shows representative surface flow cytometry scatterplots for expression of SARS-COV2 spike protein domains from recombinant rotavirus encoding SARS-COV2 S protein RBD (CoV2-S-RBD, left panel) and S1 domain (CoV2-S-S1, right panel).

FIG. 5D shows representative intracellular flow cytometry scatterplots of the intracellular staining of RSV fusion proteins from recombinant rotavirus rSA11 strains expressing RSV-T4PreF, RSVT4scPreF, RSVA2PreF, or RSV-A2scPreF. FIG. 5E shows representative surface flow cytometry scatterplots of the cell surface staining of RSV fusion proteins from recombinant rotavirus rSA11 strains expressing RSV-T4PreF, RSVT4scPreF, RSVA2PreF, or RSV-A2scPreF.

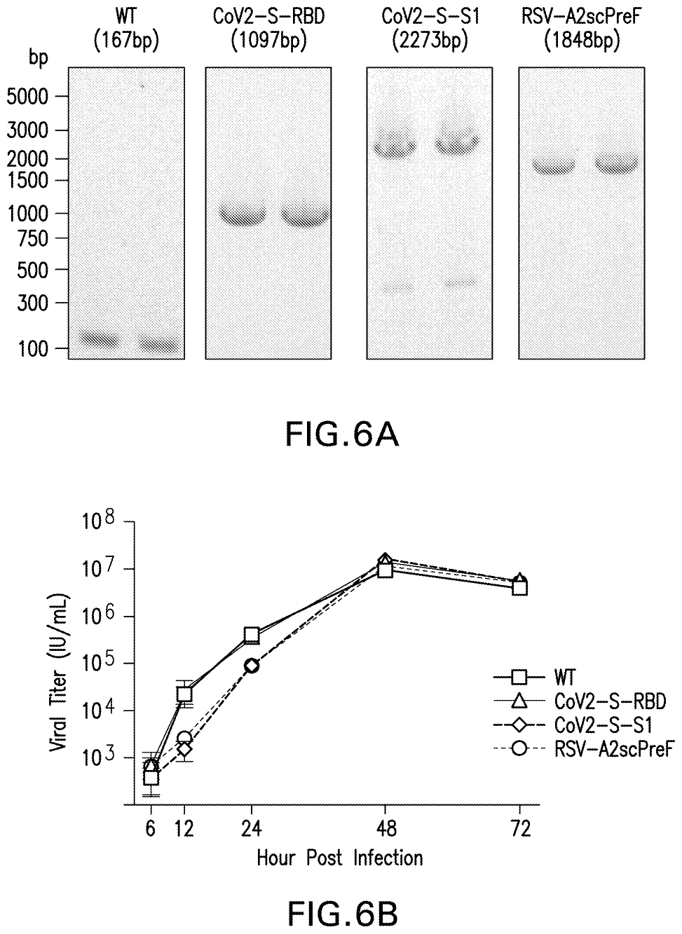

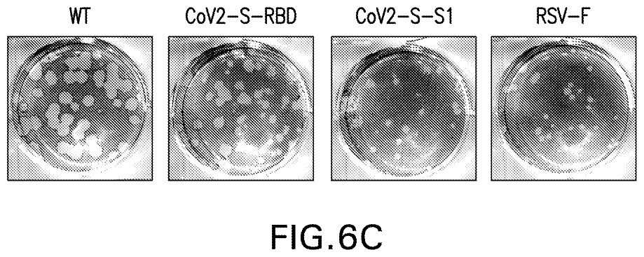

FIG. 6A shows photographs of a series of gels comparing RT-PCR amplification products from recombinant rotavirus containing CoV2-S-RBD, CoV2-S-S1 and RSV-A2scPreF compared to wild type rotavirus, using primers flanking the insertion site. The expected band sizes are indicated in parentheses. FIG. 6B is a line chart of the growth kinetics of various recombinant rotaviruses compared to wild type rotavirus (CoV2-S-RBD, CoV2-S-S1, and RSV-A2scPreF). MA104 cells were infected with viruses at an MOI of 0.01 IU/cell and harvested at 6, 12, 24, 48, and 72 hours post-infection. Virus titer was determined in an infectivity assay using CV1 cells. Data are expressed as the mean and range of duplicate samples. Growth kinetics of the recombinant rotavirus did not differ significantly from wild type rotavirus, indicating the insertion of foreign genes was not detrimental to the rotavirus. FIG. 5C shows photographs of plaque formation on MA104 cells by various recombinant rotaviruses (Cov2-S-RBD, CoV2-S-S1, and RSV-A2scPreF) and wild type SA1l rotavirus. The data is representative of three independent experiments.

### Example 4: Genetic Stability of rSA11-GFP

To examine the genetic stability of rSA11-GFP, the inventors passaged rSA11-GFP and rSA11-WT on MA104 cells ten times, extracted viral RNA from passage one (reverse genetics product) and ten, and performed RT-PCR using primers flanking the insertion site.

Viruses were serially passaged on MA104 cells. Monolayers of MA104 cells were infected with viruses and cultured in serum-free DMEM containing 1 μg/ml trypsin. When CPE reached completion, cell culture supernatant was used directly for the next round of infection with 1:1000 final dilution. Viral RNA was extracted from 140 μl supernatant using QIAamp viral RNA kit, and 15 μl RNA was used in the SuperScript IV one-step RA-PCR system with forward primer of SEQ ID NO: 51 and reverse primer of SEQ ID NO: 52 following manufacturers' instructions. PCR reactions were analyzed by 1.2% E-gel (ThermoFisher) along with E-Gel 1 Kb Plus Express DNA Ladder. Sanger sequencing reactions were conducted by Genewiz using primers of SEQ ID NO: 53 and 54.

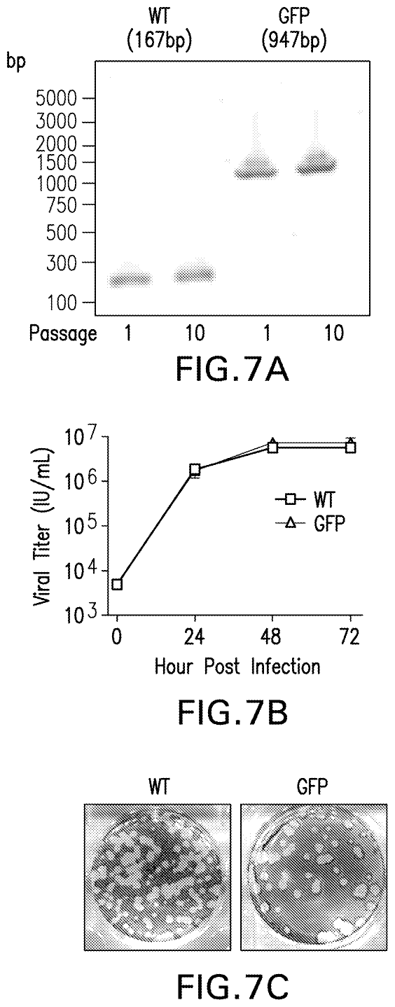

RT-PCR products were visualized by gel electrophoresis and sequenced by Sanger sequencing. Fragments migrated to expected sizes (FIG. 7A) and sequencing reactions showed that no DNA mutations were generated for 10 passages. The results indicated that rSA11-GFP was genetically stable.

The inventors also compared the growth kinetics of rSA11-GFP with rSA11-WT. MA104 cells were infected with recombinant viruses at a multiplicity of infection (MOI) of 0.01 IU/cell and cultured in serum free DMEM containing 1 μg/ml trypsin. Viruses were harvested at 24, 48, and 72 h post-infection by three freeze-thaw cycles. Virus titer was determined by a flow-cytometry based infectivity assay. The growth curves of rSA11-GFP and rSA11-WT were indistinguishable (FIG. 7B), indicating that the insertion of GFP did not affect the fitness of the recombinant virus. In addition, plaques formed by rSA11-GFP and rSA11-WT were of similar sizes (FIG. 7C), further supporting that the insertion of GFP downstream of NSP1 had no effects on rotavirus replication.

### Example 5: rSA11-Based Microneutralization Assay

Because traditional neutralization assays such as plaque reduction neutralization test (PRNT) and fluorescent foci reduction neutralization test (FRNT) that relies on antibody staining are time consuming and labor intensive, the inventors developed a microneutralization assay based on GFP signal using rSA11-GFP.

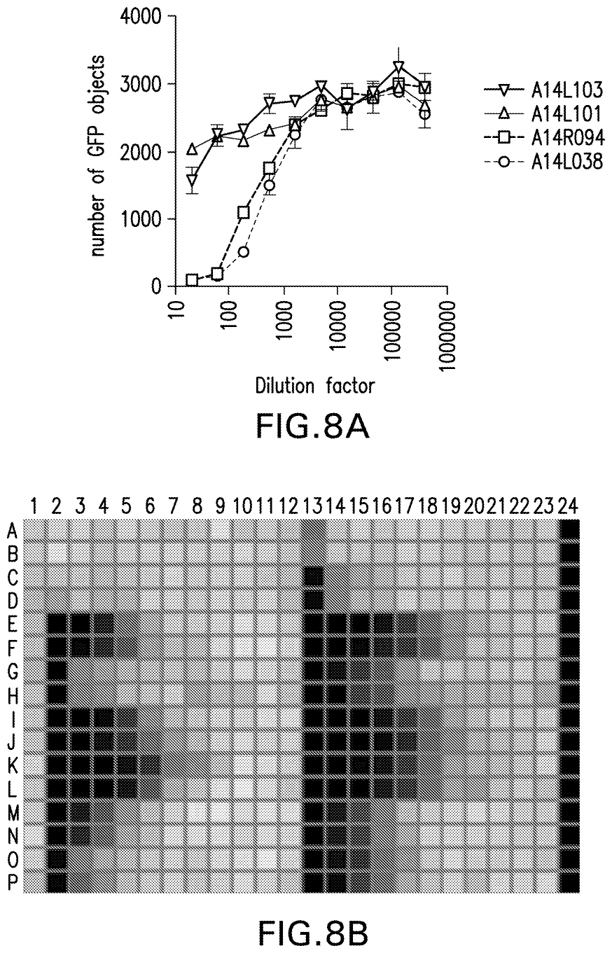

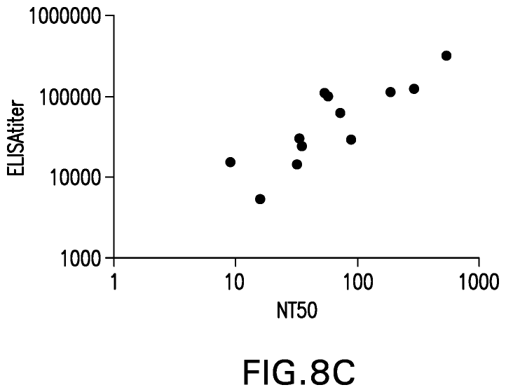

In the 96-well plate format, CV-1 cells were cultured overnight before virus infection. rSA11-GFP virus with an MOI of one was mixed with serial diluted animal serum samples for 1 h at 37° C. and then the virus/serum mixtures were applied to CV-1 cells for absorption. After overnight incubation, numbers of GFP positive cells were determined to generate neutralization curves. Percentages of inhibition were calculated based on control wells where no animal serum was added. It is known that immunity against rotavirus exists in some monkey colonies naturally (Shambaugh, C. et al. Development of a High-Throughput Respiratory Syncytial Virus Fluorescent Focus-Based Microneutralization Assay. Clin Vaccine Immunol 24, 2017). The inventors examined four rhesus monkey serum samples in the 96-well format microneutralization assay and found that, as expected, all four neutralized rSA11-GFP with two showing higher neutralizing capacity (FIG. 8A).

The inventors then converted the assay to a high-throughput format by adapting the assay to 384 well plates and eliminating the CV-1 pre-seeding step. This high-throughput assay was used to examine twelve African green monkeys. The higher-throughput assay was also compared to an ELISA assay.

For the 384 well neutralization assay, CV1 cells were harvested and washed in serum-free DMEM. 1×10+CV1 cells in suspension were added into virus/serum mixtures directly and incubated at 37° C. for 1 h. FBS was then added to the plate so the final concentration of FBS is 10%. For both 96 well and 384 well plates, after overnight incubation at 37° C., plates were read by an Acumen HCS reader at 488 nm to determine numbers of GFP positive cells in each well. NT50 was calculated by nonlinear four-parameter curve fitting using Prism 8 (GraphPad).

For ELISA, 96-well assay plates were coated with SA1l (105 PFU/well) in DMEM at 4° C. overnight. The plates were washed once with 300 μL/well Washing Buffer (PBS+0.05% Tween 20), and then blocked with 200 L/well Blocking Buffer (Alfa Aesar) at 4° C. overnight. Blocked plates were incubated with 100 μL/well a series of 3-fold diluted monkey sera in Blocking Buffer at 4° C. overnight. Upon the completion of sera incubation, the plates were washed three times with 300 μL/well Washing Buffer and incubated with 100 μL/well 1:4000 diluted alkaline phosphatase conjugated Goat anti-Rhesus IgG H&L (Southern Tech) in Blocking buffer with 0.1% Tween 20 for 1.5 h at room temperature. After washing the plates three times with Washing Buffer, 100 μL/well Tropix CDP-Star Sapphire II™ substrate (Applied Biosystem) were added. After incubation at room temperature for 10 min, chemiluminescent signal from each well was read on PHERAstar™. The threshold value was 25 times the mean plate background. Interpolated titers were calculated by drawing a line between the last point above the threshold and the first point below the threshold and solving for the fold dilution where that line crosses the threshold.

The inventors observed a wide range of antibody titers with NT50 titers ranging from 9 to 545 and ELISA titers ranging from 5,444 to 323,096. The results indicated that all African green monkeys examined were infected with rotavirus naturally. It is unlikely that the inventors are detecting maternal antibodies as all monkeys are 2-3 years old. The high level of correlation (r=0.9247, P<0.0001) between neutralization titer and ELISA titer (FIG. 8C) suggested that either almost all antibodies captured by ELISA were neutralizing antibodies or the proportions of rotavirus antibodies with neutralization capacity were consistent among African green monkeys.

The statistical analysis of FIG. 8C is shown in Table 2 below.

TABLE 2
Statistical analysis of FIG. 8C

| Pearson r | |
|---|---|
| r | 0.9247 |
| 95% confidence interval | 0.7474 to 0.9790 |
| R squared | 0.8550 |
| P value | |
| P (two-tailed) | <0.0001 |
| P value summary | **** |
| Significant? (alpha = 0.05) | Yes |
| Number of XY Pairs | 12 |

### Example 6: Pre-Existing Immunity in Human and Other Animal Species

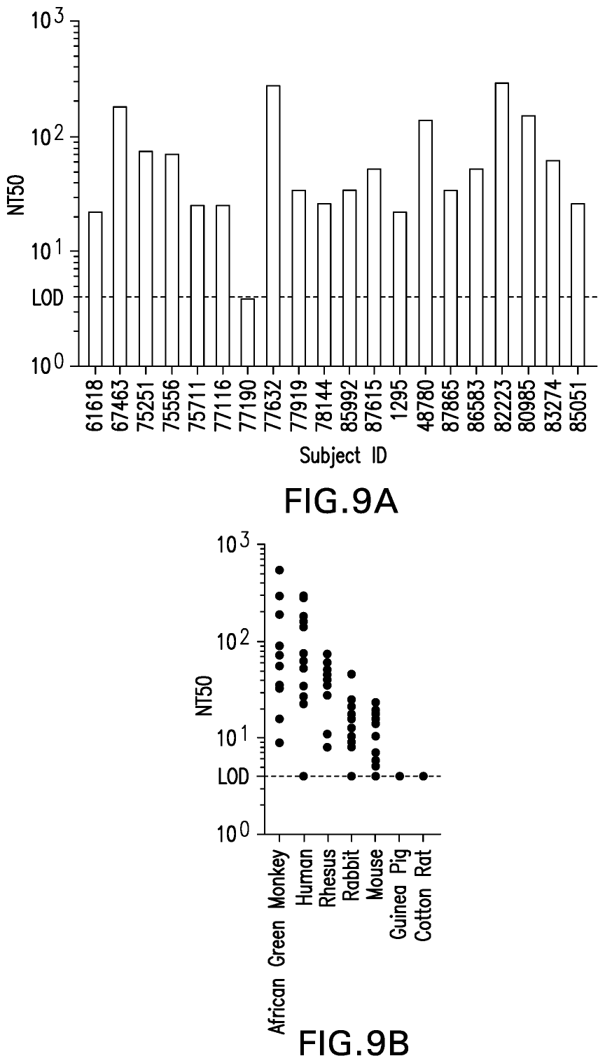

The inventors next determined neutralizing antibodies in human donors by the rSA11-GFP based microneutralization assay (FIG. 9A). Group A rotavirus contains more than twenty VP7 (G) serotypes and more than ten VP4 [P] serotypes (Fields, B. N. & Knipe, D. M. Fields virology Vol. 2, 2013). SA1l was originally isolated from a healthy African green monkey and belongs to G3P5B[2]. Serotypes G1, 2, 3, 4, 9 and 12 are epidemiologically important for human. G3 specific antibodies thus can be revealed by this assay as there is no known P5B human strain (Ibid.). Out of 20 samples examined, only one did not show neutralization titer above the limit of detection, suggesting the wild prevalence of G3 antibodies in human population. The titers were similar to those of African green monkeys in animal facility and higher than those of 11 rhesus monkeys examined. All rhesus monkeys examined showed neutralization titers as rhesus monkey specific rotavirus RRV also belongs to G3 (Ibid.).

The microneutralization assay also allowed evaluation of rabbit, mouse, guinea pig and cotton rat serum samples (FIG. 9B). Several rabbit rotaviruses are G3 serotype viruses and neutralizing antibodies were observed in many of the rabbits (15 out of 22) although the titers were much lower than those of human or simian. Surprisingly, although there is no known mouse rotavirus strain in the same serogroup as SA11, the inventors observed neutralization titers in some of the mouse serum samples (16 out of 25). Guinea pig and cotton rat are being used widely in infectious disease and vaccine research. No rotavirus has been reported in those two species. The inventors did not discover any neutralizing antibodies against SA11 in any guinea pig or cotton rat examined. In summary, the rSA11-GFP-based microneutralization assay enabled evaluation of pre-existing immunity in several animal species including human in a high-throughput manner.

TABLE 3
Relevant Sequences

| SEQ | | |
|---|---|---|
| ID NO | Description | Sequence |
| 1 | pT7/VP1SA11 | AAGCTTTAATACGACTCACTATAGGCTATTAAAGCTGTA |
| | T7 promoter: nt | CAATGGGGAAGTACAATCTAATCTTGTCAGAATATCTA |
| | 7-24 | TCATTTATATATAATTCACAATCTGCAGTTCAAATTCCA |
| | VP1 CDS: nt | ATATATTACTCTTCCAACAGTGAATTAGAAAATAGATGT |
| | 42-3,308 | ATTGAATTTCATTCCAAGTGTTTAGAGAACTCAAAGAAT |
| | HDV Ribozyme: | GGGTTATCGTTAAGAAAGTTGTTTGTTGAATATAATGAT |
| | nt 3,326-3,414 | GTCATAGAAAATGCCACATTACTGTCAATACTATCATAT |
| | T7 terminator: | TCTTACGACAAGTATAACGCTGTTGAAAGAAAATTGGT |
| | nt 3,423-3,465 | GAAGTATGCGAAAGGCAAACCATTGGAGGCAGACTTAA |
| | | CAGTGAATGAATTGGANTATGAGAACAATAAAATAACA |
| | | TCTGAATTATTTCCAACAGCGGAGGAATATACGGACTC |
| | | ACTAATGGATCCAGCAATTTTAACTTCGCTATCATCAAA |
| | | TTTAAATGCAGTCATGTTCTGGTTGGAAAAACATGAAA |
| | | ATGATGTCGCTGAAAAACTTAAAGTTTATAAAAGGAGA |
| | | TTAGACCTATTCACCATAGTAGCCTCAACGATAAATAA |
| | | ATATGGCGTACCAAGGCATAACGCAAAGTACAGATATG |
| | | AATACGACGTAATGAAAGATAAACCGTACTACTTAGTG |
| | | ACATGGGCAAATTCTTCAATTGAAATGTTAATGTCAGTT |
| | | TTCTCTCATGACGACTATTTGATAGCAAAAGAGTTAATA |
| | | GTGTTATCATATTCTAATAGATCTACTCTAGCAAAGTTA |
| | | GTGTCATCACCAATGTCGATTTTGGTAGCCTTGGTGGAT |
| | | ATTAATGGAACATTTATTACAAATGAAGAATTAGAATT |
| | | GGAATTTTCAAATAAATATGTACGAGCAATAGTTCCGG |
| | | ATCAAACATTTGACGAATTAAATCAAATGCTTGACAAT |
| | | ATGAGGAAAGCTGGATTAGTTGACATACCTAAGATGAT |
| | | ACAGGACTGGTTAGTTGATCGTTCTATCGAAAAATTTCC |
| | | ATTAATGGCTAAGATATATTCATGGTCGTTTCATGTTGG |
| | | ATTTAGAAAGCAAAAAATGCTAGATGCTGCGCTGGATC |
| | | AATTGAAAACTGAGTATACAGAAAATGTGGACGATGAA |
| | | ATGTATCGGGAATATACAATGTTAATAAGAGATGAAGT |
| | | AGTTAAAATGCTTGAAGAACCAGTTAAACATGATGATC |
| | | ACTTGCTACGAGATTCTGAGTTAGCTGGTTTACTATCAA |
| | | TGTCGTCAGCATCGAATGGTGAGTCAAGGCAGCTAAAG |
| | | TTTGGTAGGAAAACAATTTTTTCAACTAAAAAGAATAT |
| | | GCATGTCATGGATGATATGGCTAACGAAAGATACACGC |
| | | CTGGTATAATACCACCAGTGAATGTTGATAAACCAATA |
| | | CCATTAGGAAGAAGAGATGTTCCAGGAAGAAGGACTA |
| | | GAATAATATTCATTCTGCCATACGAATATTTCATAGCAC |
| | | AGCACGCTGTAGTTGAAAAAATGTTGATTTACGCAAAA |
| | | CATACGAGAGAATACGCTGAATTTTATTCACAATCAAA |
| | | CCAATTATTGTCATACGGCGATGTAACGCGTTTTTTGTC |
| | | TAATAACACAATGGTCTTGTATACGGATGTATCTCAGTG |
| | | GGATTCGTCTCAGCATAATACACAGCCATTTAGGAAAG |
| | | GAATAATAATGGGACTGGACATATTAGCTAACATGACT |
| | | AATGATGCTAAAGTTCTTCAGACATTAAACTTATACAA |
| | | ACAAACACAAATCAATCTCATGGATTCATACGTTCAAA |
| | | TACCAGATGGCAACGTCATTAAGAAAATACAATACGGG |
| | | GCAGTAGCATCAGGAGAGAAACAAACGAAAGCAGCAA |
| | | ATTCAATAGCAAATTTGGCACTGATTAAAACGGTTTTGT |
| | | CACGTATTTCTAACAAACATTCATTCGCAACAAAAATA |
| | | ATAAGAGTTGATGGAGATGATAACTATGCGGTGCTACA |
| | | ATTTAATACAGAGGTGACTAAGCAGATGATCCAAGACG |
| | | TATCGAACGATGTAAGAGAAACTTATGCACGCATGAAT |
| | | GCTAAAGTTAAAGCTCTGGTATCCACAGTAGGAATAGA |
| | | AATTGCTAAAAGGTACATTGCAGGTGGAAAAATATTTT |
| | | TTCGAGCTGGAATAAATCTACTTAATAATGAAAAGAGA |
| | | GGGCAGAGTACGCAGTGGGATCAAGCAGCAATTTTATA |
| | | TTCAAATTATATTGTAAATAGACTTAGAGGATTTGAAAC |
| | | TGATAGGGAGTTTATTTTAACTAAGATAATGCAGATGA |
| | | CGTCAGTCGCAATTACTGGATCATTAAGACTATTTCCTT |
| | | CTGAACGCGTATTAACTACGAATTCAACATTTAAAGTAT |
| | | TTGACTCGGAGGATTTTATTATAGAGTACGGAACGACT |
| | | GATGACGAAGTATATATACAAAGAGCGTTCATGTCTTT |
| | | ATCAAGTCAGAAATCAGGAATAGCCGATGAGATAGCGG |
| | | CATCATCAACATTTAAAAATTACGTCACGAGACTATCTG |
| | | AACAGTTATTATTTTCAAAGAATAATATAGTGTCCAGA |
| | | GGAATAGCTTTGACTGAAAAAGCGAAATTGAATTCATA |
| | | CGCTCCAATATCGCTTGAGAAAAGACGTGCACAGATAT |
| | | CAGCTTTATTGACTATGTTGCAGAAACCGGTCACCTTCA |
| | | AATCAAGTAAAATAACAATAAATGACATACTCAGAGAT |
| | | ATAAAACCATTTTTTACAGTAAGTGATGCACACTTACCT |
| | | ATACAATACCAAAAATTTATGCCAACTTTGCCAGATAA |
| | | CGTACAGTATATAATTCAATGTATAGGATCCAGAACTT |
| | | ATCAAATTGAAGATGACGGTTCGAAGTCAGCCATATCT |
| | | AGACTAATATCAAAGTATTCAGTTTATAAGCCATCAATT |
| | | GAAGAATTGTATAAAGTGATTTCATTGCATGAAAACGA |
| | | AATACAATTATATCTGATTTCATTAGGAATACCGAAAAT |
| | | AGACGCTGACACGTATGTTGGATCAAAGATTTATTCTCA |
| | | AGATAAGTATAGAATACTAGAATCATACGTGTACAATT |
| | | TATTGTCCATTAATTATGGATGCTATCAATTATTTGATTT |
| | | CAATTCACCGGACTTGGAGAAGCTGATAAGAATACCAT |
| | | TTAAGGGAAAAATACCAGCTGTTACATTCATATTACACT |
| | | TATATGCAAAGCTAGAAGTTATAAACTACGCTATAAAA |
| | | AATGGTTCATGGATAAGCCTATTTTGCAATTACCCTAAA |
| | | TCAGAAATGATAAAATTATGGAAGAAGATGTGGAACAT |
| | | CACGTCATTACGTTCGCCGTACACTAACGCGAACTTCTT |
| | | TCAAGATTAGAACGCTTAGATGTGACCGGGTCGGCATG |
| | | GCATCTCCACCTCCTCGCGGTCCGACCTGGGCATCCGAA |
| | | GGAGGACGCACGTCCACTCGGATGGCTAAGGGAGAGCC |
| | | TGCAGTAGCATAACCCCTTGGGGCCTCTAAACGGGTCTT |
| | | GAGGGGTTTTTTGGGTACC |
| 2 | VP1 protein, | MGKYNLILSEYLSFIYNSQSAVQIPIYYSSNSELENRCIEFHS |
| | strain SA11 | KCLENSKNGLSLRKLFVEYNDVIENATLLSILSYSYDKYN |
| | | AVERKLVKYAKGKPLEADLTVNELXYENNKITSELFPTAE |
| | | EYTDSLMDPAILTSLSSNLNAVMFWLEKHENDVAEKLKV |
| | | YKRRLDLFTIVASTINKYGVPRHNAKYRYEYDVMKDKPY |
| | | YLVTWANSSIEMLMSVFSHDDYLIAKELIVLSYSNRSTLA |
| | | KLVSSPMSILVALVDINGTFITNEELELEFSNKYVRAIVPDQ |
| | | TFDELNQMLDNMRKAGLVDIPKMIQDWLVDRSIEKFPLM |
| | | AKIYSWSFHVGFRKQKMLDAALDQLKTEYTENVDDEMY |
| | | REYTMLIRDEVVKMLEEPVKHDDHLLRDSELAGLLSMSS |
| | | ASNGESRQLKFGRKTIFSTKKNMHVMDDMANERYTPGIIP |
| | | PVNVDKPIPLGRRDVPGRRTRIIFILPYEYFIAQHAVVEKML |
| | | IYAKHTREYAEFYSQSNQLLSYGDVTRFLSNNTMVLYTDV |
| | | SQWDSSQHNTQPFRKGIIMGLDILANMTNDAKVLQTLNL |
| | | YKQTQINLMDSYVQIPDGNVIKKIQYGAVASGEKQTKAA |
| | | NSIANLALIKTVLSRISNKHSFATKIIRVDGDDNYAVLQFN |
| | | TEVTKQMIQDVSNDVRETYARMNAKVKALVSTVGIEIAK |
| | | RYIAGGKIFFRAGINLLNNEKRGQSTQWDQAAILYSNYIV |
| | | NRLRGFETDREFILTKIMQMTSVAITGSLRLFPSERVLTTNS |
| | | TFKVFDSEDFIIEYGTTDDEVYIQRAFMSLSSQKSGIADEIA |
| | | ASSTFKNYVTRLSEQLLFSKNNIVSRGIALTEKAKLNSYAPI |
| | | SLEKRRAQISALLTMLQKPVTFKSSKITINDILRDIKPFFTVS |
| | | DAHLPIQYQKFMPTLPDNVQYIIQCIGSRTYQIEDDGSKSAI |
| | | SRLISKYSVYKPSIEELYKVISLHENEIQLYLISLGIPKIDADT |
| | | YVGSKIYSQDKYRILESYVYNLLSINYGCYQLFDFNSPDLE |
| | | KLIRIPFKGKIPAVTFILHLYAKLEVINYAIKNGSWISLFCN |
| | | YPKSEMIKLWKKMWNITSLRSPYTNANFFQD |
| 3 | pT7/VP2SA11 | ACTAGTTAATACGACTCACTATAGGCTATTAAAGGCTC |
| | T7 promoter: nt | AATGGCGTATCGAAAACGTGGAGCGCGTCGTGAGACGA |
| | 7-24 | ATCTAAAACAAGATGAACGAATGCAAGAAAAAGAAGA |
| | VP2 CDS: nt | TAGCAAGAACATTAATAATGACAGTCCTAAATCACAAT |
| | 40-2,688 | TATCAGAAAAAGTATTATCTAAGAAAGAAGAGATAATT |
| | HDV Ribozyme: | ACAGATAATCAAGAAGAAGTTAAGATATCTGATGAGGT |
| | nt 2,717-2,805 | AAAAAAATCTAATAAAGAAGAATCGAAACAGTTGTTAG |
| | T7 Terminator: | AAGTACTTAAAACAAAAGAGGAACATCAAAAAGAAGT |
| | nt 2,814-2,856 | TCAGTATGAAATATTACAAAAAACTATCCCTACATTTGA |
| | | ACCAAAAGAGTCAATACTCAAAAAATTAGAAGACATAA |
| | | AACCAGAACAAGCAAAGAAACAAACTAAACTGTTTCGA |
| | | ATATTTGAACCGAAACAATTGCCTATTTATAGAGCTAAT |
| | | GGAGAAAGAGAGCTTCGTAATAGATGGTATTGGAAATT |
| | | GAAACGAGATACTCTTCCTGATGGAGATTATGATGTTA |
| | | GAGAGTATTTTTTAAATTTATATGATCAAGTATTAATGG |
| | | AAATGCCGGATTATCTATTACTTAAAGATATGGCTGTAG |
| | | AGAATAAAAATTCAAGGGATGCTGGCAAAGTAGTTGAT |
| | | TCTGAAACAGCCGCAATATGCGATGCTATTTTTCAAGAT |
| | | GAAGAAACCGAAGGTGCAGTAAGAAGATTCATAGCTG |
| | | AGATGAGACAACGAGTTCAAGCTGATCGAAATGTAGTC |
| | | AATTATCCATCTATATTGCATCCAATTGACCATGCGTTT |
| | | AACGAATACTTCTTACAACATCAGTTGGTAGAACCATT |
| | | AAATAATGATATCATTTTCAATTACATACCAGAGAGAA |
| | | TAAGAAATGATGTCAACTATATATTAAATATGGACAGG |
| | | AATTTACCGTCTACTGCTAGATATATCAGACCAAACTTG |
| | | CTACAAGATAGGTTAAATTTACATGATAATTTTGAGTCA |
| | | CTCTGGGATACTATAACTACATCTAATTATATTTTAGCA |
| | | AGATCTGTGGTGCCAGACCTAAAAGAATTAGTATCTAC |
| | | TGAGGCACAAATCCAGAAAATGTCACAAGATTTGCAAT |
| | | TGGAAGCTTTGACAATACAATCAGAGACTCAGTTTTTA |
| | | ACAGGTATAAACTCACAAGCCGCTAATGATTGTTTTAA |
| | | AACTTTGATTGCTGCTATGTTGAGTCAGAGAACCATGTC |
| | | ATTAGATTTCGTAACGACAAATTACATGTCACTTATTTC |
| | | AGGCATGTGGTTACTCACTGTGATTCCAAATGATATGTT |
| | | TATAAGAGAATCATTAGTAGCATGTCAACTAGCCATAA |
| | | TAAATACCATTGTTTATCCGGCATTCGGAATGCAAAGA |
| | | ATGCATTATAGGAATGGTGATCCACAGACTCCCTTTCAA |
| | | ATTGCAGAGCAACAGATTCAAAATTTTCAGGTAGCTAA |
| | | TTGGTTACATTTTGTTAATTATAATCAGTTTAGACAAGT |
| | | AGTGATTGATGGAGTGTTAAATCAAGTCTTGAATGATA |
| | | ATATAAGAAATGGTCATGTAGTCAACCAATTAATGGAA |
| | | GCTCTGATGCAATTATCTAGACAACAGTTTCCCACAATG |
| | | CCAGTTGATTATAAAAGATCTATACAGAGAGGAATTTT |
| | | GCTGCTTTCTAACAGACTTGGTCAGCTTGTCGATTTAAC |
| | | AAGATTGTTATCATACAATTATGAGACATTAATGGCAT |
| | | GCATAACAATGAATATGCAGCATGTTCAAACATTAACA |
| | | ACTGAAAAATTGCAATTAACATCAGTAACATCATTATG |
| | | TATGCTAATTGGAAATGCTACGGTTATACCGAGTCCGC |
| | | AAACATTGTTCCATTACTATAATGTGAATGTCAATTTTC |
| | | ATTCAAATTATAATGAAAGAATTAATGACGCAGTTGCA |
| | | ATTATAACTGCGGCAAATAGATTAAATTTATATCAAAA |
| | | GAAAATGAAATCAATAGTTGAGGACTTTCTGAAAAGAT |
| | | TACAGATATTTGATGTTGCGAGAGTACCAGATGACCAA |
| | | ATGTATAGATTGAGAGATAGATTAAGACTATTACCAGT |
| | | TGAAATAAGAAGATTAGATATTTTTAATTTGATAGCAAT |
| | | GAATATGGAACAGATTGAACGTGCATCAGATAAAATTG |
| | | CACAAGGAGTTATAATAGCATACCGAGATATGCAGTTA |
| | | GAACGAGATGAGATGTATGGTTACGTCAATATTGCCAG |
| | | AAACTTGGACGGATTTCAACAAATAAATCTTGAAGAAT |
| | | TGATGAGATCAGGAGATTATGCTCAAATTACTAACATG |
| | | CTACTTAATAATCAACCAGTAGCTTTAGTTGGAGCGCTA |
| | | CCATTTATAACGGATTCATCAGTGATTTCGTTAATAGCT |
| | | AAACTAGATGCAACCGTTTTTGCACAGATTGTCAAACTT |
| | | AGAAAGGTCGACACGTTAAAACCCATCCTATATAAGAT |
| | | AAATTCAGATTCTAATGACTTTTATTTGGTGGCTAATTA |
| | | TGATTGGATTCCTACATCTACTACAAAAGTGTATAAACA |
| | | AGTTCCACAACAATTTGATTTTAGAGCGTCAATGCATAT |
| | | GTTAACGTCTAACCTAACATTTACCGTATATTCAGATTT |
| | | GCTTGCGTTCGTTTCAGCTGATACTGTTGAACCAATTAA |
| | | TGCTGTTGCTTTTGATAATATGCGCATCATGAACGAACT |
| | | GTAAACGCCAACCCCATTGTGGAGATATGACCGGGTCG |
| | | GCATGGCATCTCCACCTCCTCGCGGTCCGACCTGGGCAT |
| | | CCGAAGGAGGACGCACGTCCACTCGGATGGCTAAGGGA |
| | | GAGCCTGCAGTAGCATAACCCCTTGGGGCCTCTAAACG |
| | | GGTCTTGAGGGGTTTTTTGGGTACC |
| 4 | VP2 protein, | MAYRKRGARRETNLKQDERMQEKEDSKNINNDSPKSQLS |
| | strain SA11 | EKVLSKKEEIITDNQEEVKISDEVKKSNKEESKQLLEVLKT |
| | | KEEHQKEVQYEILQKTIPTFEPKESILKKLEDIKPEQAKKQT |
| | | KLFRIFEPKQLPIYRANGERELRNRWYWKLKRDTLPDGDY |
| | | DVREYFLNLYDQVLMEMPDYLLLKDMAVENKNSRDAGK |
| | | VVDSETAAICDAIFQDEETEGAVRRFIAEMRQRVQADRNV |
| | | VNYPSILHPIDHAFNEYFLQHQLVEPLNNDIIFNYIPERIRN |
| | | DVNYILNMDRNLPSTARYIRPNLLQDRLNLHDNFESLWDT |
| | | ITTSNYILARSVVPDLKELVSTEAQIQKMSQDLQLEALTIQS |
| | | ETQFLTGINSQAANDCFKTLIAAMLSQRTMSLDFVTTNYM |
| | | SLISGMWLLTVIPNDMFIRESLVACQLAIINTIVYPAFGMQ |
| | | RMHYRNGDPQTPFQIAEQQIQNFQVANWLHFVNYNQFRQ |
| | | VVIDGVLNQVLNDNIRNGHVVNQLMEALMQLSRQQFPT |
| | | MPVDYKRSIQRGILLLSNRLGQLVDLTRLLSYNYETLMAC |
| | | ITMNMQHVQTLTTEKLQLTSVTSLCMLIGNATVIPSPQTLF |
| | | HYYNVNVNFHSNYNERINDAVAIITAANRLNLYQKKMKSI |
| | | VEDFLKRLQIFDVARVPDDQMYRLRDRLRLLPVEIRRLDIF |
| | | NLIAMNMEQIERASDKIAQGVIIAYRDMQLERDEMYGYV |
| | | NIARNLDGFQQINLEELMRSGDYAQITNMLLNNQPVALVG |
| | | ALPFITDSSVISLIAKLDATVFAQIVKLRKVDTLKPILYKINS |
| | | DSNDFYLVANYDWIPTSTTKVYKQVPQQFDFRASMHMLT |
| | | SNLTFTVYSDLLAFVSADTVEPINAVAFDNMRIMNEL |
| 5 | pT7/VP3SA11 | TCTAGATAATACGACTCACTATAGGCTATTAAAGCAGT |
| | T7 promoter: nt | ACCAGTAGTGTGTTTTACCTCTGATGGTGTAAACATGAA |
| | 7-24 | AGTACTAGCTTTAAGACACAGTGTGGCTCAAGTGTATG |
| | VP3 CDS: nt | CAGACACTCAAGTCTACGTTCATGATGATACAAAAGAT |
| | 73-2,580 | AGTTATGAAAACGCTTTTTTAATCTCTAATCTTACGACC |
| | HDV Ribozyme: | CATAATATTTTATACTTAAATTATAGCATTAAAACATTA |
| | nt 2,615-2,703 | GAAATATTAAATAAGTCAGGAATAGCTGCAATTGCTTT |
| | T7 Terminator: | ACAATCACTTGAAGAATTATTCACATTAATAAGGTGTA |
| | nt 2,712-2,754 | ATTTCACTTATGATTATGAACTTGATATAATATATTTAC |
| | | ATGATTATTCATATTATACCAATAATGAAATTAGAACA |
| | | GACCAACATTGGATAACAAAAACAAATATTGAAGAATA |
| | | TTTACTACCTGGATGGAAATTAACATATGTTGGTTATAA |
| | | TGGAAGTGAAACTAGAGGACATTATAACTTTTCATTTA |
| | | AATGTCAAAACGCTGCAACAGATGATGATCTAATAATT |
| | | GAATACATTTATTCAGAAGCGTTGGACTTCCAAAATTTT |
| | | ATGTTAAAAAAGATAAAGGAAAGAATGACTACATCGTT |
| | | GCCTATAGCTAGATTATCTAACAGAGTATTTAGGGATA |
| | | AGTTATTCCCATCATTATTGAAAGAACATAAGAATGTA |
| | | GTGAACGTTGGTCCGCGTAATGAATCTATGTTTACATTT |
| | | TTAAATTATCCAACTATAAAACAATTTTCAAATGGTGCG |
| | | TATTTAGTAAAAGATACTATAAAATTAAAACAAGAACG |
| | | ATGGTTAGGTAAAAGGATATCTCAGTTTGATATTGGTCA |
| | | GTATAAAAATATGCTGAATGTTCTTACAGCAATTTATTA |
| | | TTACTATAATTTATATAAAAGTAAACCAATTATATATAT |
| | | GATCGGATCTGCTCCATCTTATTGGATATATGACGTTAG |
| | | GCATTATTCCGATTTTTTCTTTGAAACTTGGGATCCATT |
| | | GGACACACCATATTCATCAATCCATCACAAAGAATTAT |
| | | TTTTTATAAATGATGTGAAGAAACTGAAGGATAACTCA |
| | | ATATTGTATATTGATATAAGAACCGATAGGGGCAATGC |
| | | TGATTGGAAAAAATGGAGAAAGACAGTAGAAGAACAA |
| | | ACTATTAATAATTTGGACATAGCTTATGAATATTTACGA |
| | | ACGGGTAAAGCGAAGGTGTGTTGTGTTAAGATGACAGC |
| | | TATGGATTTGGAACTGCCAATTTCAGCTAAATTACTGCA |
| | | CCACCCAACTACGGAAATAAGATCAGAATTTTATTTATT |
| | | ACTAGATACTTGGGATTTAACTAACATTAGGAGGTTCAT |
| | | TCCTAAAGGCGTGTTATATTCATTTATAAACAATATAAT |
| | | AACTGAAAATGTGTTTATTCAACAACCATTTAAAGTAA |
| | | AAGTACTGAATGATAGTTATATTGTAGCGTTATATGCAT |
| | | TATCAAATGATTTTAATAATAGATCAGAAGTAATTAAA |
| | | TTAATTAATAATCAGAAACAATCTCTAATAACTGTTAGA |
| | | ATAAATAATACGTTTAAGGATGAACCAAAAGTTGGGTT |
| | | CAAAAATATCTATGATTGGACCTTTCTTCCAACCGACTT |
| | | TGATACCAAAGAAGCTATAATTACTTCATACGACGGTT |
| | | GTTTAGGACTCTTTGGTTTGTCTATATCGTTAGCATCAA |
| | | AACCAACAGGGAATAATCATTTATTCATTTTAAGTGGTA |
| | | CAGATAAGTATTATAAATTGGATCAATTTGCTAATCACA |
| | | CCAGTATATCGAGAAGATCACACCAAATTAGGTTTTCG |
| | | GAATCTGCTACTTCATATTCAGGTTATATATTTAGAGAT |
| | | TTGTCCAATAATAATTTTAATCTAATTGGTACTAATATA |
| | | GAGAATTCAGTATCAGGTCATGTATATAATGCTTTAATT |
| | | TATTATAGATATAATTATTCATTTGATCTTAAACGCTGG |
| | | ATTTATTTACATTCTATAGATAAAGTTGATATAGAAGGA |
| | | GGAAAGTATTATGAACACGCACCAATAGAATTAATTTA |
| | | TGCATGTAGATCAGCAAAAGAATTTGCTACATTGCAGG |
| | | ATGACTTAACTGTATTGAGATATTCAAACGAAATAGAG |
| | | AATTATATTAATACAGTATATAGTATAACATACGCTGAT |
| | | GATCCGAATTACTTTATCGGAATACAATTTAGAAATATA |
| | | CCATATAAATATGATGTTAAAATACCGCATTTAACCTTC |
| | | GGAGTATTACATATTTCTGATAACATGGTGCCAGACGT |
| | | GATTGACATACTAAAGATAATGAAGAATGAATTATTTA |
| | | AAATGGATATTACGACCAGTTATACATATATGTTATCAG |
| | | ATGGAATCTACGTAGCAAATGTTAGTGGAGTATTATCT |
| | | ACATACTTTAAAATCTATAACGTATTTTATAAAAATCAA |
| | | ATAACTTTTGGCCAATCCAGAATGTTTATTCCGCACATA |
| | | ACATTAAGCTTCAATAACATGAGAACAGTAAGGATAGA |
| | | GACTACTAAATTACAAATTAAATCCATTTATTTAAGAAA |
| | | GATTAAGGGTGATACAGTGTTTGATATGGTTGAGTGAG |
| | | CTAAAAACTTAACACACTAGTCATGATGTGACCGGGTC |
| | | GGCATGGCATCTCCACCTCCTCGCGGTCCGACCTGGGC |
| | | ATCCGAAGGAGGACGCACGTCCACTCGGATGGCTAAGG |
| | | GAGAGCCTGCAGTAGCATAACCCCTTGGGGCCTCTAAA |
| | | CGGGTCTTGAGGGGTTTTTTGGGTACC |
| 6 | VP3 protein, | MKVLALRHSVAQVYADTQVYVHDDTKDSYENAFLISNLT |
| | strain SA11 | THNILYLNYSIKTLEILNKSGIAAIALQSLEELFTLIRCNFTY |
| | | DYELDIIYLHDYSYYTNNEIRTDQHWITKTNIEEYLLPGWK |
| | | LTYVGYNGSETRGHYNFSFKCQNAATDDDLIIEYIYSEAL |
| | | DFQNFMLKKIKERMTTSLPIARLSNRVFRDKLFPSLLKEHK |
| | | NVVNVGPRNESMFTFLNYPTIKQFSNGAYLVKDTIKLKQE |
| | | RWLGKRISQFDIGQYKNMLNVLTAIYYYYNLYKSKPIIYM |
| | | IGSAPSYWIYDVRHYSDFFFETWDPLDTPYSSIHHKELFFIN |
| | | DVKKLKDNSILYIDIRTDRGNADWKKWRKTVEEQTINNL |
| | | DIAYEYLRTGKAKVCCVKMTAMDLELPISAKLLHHPTTEI |
| | | RSEFYLLLDTWDLTNIRRFIPKGVLYSFINNIITENVFIQQPF |
| | | KVKVLNDSYIVALYALSNDFNNRSEVIKLINNQKQSLITVR |
| | | INNTFKDEPKVGFKNIYDWTFLPTDFDTKEAIITSYDGCLG |
| | | LFGLSISLASKPTGNNHLFILSGTDKYYKLDQFANHTSISRR |
| | | SHQIRFSESATSYSGYIFRDLSNNNENLIGTNIENSVSGHVY |
| | | NALIYYRYNYSFDLKRWIYLHSIDKVDIEGGKYYEHAPIEL |
| | | IYACRSAKEFATLQDDLTVLRYSNEIENYINTVYSITYADD |
| | | PNYFIGIQFRNIPYKYDVKIPHLTFGVLHISDNMVPDVIDIL |
| | | KIMKNELFKMDITTSYTYMLSDGIYVANVSGVLSTYFKIY |
| | | NVFYKNQITFGQSRMFIPHITLSFNNMRTVRIETTKLQIKSI |
| | | YLRKIKGDTVFDMVE |
| 7 | pT7/VP4SA11 | ACTAGTTAATACGACTCACTATAGGCTATAAAATGGCTT |
| | T7 promoter: nt | CGCTCATTTATAGACAATTGCTCACGAATTCTTATACAG |
| | 7-24 | TAGATTTATCCGATGAGATACAAGAGATTGGATCAACT |
| | VP4 CDS: nt | AAATCACAAAATGTCACAATTAATCCTGGACCATTTGC |
| | 33-2,363 | GCAAACAGGTTATGCTCCAGTTAACTGGGGACCTGGAG |
| | HDV Ribozyme: | AAATTAATGATTCTACGACAGTTGGACCATTGCTGGAT |
| | nt 2,386-2,474 | GGGCCTTATCAACCAACGACATTCAATCCACCAGTCGA |
| | T7 Terminator: | TTATTGGATGTTACTGGCTCCAACGACACCTGGCGTAAT |
| | nt 2,483-2,525 | TGTTGAAGGTACAAATAATACAGATAGATGGTTAGCCA |
| | | CAATTTTAATCGAGCCAAATGTTCAGTCTGAAAATAGA |
| | | ACTTACACTATATTTGGTATTCAAGAACAATTAACGGTA |
| | | TCCAATACTTCACAAGACCAGTGGAAATTTATTGATGTC |
| | | GTAAAAACAACTGCAAATGGAAGTATAGGACAATATGG |
| | | ACCATTACTATCCAGTCCGAAATTATATGCAGTTATGAA |
| | | GCATAATGAAAAATTATATACATATGAAGGACAGACAC |
| | | CTAACGCTAGGACAGCACATTATTCAACAACGAATTAT |
| | | GATTCTGTTAACATGACTGCTTTTTGTGACTTTTATATA |
| | | ATTCCTAGATCTGAAGAGTCTAAATGTACGGAATACAT |
| | | TAATAATGGATTACCACCAATACAAAATACTAGAAATG |
| | | TTGTACCATTATCGTTGACTGCTAGAGATGTAATACACT |
| | | ATAGAGCTCAAGCTAATGAAGATATTGTGATATCCAAG |
| | | ACATCATTATGGAAAGAAATGCAATATAATAGAGATAT |
| | | AACTATTAGATTTAAATTTGCAAATACAATTATAAAATC |
| | | AGGAGGGCTGGGATATAAGTGGTCAGAAATATCATTTA |
| | | AGCCAGCGAATTATCAATACACATATACTCGTGATGGT |
| | | GAAGAAGTTACCGCACATACTACTTGTTCAGTGAATGG |
| | | CGTTAATGACTTCAGTTTTAATGGAGGATATTTACCAAC |
| | | TGATTTTGTTGTATCTAAATTTGAAGTAATTAAAGAGAA |
| | | TTCATACGTCTATATCGATTACTGGGATGATTCACAAGC |
| | | ATTTCGTAACGTGGTGTATGTCCGATCGTTAGCAGCAAA |
| | | CTTGAATTCAGTTATGTGTACTGGAGGCAGCTATAATTT |
| | | TAGTCTACCAGTTGGACAATGGCCTGTTTTAACTGGGGG |
| | | AGCAGTTTCTTTACATTCAGCTGGTGTAACACTATCTAC |
| | | TCAATTTACAGATTTCGTATCATTAAATTCATTAAGATT |
| | | TAGATTTAGACTAGCTGTCGAAGAACCACACTTTAAAC |
| | | TGACTAGAACTAGATTAGATAGATTGTATGGTCTGCCTG |
| | | CTGCAGATCCAAATAATGGTAAAGAATATTATGAAATT |
| | | GCTGGACGATTTTCACTTATATCATTAGTGCCATCAAAT |
| | | GATGACTATCAGACTCCTATAGCAAACTCAGTTACTGTA |
| | | CGACAAGATTTAGAAAGGCAGTTAGGAGAACTAAGAG |
| | | AAGAGTTTAACGCTTTGTCTCAAGAAATTGCAATGTCGC |
| | | AGTTAATCGATTTAGCGCTTCTACCATTAGATATGTTCT |
| | | CAATGTTTTCTGGCATTAAAAGTACTATTGATGCTGCAA |
| | | AATCAATGGCTACTAATGTTATGAAAAAATTCAAAAAG |
| | | TCAGGATTAGCGAATTCAGTTTCAACACTGACAGATTCT |
| | | TTATCAGACGCAGCATCATCAATATCAAGAGGTTCATCT |
| | | ATACGTTCGATTGGATCTTCAGCATCAGCATGGACGGA |
| | | TGTATCAACACAAATAACTGATATATCGTCATCAGTAA |
| | | GTTCAGTTTCGACACAAACGTCAACTATCAGTAGAAGA |
| | | TTGAGACTAAAGGAAATGGCAACACAAACTGAGGGTAT |
| | | GAATTTTGATGATATATCAGCGGCTGTTTTGAAGACTAA |
| | | GATAGATAAATCGACTCAAATATCACCAAACACAATAC |
| | | CTGACATTGTTACTGAAGCATCGGAAAAATTCATACCA |
| | | AATAGGGCTTACCGCGTTATAAACAACGATGATGTGTT |
| | | TGAAGCTGGAATTGATGGGAAATTTTTTGCTTATAAAGT |
| | | GGATACATTTGAGGAAATACCATTTGATGTACAAAAAT |
| | | TCGCTGACTTAGTTACAGATTCTCCAGTAATATCCGCTA |
| | | TAATTGATTTTAAAACACTTAAAAATTTGAACGATAATT |
| | | ACGGCATTACTAAGCAACAAGCATTTAATCTTTTAAGAT |
| | | CTGACCCAAGAGTTTTACGTGAATTCATTAATCAGGAC |
| | | AATCCTATAATTAGAAATAGAATTGAACAACTGATTAT |
| | | GCAATGCAGGTTGTGAGTAATTTCTAGAGGATGTGACC |
| | | GGGTCGGCATGGCATCTCCACCTCCTCGCGGTCCGACCT |
| | | GGGCATCCGAAGGAGGACGCACGTCCACTCGGATGGCT |
| | | AAGGGAGAGCCTGCAGTAGCATAACCCCTTGGGGCCTC |
| | | TAAACGGGTCTTGAGGGGTTTTTTGGGTACC |
| 8 | VP4 protein, | MASLIYRQLLTNSYTVDLSDEIQEIGSTKSQNVTINPGPFAQ |
| | strain SA11 | TGYAPVNWGPGEINDSTTVGPLLDGPYQPTTFNPPVDYW |
| | | MLLAPTTPGVIVEGTNNTDRWLATILIEPNVQSENRTYTIF |
| | | GIQEQLTVSNTSQDQWKFIDVVKTTANGSIGQYGPLLSSPK |
| | | LYAVMKHNEKLYTYEGQTPNARTAHYSTTNYDSVNMTA |
| | | FCDFYIIPRSEESKCTEYINNGLPPIQNTRNVVPLSLTARDVI |
| | | HYRAQANEDIVISKTSLWKEMQYNRDITIRFKFANTIIKSG |
| | | GLGYKWSEISFKPANYQYTYTRDGEEVTAHTTCSVNGVN |
| | | DFSFNGGYLPTDFVVSKFEVIKENSYVYIDYWDDSQAFRN |
| | | VVYVRSLAANLNSVMCTGGSYNFSLPVGQWPVLTGGAVS |
| | | LHSAGVTLSTQFTDFVSLNSLRFRFRLAVEEPHFKLTRTRL |
| | | DRLYGLPAADPNNGKEYYEIAGRFSLISLVPSNDDYQTPIA |
| | | NSVTVRQDLERQLGELREEFNALSQEIAMSQLIDLALLPLD |
| | | MFSMFSGIKSTIDAAKSMATNVMKKFKKSGLANSVSTLTD |
| | | SLSDAASSISRGSSIRSIGSSASAWTDVSTQITDISSSVSSVST |
| | | QTSTISRRLRLKEMATQTEGMNFDDISAAVLKTKIDKSTQI |
| | | SPNTIPDIVTEASEKFIPNRAYRVINNDDVFEAGIDGKFFAY |
| | | KVDTFEEIPFDVQKFADLVTDSPVISAIIDFKTLKNLNDNY |
| | | GITKQQAFNLLRSDPRVLREFINQDNPIIRNRIEQLIMQCRL |
| 9 | pT7/VP6SA11 | AAGCTTTAATACGACTCACTATAGGCTTTTAAACGAAGT |
| | T7 promoter: nt | CTTCAACATGGATGTCCTATACTCTTTGTCAAAGACTCT |
| | 7-24 | TAAAGACGCTAGAGACAAAATTGTCGAAGGCACATTGT |
| | VP6 CDS: nt | ATTCTAACGTGAGTGATCTAATTCAACAATTTAATCAAA |
| | 47-1,240 | TGATAATTACTATGAATGGAAATGAATTTCAAACTGGA |
| | HDV Ribozyme: | GGAATCGGTAATTTGCCAATTAGAAACTGGAATTTTAA |
| | nt 1,380-1,468 | TTTCGGGTTACTTGGAACAACTTTGCTGAACTTAGACGC |
| | T7 Terminator: | TAATTATGTTGAAACGGCAAGAAATACAATTGATTATTT |
| | nt 1,477-1,519 | CGTGGATTTTGTAGACAATGTATGCATGGATGAGATGG |
| | | TTAGAGAATCACAAAGGAACGGAATTGCACCTCAATCA |
| | | GACTCGCTAAGAAAGCTGTCAGCCATTAAATTCAAAAG |
| | | AATAAATTTTGATAATTCGTCGGAATACATAGAAAACT |
| | | GGAATTTGCAAAATAGAAGACAGAGGACAGGTTTCACT |
| | | TTTCATAAACCAAACATTTTTCCTTATTCAGCATCATTT |
| | | ACACTAAATAGATCACAACCCGCTCATGATAATTTGAT |
| | | GGGCACAATGTGGTTAAACGCAGGATCGGAAATTCAAG |
| | | TCGCTGGATTTGACTACTCATGTGCTATTAACGCACCAG |
| | | CCAATATACAACAATTTGAGCATATTGTGCCACTCCGA |
| | | AGAGTGTTAACTACAGCTACGATAACTCTTCTACCAGA |
| | | CGCGGAAAGGTTTAGTTTTCCAAGAGTGATCAATTCAG |
| | | CTGACGGGGCAACTACATGGTTTTTCAACCCAGTGATTC |
| | | TCAGGCCGAATAACGTTGAAGTGGAGTTTCTATTGAAT |
| | | GGACAGATAATAAACACTTATCAAGCAAGATTTGGAAC |
| | | TATCGTAGCTAGAAATTTTGATACTATTAGACTATCATT |
| | | CCAGTTAATGAGACCACCAAACATGACACCAGCAGTAG |
| | | CAGTACTATTCCCGAATGCACAGCCATTCGAACATCAT |
| | | GCAACAGTGGGATTGACACTTAGAATTGAGTCTGCAGT |
| | | TTGTGAGTCTGTACTCGCCGATGCAAGTGAAACTCTATT |
| | | AGCAAATGTAACATCCGTTAGGCAAGAGTACGCAATAC |
| | | CAGTTGGACCAGTCTTTCCACCAGGTATGAACTGGACT |
| | | GATTTAATCACCAATTATTCACCGTCTAGGGAGGACAA |
| | | TTTGCAACGCGTATTTACAGTGGCTTCCATTAGAAGCAT |
| | | GCTCATTAAATGAGGACCAAGCTAACAACTTGGTATCC |
| | | AACTTTGGTGAGTATGTAGCTATATCAAGCTGTTTGAAC |
| | | TCTGTAAGTAAGGATGCGTATACGCATTCGCTACACAG |
| | | AGTAATCACTCAGATGGTATAGTGAGAGGATGTGACCG |
| | | GGTCGGCATGGCATCTCCACCTCCTCGCGGTCCGACCTG |
| | | GGCATCCGAAGGAGGACGCACGTCCACTCGGATGGCTA |
| | | AGGGAGAGCCTGCAGTAGCATAACCCCTTGGGGCCTCT |
| | | AAACGGGTCTTGAGGGGTTTTTTGGGTACC |
| 10 | VP6 protein, | MDVLYSLSKTLKDARDKIVEGTLYSNVSDLIQQFNQMIIT |
| | strain SA11 | MNGNEFQTGGIGNLPIRNWNFNFGLLGTTLLNLDANYVET |
| | | ARNTIDYFVDFVDNVCMDEMVRESQRNGIAPQSDSLRKLS |
| | | AIKFKRINFDNSSEYIENWNLQNRRQRTGFTFHKPNIFPYS |
| | | ASFTLNRSQPAHDNLMGTMWLNAGSEIQVAGFDYSCAIN |
| | | APANIQQFEHIVPLRRVLTTATITLLPDAERFSFPRVINSAD |
| | | GATTWFFNPVILRPNNVEVEFLLNGQIINTYQARFGTIVAR |
| | | NFDTIRLSFQLMRPPNMTPAVAVLFPNAQPFEHHATVGLT |
| | | LRIESAVCESVLADASETLLANVTSVRQEYAIPVGPVFPPG |
| | | MNWTDLITNYSPSREDNLQRVFTVASIRSMLIK |
| 11 | pT7/VP7SA11 | AAGCTTTAATACGACTCACTATAGGCTTTAAAAAGAGA |
| | T7 promoter: nt | GAATTTCCGTTTGGCTAGCGGTTAGCTCCTTTTAATGTA |
| | 7-24 | TGGTATTGAATATACCACAGTTCTAACCTTTCTGATATC |
| | VP7 CDS: nt | GATTATTCTACTAAATTACATACTTAAATCATTAACTAG |
| | 73-1,053 | AATAATGGACTTTATAATTTATAGATTTCTTTTTATAATT |
| | HDV Ribozyme: | GTGATATTGTCACCATTTCTCAGAGCACAAAATTATGGT |
| | nt 1,087-1,185 | ATTAATCTTCCAATCACAGGCTCCATGGACATTGCATAC |
| | T7 Terminator: | GCTAATTCAACGCAAGAAGAACCATTCCTCACTTCTAC |
| | nt 1,184-1,226 | ACTTTGCCTATATTATCCGACTGAGGCTGCGACTGAAAT |
| | | AAACGATAATTCATGGAAAGACACACTGTCACAACTAT |
| | | TTCTTACGAAAGGGTGGCCAACTGGATCCGTATATTTTA |
| | | AAGAATATACTAACATTGCATCGTTTTCTGTTGATCCGC |
| | | AGTTGTATTGTGATTATAACGTAGTACTAATGAAATATG |
| | | ACGCGACGTTGCAATTGGATATGTCAGAACTTGCGGAT |
| | | CTAATATTAAACGAATGGTTGTGTAATCCAATGGATATT |
| | | ACTCTGTATTATTATCAGCAAACTGACGAAGCGAATAA |
| | | ATGGATATCAATGGGCTCATCATGTACAATTAAAGTAT |
| | | GTCCACTTAATACACAAACTCTTGGAATTGGATGCTTGA |
| | | CAACTGATGCTACAACTTTTGAAGAAGTTGCGACAGCT |
| | | GAAAAGTTGGTAATTACTGACGTGGTTGATGGCGTTAA |
| | | TCATAAGCTGGATGTCACAACAGCAACGTGTACTATTA |
| | | GAAACTGTAAGAAATTGGGACCAAGAGAAAACGTAGC |
| | | CGTTATACAAGTTGGTGGTTCTGACATCCTCGATATAAC |
| | | TGCTGATCCAACTACTGCACCACAGACAGAACGGATGA |
| | | TGCGAATTAACTGGAAAAAATGGTGGCAAGTTTTTTAT |
| | | ACTGTAGTAGACTATGTAGATCAGATAATACAAGTTAT |
| | | GTCCAAAAGATCAAGATCACTAAATTCAGCAGCATTTT |
| | | ATTACAGAGTGTAGGTATAACTTAGGTTAGAATTGTAT |
| | | GATGTGACCGGGTCGGCATGGCATCTCCACCTCCTCGC |
| | | GGTCCGACCTGGGCATCCGAAGGAGGACGCACGTCCAC |
| | | TCGGATGGCTAAGGGAGAGCCTGCAGTAGCATAACCCC |
| | | TTGGGGCCTCTAAACGGGTCTTGAGGGGTTTTTTGGGTA |
| | | CC |
| 12 | VP7 protein, | MYGIEYTTVLTFLISIILLNYILKSLTRIMDFIIYRFLFIIVILSP |
| | strain SA11 | FLRAQNYGINLPITGSMDIAYANSTQEEPFLTSTLCLYYPTE |
| | | AATEINDNSWKDTLSQLFLTKGWPTGSVYFKEYTNIASFS |
| | | VDPQLYCDYNVVLMKYDATLQLDMSELADLILNEWLCN |
| | | PMDITLYYYQQTDEANKWISMGSSCTIKVCPLNTQTLGIG |
| | | CLTTDATTFEEVATAEKLVITDVVDGVNHKLDVTTATCTI |
| | | RNCKKLGPRENVAVIQVGGSDILDITADPTTAPQTERMMR |
| | | INWKKWWQVFYTVVDYVDQIIQVMSKRSRSLNSAAFYYR |
| | | V |
| 13 | pT7/NSPISA11 | ACTAGTTAATACGACTCACTATAGGCTTTTTTTTGAAAA |
| | T7 promoter: nt | GTCTTGTGTTAGCCATGGCTACTTTTAAAGATGCATGCT |
| | 7-24 | TTCATTATCGTAGATTAACTGCTTTAAATCGGAGATTAT |
| | NSP1 CDS: nt | GCAACATTGGTGCAAATTCTATTTGGATGCCAGTTCCTG |
| | 54-1,544 | ATGCGAAAATTAAGGGGTGGTGTTTAGAATGTTGTCAA |
| | HDV Ribozyme: | ATAGCTGATTTAACCCATTGTTATGGTTGCTCATTGCCG |
| | nt 1,634-1,722 | CATGTTTGCAAATGGTGTGTTCAGAACAGAAGATGCTT |
| | T7 Terminator: | CCTTGACAATGAACCTCATTTGCTTAAGCTTAGAACTGT |
| | nt 1,731-1,773 | GAAACATCCAATTACCAAAGACAAATTACAGTGTATCA |
| | | TAGACTTGTACAATATAATATTTCCAATTAATGATAAAG |
| | | TAATTAGAAAATTTGAAAGAATGATAAAGCAAAGAGA |
| | | ATGTAGGAATCAATATAAAATTGAATGGTATAATCATT |
| | | TGCTGCTCCCAATTACATTAAATGCTGCTGCATTTAAGT |
| | | TTGATGAAAATAATCTTTATTATGTTTTTGGGTTATATG |
| | | AGAAATCAGTCAGTGATATATATGCTCCATATAGAATT |
| | | GTTAACTTTATAAATGAATTTGATAAATTATTGCTTGAT |
| | | CATATTAACTTTACAAGAATGTCCAATCTACCAATAGA |
| | | GTTGAGAAACCATTACGCAAAGAAATACTTCCAATTAT |
| | | CAAGACTGCCATCATCAAAACTAAAGCAAATTTACTTTT |
| | | CAGATTTTACTAAAGAAACTGTGATTTTTAATACTTATA |
| | | CAAAAACGCCAGGAAGATCAATATACAGAAATGTAACT |
| | | GAATTTAATTGGAGAGATGAATTGGAGCTTTATTCTGAT |
| | | TTAAAAAATGATAAGAATAAATTAATTGCTGCAATGAT |
| | | GACGAGTAAGTATACTCGGTTCTATGCTCATGATAATA |
| | | ATTTTGGAAGGTTGAAAATGACAATATTTGAGTTGGGA |
| | | CATCATTGTCAGCCTAACTACGTGGCATCTAATCACCCA |
| | | GGCAATGCTTCCGATATCCAGTACTGTAAATGGTGTAAT |
| | | ATAAAATATTTTCTTAGTAAAATTGATTGGCGGATTCGT |
| | | GATATGTATAATTTATTGATGGAATTTATTAAGGATTGT |
| | | TATAAAAGTAATGTTAACGTTGGACATTGTAGTTCTGTT |
| | | GAAAACATATATCCTTTAATTAAAAGATTAATTTGGAGT |
| | | TTGTTTACTAATCACATGGATCAAACAATTGAAGAAGT |
| | | GTTTAATCACATGTCGCCAGTGTCAGTTGAAGGTACGA |
| | | ATGTCATCATGTTGATTCTTGGATTGAATATTAGTTTGT |
| | | ATAATGAAATTAAGCGCACTTTGAATGTAGATAGCATA |
| | | CCAATGGTACTTAATTTAAATGAATTCAGTAGTATAGTT |
| | | AAATCAATTAGCAGTAAATGGTATAATGTTGATGAATT |
| | | GGATAAATTGCCAATGTCAATAAAATCAACGGAGGAAC |
| | | TGATTGAAATGAAGAATTCTGGAACTTTAACTGAAGAA |
| | | TTTGAGCTACTGATCTCCAACTCAGAAGATGACAATGA |
| | | GTGAAATTATGTCACTATCTAATTATACAGTATTTAGCC |
| | | ATCACAAGACCGTCCAGACTAGAGTAGCGCCTAGCTGG |
| | | CAAAATACTGTGAACCGGGTCGGCATGGCATCTCCACC |
| | | TCCTCGCGGTCCGACCTGGGCATCCGAAGGAGGACGCA |
| | | CGTCCACTCGGATGGCTAAGGGAGAGCCTGCAGTAGCA |
| | | TAACCCCTTGGGGCCTCTAAACGGGTCTTGAGGGGTTTT |
| | | TTGGGTACC |
| 14 | NSP1 protein, | MATFKDACFHYRRLTALNRRLCNIGANSIWMPVPDAKIK |
| | strain SA11 | GWCLECCQIADLTHCYGCSLPHVCKWCVQNRRCFLDNEP |
| | | HLLKLRTVKHPITKDKLQCIIDLYNIIFPINDKVIRKFERMIK |
| | | QRECRNQYKIEWYNHLLLPITLNAAAFKFDENNLYYVFGL |
| | | YEKSVSDIYAPYRIVNFINEFDKLLLDHINFTRMSNLPIELR |
| | | NHYAKKYFQLSRLPSSKLKQIYFSDFTKETVIFNTYTKTPG |
| | | RSIYRNVTEFNWRDELELYSDLKNDKNKLIAAMMTSKYT |
| | | RFYAHDNNFGRLKMTIFELGHHCQPNYVASNHPGNASDI |
| | | QYCKWCNIKYFLSKIDWRIRDMYNLLMEFIKDCYKSNVN |
| | | VGHCSSVENIYPLIKRLIWSLFTNHMDQTIEEVENHMSPVS |
| | | VEGTNVIMLILGLNISLYNEIKRTLNVDSIPMVLNLNEFSSI |
| | | VKSISSKWYNVDELDKLPMSIKSTEELIEMKNSGTLTEEFE |
| | | LLISNSEDDNE |
| 15 | pT7/NSP2SA11 | AAGCTTTAATACGACTCACTATAGGCTTTTAAAGCGTCT |
| | T7 promoter: nt | CAGTCGCCGTTTGAGCCTTGCGGTGTAGCCATGGCTGA |
| | 7-24 | GCTAGCTTGCTTTTGCTATCCTCATTTGGAGAATGATAG |
| | NSP2 CDS: nt | CTATAAATTTATTCCTTTTAATAATTTAGCTATTAAAGC |
| | 70-1,023 | TATGCTGACAGCTAAAGTAGACAAAAAGGACATGGATA |
| | HDV Ribozyme: | AGTTTTATGATTCAATTATTTATGGAATAGCACCGCCTC |
| | nt 1,083-1,171 | CTCAATTTAAGAAACGGTATAATACTAATGATAATTCA |
| | T7 Terminator: | AGAGGCATGAATTTTGAAACAATTATGTTTACTAAGGT |
| | nt 1,180-1,222 | GGCTATGTTGATATGTGAAGCTCTAAATTCATTGAAAGT |
| | | GACGCAAGCAAACGTCTCTAATGTATTATCACGAGTAG |
| | | TATCAATAAGGCATTTAGAAAATTTGGTGATACGTAAA |
| | | GAAAATCCACAGGATATTCTATTTCATTCAAAAGATTTA |
| | | CTTTTGAAATCAACACTGATTGCTATTGGACAGTCTAAA |
| | | GAAATTGAAACTACAATAACTGCAGAAGGAGGAGAAA |
| | | TTGTATTTCAAAACGCTGCCTTCACCATGTGGAAACTAA |
| | | CTTATTTAGAACATCAATTGATGCCAATTCTGGATCAGA |
| | | ATTTTATTGAATATAAAGTTACATTGAACGAAGATAAA |
| | | CCAATTTCAGATGTTCATGTTAAAGAATTAGTCGCTGAA |
| | | CTTCGATGGCAATATAACAAGTTTGCTGTAATCACACAT |
| | | GGTAAGGGTCATTATAGAATTGTAAAGTATTCATCAGTT |
| | | GCTAATCACGCTGACAGAGTATATGCAACTTTCAAGAG |
| | | TAATGTTAAAACTGGAGTTAATAATGATTTTAACCTACT |
| | | TGATCAAAGAATTATTTGGCAAAACTGGTATGCATTTAC |
| | | ATCATCAATGAAACAGGGTAATACACTTGACGTGTGTA |
| | | AAAGGTTGCTTTTCCAAAAAATGAAACCAGAAAAAAAT |
| | | CCATTTAAAGGGCTGTCAACGGATAGAAAAATGGACGA |
| | | AGTTTCTCAAGTTGGCGTTTAATTCGCTATCAATTTGAG |
| | | GATGATGATGGCTTAGCAAGAATAGAAAGCGCTTATGT |
| | | GACCGGGTCGGCATGGCATCTCCACCTCCTCGCGGTCC |
| | | GACCTGGGCATCCGAAGGAGGACGCACGTCCACTCGGA |
| | | TGGCTAAGGGAGAGCCTGCAGTAGCATAACCCCTTGGG |
| | | GCCTCTAAACGGGTCTTGAGGGGTTTTTTGGGTACC |
| 16 | NSP 2 protein, | MAELACFCYPHLENDSYKFIPFNNLAIKAMLTAKVDKKD |
| | strain SA11 | MDKFYDSIIYGIAPPPQFKKRYNTNDNSRGMNFETIMFTK |
| | | VAMLICEALNSLKVTQANVSNVLSRVVSIRHLENLVIRKE |
| | | NPQDILFHSKDLLLKSTLIAIGQSKEIETTITAEGGEIVFQNA |
| | | AFTMWKLTYLEHQLMPILDQNFIEYKVTLNEDKPISDVHV |
| | | KELVAELRWQYNKFAVITHGKGHYRIVKYSSVANHADRV |
| | | YATFKSNVKTGVNNDFNLLDQRIIWQNWYAFTSSMKQGN |
| | | TLDVCKRLLFQKMKPEKNPFKGLSTDRKMDEVSQVGV |
| 17 | pT7/NSP3SA11 | ACTAGTTAATACGACTCACTATAGGCATTTAATGCTTTT |
| | T7 promoter: nt | CAGTGGTTGATGCTCAAGATGGAGTCTACGCAACAGAT |
| | 7-24 | GGCCGTCTCAATTATTAACTCTTCTTTTGAAGCTGCAGT |
| | NSP3 CDS: nt | TGTAGCTGCAACCTCAGCTCTTGAGAATATGGGAATAG |
| | 49-996 | AATATGATTATCAGGATATATATTCTAGAGTAAAGAAT |
| | HDV Ribozyme: | AAATTTGATTTTGTGATGGACGATTCTGGTGTTAAAAAT |
| | nt 1,129-1,217 | AATCTGATTGGTAAAGCAATAACTATTGATCAAGCTTTG |
| | T7 Terminator: | AATAATAAATTTGGATCTGCTATAAGAAATAGAAACTG |
| | nt 1,226-1,268 | GCTTGCTGATACTTCTAGAGCAGCTAAATTAGATGAGG |
| | | ATGTAAACAAACTAAGAATGATGTTATCATCAAAAGGA |
| | | ATTGATCAAAAAATGAGAGTTTTAAACGCATGCTTCAG |
| | | TGTAAAAAGAATACCTGGAAAATCATCATCTATTATTA |
| | | AATGCACAAAATTGATGCGTGATAAATTGGAACGTGGT |
| | | GAAGTTGAAGTGGATGATTCATTTGTGGATGAAAAAAT |
| | | GGAAGTGGATACCATTGACTGGAAATCGCGCTATGAGC |
| | | AATTGGAGCAAAGGTTTGAATCATTGAAATCCAGGGTA |
| | | AATGAAAAATATAATAATTGGGTGTTGAAAGCAAGAAA |
| | | AATGAATGAAAATATGCATTCTCTTCAAAATGTCATCTC |
| | | TCAACAGCAAGCACATATAGCTGAGCTTCAAGTGTACA |
| | | ATAATAAACTAGAACGTGATTTGCAAAATAAAATTGGA |
| | | TCCCTTACTTCTTCGATTGAATGGTATTTAAGATCAATG |
| | | GAATTAGACCCTGAAATAAAGGCAGACATTGAACAGCA |
| | | AATTAACTCAATTGATGCGATAAATCCATTGCACGCTTT |
| | | TGATGACTTAGAATCAGTAATACGTAATTTGATATCTGA |
| | | TTATGACAAATTATTCCTTATGTTCAAAGGATTAATACA |
| | | GAGATGTAATTATCAATATTCATTTGGTTGCGAATAACC |
| | | ATTTTGATACATGTTGAACAATCAAATACAGTGTTAGTA |
| | | TGTTGTCATCTATGCATAACCCTCTATGAGCACAATAGT |
| | | TAAAAGCTAACACTGTCAAAAACCTAAATGGCTATAGG |
| | | GGCGTTATGTGGCCGGGTCGGCATGGCATCTCCACCTCC |
| | | TCGCGGTCCGACCTGGGCATCCGAAGGAGGACGCACGT |
| | | CCACTCGGATGGCTAAGGGAGAGCCTGCAGTAGCATAA |
| | | CCCCTTGGGGCCTCTAAACGGGTCTTGAGGGGTTTTTTG |
| | | GGTACC |
| 18 | NSP3 protein, | MLKMESTQQMAVSIINSSFEAAVVAATSALENMGIEYDY |
| | strain SA11 | QDIYSRVKNKFDFVMDDSGVKNNLIGKAITIDQALNNKFG |
| | | SAIRNRNWLADTSRAAKLDEDVNKLRMMLSSKGIDQKM |
| | | RVLNACFSVKRIPGKSSSIIKCTKLMRDKLERGEVEVDDSF |
| | | VDEKMEVDTIDWKSRYEQLEQRFESLKSRVNEKYNNWVL |
| | | KARKMNENMHSLQNVISQQQAHIAELQVYNNKLERDLQ |
| | | NKIGSLTSSIEWYLRSMELDPEIKADIEQQINSIDAINPLHAF |
| | | DDLESVIRNLISDYDKLFLMFKGLIQRCNYQYSFGCE |
| 19 | pT7/NSP4SA11 | ACTAGTTAATACGACTCACTATAGGCTTTTAAAAGTTCT |
| | T7 promoter: nt | GTTCCGAGAGAGCGCGTGCGGAAAGATGGAAAAGCTTA |
| | 7-24 | CCGACCTCAATTATACATTGAGTGTAATCACTCTAATGA |
| | NSP4 CDS: nt | ACAATACATTGCACACAATACTTGAGGATCCAGGAATG |
| | 65-592 | GCGTATTTTCCTTATATAGCATCTGTCTTAACAGTTTTGT |
| | HDV Ribozyme: | TTGCGCTACATAAAGCATCCATTCCAACAATGAAAATT |
| | nt 775-863 | GCATTGAAAACGTCAAAATGTTCATATAAAGTGGTGAA |
| | T7 Terminator: | ATATTGTATTGTAACAATTTTTAATACGTTGTTAAAATT |
| | nt 872-914 | GGCAGGTTATAAAGAGCAGATAACTACTAAAGATGAGA |
| | | TAGAAAAGCAAATGGACAGAGTAGTCAAAGAAATGAG |
| | | ACGCCAGCTAGAAATGATTGACAAATTGACTACACGTG |
| | | AAATTGAACAAGTAGAGTTGCTTAAACGCATTTACGAT |
| | | AAATTGACGGTGCAAACGACAGGTGAAATAGATATGAC |
| | | AAAAGAGATCAATCAAAAAAACGTGAGAACGCTAGAA |
| | | GAATGGGAAAGTGGAAAAAATCCTTATGAACCAAGAG |
| | | AAGTGACTGCAGCAATGTAAGAGGTTGAGCTGCCGTCG |
| | | ACTGTCCTCGGAAGCGGCGGAGTTCTTTACAGTAAGCA |
| | | CCATCGGACCTGATGGCTGACTGAGAAGCCACAGTCAG |
| | | CCATATCGCGTGTGGCTCAAGCCTTAATCCCGTTTAACC |
| | | AATCCGGTCAGCACCGGACGTTAATGGAAGGAACGGTC |
| | | TTAATGTGACCGGGTCGGCATGGCATCTCCACCTCCTCG |
| | | CGGTCCGACCTGGGCATCCGAAGGAGGACGCACGTCCA |
| | | CTCGGATGGCTAAGGGAGAGCCTGCAGTAGCATAACCC |
| | | CTTGGGGCCTCTAAACGGGTCTTGAGGGGTTTTTTGGGT |
| | | ACC |
| 20 | NSP4 protein, | MEKLTDLNYTLSVITLMNNTLHTILEDPGMAYFPYIASVLT |
| | strain SA11 | VLFALHKASIPTMKIALKTSKCSYKVVKYCIVTIFNTLLKL |
| | | AGYKEQITTKDEIEKQMDRVVKEMRRQLEMIDKLTTREIE |
| | | QVELLKRIYDKLTVQTTGEIDMTKEINQKNVRTLEEWESG |
| | | KNPYEPREVTAAM |
| 21 | pT7/NSP5SA11 | AAGCTTTAATACGACTCACTATAGGCTTTTAAAGCGCTA |
| | T7 promoter: nt | CAGTGATGTCTCTCAGTATTGACGTGACGAGTCTTCCTT |
| | 7-24 | CTATTCCTTCAACTATATATAAGAATGAATCGTCTTCAA |
| | NSP5 CDS: nt | CAACGTCAACTCTTTCTGGAAAATCTATTGGTAGGAGTG |
| | 45-641 | AACAGTACATTTCACCAGATGCAGAAGCATTCAATAAA |
| | HDV Ribozyme: | TACATGCTGTCGAAGTCTCCAGAGGATATTGGACCATCT |
| | nt 691-779 | GATTCTGCTTCAAACGATCCACTCACCAGTTTTTCGATT |
| | T7 Terminator: | AGATCGAATGCAGTTAAGACAAACGCAGACGCTGGCGT |
| | nt 788-830 | GTCTATGGATTCATCAGCACAATCACGACCTTCAAGTA |
| | | ATGTCGGATGCGATCAAGTGGATTTCTCCTTAAATAAA |
| | | GGCTTAAAAGTAAAAGCTAATTTGGACTCATCAATATC |
| | | AATATCTACGGATACTAAAAAGGAGAAATCAAAACAA |
| | | AACCATAAAAGTAGGAAGCACTACCCAAGAATTGAAGC |
| | | AGAGTCTGATTCAGATGATTATGTACTGGATGATTCAG |
| | | ATAGTGATGATGGTAAATGTAAGAACTGTAAATATAAG |
| | | AAGAAATACTTCGCATTAAGAATGAGAATGAAACAAGT |
| | | CGCAATGCAATTGATTGAAGATTTGTAAGTCTGACCTG |
| | | GGAACACACTAGGGAGCTCCCCACTCCCGTTTTGTGAC |
| | | CGGGTCGGCATGGCATCTCCACCTCCTCGCGGTCCGACC |
| | | TGGGCATCCGAAGGAGGACGCACGTCCACTCGGATGGC |
| | | TAAGGGAGAGCCTGCAGTAGCATAACCCCTTGGGGCCT |
| | | CTAAACGGGTCTTGAGGGGTTTTTTGGGTACC |
| 22 | NSP5 protein, | MSLSIDVTSLPSIPSTIYKNESSSTTSTLSGKSIGRSEQYISPD |
| | strain SA11 | AEAFNKYMLSKSPEDIGPSDSASNDPLTSFSIRSNAVKTNA |
| | | DAGVSMDSSAQSRPSSNVGCDQVDFSLNKGLKVKANLDS |
| | | SISISTDTKKEKSKQNHKSRKHYPRIEAESDSDDYVLDDSD |
| | | SDDGKCKNCKYKKKYFALRMRMKQVAMQLIEDL |
| 23 | pCMV vector | AGATCTGCTGTGCCTTCTAGTTGCCAGCCATCTGTTGTT |
| | (V1Jns) | TGCCCCTCCCCCGTGCCTTCCTTGACCCTGGAAGGTGCC |
| | BglII site: nt 1-6 | ACTCCCACTGTCCTTTCCTAATAAAATGAGGAAATTGCA |
| | bovine growth | TCGCATTGTCTGAGTAGGTGTCATTCTATTCTGGGGGGT |
| | hormone | GGGGTGGGGCAGGACAGCAAGGGGGAGGATTGGGAAG |
| | polyadenylation | ACAATAGCAGGCATGCTGGGGATGCGGTGGGCTCTATG |
| | signal (BGH | GCCGCTGCGGCCAGGTGCTGAAGAATTGACCCGGTTCC |
| | pA; terminator): | TCCTGGGCCAGAAAGAAGCAGGCACATCCCCTTCTCTG |
| | nt 7-588 | TGACACACCCTGTCCACGCCCCTGGTTCTTAGTTCCAGC |
| | polyadenylation | CCCACTCATAGGACACTCATAGCTCAGGAGGGCTCCGC |
| | signal: nt 98-104 | CTTCAATCCCACCCGCTAAAGTACTTGGAGCGGTCTCTC |
| | Col E1 ori: nt | CCTCCCTCATCAGCCCACCAAACCAAACCTAGCCTCCA |
| | 559-1,574 | AGAGTGGGAAGAAATTAAAGCAAGATAGGCTATTAAGT |
| | Neo/Kan-R: nt | GCAGAGGGAGAGAAAATGCCTCCAACATGTGAGGAAG |
| | 2,809-1,575 | TAATGAGAGAAATCATAGAATTTCTTCCGCTTCCTCGCT |
| | (reverse) | CACTGACTCGCTGCGCTCGGTCGTTCGGCTGCGGCGAG |
| | CMV promoter: | CGGTATCAGCTCACTCAAAGGCGGTAATACGGTTATCC |
| | nt 3,228-4,029 | ACAGAATCAGGGGATAACGCAGGAAAGAACATGTGAG |
| | Intron A: nt | CAAAAGGCCAGCAAAAGGCCAGGAACCGTAAAAAGGC |
| | 4,030-4,861 | CGCGTTGCTGGCGTTTTTCCATAGGCTCCGCCCCCCTGA |
| | | CGAGCATCACAAAAATCGACGCTCAAGTCAGAGGTGGC |
| | | GAAACCCGACAGGACTATAAAGATACCAGGCGTTTCCC |
| | | CCTGGAAGCTCCCTCGTGCGCTCTCCTGTTCCGACCCTG |
| | | CCGCTTACCGGATACCTGTCCGCCTTTCTCCCTTCGGGA |
| | | AGCGTGGCGCTTTCTCATAGCTCACGCTGTAGGTATCTC |
| | | AGTTCGGTGTAGGTCGTTCGCTCCAAGCTGGGCTGTGTG |
| | | CACGAACCCCCCGTTCAGCCCGACCGCTGCGCCTTATCC |
| | | GGTAACTATCGTCTTGAGTCCAACCCGGTAAGACACGA |
| | | CTTATCGCCACTGGCAGCAGCCACTGGTAACAGGATTA |
| | | GCAGAGCGAGGTATGTAGGCGGTGCTACAGAGTTCTTG |
| | | AAGTGGTGGCCTAACTACGGCTACACTAGAAGAACAGT |
| | | ATTTGGTATCTGCGCTCTGCTGAAGCCAGTTACCTTCGG |
| | | AAAAAGAGTTGGTAGCTCTTGATCCGGCAAACAAACCA |
| | | CCGCTGGTAGCGGTGGTTTTTTTGTTTGCAAGCAGCAGA |
| | | TTACGCGCAGAAAAAAAGGATCTCAAGAAGATCCTTTG |
| | | ATCTTTTCTACGGGGTCTGACGCTCAGTGGAACGAAAA |
| | | CTCACGTTAAGGGATTTTGGTCATGAGATTATCAAAAA |
| | | GGATCTTCACCTAGATCCTTTTAAATTAAAAATGAAGTT |
| | | TTAAATCAATCTAAAGTATATATGAGTAAACTTGGTCTG |
| | | ACAGTTACCAATGCTTAATCAGTGAGGCACCTATCTCA |
| | | GCGATCTGTCTATTTCGTTCATCCATAGTTGCCTGACTC |
| | | GGGGGGGGGGGGCGCTGAGGTCTGCCTCGTGAAGAAG |
| | | GTGTTGCTGACTCATACCAGGCCTGAATCGCCCCATCAT |
| | | CCAGCCAGAAAGTGAGGGAGCCACGGTTGATGAGAGCT |
| | | TTGTTGTAGGTGGACCAGTTGGTGATTTTGAACTTTTGC |
| | | TTTGCCACGGAACGGTCTGCGTTGTCGGGAAGATGCGT |
| | | GATCTGATCCTTCAACTCAGCAAAAGTTCGATTTATTCA |
| | | ACAAAGCCGCCGTCCCGTCAAGTCAGCGTAATGCTCTG |
| | | CCAGTGTTACAACCAATTAACCAATTCTGATTAGAAAA |
| | | ACTCATCGAGCATCAAATGAAACTGCAATTTATTCATAT |
| | | CAGGATTATCAATACCATATTTTTGAAAAAGCCGTTTCT |
| | | GTAATGAAGGAGAAAACTCACCGAGGCAGTTCCATAGG |
| | | ATGGCAAGATCCTGGTATCGGTCTGCGATTCCGACTCGT |
| | | CCAACATCAATACAACCTATTAATTTCCCCTCGTCAAAA |
| | | ATAAGGTTATCAAGTGAGAAATCACCATGAGTGACGAC |
| | | TGAATCCGGTGAGAATGGCAAAAGCTTATGCATTTCTTT |
| | | CCAGACTTGTTCAACAGGCCAGCCATTACGCTCGTCATC |
| | | AAAATCACTCGCATCAACCAAACCGTTATTCATTCGTGA |
| | | TTGCGCCTGAGCGAGACGAAATACGCGATCGCTGTTAA |
| | | AAGGACAATTACAAACAGGAATCGAATGCAACCGGCG |
| | | CAGGAACACTGCCAGCGCATCAACAATATTTTCACCTG |
| | | AATCAGGATATTCTTCTAATACCTGGAATGCTGTTTTCC |
| | | CGGGGATCGCAGTGGTGAGTAACCATGCATCATCAGGA |
| | | GTACGGATAAAATGCTTGATGGTCGGAAGAGGCATAAA |
| | | TTCCGTCAGCCAGTTTAGTCTGACCATCTCATCTGTAAC |
| | | ATCATTGGCAACGCTACCTTTGCCATGTTTCAGAAACAA |
| | | CTCTGGCGCATCGGGCTTCCCATACAATCGATAGATTGT |
| | | CGCACCTGATTGCCCGACATTATCGCGAGCCCATTTATA |
| | | CCCATATAAATCAGCATCCATGTTGGAATTTAATCGCGG |
| | | CCTCGAGCAAGACGTTTCCCGTTGAATATGGCTCATAAC |
| | | ACCCCTTGTATTACTGTTTATGTAAGCAGACAGTTTTAT |
| | | TGTTCATGATGATATATTTTTATCTTGTGCAATGTAACA |
| | | TCAGAGATTTTGAGACACAACGTGGCTTTCCCCCCCCCC |
| | | CCATTATTGAAGCATTTATCAGGGTTATTGTCTCATGAG |
| | | CGGATACATATTTGAATGTATTTAGAAAAATAAACAAA |
| | | TAGGGGTTCCGCGCACATTTCCCCGAAAAGTGCCACCT |
| | | GACGTCTAAGAAACCATTATTATCATGACATTAACCTAT |
| | | AAAAATAGGCGTATCACGAGGCCCTTTCGTCTCGCGCG |
| | | TTTCGGTGATGACGGTGAAAACCTCTGACACATGCAGC |
| | | TCCCGGAGACGGTCACAGCTTGTCTGTAAGCGGATGCC |
| | | GGGAGCAGACAAGCCCGTCAGGGCGCGTCAGCGGGTGT |
| | | TGGCGGGTGTCGGGGCTGGCTTAACTATGCGGCATCAG |
| | | AGCAGATTGTACTGAGAGTGCACCATATGCGGTGTGAA |
| | | ATACCGCACAGATGCGTAAGGAGAAAATACCGCATCAG |
| | | ATTGGCTATTGGCCATTGCATACGTTGTATCCATATCAT |
| | | AATATGTACATTTATATTGGCTCATGTCCAACATTACCG |
| | | CCATGTTGACATTGATTATTGACTAGTTATTAATAGTAA |
| | | TCAATTACGGGGTCATTAGTTCATAGCCCATATATGGAG |
| | | TTCCGCGTTACATAACTTACGGTAAATGGCCCGCCTGGC |
| | | TGACCGCCCAACGACCCCCGCCCATTGACGTCAATAAT |
| | | GACGTATGTTCCCATAGTAACGCCAATAGGGACTTTCC |
| | | ATTGACGTCAATGGGTGGAGTATTTACGGTAAACTGCC |
| | | CACTTGGCAGTACATCAAGTGTATCATATGCCAAGTAC |
| | | GCCCCCTATTGACGTCAATGACGGTAAATGGCCCGCCT |
| | | GGCATTATGCCCAGTACATGACCTTATGGGACTTTCCTA |
| | | CTTGGCAGTACATCTACGTATTAGTCATCGCTATTACCA |
| | | TGGTGATGCGGTTTTGGCAGTACATCAATGGGCGTGGA |
| | | TAGCGGTTTGACTCACGGGGATTTCCAAGTCTCCACCCC |
| | | ATTGACGTCAATGGGAGTTTGTTTTGGCACCAAAATCA |
| | | ACGGGACTTTCCAAAATGTCGTAACAACTCCGCCCCATT |
| | | GACGCAAATGGGCGGTAGGCGTGTACGGTGGGAGGTCT |
| | | ATATAAGCAGAGCTCGTTTAGTGAACCGTCAGATCGCC |
| | | TGGAGACGCCATCCACGCTGTTTTGACCTCCATAGAAG |
| | | ACACCGGGACCGATCCAGCCTCCGCGGCCGGGAACGGT |
| | | GCATTGGAACGCGGATTCCCCGTGCCAAGAGTGACGTA |
| | | AGTACCGCCTATAGACTCTATAGGCACACCCCTTTGGCT |
| | | CTTATGCATGCTATACTGTTTTTGGCTTGGGGCCTATAC |
| | | ACCCCCGCTTCCTTATGCTATAGGTGATGGTATAGCTTA |
| | | GCCTATAGGTGTGGGTTATTGACCATTATTGACCACTCC |
| | | CCTATTGGTGACGATACTTTCCATTACTAATCCATAACA |
| | | TGGCTCTTTGCCACAACTATCTCTATTGGCTATATGCCA |
| | | ATACTCTGTCCTTCAGAGACTGACACGGACTCTGTATTT |
| | | TTACAGGATGGGGTCCCATTTATTATTTACAAATTCACA |
| | | TATACAACAACGCCGTCCCCCGTGCCCGCAGTTTTTATT |
| | | AAACATAGCGTGGGATCTCCACGCGAATCTCGGGTACG |
| | | TGTTCCGGACATGGGCTCTTCTCCGGTAGCGGCGGAGCT |
| | | TCCACATCCGAGCCCTGGTCCCATGCCTCCAGCGGCTCA |
| | | TGGTCGCTCGGCAGCTCCTTGCTCCTAACAGTGGAGGCC |
| | | AGACTTAGGCACAGCACAATGCCCACCACCACCAGTGT |
| | | GCCGCACAAGGCCGTGGCGGTAGGGTATGTGTCTGAAA |
| | | ATGAGCGTGGAGATTGGGCTCGCACGGCTGACGCAGAT |
| | | GGAAGACTTAAGGCAGCGGCAGAAGAAGATGCAGGCA |
| | | GCTGAGTTGTTGTATTCTGATAAGAGTCAGAGGTAACTC |
| | | CCGTTGCGGTGCTGTTAACGGTGGAGGGCAGTGTAGTC |
| | | TGAGCAGTACTCGTTGCTGCCGCGCGCGCCACCAGACA |
| | | TAATAGCTGACAGACTAACAGACTGTTCCTTTCCATGGG |
| | | TCTTTTCTGCAGTCACCGTCCTT |
| 24 | Insert | AGATCTGCCACCATGGCTGAGCTAGCTTGCTTTTGCTAT |
| | CMV/NSP2 | |
| | (NSP2 CDS | CCTCATTTGGAGAATGATAGCTATAAATTTATTCCTTTT |
| | flanked by BglII | AATAATTTAGCTATTAAAGCTATGCTGACAGCTAAAGT |
| | restriction sites) | AGACAAAAAGGACATGGATAAGTTTTATGATTCAATTA |
| | BglII site: nt 1-6 | TTTATGGAATAGCACCGCCTCCTCAATTTAAGAAACGGT |
| | NSP2: nt 7-966 | ATAATACTAATGATAATTCAAGAGGCATGAATTTTGAA |
| | BglII site: nt | ACAATTATGTTTACTAAGGTGGCTATGTTGATATGTGAA |
| | 967-972 | GCTCTAAATTCATTGAAAGTGACGCAAGCAAACGTCTC |
| | | TAATGTATTATCACGAGTAGTATCAATAAGGCATTTAG |
| | | AAAATTTGGTGATACGTAAAGAAAATCCACAGGATATT |
| | | CTATTTCATTCAAAAGATTTACTTTTGAAATCAACACTG |
| | | ATTGCTATTGGACAGTCTAAAGAAATTGAAACTACAAT |
| | | AACTGCAGAAGGAGGAGAAATTGTATTTCAAAACGCTG |
| | | CCTTCACCATGTGGAAACTAACTTATTTAGAACATCAAT |
| | | TGATGCCAATTCTGGATCAGAATTTTATTGAATATAAAG |
| | | TTACATTGAACGAAGATAAACCAATTTCAGATGTTCAT |
| | | GTTAAAGAATTAGTCGCTGAACTTCGATGGCAATATAA |
| | | CAAGTTTGCTGTAATCACACATGGTAAGGGTCATTATA |
| | | GAATTGTAAAGTATTCATCAGTTGCTAATCACGCTGACA |
| | | GAGTATATGCAACTTTCAAGAGTAATGTTAAAACTGGA |
| | | GTTAATAATGATTTTAACCTACTTGATCAAAGAATTATT |
| | | TGGCAAAACTGGTATGCATTTACATCATCAATGAAACA |
| | | GGGTAATACACTTGACGTGTGTAAAAGGTTGCTTTTCCA |
| | | AAAAATGAAACCAGAAAAAAATCCATTTAAAGGGCTGT |
| | | CAACGGATAGAAAAATGGACGAAGTTTCTCAAGTTGGC |
| | | GTTTAAAGATCT |
| 25 | Insert | AGATCTGCCACCATGTCTCTCAGTATTGACGTGACGAGT |
| | CMV/NSP5 | CTTCCTTCTATTCCTTCAACTATATATAAGAATGAATCG |
| | (NSP5 CDS | TCTTCAACAACGTCAACTCTTTCTGGAAAATCTATTGGT |
| | flanked by BglII | AGGAGTGAACAGTACATTTCACCAGATGCAGAAGCATT |
| | restriction sites) | CAATAAATACATGCTGTCGAAGTCTCCAGAGGATATTG |
| | BglII site: nt 1-6 | GACCATCTGATTCTGCTTCAAACGATCCACTCACCAGTT |
| | NSP5: nt 7-609 | TTTCGATTAGATCGAATGCAGTTAAGACAAACGCAGAC |
| | BglII site: 610- | GCTGGCGTGTCTATGGATTCATCAGCACAATCACGACCT |
| | 615 | TCAAGTAATGTCGGATGCGATCAAGTGGATTTCTCCTTA |
| | | AATAAAGGCTTAAAAGTAAAAGCTAATTTGGACTCATC |
| | | AATATCAATATCTACGGATACTAAAAAGGAGAAATCAA |
| | | AACAAAACCATAAAAGTAGGAAGCACTACCCAAGAATT |
| | | GAAGCAGAGTCTGATTCAGATGATTATGTACTGGATGA |
| | | TTCAGATAGTGATGATGGTAAATGTAAGAACTGTAAAT |
| | | ATAAGAAGAAATACTTCGCATTAAGAATGAGAATGAAA |
| | | CAAGTCGCAATGCAATTGATTGAAGATTTGTAAAGATC |
| | | T |
| 26 | Insert CMV/ | AGATCTGCCACCATGAGCGGAGATTGTGCCGGCCTGGT |
| | NBVFAST | GTCTGTGTTTGGCAGCGTGCACTGTCAGAGCAGCAAGA |
| | (FAST CDS | ACAAAGCTGGCGGCGATCTGCAGGCCACCAGCGTGTTG |
| | flanked by BglII | ACAACATACTGGCCTCACCTGGCCATCGGCGGCAGCAT |
| | restriction sites) | CATCCTGATCATTCTGCTGCTGGGCCTGTTCTACTGCTG |
| | BglII site: nt 1-6 | CTACCTGAAGTGGAAAACCAGCCACATCCGGCGGACCT |
| | FAST: nt 13- | ACCACAAAGAACTGGTGGCCCTGACCAGAGGCTACGTG |
| | 300 | CGACCTATTCCTGCCGATGTGACCTCCGTGTGAAGATCT |
| | BglII site: 301- | |
| | 306 | |
| 27 | FAST protein | MSGDCAGLVSVFGSVHCQSSKNKAGGDLQATSVLTTYWP |
| | | HLAIGGSIILIILLLGLFYCCYLKWKTSHIRRTYHKELVALT |
| | | RGYVRPIPADVTSV |
| 28 | Insert | AGATCTGCCACCATGGACGAGATCGTGAAGAACATCCG |
| | CMV/D12L | CGAGGGCACACACGTGCTGCTGCCTTTCTATGAGACAC |
| | (D12L CDS | TGCCCGAGCTGAACCTGAGCCTGGGAAAGTCTCCTCTG |
| | flanked by BglII | CCTAGCCTGGAATACGGCGCCAACTACTTCCTGCAGAT |
| | restriction sites) | CAGCAGAGTGAACGACCTGAACAGAATGCCCACCGACA |
| | BglII site: nt 1-6 | TGCTGAAGCTGTTCACCCACGACATCATGCTGCCCGAG |
| | D12L CDS: nt | AGCGACCTGGACAAGGTGTACGAGATTCTGAAGATCAA |
| | 13-876 | CAGCGTGAAGTACTACGGCAGAAGCACCAAGGCCGAC |
| | BglII site: 877- | GCCGTTGTGGCTGATCTGAGCGCCAGAAACAAGCTGTT |
| | 882 | TAAGAGAGAGCGGGACGCCATCAAGAGCAACAACCAC |
| | | CTGACCGAGAACAACCTGTACATCAGCGACTACAAGAT |
| | | GCTGACCTTCGACGTGTTCAGACCCCTGTTCGACTTCGT |
| | | GAACGAGAAGTACTGCATCATCAAGCTGCCCACACTGT |
| | | TCGGCAGAGGCGTGATCGACACCATGCGGATCTACTGC |
| | | AGCCTGTTCAAGAACGTGCGGCTGCTGAAGTGCGTGTC |
| | | CGACAGCTGGCTGAAGGACAGCGCCATTATGGTGGCCA |
| | | GCGACGTGTGCAAGAAGAACCTGGACCTGTTCATGAGC |
| | | CACGTGAAGTCCGTGACCAAGAGCAGCAGCTGGAAGG |
| | | ACGTGAACAGCGTGCAGTTCAGCATCCTGAACAACCCC |
| | | GTGGACACCGAGTTCATCAACAAGTTCCTCGAGTTCAG |
| | | CAACCGCGTGTACGAGGCCCTGTACTACGTGCACAGCC |
| | | TGCTGTACTCCAGCATGACCAGCGACAGCAAGAGCATC |
| | | GAGAACAAGCACCAGCGGCGGCTGGTCAAACTGCTGCT |
| | | GTAAAGATCT |
| 29 | D12L protein | MDEIVKNIREGTHVLLPFYETLPELNLSLGKSPLPSLEYGA |
| | | NYFLQISRVNDLNRMPTDMLKLFTHDIMLPESDLDKVYEI |
| | | LKINSVKYYGRSTKADAVVADLSARNKLFKRERDAIKSN |
| | | NHLTENNLYISDYKMLTFDVFRPLFDFVNEKYCIIKLPTLF |
| | | GRGVIDTMRIYCSLFKNVRLLKCVSDSWLKDSAIMVASDV |
| | | CKKNLDLFMSHVKSVTKSSSWKDVNSVQFSILNNPVDTEF |
| | | INKFLEFSNRVYEALYYVHSLLYSSMTSDSKSIENKHQRRL |
| | | VKLLL |
| 30 | Insert | AGATCTGCCACCATGGACGCCAATGTGGTGTCCAGCTC |
| | CMV/DIR | TACAATCGCCACCTACATCGACGCCCTGGCCAAGAATG |
| | (DIR CDS | CCAGCGAGCTGGAACAGAGAAGCACCGCCTACGAGATC |
| | flanked by BglII | AACAACGAACTGGAACTGGTGTTCATCAAGCCTCCACT |
| | restriction sites) | GATCACCCTGACCAACGTGGTCAACATCAGCACCATCC |
| | BglII site: nt 1-6 | AAGAGAGCTTCATCCGGTTTACCGTGACCAACAAAGAA |
| | DIR CDS: nt | GGCGTGAAGATCCGGACCAAGATTCCCCTGTCTAAGGT |
| | 13-2,547 | GCACGGCCTGGACGTGAAGAACGTGCAGCTGGTGGACG |
| | BglII site: | CCATCGACAACATCGTGTGGGAGAAGAAGTCCCTGGTC |
| | 2,547-2,553 | ACCGAGAACCGGCTGCACAAAGAGTGCCTGCTGAGACT |
| | | GAGCACCGAGGAACGGCACATCTTTCTGGACTACAAGA |
| | | AGTACGGCAGCAGCATCCGGCTGGAACTCGTGAATCTG |
| | | ATCCAGGCCAAGACCAAGAACTTCACCATCGACTTCAA |
| | | GCTCAAGTACTTCCTCGGCAGCGGAGCCCAGAGCAAGT |
| | | CTAGTCTGCTGCACGCCATCAATCACCCCAAGAGCAGA |
| | | CCCAACACCAGCCTGGAAATCGAGTTCACCCCTCGGGA |
| | | CAACGAGACAGTGCCCTACGACGAGCTGATCAAAGAGC |
| | | TGACCACACTGAGCCGCCACATCTTCATGGCTAGCCCC |
| | | GAGAATGTGATCCTGTCTCCTCCTATCAACGCCCCTATC |
| | | AAGACCTTCATGCTGCCCAAGCAGGACATCGTGGGCCT |
| | | CGACCTGGAAAACCTGTACGCCGTGACAAAGACCGACG |
| | | GCATCCCCATCACCATCAGAGTGACCAGCAACGGCCTG |
| | | TACTGCTACTTCACCCACCTGGGCTACATCATCAGATAC |
| | | CCCGTGAAGCGGATCATCGACAGCGAGGTGGTGGTGTT |
| | | TGGAGAGGCCGTGAAGGACAAGAACTGGACCGTGTACC |
| | | TGATCAAGCTGATCGAGCCCGTGAATGCCATCAACGAC |
| | | AGACTGGAAGAGAGCAAATACGTCGAGAGCAAACTGG |
| | | TGGACATCTGCGACCGGATCGTGTTCAAGAGCAAGAAG |
| | | TATGAGGGCCCCTTTACCACCACCAGTGAAGTGGTGGA |
| | | TATGCTGAGCACCTACCTGCCTAAGCAGCCCGAGGGCG |
| | | TCATCCTGTTTTACAGCAAGGGCCCCAAGTCCAACATCG |
| | | ATTTCAAGATTAAGAAAGAGAACACCATCGATCAGACC |
| | | GCCAACGTCGTGTTCCGGTACATGAGCAGCGAGCCCAT |
| | | CATCTTCGGCGAGAGCAGCATCTTCGTCGAGTATAAGA |
| | | AGTTCAGCAACGACAAGGGCTTCCCCAAAGAGTACGGC |
| | | TCCGGCAAGATCGTGCTGTACAACGGCGTGAACTACCT |
| | | GAACAACATCTACTGCCTCGAGTACATCAACACCCACA |
| | | ACGAAGTGGGCATCAAGAGCGTGGTGGTGCCCATCAAG |
| | | TTTATCGCCGAGTTCCTGGTCAACGGCGAGATCCTGAA |
| | | GCCTCGGATCGACAAGACCATGAAGTATATCAACAGCG |
| | | AGGACTACTACGGCAACCAGCACAACATCATCGTGGAA |
| | | CACCTGAGGGACCAGAGCATCAAGATCGGCGACATCTT |
| | | CAACGAGGACAAGCTGAGCGACGTGGGCCACCAGTAC |
| | | GCCAACAACGATAAGTTCCGGCTGAACCCCGAGGTGTC |
| | | CTACTTTACCAACAAGCGGACCAGAGGACCCCTGGGCA |
| | | TCCTGAGCAACTACGTGAAAACCCTGCTGATCTCCATGT |
| | | ACTGCAGCAAGACATTCCTGGACGACAGCAACAAGAGA |
| | | AAGGTGCTGGCCATTGACTTCGGCAACGGCGCCGATCT |
| | | GGAAAAGTACTTCTATGGCGAGATCGCCCTGCTGGTGG |
| | | CCACAGATCCTGATGCCGATGCCATTGCCAGAGGCAAC |
| | | GAGCGGTACAACAAGCTGAACAGCGGCATTAAGACCA |
| | | AGTACTACAAGTTCGACTACATCCAAGAAACCATTCGG |
| | | AGCGACACCTTCGTGTCCAGCGTGCGCGAGGTGTTCTAT |
| | | TTCGGCAAGTTCAATATCATCGACTGGCAGTTCGCCATC |
| | | CACTACAGCTTTCACCCCAGACACTACGCCACCGTGAT |
| | | GAACAACCTGAGCGAGCTGACAGCCTCTGGCGGCAAGG |
| | | TGCTGATCACCACAATGGACGGCGACAAGCTGTCCAAG |
| | | CTGACCGACAAGAAAACCTTCATCATCCACAAGAATCT |
| | | GCCCAGCAGCGAGAACTACATGAGCGTGGAAAAGATC |
| | | GCCGACGACAGAATCGTGGTGTATAACCCCAGCACCAT |
| | | GAGCACCCCTATGACCGAGTATATCATCAAGAAGAACG |
| | | ACATCGTCCGGGTGTTCAACGAGTATGGCTTCGTGCTGG |
| | | TGGATAACGTGGACTTCGCCACCATCATCGAGCGGTCC |
| | | AAGAAGTTTATCAACGGGGCCAGCACAATGGAAGATCG |
| | | GCCCTCCACCAGAAACTTTTTCGAGCTGAATAGAGGCG |
| | | CCATCAAGTGCGAAGGCCTGGATGTCGAGGACCTGCTG |
| | | TCCTACTACGTGGTGTACGTGTTCAGCAAGCGCTGAAG |
| | | ATCT |
| 31 | DIR protein | MDANVVSSSTIATYIDALAKNASELEQRSTAYEINNELELV |
| | | FIKPPLITLTNVVNISTIQESFIRFTVTNKEGVKIRTKIPLSKV |
| | | HGLDVKNVQLVDAIDNIVWEKKSLVTENRLHKECLLRLS |
| | | TEERHIFLDYKKYGSSIRLELVNLIQAKTKNFTIDFKLKYFL |
| | | GSGAQSKSSLLHAINHPKSRPNTSLEIEFTPRDNETVPYDEL |
| | | IKELTTLSRHIFMASPENVILSPPINAPIKTFMLPKQDIVGLD |
| | | LENLYAVTKTDGIPITIRVTSNGLYCYFTHLGYIIRYPVKRII |
| | | DSEVVVFGEAVKDKNWTVYLIKLIEPVNAINDRLEESKYV |
| | | ESKLVDICDRIVFKSKKYEGPFTTTSEVVDMLSTYLPKQPE |
| | | GVILFYSKGPKSNIDFKIKKENTIDQTANVVFRYMSSEPIIF |
| | | GESSIFVEYKKFSNDKGFPKEYGSGKIVLYNGVNYLNNIY |
| | | CLEYINTHNEVGIKSVVVPIKFIAEFLVNGEILKPRIDKTMK |
| | | YINSEDYYGNQHNIIVEHLRDQSIKIGDIFNEDKLSDVGHQ |
| | | YANNDKFRLNPEVSYFTNKRTRGPLGILSNYVKTLLISMY |
| | | CSKTFLDDSNKRKVLAIDFGNGADLEKYFYGEIALLVATD |
| | | PDADAIARGNERYNKLNSGIKTKYYKFDYIQETIRSDTFVS |
| | | SVREVFYFGKFNIIDWQFAIHYSFHPRHYATVMNNLSELT |
| | | ASGGKVLITTMDGDKLSKLTDKKTFIIHKNLPSSENYMSV |
| | | EKIADDRIVVYNPSTMSTPMTEYIIKKNDIVRVFNEYGFVL |
| | | VDNVDFATIIERSKKFINGASTMEDRPSTRNFFELNRGAIK |
| | | CEGLDVEDLLSYYVVYVFSKR |
| 32 | T2A peptide | GSGEGRGSLLTCGDVEENPGP |
| 33 | P2A peptide | GSGATNFSLLKQAGDVEENPGP |
| 34 | E2A peptide | GSGQCTNYALLKLAGDVESNPGP |
| 35 | F2A peptide | GSGVKQTLNFDLLKLAGDVESNPGP |
| 36 | S1 domain of | MFVFLVLLPLVSSQCVNLTTRTQLPPAYTNSFTRGVYYPD |
| | SARS-CoV-2 | KVFRSSVLHSTQDLFLPFFSNVTWFHAIHVSGTNGTKRFD |
| | Spike protein | NPVLPFNDGVYFASTEKSNIIRGWIFGTTLDSKTQSLLIVNN |
| | | ATNVVIKVCEFQFCNDPFLGVYYHKNNKSWMESEFRVYS |
| | | SANNCTFEYVSQPFLMDLEGKQGNFKNLREFVFKNIDGYF |
| | | KIYSKHTPINLVRDLPQGFSALEPLVDLPIGINITRFQTLLAL |
| | | HRSYLTPGDSSSGWTAGAAAYYVGYLQPRTFLLKYNENG |
| | | TITDAVDCALDPLSETKCTLKSFTVEKGIYQTSNFRVQPTE |
| | | SIVRFPNITNLCPFGEVFNATRFASVYAWNRKRISNCVADY |
| | | SVLYNSASFSTFKCYGVSPTKLNDLCFTNVYADSFVIRGDE |
| | | VRQIAPGQTGKIADYNYKLPDDFTGCVIAWNSNNLDSKV |
| | | GGNYNYLYRLFRKSNLKPFERDISTEIYQAGSTPCNGVEGF |
| | | NCYFPLQSYGFQPTNGVGYQPYRVVVLSFELLHAPATVCG |
| | | PKKSTNLVKNKCVNFNFNGLTGTGVLTESNKKFLPFQQFG |
| | | RDIADTTDAVRDPQTLEILDITPCSFGGVSVITPGTNTSNQV |
| | | AVLYQDVNCTEVPVAIHADQLTPTWRVYSTGSNVFQTRA |
| | | GCLIGAEHVNNSYECDIPIGAGICASYQTQTNSP |
| 37 | RBD of SARS- | MFVFLVLLPLVSSQCRVQPTESIVRFPNITNLCPFGEVFNAT |
| | CoV-2 Spike | RFASVYAWNRKRISNCVADYSVLYNSASFSTFKCYGVSPT |
| | protein | KLNDLCFTNVYADSFVIRGDEVRQIAPGQTGKIADYNYKL |
| | | PDDFTGCVIAWNSNNLDSKVGGNYNYLYRLFRKSNLKPF |
| | | ERDISTEIYQAGSTPCNGVEGFNCYFPLQSYGFQPTNGVGY |
| | | QPYRVVVLSFELLHAPATVCGPKKSTNLVKNKCVNFNFN |
| | | GLTGTGVLTESNKKFLPFQQFGRDIADTTDAVRDPQTLEIL |
| | | DITPCS |
| 38 | SARS-CoV-2 | AUUAAAGGUUUAUACCUUCCCAGGUAACAAACCAACC |
| | GenBank:MN90 | AACUUUCGAUCUCUUGUAGAUCUGUUCUCUAAACGAA |
| | 8947 | CUUUAAAAUCUGUGUGGCUGUCACUCGGCUGCAUGCU |
| | | UAGUGCACUCACGCAGUAUAAUUAAUAACUAAUUACU |
| | | GUCGUUGACAGGACACGAGUAACUCGUCUAUCUUCUG |
| | | CAGGCUGCUUACGGUUUCGUCCGUGUUGCAGCCGAUC |
| | | AUCAGCACAUCUAGGUUUCGUCCGGGUGUGACCGAAA |
| | | GGUAAGAUGGAGAGCCUUGUCCCUGGUUUCAACGAGA |
| | | AAACACACGUCCAACUCAGUUUGCCUGUUUUACAGGU |
| | | UCGCGACGUGCUCGUACGUGGCUUUGGAGACUCCGUG |
| | | GAGGAGGUCUUAUCAGAGGCACGUCAACAUCUUAAAG |
| | | AUGGCACUUGUGGCUUAGUAGAAGUUGAAAAAGGCG |
| | | UUUUGCCUCAACUUGAACAGCCCUAUGUGUUCAUCAA |
| | | ACGUUCGGAUGCUCGAACUGCACCUCAUGGUCAUGUU |
| | | AUGGUUGAGCUGGUAGCAGAACUCGAAGGCAUUCAGU |
| | | ACGGUCGUAGUGGUGAGACACUUGGUGUCCUUGUCCC |
| | | UCAUGUGGGCGAAAUACCAGUGGCUUACCGCAAGGUU |
| | | CUUCUUCGUAAGAACGGUAAUAAAGGAGCUGGUGGCC |
| | | AUAGUUACGGCGCCGAUCUAAAGUCAUUUGACUUAGG |
| | | CGACGAGCUUGGCACUGAUCCUUAUGAAGAUUUUCAA |
| | | GAAAACUGGAACACUAAACAUAGCAGUGGUGUUACCC |
| | | GUGAACUCAUGCGUGAGCUUAACGGAGGGGCAUACAC |
| | | UCGCUAUGUCGAUAACAACUUCUGUGGCCCUGAUGGC |
| | | UACCCUCUUGAGUGCAUUAAAGACCUUCUAGCACGUG |
| | | CUGGUAAAGCUUCAUGCACUUUGUCCGAACAACUGGA |
| | | CUUUAUUGACACUAAGAGGGGUGUAUACUGCUGCCGU |
| | | GAACAUGAGCAUGAAAUUGCUUGGUACACGGAACGUU |
| | | CUGAAAAGAGCUAUGAAUUGCAGACACCUUUUGAAAU |
| | | UAAAUUGGCAAAGAAAUUUGACACCUUCAAUGGGGAA |
| | | UGUCCAAAUUUUGUAUUUCCCUUAAAUUCCAUAAUCA |
| | | AGACUAUUCAACCAAGGGUUGAAAAGAAAAAGCUUGA |
| | | UGGCUUUAUGGGUAGAAUUCGAUCUGUCUAUCCAGUU |
| | | GCGUCACCAAAUGAAUGCAACCAAAUGUGCCUUUCAA |
| | | CUCUCAUGAAGUGUGAUCAUUGUGGUGAAACUUCAUG |
| | | GCAGACGGGCGAUUUUGUUAAAGCCACUUGCGAAUUU |
| | | UGUGGCACUGAGAAUUUGACUAAAGAAGGUGCCACUA |
| | | CUUGUGGUUACUUACCCCAAAAUGCUGUUGUUAAAAU |
| | | UUAUUGUCCAGCAUGUCACAAUUCAGAAGUAGGACCU |
| | | GAGCAUAGUCUUGCCGAAUACCAUAAUGAAUCUGGCU |
| | | UGAAAACCAUUCUUCGUAAGGGUGGUCGCACUAUUGC |
| | | CUUUGGAGGCUGUGUGUUCUCUUAUGUUGGUUGCCAU |
| | | AACAAGUGUGCCUAUUGGGUUCCACGUGCUAGCGCUA |
| | | ACAUAGGUUGUAACCAUACAGGUGUUGUUGGAGAAG |
| | | GUUCCGAAGGUCUUAAUGACAACCUUCUUGAAAUACU |
| | | CCAAAAAGAGAAAGUCAACAUCAAUAUUGUUGGUGAC |
| | | UUUAAACUUAAUGAAGAGAUCGCCAUUAUUUUGGCAU |
| | | CUUUUUCUGCUUCCACAAGUGCUUUUGUGGAAACUGU |
| | | GAAAGGUUUGGAUUAUAAAGCAUUCAAACAAAUUGU |
| | | UGAAUCCUGUGGUAAUUUUAAAGUUACAAAAGGAAA |
| | | AGCUAAAAAAGGUGCCUGGAAUAUUGGUGAACAGAA |
| | | AUCAAUACUGAGUCCUCUUUAUGCAUUUGCAUCAGAG |
| | | GCUGCUCGUGUUGUACGAUCAAUUUUCUCCCGCACUC |
| | | UUGAAACUGCUCAAAAUUCUGUGCGUGUUUUACAGAA |
| | | GGCCGCUAUAACAAUACUAGAUGGAAUUUCACAGUAU |
| | | UCACUGAGACUCAUUGAUGCUAUGAUGUUCACAUCUG |
| | | AUUUGGCUACUAACAAUCUAGUUGUAAUGGCCUACAU |
| | | UACAGGUGGUGUUGUUCAGUUGACUUCGCAGUGGCUA |
| | | ACUAACAUCUUUGGCACUGUUUAUGAAAAACUCAAAC |
| | | CCGUCCUUGAUUGGCUUGAAGAGAAGUUUAAGGAAGG |
| | | UGUAGAGUUUCUUAGAGACGGUUGGGAAAUUGUUAA |
| | | AUUUAUCUCAACCUGUGCUUGUGAAAUUGUCGGUGGA |
| | | CAAAUUGUCACCUGUGCAAAGGAAAUUAAGGAGAGUG |
| | | UUCAGACAUUCUUUAAGCUUGUAAAUAAAUUUUUGGC |
| | | UUUGUGUGCUGACUCUAUCAUUAUUGGUGGAGCUAAA |
| | | CUUAAAGCCUUGAAUUUAGGUGAAACAUUUGUCACGC |
| | | ACUCAAAGGGAUUGUACAGAAAGUGUGUUAAAUCCAG |
| | | AGAAGAAACUGGCCUACUCAUGCCUCUAAAAGCCCCA |
| | | AAAGAAAUUAUCUUCUUAGAGGGAGAAACACUUCCCA |
| | | CAGAAGUGUUAACAGAGGAAGUUGUCUUGAAAACUG |
| | | GUGAUUUACAACCAUUAGAACAACCUACUAGUGAAGC |
| | | UGUUGAAGCUCCAUUGGUUGGUACACCAGUUUGUAUU |
| | | AACGGGCUUAUGUUGCUCGAAAUCAAAGACACAGAAA |
| | | AGUACUGUGCCCUUGCACCUAAUAUGAUGGUAACAAA |
| | | CAAUACCUUCACACUCAAAGGCGGUGCACCAACAAAG |
| | | GUUACUUUUGGUGAUGACACUGUGAUAGAAGUGCAA |
| | | GGUUACAAGAGUGUGAAUAUCACUUUUGAACUUGAU |
| | | GAAAGGAUUGAUAAAGUACUUAAUGAGAAGUGCUCU |
| | | GCCUAUACAGUUGAACUCGGUACAGAAGUAAAUGAGU |
| | | UCGCCUGUGUUGUGGCAGAUGCUGUCAUAAAAACUUU |
| | | GCAACCAGUAUCUGAAUUACUUACACCACUGGGCAUU |
| | | GAUUUAGAUGAGUGGAGUAUGGCUACAUACUACUUA |
| | | UUUGAUGAGUCUGGUGAGUUUAAAUUGGCUUCACAU |
| | | AUGUAUUGUUCUUUCUACCCUCCAGAUGAGGAUGAAG |
| | | AAGAAGGUGAUUGUGAAGAAGAAGAGUUUGAGCCAU |
| | | CAACUCAAUAUGAGUAUGGUACUGAAGAUGAUUACCA |
| | | AGGUAAACCUUUGGAAUUUGGUGCCACUUCUGCUGCU |
| | | CUUCAACCUGAAGAAGAGCAAGAAGAAGAUUGGUUAG |
| | | AUGAUGAUAGUCAACAAACUGUUGGUCAACAAGACGG |
| | | CAGUGAGGACAAUCAGACAACUACUAUUCAAACAAUU |
| | | GUUGAGGUUCAACCUCAAUUAGAGAUGGAACUUACAC |
| | | CAGUUGUUCAGACUAUUGAAGUGAAUAGUUUUAGUG |
| | | GUUAUUUAAAACUUACUGACAAUGUAUACAUUAAAA |
| | | AUGCAGACAUUGUGGAAGAAGCUAAAAAGGUAAAACC |
| | | AACAGUGGUUGUUAAUGCAGCCAAUGUUUACCUUAAA |
| | | CAUGGAGGAGGUGUUGCAGGAGCCUUAAAUAAGGCUA |
| | | CUAACAAUGCCAUGCAAGUUGAAUCUGAUGAUUACAU |
| | | AGCUACUAAUGGACCACUUAAAGUGGGUGGUAGUUGU |
| | | GUUUUAAGCGGACACAAUCUUGCUAAACACUGUCUUC |
| | | AUGUUGUCGGCCCAAAUGUUAACAAAGGUGAAGACAU |
| | | UCAACUUCUUAAGAGUGCUUAUGAAAAUUUUAAUCAG |
| | | CACGAAGUUCUACUUGCACCAUUAUUAUCAGCUGGUA |
| | | UUUUUGGUGCUGACCCUAUACAUUCUUUAAGAGUUUG |
| | | UGUAGAUACUGUUCGCACAAAUGUCUACUUAGCUGUC |
| | | UUUGAUAAAAAUCUCUAUGACAAACUUGUUUCAAGCU |
| | | UUUUGGAAAUGAAGAGUGAAAAGCAAGUUGAACAAA |
| | | AGAUCGCUGAGAUUCCUAAAGAGGAAGUUAAGCCAUU |
| | | UAUAACUGAAAGUAAACCUUCAGUUGAACAGAGAAAA |
| | | CAAGAUGAUAAGAAAAUCAAAGCUUGUGUUGAAGAA |
| | | GUUACAACAACUCUGGAAGAAACUAAGUUCCUCACAG |
| | | AAAACUUGUUACUUUAUAUUGACAUUAAUGGCAAUCU |
| | | UCAUCCAGAUUCUGCCACUCUUGUUAGUGACAUUGAC |
| | | AUCACUUUCUUAAAGAAAGAUGCUCCAUAUAUAGUGG |
| | | GUGAUGUUGUUCAAGAGGGUGUUUUAACUGCUGUGG |
| | | UUAUACCUACUAAAAAGGCUGGUGGCACUACUGAAAU |
| | | GCUAGCGAAAGCUUUGAGAAAAGUGCCAACAGACAAU |
| | | UAUAUAACCACUUACCCGGGUCAGGGUUUAAAUGGUU |
| | | ACACUGUAGAGGAGGCAAAGACAGUGCUUAAAAAGUG |
| | | UAAAAGUGCCUUUUACAUUCUACCAUCUAUUAUCUCU |
| | | AAUGAGAAGCAAGAAAUUCUUGGAACUGUUUCUUGG |
| | | AAUUUGCGAGAAAUGCUUGCACAUGCAGAAGAAACAC |
| | | GCAAAUUAAUGCCUGUCUGUGUGGAAACUAAAGCCAU |
| | | AGUUUCAACUAUACAGCGUAAAUAUAAGGGUAUUAA |
| | | AAUACAAGAGGGUGUGGUUGAUUAUGGUGCUAGAUU |
| | | UUACUUUUACACCAGUAAAACAACUGUAGCGUCACUU |
| | | AUCAACACACUUAACGAUCUAAAUGAAACUCUUGUUA |
| | | CAAUGCCACUUGGCUAUGUAACACAUGGCUUAAAUUU |
| | | GGAAGAAGCUGCUCGGUAUAUGAGAUCUCUCAAAGUG |
| | | CCAGCUACAGUUUCUGUUUCUUCACCUGAUGCUGUUA |
| | | CAGCGUAUAAUGGUUAUCUUACUUCUUCUUCUAAAAC |
| | | ACCUGAAGAACAUUUUAUUGAAACCAUCUCACUUGCU |
| | | GGUUCCUAUAAAGAUUGGUCCUAUUCUGGACAAUCUA |
| | | CACAACUAGGUAUAGAAUUUCUUAAGAGAGGUGAUA |
| | | AAAGUGUAUAUUACACUAGUAAUCCUACCACAUUCCA |
| | | CCUAGAUGGUGAAGUUAUCACCUUUGACAAUCUUAAG |
| | | ACACUUCUUUCUUUGAGAGAAGUGAGGACUAUUAAGG |
| | | UGUUUACAACAGUAGACAACAUUAACCUCCACACGCA |
| | | AGUUGUGGACAUGUCAAUGACAUAUGGACAACAGUUU |
| | | GGUCCAACUUAUUUGGAUGGAGCUGAUGUUACUAAAA |
| | | UAAAACCUCAUAAUUCACAUGAAGGUAAAACAUUUUA |
| | | UGUUUUACCUAAUGAUGACACUCUACGUGUUGAGGCU |
| | | UUUGAGUACUACCACACAACUGAUCCUAGUUUUCUGG |
| | | GUAGGUACAUGUCAGCAUUAAAUCACACUAAAAAGUG |
| | | GAAAUACCCACAAGUUAAUGGUUUAACUUCUAUUAAA |
| | | UGGGCAGAUAACAACUGUUAUCUUGCCACUGCAUUGU |
| | | UAACACUCCAACAAAUAGAGUUGAAGUUUAAUCCACC |
| | | UGCUCUACAAGAUGCUUAUUACAGAGCAAGGGCUGGU |
| | | GAAGCUGCUAACUUUUGUGCACUUAUCUUAGCCUACU |
| | | GUAAUAAGACAGUAGGUGAGUUAGGUGAUGUUAGAG |
| | | AAACAAUGAGUUACUUGUUUCAACAUGCCAAUUUAGA |
| | | UUCUUGCAAAAGAGUCUUGAACGUGGUGUGUAAAACU |
| | | UGUGGACAACAGCAGACAACCCUUAAGGGUGUAGAAG |
| | | CUGUUAUGUACAUGGGCACACUUUCUUAUGAACAAUU |
| | | ACAAGCUACAAAAUAUCUAGUACAACAGGAGUCACCU |
| | | UUUGUUAUGAUGUCAGCACCACCUGCUCAGUAUGAAC |
| | | UUAAGCAUGGUACAUUUACUUGUGCUAGUGAGUACAC |
| | | UGGUAAUUACCAGUGUGGUCACUAUAAACAUAUAACU |
| | | UCUAAAGAAACUUUGUAUUGCAUAGACGGUGCUUUAC |
| | | UUACAAAGUCCUCAGAAUACAAAGGUCCUAUUACGGA |
| | | UGUUUUCUACAAAGAAAACAGUUACACAACAACCAUA |
| | | AAACCAGUUACUUAUAAAUUGGAUGGUGUUGUUUGU |
| | | ACAGAAAUUGACCCUAAGUUGGACAAUUAUUAUAAGA |
| | | AAGACAAUUCUUAUUUCACAGAGCAACCAAUUGAUCU |
| | | UGUACCAAACCAACCAUAUCCAAACGCAAGCUUCGAU |
| | | AAUUUUAAGUUUGUAUGUGAUAAUAUCAAAUUUGCU |
| | | GAUGAUUUAAACCAGUUAACUGGUUAUAAGAAACCUG |
| | | CUUCAAGAGAGCUUAAAGUUACAUUUUUCCCUGACUU |
| | | AAAUGGUGAUGUGGUGGCUAUUGAUUAUAAACACUA |
| | | CACACCCUCUUUUAAGAAAGGAGCUAAAUUGUUACAU |
| | | AAACCUAUUGUUUGGCAUGUUAACAAUGCAACUAAUA |
| | | AAGCCACGUAUAAACCAAAUACCUGGUGUAUACGUUG |
| | | UCUUUGGAGCACAAAACCAGUUGAAACAUCAAAUUCG |
| | | UUUGAUGUACUGAAGUCAGAGGACGCGCAGGGAAUGG |
| | | AUAAUCUUGCCUGCGAAGAUCUAAAACCAGUCUCUGA |
| | | AGAAGUAGUGGAAAAUCCUACCAUACAGAAAGACGUU |
| | | CUUGAGUGUAAUGUGAAAACUACCGAAGUUGUAGGA |
| | | GACAUUAUACUUAAACCAGCAAAUAAUAGUUUAAAAA |
| | | UUACAGAAGAGGUUGGCCACACAGAUCUAAUGGCUGC |
| | | UUAUGUAGACAAUUCUAGUCUUACUAUUAAGAAACCU |
| | | AAUGAAUUAUCUAGAGUAUUAGGUUUGAAAACCCUU |
| | | GCUACUCAUGGUUUAGCUGCUGUUAAUAGUGUCCCUU |
| | | GGGAUACUAUAGCUAAUUAUGCUAAGCCUUUUCUUAA |
| | | CAAAGUUGUUAGUACAACUACUAACAUAGUUACACGG |
| | | UGUUUAAACCGUGUUUGUACUAAUUAUAUGCCUUAUU |
| | | UCUUUACUUUAUUGCUACAAUUGUGUACUUUUACUAG |
| | | AAGUACAAAUUCUAGAAUUAAAGCAUCUAUGCCGACU |
| | | ACUAUAGCAAAGAAUACUGUUAAGAGUGUCGGUAAA |
| | | UUUUGUCUAGAGGCUUCAUUUAAUUAUUUGAAGUCAC |
| | | CUAAUUUUUCUAAACUGAUAAAUAUUAUAAUUUGGU |
| | | UUUUACUAUUAAGUGUUUGCCUAGGUUCUUUAAUCUA |
| | | CUCAACCGCUGCUUUAGGUGUUUUAAUGUCUAAUUUA |
| | | GGCAUGCCUUCUUACUGUACUGGUUACAGAGAAGGCU |
| | | AUUUGAACUCUACUAAUGUCACUAUUGCAACCUACUG |
| | | UACUGGUUCUAUACCUUGUAGUGUUUGUCUUAGUGGU |
| | | UUAGAUUCUUUAGACACCUAUCCUUCUUUAGAAACUA |
| | | UACAAAUUACCAUUUCAUCUUUUAAAUGGGAUUUAAC |
| | | UGCUUUUGGCUUAGUUGCAGAGUGGUUUUUGGCAUA |
| | | UAUUCUUUUCACUAGGUUUUUCUAUGUACUUGGAUUG |
| | | GCUGCAAUCAUGCAAUUGUUUUUCAGCUAUUUUGCAG |
| | | UACAUUUUAUUAGUAAUUCUUGGCUUAUGUGGUUAA |
| | | UAAUUAAUCUUGUACAAAUGGCCCCGAUUUCAGCUAU |
| | | GGUUAGAAUGUACAUCUUCUUUGCAUCAUUUUAUUAU |
| | | GUAUGGAAAAGUUAUGUGCAUGUUGUAGACGGUUGU |
| | | AAUUCAUCAACUUGUAUGAUGUGUUACAAACGUAAUA |
| | | GAGCAACAAGAGUCGAAUGUACAACUAUUGUUAAUGG |
| | | UGUUAGAAGGUCCUUUUAUGUCUAUGCUAAUGGAGG |
| | | UAAAGGCUUUUGCAAACUACACAAUUGGAAUUGUGUU |
| | | AAUUGUGAUACAUUCUGUGCUGGUAGUACAUUUAUU |
| | | AGUGAUGAAGUUGCGAGAGACUUGUCACUACAGUUUA |
| | | AAAGACCAAUAAAUCCUACUGACCAGUCUUCUUACAU |
| | | CGUUGAUAGUGUUACAGUGAAGAAUGGUUCCAUCCAU |
| | | CUUUACUUUGAUAAAGCUGGUCAAAAGACUUAUGAAA |
| | | GACAUUCUCUCUCUCAUUUUGUUAACUUAGACAACCU |
| | | GAGAGCUAAUAACACUAAAGGUUCAUUGCCUAUUAAU |
| | | GUUAUAGUUUUUGAUGGUAAAUCAAAAUGUGAAGAA |
| | | UCAUCUGCAAAAUCAGCGUCUGUUUACUACAGUCAGC |
| | | UUAUGUGUCAACCUAUACUGUUACUAGAUCAGGCAUU |
| | | AGUGUCUGAUGUUGGUGAUAGUGCGGAAGUUGCAGU |
| | | UAAAAUGUUUGAUGCUUACGUUAAUACGUUUUCAUCA |
| | | ACUUUUAACGUACCAAUGGAAAAACUCAAAACACUAG |
| | | UUGCAACUGCAGAAGCUGAACUUGCAAAGAAUGUGUC |
| | | CUUAGACAAUGUCUUAUCUACUUUUAUUUCAGCAGCU |
| | | CGGCAAGGGUUUGUUGAUUCAGAUGUAGAAACUAAA |
| | | GAUGUUGUUGAAUGUCUUAAAUUGUCACAUCAAUCUG |
| | | ACAUAGAAGUUACUGGCGAUAGUUGUAAUAACUAUA |
| | | UGCUCACCUAUAACAAAGUUGAAAACAUGACACCCCG |
| | | UGACCUUGGUGCUUGUAUUGACUGUAGUGCGCGUCAU |
| | | AUUAAUGCGCAGGUAGCAAAAAGUCACAACAUUGCUU |
| | | UGAUAUGGAACGUUAAAGAUUUCAUGUCAUUGUCUG |
| | | AACAACUACGAAAACAAAUACGUAGUGCUGCUAAAAA |
| | | GAAUAACUUACCUUUUAAGUUGACAUGUGCAACUACU |
| | | AGACAAGUUGUUAAUGUUGUAACAACAAAGAUAGCAC |
| | | UUAAGGGUGGUAAAAUUGUUAAUAAUUGGUUGAAGC |
| | | AGUUAAUUAAAGUUACACUUGUGUUCCUUUUUGUUGC |
| | | UGCUAUUUUCUAUUUAAUAACACCUGUUCAUGUCAUG |
| | | UCUAAACAUACUGACUUUUCAAGUGAAAUCAUAGGAU |
| | | ACAAGGCUAUUGAUGGUGGUGUCACUCGUGACAUAGC |
| | | AUCUACAGAUACUUGUUUUGCUAACAAACAUGCUGAU |
| | | UUUGACACAUGGUUUAGCCAGCGUGGUGGUAGUUAUA |
| | | CUAAUGACAAAGCUUGCCCAUUGAUUGCUGCAGUCAU |
| | | AACAAGAGAAGUGGGUUUUGUCGUGCCUGGUUUGCCU |
| | | GGCACGAUAUUACGCACAACUAAUGGUGACUUUUUGC |
| | | AUUUCUUACCUAGAGUUUUUAGUGCAGUUGGUAACAU |
| | | CUGUUACACACCAUCAAAACUUAUAGAGUACACUGAC |
| | | UUUGCAACAUCAGCUUGUGUUUUGGCUGCUGAAUGUA |
| | | CAAUUUUUAAAGAUGCUUCUGGUAAGCCAGUACCAUA |
| | | UUGUUAUGAUACCAAUGUACUAGAAGGUUCUGUUGCU |
| | | UAUGAAAGUUUACGCCCUGACACACGUUAUGUGCUCA |
| | | UGGAUGGCUCUAUUAUUCAAUUUCCUAACACCUACCU |
| | | UGAAGGUUCUGUUAGAGUGGUAACAACUUUUGAUUC |
| | | UGAGUACUGUAGGCACGGCACUUGUGAAAGAUCAGAA |
| | | GCUGGUGUUUGUGUAUCUACUAGUGGUAGAUGGGUA |
| | | CUUAACAAUGAUUAUUACAGAUCUUUACCAGGAGUUU |
| | | UCUGUGGUGUAGAUGCUGUAAAUUUACUUACUAAUA |
| | | UGUUUACACCACUAAUUCAACCUAUUGGUGCUUUGGA |
| | | CAUAUCAGCAUCUAUAGUAGCUGGUGGUAUUGUAGCU |
| | | AUCGUAGUAACAUGCCUUGCCUACUAUUUUAUGAGGU |
| | | UUAGAAGAGCUUUUGGUGAAUACAGUCAUGUAGUUG |
| | | CCUUUAAUACUUUACUAUUCCUUAUGUCAUUCACUGU |
| | | ACUCUGUUUAACACCAGUUUACUCAUUCUUACCUGGU |
| | | GUUUAUUCUGUUAUUUACUUGUACUUGACAUUUUAUC |
| | | UUACUAAUGAUGUUUCUUUUUUAGCACAUAUUCAGUG |
| | | GAUGGUUAUGUUCACACCUUUAGUACCUUUCUGGAUA |
| | | ACAAUUGCUUAUAUCAUUUGUAUUUCCACAAAGCAUU |
| | | UCUAUUGGUUCUUUAGUAAUUACCUAAAGAGACGUGU |
| | | AGUCUUUAAUGGUGUUUCCUUUAGUACUUUUGAAGA |
| | | AGCUGCGCUGUGCACCUUUUUGUUAAAUAAAGAAAUG |
| | | UAUCUAAAGUUGCGUAGUGAUGUGCUAUUACCUCUUA |
| | | CGCAAUAUAAUAGAUACUUAGCUCUUUAUAAUAAGUA |
| | | CAAGUAUUUUAGUGGAGCAAUGGAUACAACUAGCUAC |
| | | AGAGAAGCUGCUUGUUGUCAUCUCGCAAAGGCUCUCA |
| | | AUGACUUCAGUAACUCAGGUUCUGAUGUUCUUUACCA |
| | | ACCACCACAAACCUCUAUCACCUCAGCUGUUUUGCAG |
| | | AGUGGUUUUAGAAAAAUGGCAUUCCCAUCUGGUAAAG |
| | | UUGAGGGUUGUAUGGUACAAGUAACUUGUGGUACAA |
| | | CUACACUUAACGGUCUUUGGCUUGAUGACGUAGUUUA |
| | | CUGUCCAAGACAUGUGAUCUGCACCUCUGAAGACAUG |
| | | CUUAACCCUAAUUAUGAAGAUUUACUCAUUCGUAAGU |
| | | CUAAUCAUAAUUUCUUGGUACAGGCUGGUAAUGUUCA |
| | | ACUCAGGGUUAUUGGACAUUCUAUGCAAAAUUGUGUA |
| | | CUUAAGCUUAAGGUUGAUACAGCCAAUCCUAAGACAC |
| | | CUAAGUAUAAGUUUGUUCGCAUUCAACCAGGACAGAC |
| | | UUUUUCAGUGUUAGCUUGUUACAAUGGUUCACCAUCU |
| | | GGUGUUUACCAAUGUGCUAUGAGGCCCAAUUUCACUA |
| | | UUAAGGGUUCAUUCCUUAAUGGUUCAUGUGGUAGUG |
| | | UUGGUUUUAACAUAGAUUAUGACUGUGUCUCUUUUU |
| | | GUUACAUGCACCAUAUGGAAUUACCAACUGGAGUUCA |
| | | UGCUGGCACAGACUUAGAAGGUAACUUUUAUGGACCU |
| | | UUUGUUGACAGGCAAACAGCACAAGCAGCUGGUACGG |
| | | ACACAACUAUUACAGUUAAUGUUUUAGCUUGGUUGUA |
| | | CGCUGCUGUUAUAAAUGGAGACAGGUGGUUUCUCAAU |
| | | CGAUUUACCACAACUCUUAAUGACUUUAACCUUGUGG |
| | | CUAUGAAGUACAAUUAUGAACCUCUAACACAAGACCA |
| | | UGUUGACAUACUAGGACCUCUUUCUGCUCAAACUGGA |
| | | AUUGCCGUUUUAGAUAUGUGUGCUUCAUUAAAAGAA |
| | | UUACUGCAAAAUGGUAUGAAUGGACGUACCAUAUUGG |
| | | GUAGUGCUUUAUUAGAAGAUGAAUUUACACCUUUUG |
| | | AUGUUGUUAGACAAUGCUCAGGUGUUACUUUCCAAAG |
| | | UGCAGUGAAAAGAACAAUCAAGGGUACACACCACUGG |
| | | UUGUUACUCACAAUUUUGACUUCACUUUUAGUUUUAG |
| | | UCCAGAGUACUCAAUGGUCUUUGUUCUUUUUUUUGUA |
| | | UGAAAAUGCCUUUUUACCUUUUGCUAUGGGUAUUAUU |
| | | GCUAUGUCUGCUUUUGCAAUGAUGUUUGUCAAACAUA |
| | | AGCAUGCAUUUCUCUGUUUGUUUUUGUUACCUUCUCU |
| | | UGCCACUGUAGCUUAUUUUAAUAUGGUCUAUAUGCCU |
| | | GCUAGUUGGGUGAUGCGUAUUAUGACAUGGUUGGAU |
| | | AUGGUUGAUACUAGUUUGUCUGGUUUUAAGCUAAAA |
| | | GACUGUGUUAUGUAUGCAUCAGCUGUAGUGUUACUAA |
| | | UCCUUAUGACAGCAAGAACUGUGUAUGAUGAUGGUGC |
| | | UAGGAGAGUGUGGACACUUAUGAAUGUCUUGACACUC |
| | | GUUUAUAAAGUUUAUUAUGGUAAUGCUUUAGAUCAA |
| | | GCCAUUUCCAUGUGGGCUCUUAUAAUCUCUGUUACUU |
| | | CUAACUACUCAGGUGUAGUUACAACUGUCAUGUUUUU |
| | | GGCCAGAGGUAUUGUUUUUAUGUGUGUUGAGUAUUG |
| | | CCCUAUUUUCUUCAUAACUGGUAAUACACUUCAGUGU |
| | | AUAAUGCUAGUUUAUUGUUUCUUAGGCUAUUUUUGU |
| | | ACUUGUUACUUUGGCCUCUUUUGUUUACUCAACCGCU |
| | | ACUUUAGACUGACUCUUGGUGUUUAUGAUUACUUAGU |
| | | UUCUACACAGGAGUUUAGAUAUAUGAAUUCACAGGGA |
| | | CUACUCCCACCCAAGAAUAGCAUAGAUGCCUUCAAAC |
| | | UCAACAUUAAAUUGUUGGGUGUUGGUGGCAAACCUUG |
| | | UAUCAAAGUAGCCACUGUACAGUCUAAAAUGUCAGAU |
| | | GUAAAGUGCACAUCAGUAGUCUUACUCUCAGUUUUGC |
| | | AACAACUCAGAGUAGAAUCAUCAUCUAAAUUGUGGGC |
| | | UCAAUGUGUCCAGUUACACAAUGACAUUCUCUUAGCU |
| | | AAAGAUACUACUGAAGCCUUUGAAAAAAUGGUUUCAC |
| | | UACUUUCUGUUUUGCUUUCCAUGCAGGGUGCUGUAGA |
| | | CAUAAACAAGCUUUGUGAAGAAAUGCUGGACAACAGG |
| | | GCAACCUUACAAGCUAUAGCCUCAGAGUUUAGUUCCC |
| | | UUCCAUCAUAUGCAGCUUUUGCUACUGCUCAAGAAGC |
| | | UUAUGAGCAGGCUGUUGCUAAUGGUGAUUCUGAAGU |
| | | UGUUCUUAAAAAGUUGAAGAAGUCUUUGAAUGUGGC |
| | | UAAAUCUGAAUUUGACCGUGAUGCAGCCAUGCAACGU |
| | | AAGUUGGAAAAGAUGGCUGAUCAAGCUAUGACCCAAA |
| | | UGUAUAAACAGGCUAGAUCUGAGGACAAGAGGGCAAA |
| | | AGUUACUAGUGCUAUGCAGACAAUGCUUUUCACUAUG |
| | | CUUAGAAAGUUGGAUAAUGAUGCACUCAACAACAUUA |
| | | UCAACAAUGCAAGAGAUGGUUGUGUUCCCUUGAACAU |
| | | AAUACCUCUUACAACAGCAGCCAAACUAAUGGUUGUC |
| | | AUACCAGACUAUAACACAUAUAAAAAUACGUGUGAUG |
| | | GUACAACAUUUACUUAUGCAUCAGCAUUGUGGGAAAU |
| | | CCAACAGGUUGUAGAUGCAGAUAGUAAAAUUGUUCAA |
| | | CUUAGUGAAAUUAGUAUGGACAAUUCACCUAAUUUAG |
| | | CAUGGCCUCUUAUUGUAACAGCUUUAAGGGCCAAUUC |
| | | UGCUGUCAAAUUACAGAAUAAUGAGCUUAGUCCUGUU |
| | | GCACUACGACAGAUGUCUUGUGCUGCCGGUACUACAC |
| | | AAACUGCUUGCACUGAUGACAAUGCGUUAGCUUACUA |
| | | CAACACAACAAAGGGAGGUAGGUUUGUACUUGCACUG |
| | | UUAUCCGAUUUACAGGAUUUGAAAUGGGCUAGAUUCC |
| | | CUAAGAGUGAUGGAACUGGUACUAUCUAUACAGAACU |
| | | GGAACCACCUUGUAGGUUUGUUACAGACACACCUAAA |
| | | GGUCCUAAAGUGAAGUAUUUAUACUUUAUUAAAGGA |
| | | UUAAACAACCUAAAUAGAGGUAUGGUACUUGGUAGU |
| | | UUAGCUGCCACAGUACGUCUACAAGCUGGUAAUGCAA |
| | | CAGAAGUGCCUGCCAAUUCAACUGUAUUAUCUUUCUG |
| | | UGCUUUUGCUGUAGAUGCUGCUAAAGCUUACAAAGAU |
| | | UAUCUAGCUAGUGGGGGACAACCAAUCACUAAUUGUG |
| | | UUAAGAUGUUGUGUACACACACUGGUACUGGUCAGGC |
| | | AAUAACAGUUACACCGGAAGCCAAUAUGGAUCAAGAA |
| | | UCCUUUGGUGGUGCAUCGUGUUGUCUGUACUGCCGUU |
| | | GCCACAUAGAUCAUCCAAAUCCUAAAGGAUUUUGUGA |
| | | CUUAAAAGGUAAGUAUGUACAAAUACCUACAACUUGU |
| | | GCUAAUGACCCUGUGGGUUUUACACUUAAAAACACAG |
| | | UCUGUACCGUCUGCGGUAUGUGGAAAGGUUAUGGCUG |
| | | UAGUUGUGAUCAACUCCGCGAACCCAUGCUUCAGUCA |
| | | GCUGAUGCACAAUCGUUUUUAAACGGGUUUGCGGUGU |
| | | AAGUGCAGCCCGUCUUACACCGUGCGGCACAGGCACU |
| | | AGUACUGAUGUCGUAUACAGGGCUUUUGACAUCUACA |
| | | AUGAUAAAGUAGCUGGUUUUGCUAAAUUCCUAAAAAC |
| | | UAAUUGUUGUCGCUUCCAAGAAAAGGACGAAGAUGAC |
| | | AAUUUAAUUGAUUCUUACUUUGUAGUUAAGAGACAC |
| | | ACUUUCUCUAACUACCAACAUGAAGAAACAAUUUAUA |
| | | AUUUACUUAAGGAUUGUCCAGCUGUUGCUAAACAUGA |
| | | CUUCUUUAAGUUUAGAAUAGACGGUGACAUGGUACCA |
| | | CAUAUAUCACGUCAACGUCUUACUAAAUACACAAUGG |
| | | CAGACCUCGUCUAUGCUUUAAGGCAUUUUGAUGAAGG |
| | | UAAUUGUGACACAUUAAAAGAAAUACUUGUCACAUAC |
| | | AAUUGUUGUGAUGAUGAUUAUUUCAAUAAAAAGGAC |
| | | UGGUAUGAUUUUGUAGAAAACCCAGAUAUAUUACGCG |
| | | UAUACGCCAACUUAGGUGAACGUGUACGCCAAGCUUU |
| | | GUUAAAAACAGUACAAUUCUGUGAUGCCAUGCGAAAU |
| | | GCUGGUAUUGUUGGUGUACUGACAUUAGAUAAUCAA |
| | | GAUCUCAAUGGUAACUGGUAUGAUUUCGGUGAUUUCA |
| | | UACAAACCACGCCAGGUAGUGGAGUUCCUGUUGUAGA |
| | | UUCUUAUUAUUCAUUGUUAAUGCCUAUAUUAACCUUG |
| | | ACCAGGGCUUUAACUGCAGAGUCACAUGUUGACACUG |
| | | ACUUAACAAAGCCUUACAUUAAGUGGGAUUUGUUAAA |
| | | AUAUGACUUCACGGAAGAGAGGUUAAAACUCUUUGAC |
| | | CGUUAUUUUAAAUAUUGGGAUCAGACAUACCACCCAA |
| | | AUUGUGUUAACUGUUUGGAUGACAGAUGCAUUCUGCA |
| | | UUGUGCAAACUUUAAUGUUUUAUUCUCUACAGUGUUC |
| | | CCACCUACAAGUUUUGGACCACUAGUGAGAAAAAUAU |
| | | UUGUUGAUGGUGUUCCAUUUGUAGUUUCAACUGGAU |
| | | ACCACUUCAGAGAGCUAGGUGUUGUACAUAAUCAGGA |
| | | UGUAAACUUACAUAGCUCUAGACUUAGUUUUAAGGAA |
| | | UUACUUGUGUAUGCUGCUGACCCUGCUAUGCACGCUG |
| | | CUUCUGGUAAUCUAUUACUAGAUAAACGCACUACGUG |
| | | CUUUUCAGUAGCUGCACUUACUAACAAUGUUGCUUUU |
| | | CAAACUGUCAAACCCGGUAAUUUUAACAAAGACUUCU |
| | | AUGACUUUGCUGUGUCUAAGGGUUUCUUUAAGGAAG |
| | | GAAGUUCUGUUGAAUUAAAACACUUCUUCUUUGCUCA |
| | | GGAUGGUAAUGCUGCUAUCAGCGAUUAUGACUACUAU |
| | | CGUUAUAAUCUACCAACAAUGUGUGAUAUCAGACAAC |
| | | UACUAUUUGUAGUUGAAGUUGUUGAUAAGUACUUUG |
| | | AUUGUUACGAUGGUGGCUGUAUUAAUGCUAACCAAGU |
| | | CAUCGUCAACAACCUAGACAAAUCAGCUGGUUUUCCA |
| | | UUUAAUAAAUGGGGUAAGGCUAGACUUUAUUAUGAU |
| | | UCAAUGAGUUAUGAGGAUCAAGAUGCACUUUUCGCAU |
| | | AUACAAAACGUAAUGUCAUCCCUACUAUAACUCAAAU |
| | | GAAUCUUAAGUAUGCCAUUAGUGCAAAGAAUAGAGCU |
| | | CGCACCGUAGCUGGUGUCUCUAUCUGUAGUACUAUGA |
| | | CCAAUAGACAGUUUCAUCAAAAAUUAUUGAAAUCAAU |
| | | AGCCGCCACUAGAGGAGCUACUGUAGUAAUUGGAACA |
| | | AGCAAAUUCUAUGGUGGUUGGCACAACAUGUUAAAAA |
| | | CUGUUUAUAGUGAUGUAGAAAACCCUCACCUUAUGGG |
| | | UUGGGAUUAUCCUAAAUGUGAUAGAGCCAUGCCUAAC |
| | | AUGCUUAGAAUUAUGGCCUCACUUGUUCUUGCUCGCA |
| | | AACAUACAACGUGUUGUAGCUUGUCACACCGUUUCUA |
| | | UAGAUUAGCUAAUGAGUGUGCUCAAGUAUUGAGUGA |
| | | AAUGGUCAUGUGUGGCGGUUCACUAUAUGUUAAACCA |
| | | GGUGGAACCUCAUCAGGAGAUGCCACAACUGCUUAUG |
| | | CUAAUAGUGUUUUUAACAUUUGUCAAGCUGUCACGGC |
| | | CAAUGUUAAUGCACUUUUAUCUACUGAUGGUAACAAA |
| | | AUUGCCGAUAAGUAUGUCCGCAAUUUACAACACAGAC |
| | | UUUAUGAGUGUCUCUAUAGAAAUAGAGAUGUUGACA |
| | | CAGACUUUGUGAAUGAGUUUUACGCAUAUUUGCGUAA |
| | | ACAUUUCUCAAUGAUGAUACUCUCUGACGAUGCUGUU |
| | | GUGUGUUUCAAUAGCACUUAUGCAUCUCAAGGUCUAG |
| | | UGGCUAGCAUAAAGAACUUUAAGUCAGUUCUUUAUUA |
| | | UCAAAACAAUGUUUUUAUGUCUGAAGCAAAAUGUUG |
| | | GACUGAGACUGACCUUACUAAAGGACCUCAUGAAUUU |
| | | UGCUCUCAACAUACAAUGCUAGUUAAACAGGGUGAUG |
| | | AUUAUGUGUACCUUCCUUACCCAGAUCCAUCAAGAAU |
| | | CCUAGGGGCCGGCUGUUUUGUAGAUGAUAUCGUAAAA |
| | | ACAGAUGGUACACUUAUGAUUGAACGGUUCGUGUCUU |
| | | UAGCUAUAGAUGCUUACCCACUUACUAAACAUCCUAA |
| | | UCAGGAGUAUGCUGAUGUCUUUCAUUUGUACUUACAA |
| | | UACAUAAGAAAGCUACAUGAUGAGUUAACAGGACACA |
| | | UGUUAGACAUGUAUUCUGUUAUGCUUACUAAUGAUA |
| | | ACACUUCAAGGUAUUGGGAACCUGAGUUUUAUGAGGC |
| | | UAUGUACACACCGCAUACAGUCUUACAGGCUGUUGGG |
| | | GCUUGUGUUCUUUGCAAUUCACAGACUUCAUUAAGAU |
| | | GUGGUGCUUGCAUACGUAGACCAUUCUUAUGUUGUAA |
| | | AUGCUGUUACGACCAUGUCAUAUCAACAUCACAUAAA |
| | | UUAGUCUUGUCUGUUAAUCCGUAUGUUUGCAAUGCUC |
| | | CAGGUUGUGAUGUCACAGAUGUGACUCAACUUUACUU |
| | | AGGAGGUAUGAGCUAUUAUUGUAAAUCACAUAAACCA |
| | | CCCAUUAGUUUUCCAUUGUGUGCUAAUGGACAAGUUU |
| | | UUGGUUUAUAUAAAAAUACAUGUGUUGGUAGCGAUA |
| | | AUGUUACUGACUUUAAUGCAAUUGCAACAUGUGACUG |
| | | GACAAAUGCUGGUGAUUACAUUUUAGCUAACACCUGU |
| | | ACUGAAAGACUCAAGCUUUUUGCAGCAGAAACGCUCA |
| | | AAGCUACUGAGGAGACAUUUAAACUGUCUUAUGGUAU |
| | | UGCUACUGUACGUGAAGUGCUGUCUGACAGAGAAUUA |
| | | CAUCUUUCAUGGGAAGUUGGUAAACCUAGACCACCAC |
| | | UUAACCGAAAUUAUGUCUUUACUGGUUAUCGUGUAAC |
| | | UAAAAACAGUAAAGUACAAAUAGGAGAGUACACCUUU |
| | | GAAAAAGGUGACUAUGGUGAUGCUGUUGUUUACCGA |
| | | GGUACAACAACUUACAAAUUAAAUGUUGGUGAUUAU |
| | | UUUGUGCUGACAUCACAUACAGUAAUGCCAUUAAGUG |
| | | CACCUACACUAGUGCCACAAGAGCACUAUGUUAGAAU |
| | | UACUGGCUUAUACCCAACACUCAAUAUCUCAGAUGAG |
| | | UUUUCUAGCAAUGUUGCAAAUUAUCAAAAGGUUGGU |
| | | AUGCAAAAGUAUUCUACACUCCAGGGACCACCUGGUA |
| | | CUGGUAAGAGUCAUUUUGCUAUUGGCCUAGCUCUCUA |
| | | CUACCCUUCUGCUCGCAUAGUGUAUACAGCUUGCUCU |
| | | CAUGCCGCUGUUGAUGCACUAUGUGAGAAGGCAUUAA |
| | | AAUAUUUGCCUAUAGAUAAAUGUAGUAGAAUUAUAC |
| | | CUGCACGUGCUCGUGUAGAGUGUUUUGAUAAAUUCAA |
| | | AGUGAAUUCAACAUUAGAACAGUAUGUCUUUUGUACU |
| | | GUAAAUGCAUUGCCUGAGACGACAGCAGAUAUAGUUG |
| | | UCUUUGAUGAAAUUUCAAUGGCCACAAAUUAUGAUUU |
| | | GAGUGUUGUCAAUGCCAGAUUACGUGCUAAGCACUAU |
| | | GUGUACAUUGGCGACCCUGCUCAAUUACCUGCACCAC |
| | | UUUCAAUUCAGUGUGUAGACUUAUGAAAACUAUAGG |
| | | UCCAGACAUGUUCCUCGGAACUUGUCGGCGUUGUCCU |
| | | GCUGAAAUUGUUGACACUGUGAGUGCUUUGGUUUAU |
| | | GAUAAUAAGCUUAAAGCACAUAAAGACAAAUCAGCUC |
| | | AAUGCUUUAAAAUGUUUUAUAAGGGUGUUAUCACGC |
| | | AUGAUGUUUCAUCUGCAAUUAACAGGCCACAAAUAGG |
| | | CGUGGUAAGAGAAUUCCUUACACGUAACCCUGCUUGG |
| | | AGAAAAGCUGUCUUUAUUUCACCUUAUAAUUCACAGA |
| | | AUGCUGUAGCCUCAAAGAUUUUGGGACUACCAACUCA |
| | | AACUGUUGAUUCAUCACAGGGCUCAGAAUAUGACUAU |
| | | GUCAUAUUCACUCAAACCACUGAAACAGCUCACUCUU |
| | | GUAAUGUAAACAGAUUUAAUGUUGCUAUUACCAGAGC |
| | | AAAAGUAGGCAUACUUUGCAUAAUGUCUGAUAGAGAC |
| | | CUUUAUGACAAGUUGCAAUUUACAAGUCUUGAAAUUC |
| | | CACGUAGGAAUGUGGCAACUUUACAAGCUGAAAAUGU |
| | | AACAGGACUCUUUAAAGAUUGUAGUAAGGUAAUCACU |
| | | GGGUUACAUCCUACACAGGCACCUACACACCUCAGUG |
| | | UUGACACUAAAUUCAAAACUGAAGGUUUAUGUGUUG |
| | | ACAUACCUGGCAUACCUAAGGACAUGACCUAUAGAAG |
| | | ACUCAUCUCUAUGAUGGGUUUUAAAAUGAAUUAUCAA |
| | | GUUAAUGGUUACCCUAACAUGUUUAUCACCCGCGAAG |
| | | AAGCUAUAAGACAUGUACGUGCAUGGAUUGGCUUCGA |
| | | UGUCGAGGGGUGUCAUGCUACUAGAGAAGCUGUUGGU |
| | | ACCAAUUUACCUUUACAGCUAGGUUUUUCUACAGGUG |
| | | UUAACCUAGUUGCUGUACCUACAGGUUAUGUUGAUAC |
| | | ACCUAAUAAUACAGAUUUUUCCAGAGUUAGUGCUAAA |
| | | CCACCGCCUGGAGAUCAAUUUAAACACCUCAUACCAC |
| | | UUAUGUACAAAGGACUUCCUUGGAAUGUAGUGCGUAU |
| | | AAAGAUUGUACAAAUGUUAAGUGACACACUUAAAAA |
| | | UCUCUCUGACAGAGUCGUAUUUGUCUUAUGGGCACAU |
| | | GGCUUUGAGUUGACAUCUAUGAAGUAUUUUGUGAAA |
| | | AUAGGACCUGAGCGCACCUGUUGUCUAUGUGAUAGAC |
| | | GUGCCACAUGCUUUUCCACUGCUUCAGACACUUAUGC |
| | | CUGUUGGCAUCAUUCUAUUGGAUUUGAUUACGUCUAU |
| | | AAUCCGUUUAUGAUUGAUGUUCAACAAUGGGGUUUU |
| | | ACAGGUAACCUACAAAGCAACCAUGAUCUGUAUUGUC |
| | | AAGUCCAUGGUAAUGCACAUGUAGCUAGUUGUGAUGC |
| | | AAUCAUGACUAGGUGUCUAGCUGUCCACGAGUGCUUU |
| | | GUUAAGCGUGUUGACUGGACUAUUGAAUAUCCUAUAA |
| | | UUGGUGAUGAACUGAAGAUUAAUGCGGCUUGUAGAA |
| | | AGGUUCAACACAUGGUUGUUAAAGCUGCAUUAUUAGC |
| | | AGACAAAUUCCCAGUUCUUCACGACAUUGGUAACCCU |
| | | AAAGCUAUUAAGUGUGUACCUCAAGCUGAUGUAGAAU |
| | | GGAAGUUCUAUGAUGCACAGCCUUGUAGUGACAAAGC |
| | | UUAUAAAAUAGAAGAAUUAUUCUAUUCUUAUGCCACA |
| | | CAUUCUGACAAAUUCACAGAUGGUGUAUGCCUAUUUU |
| | | GGAAUUGCAAUGUCGAUAGAUAUCCUGCUAAUUCCAU |
| | | UGUUUGUAGAUUUGACACUAGAGUGCUAUCUAACCUU |
| | | AACUUGCCUGGUUGUGAUGGUGGCAGUUUGUAUGUA |
| | | AAUAAACAUGCAUUCCACACACCAGCUUUUGAUAAAA |
| | | GUGCUUUUGUUAAUUUAAAACAAUUACCAUUUUUCUA |
| | | UUACUCUGACAGUCCAUGUGAGUCUCAUGGAAAACAA |
| | | GUAGUGUCAGAUAUAGAUUAUGUACCACUAAAGUCUG |
| | | CUACGUGUAUAACACGUUGCAAUUUAGGUGGUGCUGU |
| | | CUGUAGACAUCAUGCUAAUGAGUACAGAUUGUAUCUC |
| | | GAUGCUUAUAACAUGAUGAUCUCAGCUGGCUUUAGCU |
| | | UGUGGGUUUACAAACAAUUUGAUACUUAUAACCUCUG |
| | | GAACACUUUUACAAGACUUCAGAGUUUAGAAAAUGUG |
| | | GCUUUUAAUGUUGUAAAUAAGGGACACUUUGAUGGA |
| | | CAACAGGGUGAAGUACCAGUUUCUAUCAUUAAUAACA |
| | | CUGUUUACACAAAAGUUGAUGGUGUUGAUGUAGAAU |
| | | UGUUUGAAAAUAAAACAACAUUACCUGUUAAUGUAGC |
| | | AUUUGAGCUUUGGGCUAAGCGCAACAUUAAACCAGUA |
| | | CCAGAGGUGAAAAUACUCAAUAAUUUGGGUGUGGACA |
| | | UUGCUGCUAAUACUGUGAUCUGGGACUACAAAAGAGA |
| | | UGCUCCAGCACAUAUAUCUACUAUUGGUGUUUGUUCU |
| | | AUGACUGACAUAGCCAAGAAACCAACUGAAACGAUUU |
| | | GUGCACCACUCACUGUCUUUUUUGAUGGUAGAGUUGA |
| | | UGGUCAAGUAGACUUAUUUAGAAAUGCCCGUAAUGGU |
| | | GUUCUUAUUACAGAAGGUAGUGUUAAAGGUUUACAA |
| | | CCAUCUGUAGGUCCCAAACAAGCUAGUCUUAAUGGAG |
| | | UCACAUUAAUUGGAGAAGCCGUAAAAACACAGUUCAA |
| | | UUAUUAUAAGAAAGUUGAUGGUGUUGUCCAACAAUU |
| | | ACCUGAAACUUACUUUACUCAGAGUAGAAAUUUACAA |
| | | GAAUUUAAACCCAGGAGUCAAAUGGAAAUUGAUUUCU |
| | | UAGAAUUAGCUAUGGAUGAAUUCAUUGAACGGUAUA |
| | | AAUUAGAAGGCUAUGCCUUCGAACAUAUCGUUUAUGG |
| | | AGAUUUUAGUCAUAGUCAGUUAGGUGGUUUACAUCU |
| | | ACUGAUUGGACUAGCUAAACGUUUUAAGGAAUCACCU |
| | | UUUGAAUUAGAAGAUUUUAUUCCUAUGGACAGUACA |
| | | GUUAAAAACUAUUUCAUAACAGAUGCGCAAACAGGUU |
| | | CAUCUAAGUGUGUGUGUUCUGUUAUUGAUUUAUUAC |
| | | UUGAUGAUUUUGUUGAAAUAAUAAAAUCCCAAGAUU |
| | | UAUCUGUAGUUUCUAAGGUUGUCAAAGUGACUAUUG |
| | | ACUAUACAGAAAUUUCAUUUAUGCUUUGGUGUAAAG |
| | | AUGGCCAUGUAGAAACAUUUUACCCAAAAUUACAAUC |
| | | UAGUCAAGCGUGGCAACCGGGUGUUGCUAUGCCUAAU |
| | | CUUUACAAAAUGCAAAGAAUGCUAUUAGAAAAGUGU |
| | | GACCUUCAAAAUUAUGGUGAUAGUGCAACAUUACCUA |
| | | AAGGCAUAAUGAUGAAUGUCGCAAAAUAUACUCAACU |
| | | GUGUCAAUAUUUAAACACAUUAACAUUAGCUGUACCC |
| | | UAUAAUAUGAGAGUUAUACAUUUUGGUGCUGGUUCU |
| | | GAUAAAGGAGUUGCACCAGGUACAGCUGUUUUAAGAC |
| | | AGUGGUUGCCUACGGGUACGCUGCUUGUCGAUUCAGA |
| | | UCUUAAUGACUUUGUCUCUGAUGCAGAUUCAACUUUG |
| | | AUUGGUGAUUGUGCAACUGUACAUACAGCUAAUAAAU |
| | | GGGAUCUCAUUAUUAGUGAUAUGUACGACCCUAAGAC |
| | | UAAAAAUGUUACAAAAGAAAAUGACUCUAAAGAGGG |
| | | UUUUUUCACUUACAUUUGUGGGUUUAUACAACAAAAG |
| | | CUAGCUCUUGGAGGUUCCGUGGCUAUAAAGAUAACAG |
| | | AACAUUCUUGGAAUGCUGAUCUUUAUAAGCUCAUGGG |
| | | ACACUUCGCAUGGUGGACAGCCUUUGUUACUAAUGUG |
| | | AAUGCGUCAUCAUCUGAAGCAUUUUUAAUUGGAUGUA |
| | | AUUAUCUUGGCAAACCACGCGAACAAAUAGAUGGUUA |
| | | UGUCAUGCAUGCAAAUUACAUAUUUUGGAGGAAUACA |
| | | AAUCCAAUUCAGUUGUCUUCCUAUUCUUUAUUUGACA |
| | | UGAGUAAAUUUCCCCUUAAAUUAAGGGGUACUGCUGU |
| | | UAUGUCUUUAAAAGAAGGUCAAAUCAAUGAUAUGAU |
| | | UUUAUCUCUUCUUAGUAAAGGUAGACUUAUAAUUAG |
| | | AGAAAACAACAGAGUUGUUAUUUCUAGUGAUGUUCU |
| | | UGUUAACAACUAAACGAACAAUGUUUGUUUUUCUUGU |
| | | UUUAUUGCCACUAGUCUCUAGUCAGUGUGUUAAUCUU |
| | | ACAACCAGAACUCAAUUACCCCCUGCAUACACUAAUU |
| | | CUUUCACACGUGGUGUUUAUUACCCUGACAAAGUUUU |
| | | CAGAUCCUCAGUUUUACAUUCAACUCAGGACUUGUUC |
| | | UUACCUUUCUUUUCCAAUGUUACUUGGUUCCAUGCUA |
| | | UACAUGUCUCUGGGACCAAUGGUACUAAGAGGUUUGA |
| | | UAACCCUGUCCUACCAUUUAAUGAUGGUGUUUAUUUU |
| | | GCUUCCACUGAGAAGUCUAACAUAAUAAGAGGCUGGA |
| | | UUUUUGGUACUACUUUAGAUUCGAAGACCCAGUCCCU |
| | | ACUUAUUGUUAAUAACGCUACUAAUGUUGUUAUUAA |
| | | AGUCUGUGAAUUUCAAUUUUGUAAUGAUCCAUUUUU |
| | | GGGUGUUUAUUACCACAAAAACAACAAAAGUUGGAUG |
| | | GAAAGUGAGUUCAGAGUUUAUUCUAGUGCGAAUAAU |
| | | UGCACUUUUGAAUAUGUCUCUCAGCCUUUUCUUAUGG |
| | | ACCUUGAAGGAAAACAGGGUAAUUUCAAAAAUCUUAG |
| | | GGAAUUUGUGUUUAAGAAUAUUGAUGGUUAUUUUAA |
| | | AAUAUAUUCUAAGCACACGCCUAUUAAUUUAGUGCGU |
| | | GAUCUCCCUCAGGGUUUUUCGGCUUUAGAACCAUUGG |
| | | UAGAUUUGCCAAUAGGUAUUAACAUCACUAGGUUUCA |
| | | AACUUUACUUGCUUUACAUAGAAGUUAUUUGACUCCU |
| | | GGUGAUUCUUCUUCAGGUUGGACAGCUGGUGCUGCAG |
| | | CUUAUUAUGUGGGUUAUCUUCAACCUAGGACUUUUCU |
| | | AUUAAAAUAUAAUGAAAAUGGAACCAUUACAGAUGC |
| | | UGUAGACUGUGCACUUGACCCUCUCUCAGAAACAAAG |
| | | UGUACGUUGAAAUCCUUCACUGUAGAAAAAGGAAUCU |
| | | AUCAAACUUCUAACUUUAGAGUCCAACCAACAGAAUC |
| | | UAUUGUUAGAUUUCCUAAUAUUACAAACUUGUGCCCU |
| | | UUUGGUGAAGUUUUUAACGCCACCAGAUUUGCAUCUG |
| | | UUUAUGCUUGGAACAGGAAGAGAAUCAGCAACUGUGU |
| | | UGCUGAUUAUUCUGUCCUAUAUAAUUCCGCAUCAUUU |
| | | UCCACUUUUAAGUGUUAUGGAGUGUCUCCUACUAAAU |
| | | UAAAUGAUCUCUGCUUUACUAAUGUCUAUGCAGAUUC |
| | | AUUUGUAAUUAGAGGUGAUGAAGUCAGACAAAUCGC |
| | | UCCAGGGCAAACUGGAAAGAUUGCUGAUUAUAAUUAU |
| | | AAAUUACCAGAUGAUUUUACAGGCUGCGUUAUAGCUU |
| | | GGAAUUCUAACAAUCUUGAUUCUAAGGUUGGUGGUA |
| | | AUUAUAAUUACCUGUAUAGAUUGUUUAGGAAGUCUA |
| | | AUCUCAAACCUUUUGAGAGAGAUAUUUCAACUGAAAU |
| | | CUAUCAGGCCGGUAGCACACCUUGUAAUGGUGUUGAA |
| | | GGUUUUAAUUGUUACUUUCCUUUACAAUCAUAUGGUU |
| | | UCCAACCCACUAAUGGUGUUGGUUACCAACCAUACAG |
| | | AGUAGUAGUACUUUCUUUUGAACUUCUACAUGCACCA |
| | | GCAACUGUUUGUGGACCUAAAAAGUCUACUAAUUUGG |
| | | UUAAAAACAAAUGUGUCAAUUUCAACUUCAAUGGUUU |
| | | AACAGGCACAGGUGUUCUUACUGAGUCUAACAAAAAG |
| | | UUUCUGCCUUUCCAACAAUUUGGCAGAGACAUUGCUG |
| | | ACACUACUGAUGCUGUCCGUGAUCCACAGACACUUGA |
| | | GAUUCUUGACAUUACACCAUGUUCUUUUGGUGGUGUC |
| | | AGUGUUAUAACACCAGGAACAAAUACUUCUAACCAGG |
| | | UUGCUGUUCUUUAUCAGGAUGUUAACUGCACAGAAGU |
| | | CCCUGUUGCUAUUCAUGCAGAUCAACUUACUCCUACU |
| | | UGGCGUGUUUAUUCUACAGGUUCUAAUGUUUUUCAAA |
| | | CACGUGCAGGCUGUUUAAUAGGGGCUGAACAUGUCAA |
| | | CAACUCAUAUGAGUGUGACAUACCCAUUGGUGCAGGU |
| | | AUAUGCGCUAGUUAUCAGACUCAGACUAAUUCUCCUC |
| | | GGCGGGCACGUAGUGUAGCUAGUCAAUCCAUCAUUGC |
| | | CUACACUAUGUCACUUGGUGCAGAAAAUUCAGUUGCU |
| | | UACUCUAAUAACUCUAUUGCCAUACCCACAAAUUUUA |
| | | CUAUUAGUGUUACCACAGAAAUUCUACCAGUGUCUAU |
| | | GACCAAGACAUCAGUAGAUUGUACAAUGUACAUUUGU |
| | | GGUGAUUCAACUGAAUGCAGCAAUCUUUUGUUGCAAU |
| | | AUGGCAGUUUUUGUACACAAUUAAACCGUGCUUUAAC |
| | | UGGAAUAGCUGUUGAACAAGACAAAAACACCCAAGAA |
| | | GUUUUUGCACAAGUCAAACAAAUUUACAAAACACCAC |
| | | CAAUUAAAGAUUUUGGUGGUUUUAAUUUUUCACAAA |
| | | UAUUACCAGAUCCAUCAAAACCAAGCAAGAGGUCAUU |
| | | UAUUGAAGAUCUACUUUUCAACAAAGUGACACUUGCA |
| | | GAUGCUGGCUUCAUCAAACAAUAUGGUGAUUGCCUUG |
| | | GUGAUAUUGCUGCUAGAGACCUCAUUUGUGCACAAAA |
| | | GUUUAACGGCCUUACUGUUUUGCCACCUUUGCUCACA |
| | | GAUGAAAUGAUUGCUCAAUACACUUCUGCACUGUUAG |
| | | CGGGUACAAUCACUUCUGGUUGGACCUUUGGUGCAGG |
| | | UGCUGCAUUACAAAUACCAUUUGCUAUGCAAAUGGCU |
| | | UAUAGGUUUAAUGGUAUUGGAGUUACACAGAAUGUU |
| | | CUCUAUGAGAACCAAAAAUUGAUUGCCAACCAAUUUA |
| | | AUAGUGCUAUUGGCAAAAUUCAAGACUCACUUUCUUC |
| | | CACAGCAAGUGCACUUGGAAAACUUCAAGAUGUGGUC |
| | | AACCAAAAUGCACAAGCUUUAAACACGCUUGUUAAAC |
| | | AACUUAGCUCCAAUUUUGGUGCAAUUUCAAGUGUUUU |
| | | AAAUGAUAUCCUUUCACGUCUUGACAAAGUUGAGGCU |
| | | GAAGUGCAAAUUGAUAGGUUGAUCACAGGCAGACUUC |
| | | AAAGUUUGCAGACAUAUGUGACUCAACAAUUAAUUAG |
| | | AGCUGCAGAAAUCAGAGCUUCUGCUAAUCUUGCUGCU |
| | | ACUAAAAUGUCAGAGUGUGUACUUGGACAAUCAAAAA |
| | | GAGUUGAUUUUUGUGGAAAGGGCUAUCAUCUUAUGU |
| | | CCUUCCCUCAGUCAGCACCUCAUGGUGUAGUCUUCUU |
| | | GCAUGUGACUUAUGUCCCUGCACAAGAAAAGAACUUC |
| | | ACAACUGCUCCUGCCAUUUGUCAUGAUGGAAAAGCAC |
| | | ACUUUCCUCGUGAAGGUGUCUUUGUUUCAAAUGGCAC |
| | | ACACUGGUUUGUAACACAAAGGAAUUUUUAUGAACCA |
| | | CAAAUCAUUACUACAGACAACACAUUUGUGUCUGGUA |
| | | ACUGUGAUGUUGUAAUAGGAAUUGUCAACAACACAGU |
| | | UUAUGAUCCUUUGCAACCUGAAUUAGACUCAUUCAAG |
| | | GAGGAGUUAGAUAAAUAUUUUAAGAAUCAUACAUCA |
| | | CCAGAUGUUGAUUUAGGUGACAUCUCUGGCAUUAAUG |
| | | CUUCAGUUGUAAACAUUCAAAAAGAAAUUGACCGCCU |
| | | CAAUGAGGUUGCCAAGAAUUUAAAUGAAUCUCUCAUC |
| | | GAUCUCCAAGAACUUGGAAAGUAUGAGCAGUAUAUAA |
| | | AAUGGCCAUGGUACAUUUGGCUAGGUUUUAUAGCUGG |
| | | CUUGAUUGCCAUAGUAAUGGUGACAAUUAUGCUUUGC |
| | | UGUAUGACCAGUUGCUGUAGUUGUCUCAAGGGCUGUU |
| | | GUUCUUGUGGAUCCUGCUGCAAAUUUGAUGAAGACGA |
| | | CUCUGAGCCAGUGCUCAAAGGAGUCAAAUUACAUUAC |
| | | ACAUAAACGAACUUAUGGAUUUGUUUAUGAGAAUCU |
| | | UCACAAUUGGAACUGUAACUUUGAAGCAAGGUGAAAU |
| | | CAAGGAUGCUACUCCUUCAGAUUUUGUUCGCGCUACU |
| | | GCAACGAUACCGAUACAAGCCUCACUCCCUUUCGGAU |
| | | GGCUUAUUGUUGGCGUUGCACUUCUUGCUGUUUUUCA |
| | | GAGCGCUUCCAAAAUCAUAACCCUCAAAAAGAGAUGG |
| | | CAACUAGCACUCUCCAAGGGUGUUCACUUUGUUUGCA |
| | | ACUUGCUGUUGUUGUUUGUAACAGUUUACUCACACCU |
| | | UUUGCUCGUUGCUGCUGGCCUUGAAGCCCCUUUUCUC |
| | | UAUCUUUAUGCUUUAGUCUACUUCUUGCAGAGUAUAA |
| | | ACUUUGUAAGAAUAAUAAUGAGGCUUUGGCUUUGCU |
| | | GGAAAUGCCGUUCCAAAAACCCAUUACUUUAUGAUGC |
| | | CAACUAUUUUCUUUGCUGGCAUACUAAUUGUUACGAC |
| | | UAUUGUAUACCUUACAAUAGUGUAACUUCUUCAAUUG |
| | | UCAUUACUUCAGGUGAUGGCACAACAAGUCCUAUUUC |
| | | UGAACAUGACUACCAGAUUGGUGGUUAUACUGAAAAA |
| | | UGGGAAUCUGGAGUAAAAGACUGUGUUGUAUUACAC |
| | | AGUUACUUCACUUCAGACUAUUACCAGCUGUACUCAA |
| | | CUCAAUUGAGUACAGACACUGGUGUUGAACAUGUUAC |
| | | CUUCUUCAUCUACAAUAAAAUUGUUGAUGAGCCUGAA |
| | | GAACAUGUCCAAAUUCACACAAUCGACGGUUCAUCCG |
| | | GAGUUGUUAAUCCAGUAAUGGAACCAAUUUAUGAUG |
| | | AACCGACGACGACUACUAGCGUGCCUUUGUAAGCACA |
| | | AGCUGAUGAGUACGAACUUAUGUACUCAUUCGUUUCG |
| | | GAAGAGACAGGUACGUUAAUAGUUAAUAGCGUACUUC |
| | | UUUUUCUUGCUUUCGUGGUAUUCUUGCUAGUUACACU |
| | | AGCCAUCCUUACUGCGCUUCGAUUGUGUGCGUACUGC |
| | | UGCAAUAUUGUUAACGUGAGUCUUGUAAAACCUUCUU |
| | | UUUACGUUUACUCUCGUGUUAAAAAUCUGAAUUCUUC |
| | | UAGAGUUCCUGAUCUUCUGGUCUAAACGAACUAAAUA |
| | | UUAUAUUAGUUUUUCUGUUUGGAACUUUAAUUUUAG |
| | | CCAUGGCAGAUUCCAACGGUACUAUUACCGUUGAAGA |
| | | GCUUAAAAAGCUCCUUGAACAAUGGAACCUAGUAAUA |
| | | GGUUUCCUAUUCCUUACAUGGAUUUGUCUUCUACAAU |
| | | UUGCCUAUGCCAACAGGAAUAGGUUUUUGUAUAUAAU |
| | | UAAGUUAAUUUUCCUCUGGCUGUUAUGGCCAGUAACU |
| | | UUAGCUUGUUUUGUGCUUGCUGCUGUUUACAGAAUAA |
| | | AUUGGAUCACCGGUGGAAUUGCUAUCGCAAUGGCUUG |
| | | UCUUGUAGGCUUGAUGUGGCUCAGCUACUUCAUUGCU |
| | | UCUUUCAGACUGUUUGCGCGUACGCGUUCCAUGUGGU |
| | | CAUUCAAUCCAGAAACUAACAUUCUUCUCAACGUGCC |
| | | ACUCCAUGGCACUAUUCUGACCAGACCGCUUCUAGAA |
| | | AGUGAACUCGUAAUCGGAGCUGUGAUCCUUCGUGGAC |
| | | AUCUUCGUAUUGCUGGACACCAUCUAGGACGCUGUGA |
| | | CAUCAAGGACCUGCCUAAAGAAAUCACUGUUGCUACA |
| | | UCACGAACGCUUUCUUAUUACAAAUUGGGAGCUUCGC |
| | | AGCGUGUAGCAGGUGACUCAGGUUUUGCUGCAUACAG |
| | | UCGCUACAGGAUUGGCAACUAUAAAUUAAACACAGAC |
| | | CAUUCCAGUAGCAGUGACAAUAUUGCUUUGCUUGUAC |
| | | AGUAAGUGACAACAGAUGUUUCAUCUCGUUGACUUUC |
| | | AGGUUACUAUAGCAGAGAUAUUACUAAUUAUUAUGA |
| | | GGACUUUUAAAGUUUCCAUUUGGAAUCUUGAUUACAU |
| | | CAUAAACCUCAUAAUUAAAAAUUUAUCUAAGUCACUA |
| | | ACUGAGAAUAAAUAUUCUCAAUUAGAUGAAGAGCAAC |
| | | CAAUGGAGAUUGAUUAAACGAACAUGAAAAUUAUUC |
| | | UUUUCUUGGCACUGAUAACACUCGCUACUUGUGAGCU |
| | | UUAUCACUACCAAGAGUGUGUUAGAGGUACAACAGUA |
| | | CUUUUAAAAGAACCUUGCUCUUCUGGAACAUACGAGG |
| | | GCAAUUCACCAUUUCAUCCUCUAGCUGAUAACAAAUU |
| | | UGCACUGACUUGCUUUAGCACUCAAUUUGCUUUUGCU |
| | | UGUCCUGACGGCGUAAAACACGUCUAUCAGUUACGUG |
| | | CCAGAUCAGUUUCACCUAAACUGUUCAUCAGACAAGA |
| | | GGAAGUUCAAGAACUUUACUCUCCAAUUUUUCUUAUU |
| | | GUUGCGGCAAUAGUGUUUAUAACACUUUGCUUCACAC |
| | | UCAAAAGAAAGACAGAAUGAUUGAACUUUCAUUAAU |
| | | UGACUUCUAUUUGUGCUUUUUAGCCUUUCUGCUAUUC |
| | | CUUGUUUUAAUUAUGCUUAUUAUCUUUUGGUUCUCAC |
| | | UUGAACUGCAAGAUCAUAAUGAAACUUGUCACGCCUA |
| | | AACGAACAUGAAAUUUCUUGUUUUCUUAGGAAUCAUC |
| | | ACAACUGUAGCUGCAUUUCACCAAGAAUGUAGUUUAC |
| | | AGUCAUGUACUCAACAUCAACCAUAUGUAGUUGAUGA |
| | | CCCGUGUCCUAUUCACUUCUAUUCUAAAUGGUAUAUU |
| | | AGAGUAGGAGCUAGAAAAUCAGCACCUUUAAUUGAAU |
| | | UGUGCGUGGAUGAGGCUGGUUCUAAAUCACCCAUUCA |
| | | GUACAUCGAUAUCGGUAAUUAUACAGUUUCCUGUUUA |
| | | CCUUUUACAAUUAAUUGCCAGGAACCUAAAUUGGGUA |
| | | GUCUUGUAGUGCGUUGUUCGUUCUAUGAAGACUUUUU |
| | | AGAGUAUCAUGACGUUCGUGUUGUUUUAGAUUUCAUC |
| | | UAAACGAACAAACUAAAAUGUCUGAUAAUGGACCCCA |
| | | AAAUCAGCGAAAUGCACCCCGCAUUACGUUUGGUGGA |
| | | CCCUCAGAUUCAACUGGCAGUAACCAGAAUGGAGAAC |
| | | GCAGUGGGGCGCGAUCAAAACAACGUCGGCCCCAAGG |
| | | UUUACCCAAUAAUACUGCGUCUUGGUUCACCGCUCUC |
| | | ACUCAACAUGGCAAGGAAGACCUUAAAUUCCCUCGAG |
| | | GACAAGGCGUUCCAAUUAACACCAAUAGCAGUCCAGA |
| | | UGACCAAAUUGGCUACUACCGAAGAGCUACCAGACGA |
| | | AUUCGUGGUGGUGACGGUAAAAUGAAAGAUCUCAGUC |
| | | CAAGAUGGUAUUUCUACUACCUAGGAACUGGGCCAGA |
| | | AGCUGGACUUCCCUAUGGUGCUAACAAAGACGGCAUC |
| | | AUAUGGGUUGCAACUGAGGGAGCCUUGAAUACACCAA |
| | | AAGAUCACAUUGGCACCCGCAAUCCUGCUAACAAUGC |
| | | UGCAAUCGUGCUACAACUUCCUCAAGGAACAACAUUG |
| | | CCAAAAGGCUUCUACGCAGAAGGGAGCAGAGGCGGCA |
| | | GUCAAGCCUCUUCUCGUUCCUCAUCACGUAGUCGCAA |
| | | CAGUUCAAGAAAUUCAACUCCAGGCAGCAGUAGGGGA |
| | | ACUUCUCCUGCUAGAAUGGCUGGCAAUGGCGGUGAUG |
| | | CUGCUCUUGCUUUGCUGCUGCUUGACAGAUUGAACCA |
| | | GCUUGAGAGCAAAAUGUCUGGUAAAGGCCAACAACAA |
| | | CAAGGCCAAACUGUCACUAAGAAAUCUGCUGCUGAGG |
| | | CUUCUAAGAAGCCUCGGCAAAAACGUACUGCCACUAA |
| | | AGCAUACAAUGUAACACAAGCUUUCGGCAGACGUGGU |
| | | CCAGAACAAACCCAAGGAAAUUUUGGGGACCAGGAAC |
| | | UAAUCAGACAAGGAACUGAUUACAAACAUUGGCCGCA |
| | | AAUUGCACAAUUUGCCCCCAGCGCUUCAGCGUUCUUC |
| | | GGAAUGUCGCGCAUUGGCAUGGAAGUCACACCUUCGG |
| | | GAACGUGGUUGACCUACACAGGUGCCAUCAAAUUGGA |
| | | UGACAAAGAUCCAAAUUUCAAAGAUCAAGUCAUUUUG |
| | | CUGAAUAAGCAUAUUGACGCAUACAAAACAUUCCCAC |
| | | CAACAGAGCCUAAAAAGGACAAAAAGAAGAAGGCUGA |
| | | UGAAACUCAAGCCUUACCGCAGAGACAGAAGAAACAG |
| | | CAAACUGUGACUCUUCUUCCUGCUGCAGAUUUGGAUG |
| | | AUUUCUCCAAACAAUUGCAACAAUCCAUGAGCAGUGC |
| | | UGACUCAACUCAGGCCUAAACUCAUGCAGACCACACA |
| | | AGGCAGAUGGGCUAUAUAAACGUUUUCGCUUUUCCGU |
| | | UUACGAUAUAUAGUCUACUCUUGUGCAGAAUGAAUUC |
| | | UCGUAACUACAUAGCACAAGUAGAUGUAGUUAACUUU |
| | | AAUCUCACAUAGCAAUCUUUAAUCAGUGUGUAACAUU |
| | | AGGGAGGACUUGAAAGAGCCACCACAUUUUCACCGAG |
| | | GCCACGCGGAGUACGAUCGAGUGUACAGUGAACAAUG |
| | | CUAGGGAGAGCUGCCUAUAUGGAAGAGCCCUAAUGUG |
| | | UAAAAUUAAUUUUAGUAGUGCUAUCCCCAUGUGAUUU |
| | | UAAUAGCUUCUUAGGAGAAUGACAAAAAAAAAAAAA |
| | | AAAAAAAAAAAAAAAAAAAA |
| 39 | SARS-CoV-2 | MFVFLVLLPLVSSQCVNLTTRTQLPPAYTNSFTRGVYYPD |
| | spike protein | KVFRSSVLHSTQDLFLPFFSNVTWFHAIHVSGTNGTKRFD |
| | (GenPept: | NPVLPFNDGVYFASTEKSNIIRGWIFGTTLDSKTQSLLIVNN |
| | QHD43416) | ATNVVIKVCEFQFCNDPFLGVYYHKNNKSWMESEFRVYS |
| | | SANNCTFEYVSQPFLMDLEGKQGNFKNLREFVFKNIDGYF |
| | | KIYSKHTPINLVRDLPQGFSALEPLVDLPIGINITRFQTLLAL |
| | | HRSYLTPGDSSSGWTAGAAAYYVGYLQPRTFLLKYNENG |
| | | TITDAVDCALDPLSETKCTLKSFTVEKGIYQTSNFRVQPTE |
| | | SIVRFPNITNLCPFGEVENATRFASVYAWNRKRISNCVADY |
| | | SVLYNSASFSTFKCYGVSPTKLNDLCFTNVYADSFVIRGDE |
| | | VRQIAPGQTGKIADYNYKLPDDFTGCVIAWNSNNLDSKV |
| | | GGNYNYLYRLFRKSNLKPFERDISTEIYQAGSTPCNGVEGF |
| | | NCYFPLQSYGFQPTNGVGYQPYRVVVLSFELLHAPATVCG |
| | | PKKSTNLVKNKCVNFNFNGLTGTGVLTESNKKFLPFQQFG |
| | | RDIADTTDAVRDPQTLEILDITPCSFGGVSVITPGTNTSNQV |
| | | AVLYQDVNCTEVPVAIHADQLTPTWRVYSTGSNVFQTRA |
| | | GCLIGAEHVNNSYECDIPIGAGICASYQTQTNSPRRARSVA |
| | | SQSIIAYTMSLGAENSVAYSNNSIAIPTNFTISVTTEILPVSM |
| | | TKTSVDCTMYICGDSTECSNLLLQYGSFCTQLNRALTGIA |
| | | VEQDKNTQEVFAQVKQIYKTPPIKDFGGFNFSQILPDPSKP |
| | | SKRSFIEDLLFNKVTLADAGFIKQYGDCLGDIAARDLICAQ |
| | | KFNGLTVLPPLLTDEMIAQYTSALLAGTITSGWTFGAGAA |
| | | LQIPFAMQMAYRFNGIGVTQNVLYENQKLIANQFNSAIGK |
| | | IQDSLSSTASALGKLQDVVNQNAQALNTLVKQLSSNFGAI |
| | | SSVLNDILSRLDKVEAEVQIDRLITGRLQSLQTYVTQQLIR |
| | | AAEIRASANLAATKMSECVLGQSKRVDFCGKGYHLMSFP |
| | | QSAPHGVVFLHVTYVPAQEKNFTTAPAICHDGKAHFPREG |
| | | VFVSNGTHWFVTQRNFYEPQIITTDNTFVSGNCDVVIGIVN |
| | | NTVYDPLQPELDSFKEELDKYFKNHTSPDVDLGDISGINAS |
| | | VVNIQKEIDRLNEVAKNLNESLIDLQELGKYEQYIKWPWY |
| | | IWLGFIAGLIAIVMVTIMLCCMTSCCSCLKGCCSCGSCCKF |
| | | DEDDSEPVLKGVKLHYT |
| 40 | pT7/NSPISA11- | ACTAGTTAATACGACTCACTATAGGCTTTTTTTTGAAAA |
| | 2A-GFP | GTCTTGTGTTAGCCATGGCTACTTTTAAAGATGCATGCT |
| | T7 promoter: nt | TTCATTATCGTAGATTAACTGCTTTAAATCGGAGATTAT |
| | 7-24 | GCAACATTGGTGCAAATTCTATTTGGATGCCAGTTCCTG |
| | NSP1 CDS: nt | ATGCGAAAATTAAGGGGTGGTGTTTAGAATGTTGTCAA |
| | 54-1,541 | ATAGCTGATTTAACCCATTGTTATGGTTGCTCATTGCCG |
| | 2A peptide | CATGTTTGCAAATGGTGTGTTCAGAACAGAAGATGCTT |
| | CDS: nt 1,542- | CCTTGACAATGAACCTCATTTGCTTAAGCTTAGAACTGT |
| | 1,604 | GAAACATCCAATTACCAAAGACAAATTACAGTGTATCA |
| | GFP CDS: nt | TAGACTTGTACAATATAATATTTCCAATTAATGATAAAG |
| | 1,605-2,321 | TAATTAGAAAATTTGAAAGAATGATAAAGCAAAGAGA |
| | HDV Ribozyme: | ATGTAGGAATCAATATAAAATTGAATGGTATAATCATT |
| | nt 2,414-2,502 | TGCTGCTCCCAATTACATTAAATGCTGCTGCATTTAAGT |
| | T7 Terminator: | TTGATGAAAATAATCTTTATTATGTTTTTGGGTTATATG |
| | nt 2,511-2,553 | AGAAATCAGTCAGTGATATATATGCTCCATATAGAATT |
| | | GTTAACTTTATAAATGAATTTGATAAATTATTGCTTGAT |
| | | CATATTAACTTTACAAGAATGTCCAATCTACCAATAGA |
| | | GTTGAGAAACCATTACGCAAAGAAATACTTCCAATTAT |
| | | CAAGACTGCCATCATCAAAACTAAAGCAAATTTACTTTT |
| | | CAGATTTTACTAAAGAAACTGTGATTTTTAATACTTATA |
| | | CAAAAACGCCAGGAAGATCAATATACAGAAATGTAACT |
| | | GAATTTAATTGGAGAGATGAATTGGAGCTTTATTCTGAT |
| | | TTAAAAAATGATAAGAATAAATTAATTGCTGCAATGAT |
| | | GACGAGTAAGTATACTCGGTTCTATGCTCATGATAATA |
| | | ATTTTGGAAGGTTGAAAATGACAATATTTGAGTTGGGA |
| | | CATCATTGTCAGCCTAACTACGTGGCATCTAATCACCCA |
| | | GGCAATGCTTCCGATATCCAGTACTGTAAATGGTGTAAT |
| | | ATAAAATATTTTCTTAGTAAAATTGATTGGCGGATTCGT |
| | | GATATGTATAATTTATTGATGGAATTTATTAAGGATTGT |
| | | TATAAAAGTAATGTTAACGTTGGACATTGTAGTTCTGTT |
| | | GAAAACATATATCCTTTAATTAAAAGATTAATTTGGAGT |
| | | TTGTTTACTAATCACATGGATCAAACAATTGAAGAAGT |
| | | GTTTAATCACATGTCGCCAGTGTCAGTTGAAGGTACGA |
| | | ATGTCATCATGTTGATTCTTGGATTGAATATTAGTTTGT |
| | | ATAATGAAATTAAGCGCACTTTGAATGTAGATAGCATA |
| | | CCAATGGTACTTAATTTAAATGAATTCAGTAGTATAGTT |
| | | AAATCAATTAGCAGTAAATGGTATAATGTTGATGAATT |
| | | GGATAAATTGCCAATGTCAATAAAATCAACGGAGGAAC |
| | | TGATTGAAATGAAGAATTCTGGAACTTTAACTGAAGAA |
| | | TTTGAGCTACTGATCTCCAACTCAGAAGATGACAATGA |
| | | GGGCTCCGGCGAGGGCAGGGGAAGTCTTCTAACATGCG |
| | | GGGACGTGGAGGAAAATCCCGGCCCAGTGAGCAAGGG |
| | | CGAGGAGCTGTTCACCGGGGTGGTGCCCATCCTGGTCG |
| | | AGCTGGACGGCGACGTAAACGGCCACAAGTTCAGCGTG |
| | | TCCGGCGAGGGCGAGGGCGATGCCACCTACGGCAAGCT |
| | | GACCCTGAAGTTCATCTGCACCACCGGCAAGCTGCCCG |
| | | TGCCCTGGCCCACCCTCGTGACCACCCTGACCTACGGCG |
| | | TGCAGTGCTTCAGCCGCTACCCCGACCACATGAAGCAG |
| | | CACGACTTCTTCAAGTCCGCCATGCCCGAAGGCTACGTC |
| | | CAGGAGCGCACCATCTTCTTCAAGGACGACGGCAACTA |
| | | CAAGACCCGCGCCGAGGTGAAGTTCGAGGGCGACACCC |
| | | TGGTGAACCGCATCGAGCTGAAGGGCATCGACTTCAAG |
| | | GAGGACGGCAACATCCTGGGGCACAAGCTGGAGTACA |
| | | ACTACAACAGCCACAACGTCTATATCATGGCCGACAAG |
| | | CAGAAGAACGGCATCAAGGTGAACTTCAAGATCCGCCA |
| | | CAACATCGAGGACGGCAGCGTGCAGCTCGCCGACCACT |
| | | ACCAGCAGAACACCCCCATCGGCGACGGCCCCGTGCTG |
| | | CTGCCCGACAACCACTACCTGAGCACCCAGTCCGCCCT |
| | | GAGCAAAGACCCCAACGAGAAGCGCGATCACATGGTCC |
| | | TGCTGGAGTTCGTGACCGCCGCCGGGATCACTCTCGGC |
| | | ATGGACGAGCTGTACAAGTAATGAAATTATGTCACTAT |
| | | CTAATTATACAGTATTTAGCCATCACAAGACCGTCCAG |
| | | ACTAGAGTAGCGCCTAGCTGGCAAAATACTGTGAACCG |
| | | GGTCGGCATGGCATCTCCACCTCCTCGCGGTCCGACCTG |
| | | GGCATCCGAAGGAGGACGCACGTCCACTCGGATGGCTA |
| | | AGGGAGAGCCTGCAGTAGCATAACCCCTTGGGGCCTCT |
| | | AAACGGGTCTTGAGGGGTTTTTTGGGTACC |
| 41 | pT7/NSP5SA11- | AAGCTTTAATACGACTCACTATAGGCTTTTAAAGCGCTA |
| | 2A-GFP | CAGTGATGTCTCTCAGTATTGACGTGACGAGTCTTCCTT |
| | T7 promoter: nt | CTATTCCTTCAACTATATATAAGAATGAATCGTCTTCAA |
| | 7-24 | CAACGTCAACTCTTTCTGGAAAATCTATTGGTAGGAGTG |
| | NSP5 CDS: nt | AACAGTACATTTCACCAGATGCAGAAGCATTCAATAAA |
| | 45-641 | TACATGCTGTCGAAGTCTCCAGAGGATATTGGACCATCT |
| | HDV Ribozyme: | GATTCTGCTTCAAACGATCCACTCACCAGTTTTTCGATT |
| | nt 691-779 | AGATCGAATGCAGTTAAGACAAACGCAGACGCTGGCGT |
| | T7 Terminator: | GTCTATGGATTCATCAGCACAATCACGACCTTCAAGTA |
| | nt 788-830 | ATGTCGGATGCGATCAAGTGGATTTCTCCTTAAATAAA |
| | | GGCTTAAAAGTAAAAGCTAATTTGGACTCATCAATATC |
| | | AATATCTACGGATACTAAAAAGGAGAAATCAAAACAA |
| | | AACCATAAAAGTAGGAAGCACTACCCAAGAATTGAAGC |
| | | AGAGTCTGATTCAGATGATTATGTACTGGATGATTCAG |
| | | ATAGTGATGATGGTAAATGTAAGAACTGTAAATATAAG |
| | | AAGAAATACTTCGCATTAAGAATGAGAATGAAACAAGT |
| | | CGCAATGCAATTGATTGAAGATTTGTAAGTCTGACCTG |
| | | GGAACACACTAGGGAGCTCCCCACTCCCGTTTTGTGAC |
| | | CGGGTCGGCATGGCATCTCCACCTCCTCGCGGTCCGACC |
| | | TGGGCATCCGAAGGAGGACGCACGTCCACTCGGATGGC |
| | | TAAGGGAGAGCCTGCAGTAGCATAACCCCTTGGGGCCT |
| | | CTAAACGGGTCTTGAGGGGTTTTTTGGGTACC |
| 42 | RSV F protein | MELLIHRSSAIFLTLAINALYLTSSQNITEEFYQSTCSAVSR |
| | GenPept Acc. | GYFSALRTGWYTSVITIELSNIKETKCNGTDTKVKLIKQEL |
| | No. AAR14266 | DKYKNAVTELQLLTQNTPAANNRARREAPQYMNYTINTT |
| | (hRSV B strain | KNLNVSISKKRKRRFLGFLLGVGSAIASGIAVSKVLHLEGE |
| | 9320) | VNKIKNALLSTNKAVVSLSNGVSVLTSKVLDLKSYINNQL |
| | | LPIVNQQSCRISNIETVIEFQQKNSRLLEITREFSVNAGVTTP |
| | | LSTYMLTNSELLSLINDMPITNDQKKLMSSNVQIVRQQSYS |
| | | IMSIIKEEVLAYVVQLPIYGVIDTPCWKLHTSPLCTTNIKEG |
| | | SNICLTRTDRGWYCDNAGSVSFFPQADTCKVQSNRVFCDT |
| | | MNSLTLPSEVSLCNTDIFNSKYDCKIMTSKTDISSSVITSLG |
| | | AIVSCYGKTKCTASNKNRGIIKTFSNGCDYVSNKGVDTVS |
| | | VGNTLYYVNKLEGKNLYVKGEPIINYYDPLVFPSDEFDASI |
| | | SQVNEKINQSLAFIRRSDELLHNVNTGKSTTNIMITTIIIVIIV |
| | | VLLSLIAIGLLLYCKAKNTPVTLSKDQLSGINNIAFSK |
| 43 | pT7/RSV- | ACTAGTTAATACGACTCACTATAGGCTTTTTTTTGAAAA |
| | T4PreF | GTCTTGTGTTAGCCATGGCTACTTTTAAAGATGCATGCT |
| | T7 promoter: nt | TTCATTATCGTAGATTAACTGCTTTAAATCGGAGATTAT |
| | 7-24 | GCAACATTGGTGCAAATTCTATTTGGATGCCAGTTCCTG |
| | NSP1 CDS: nt | ATGCGAAAATTAAGGGGTGGTGTTTAGAATGTTGTCAA |
| | 54-1,541 | ATAGCTGATTTAACCCATTGTTATGGTTGCTCATTGCCG |
| | 2A peptide | CATGTTTGCAAATGGTGTGTTCAGAACAGAAGATGCTT |
| | CDS: nt 1,542- | CCTTGACAATGAACCTCATTTGCTTAAGCTTAGAACTGT |
| | 1,604 | GAAACATCCAATTACCAAAGACAAATTACAGTGTATCA |
| | RSV-T4PreF | TAGACTTGTACAATATAATATTTCCAATTAATGATAAAG |
| | CDS: nt 1,605- | TAATTAGAAAATTTGAAAGAATGATAAAGCAAAGAGA |
| | 3,308 | ATGTAGGAATCAATATAAAATTGAATGGTATAATCATT |
| | HDV Ribozyme: | TGCTGCTCCCAATTACATTAAATGCTGCTGCATTTAAGT |
| | nt 3,404-3,492 | TTGATGAAAATAATCTTTATTATGTTTTTGGGTTATATG |
| | T7 terminator: | AGAAATCAGTCAGTGATATATATGCTCCATATAGAATT |
| | nt 3,501-3,543 | GTTAACTTTATAAATGAATTTGATAAATTATTGCTTGAT |
| | | CATATTAACTTTACAAGAATGTCCAATCTACCAATAGA |
| | | GTTGAGAAACCATTACGCAAAGAAATACTTCCAATTAT |
| | | CAAGACTGCCATCATCAAAACTAAAGCAAATTTACTTTT |
| | | CAGATTTTACTAAAGAAACTGTGATTTTTAATACTTATA |
| | | CAAAAACGCCAGGAAGATCAATATACAGAAATGTAACT |
| | | GAATTTAATTGGAGAGATGAATTGGAGCTTTATTCTGAT |
| | | TTAAAAAATGATAAGAATAAATTAATTGCTGCAATGAT |
| | | GACGAGTAAGTATACTCGGTTCTATGCTCATGATAATA |
| | | ATTTTGGAAGGTTGAAAATGACAATATTTGAGTTGGGA |
| | | CATCATTGTCAGCCTAACTACGTGGCATCTAATCACCCA |
| | | GGCAATGCTTCCGATATCCAGTACTGTAAATGGTGTAAT |
| | | ATAAAATATTTTCTTAGTAAAATTGATTGGCGGATTCGT |
| | | GATATGTATAATTTATTGATGGAATTTATTAAGGATTGT |
| | | TATAAAAGTAATGTTAACGTTGGACATTGTAGTTCTGTT |
| | | GAAAACATATATCCTTTAATTAAAAGATTAATTTGGAGT |
| | | TTGTTTACTAATCACATGGATCAAACAATTGAAGAAGT |
| | | GTTTAATCACATGTCGCCAGTGTCAGTTGAAGGTACGA |
| | | ATGTCATCATGTTGATTCTTGGATTGAATATTAGTTTGT |
| | | ATAATGAAATTAAGCGCACTTTGAATGTAGATAGCATA |
| | | CCAATGGTACTTAATTTAAATGAATTCAGTAGTATAGTT |
| | | AAATCAATTAGCAGTAAATGGTATAATGTTGATGAATT |
| | | GGATAAATTGCCAATGTCAATAAAATCAACGGAGGAAC |
| | | TGATTGAAATGAAGAATTCTGGAACTTTAACTGAAGAA |
| | | TTTGAGCTACTGATCTCCAACTCAGAAGATGACAATGA |
| | | GGGCTCCGGCGAGGGCAGGGGAAGTCTTCTAACATGCG |
| | | GGGACGTGGAGGAAAATCCCGGCCCAATGGAACTCCTT |
| | | ATCCTTAAAGCAAATGCTATTACCACCATACTTACTGCA |
| | | GTCACATTCTGCTTTGCATCTGGGCAGAACATAACTGAA |
| | | GAGTTCTACCAGTCAACCTGCAGTGCCGTGTCTAAAGG |
| | | CTACCTCAGCGCACTGCGAACCGGATGGTACACGTCAG |
| | | TCATAACGATCGAACTTTCAAATATAAAGGAGAATAAA |
| | | TGCAATGGGACTGACGCCAAAGTTAAACTCATCAAACA |
| | | GGAACTTGATAAATATAAGAATGCGGTCACCGAGTTGC |
| | | AGCTCCTTATGCAGAGCACGCCTGCCACGAACAATCGC |
| | | GCCCGACGAGAACTTCCACGGTTTATGAATTATACACT |
| | | GAACAACGCCAAAAAAACTAACGTAACACTTAGTAAGA |
| | | AGAGGAAGAGGCGGTTTCTTGGTTTTTTGCTGGGGGTA |
| | | GGGTCAGCAATCGCATCTGGAGTGGCCGTTTGTAAGGT |
| | | GCTGCATCTCGAAGGTGAGGTGAATAAGATCAAGTCCG |
| | | CCCTTCTTTCTACAAATAAGGCAGTGGTCAGTTTGTCAA |
| | | ATGGAGTTAGTGTGTTGACTTTCAAGGTACTGGACCTGA |
| | | AAAACTATATAGACAAGCAGCTTCTCCCAATTTTGAAT |
| | | AAACAATCTTGCTCAATAAGTAACATCGAAACAGTGAT |
| | | TGAGTTTCAGCAAAAAAATAACAGGCTGCTTGAAATCA |
| | | CACGGGAGTTTTCCGTTAACGCCGGCGTAACGACTCCG |
| | | GTCTCTACTTATATGCTCACAAATTCAGAATTGCTTTCT |
| | | TTGATAAATGATATGCCAATAACCAACGACCAAAAGAA |
| | | ACTGATGAGTAACAATGTACAGATAGTTAGACAGCAGT |
| | | CATATTCTATCATGTGTATTATAAAAGAAGAGGTCTTGG |
| | | CCTACGTAGTACAACTCCCGCTCTATGGAGTGATCGAC |
| | | ACACCGTGCTGGAAGTTGCACACCAGCCCCCTGTGTAC |
| | | TACTAACACAAAGGAGGGTTCTAATATTTGTCTCACCCG |
| | | CACGGACCGAGGCTGGTATTGCGACAACGCTGGATCCG |
| | | TAAGTTTCTTCCCCCAGGCGGAAACATGTAAAGTGCAA |
| | | AGCAACCGCGTATTTTGCGACACAATGAATAGTCTGAC |
| | | GCTTCCATCAGAGGTCAATCTTTGTAACGTGGACATCTT |
| | | CAATCCAAAGTACGACTGTAAAATTATGACATCTAAGA |
| | | CAGACGTCTCATCCAGCGTCATCACCTCCCTCGGCGCGA |
| | | TCGTAAGCTGTTATGGCAAGACTAAGTGTACAGCTAGC |
| | | AATAAGAACAGGGGGATCATCAAAACCTTTTCTAACGG |
| | | CTGTGATTACGTGTCCAACAAAGGAGTAGATACTGTAT |
| | | CAGTCGGCAATACGCTCTATTACGTGAACAAGCAAGAA |
| | | GGTAAGAGCCTTTACGTCAAAGGGGAACCCATTATTAA |
| | | CTTTTACGACCCATTGGTCTTTCCTAGTGATGAATTCGA |
| | | CGCTTCTATAAGTCAAGTAAACGAGAAGATCAATCAGA |
| | | GTCTCGCCTTCATCAGGAAATCCGATGAACTTCTTTCTG |
| | | CGATTGGAGGATATATTCCCGAAGCACCCAGGGATGGG |
| | | CAAGCATACGTAAGAAAAGATGGAGAATGGGTTCTGCT |
| | | TTCTACATTTCTTGGGGGGCTTGTCCCGAGGGGTAGCCA |
| | | TCATCATCATCACCACAGTGCATGGTCACACCCCCAATT |
| | | TGAAAAGTAATGAAATTATGTCACTATCTAATTATACA |
| | | GTATTTAGCCATCACAAGACCGTCCAGACTAGAGTAGC |
| | | GCCTAGCTGGCAAAATACTGTGAACCGGGTCGGCATGG |
| | | CATCTCCACCTCCTCGCGGTCCGACCTGGGCATCCGAAG |
| | | GAGGACGCACGTCCACTCGGATGGCTAAGGGAGAGCCT |
| | | GCAGTAGCATAACCCCTTGGGGCCTCTAAACGGGTCTT |
| | | GAGGGGTTTTTTGGGTACC |
| 44 | RSV-T4PreF | MELLILKANAITTILTAVTFCFASGQNITEEFYQSTCSAVSK |
| | | GYLSALRTGWYTSVITIELSNIKENKCNGTDAKVKLIKQEL |
| | | DKYKNAVTELQLLMQSTPATNNRARRELPRFMNYTLNNA |
| | | KKTNVTLSKKRKRRFLGFLLGVGSAIASGVAVCKVLHLEG |
| | | EVNKIKSALLSTNKAVVSLSNGVSVLTFKVLDLKNYIDKQ |
| | | LLPILNKQSCSISNIETVIEFQQKNNRLLEITREFSVNAGVTT |
| | | PVSTYMLTNSELLSLINDMPITNDQKKLMSNNVQIVRQQS |
| | | YSIMCIIKEEVLAYVVQLPLYGVIDTPCWKLHTSPLCTTNT |
| | | KEGSNICLTRTDRGWYCDNAGSVSFFPQAETCKVQSNRVF |
| | | CDTMNSLTLPSEVNLCNVDIFNPKYDCKIMTSKTDVSSSVI |
| | | TSLGAIVSCYGKTKCTASNKNRGIIKTFSNGCDYVSNKGV |
| | | DTVSVGNTLYYVNKQEGKSLYVKGEPIINFYDPLVFPSDEF |
| | | DASISQVNEKINQSLAFIRKSDELLSAIGGYIPEAPRDGQAY |
| | | VRKDGEWVLLSTFLGGLVPRGSHHHHHHSAWSHPQFEK |
| 45 | pT7/RSV- | ACTAGTTAATACGACTCACTATAGGCTTTTTTTTGAAAA |
| | T4scPreF | GTCTTGTGTTAGCCATGGCTACTTTTAAAGATGCATGCT |
| | T7 promoter: nt | TTCATTATCGTAGATTAACTGCTTTAAATCGGAGATTAT |
| | 7-24 | GCAACATTGGTGCAAATTCTATTTGGATGCCAGTTCCTG |
| | 2A peptide | ATGCGAAAATTAAGGGGTGGTGTTTAGAATGTTGTCAA |
| | CDS: nt 1,542- | ATAGCTGATTTAACCCATTGTTATGGTTGCTCATTGCCG |
| | 1,604 | CATGTTTGCAAATGGTGTGTTCAGAACAGAAGATGCTT |
| | RSV-T4scPreF | CCTTGACAATGAACCTCATTTGCTTAAGCTTAGAACTGT |
| | CDS: nt 1,605- | GAAACATCCAATTACCAAAGACAAATTACAGTGTATCA |
| | 3,203 | TAGACTTGTACAATATAATATTTCCAATTAATGATAAAG |
| | HDV Ribozyme: | TAATTAGAAAATTTGAAAGAATGATAAAGCAAAGAGA |
| | nt 3,299-3,387 | ATGTAGGAATCAATATAAAATTGAATGGTATAATCATT |
| | T7 terminator: | TGCTGCTCCCAATTACATTAAATGCTGCTGCATTTAAGT |
| | nt 3,396-3,438 | TTGATGAAAATAATCTTTATTATGTTTTTGGGTTATATG |
| | | AGAAATCAGTCAGTGATATATATGCTCCATATAGAATT |
| | | GTTAACTTTATAAATGAATTTGATAAATTATTGCTTGAT |
| | | CATATTAACTTTACAAGAATGTCCAATCTACCAATAGA |
| | | GTTGAGAAACCATTACGCAAAGAAATACTTCCAATTAT |
| | | CAAGACTGCCATCATCAAAACTAAAGCAAATTTACTTTT |
| | | CAGATTTTACTAAAGAAACTGTGATTTTTAATACTTATA |
| | | CAAAAACGCCAGGAAGATCAATATACAGAAATGTAACT |
| | | GAATTTAATTGGAGAGATGAATTGGAGCTTTATTCTGAT |
| | | TTAAAAAATGATAAGAATAAATTAATTGCTGCAATGAT |
| | | GACGAGTAAGTATACTCGGTTCTATGCTCATGATAATA |
| | | ATTTTGGAAGGTTGAAAATGACAATATTTGAGTTGGGA |
| | | CATCATTGTCAGCCTAACTACGTGGCATCTAATCACCCA |
| | | GGCAATGCTTCCGATATCCAGTACTGTAAATGGTGTAAT |
| | | ATAAAATATTTTCTTAGTAAAATTGATTGGCGGATTCGT |
| | | GATATGTATAATTTATTGATGGAATTTATTAAGGATTGT |
| | | TATAAAAGTAATGTTAACGTTGGACATTGTAGTTCTGTT |
| | | GAAAACATATATCCTTTAATTAAAAGATTAATTTGGAGT |
| | | TTGTTTACTAATCACATGGATCAAACAATTGAAGAAGT |
| | | GTTTAATCACATGTCGCCAGTGTCAGTTGAAGGTACGA |
| | | ATGTCATCATGTTGATTCTTGGATTGAATATTAGTTTGT |
| | | ATAATGAAATTAAGCGCACTTTGAATGTAGATAGCATA |
| | | CCAATGGTACTTAATTTAAATGAATTCAGTAGTATAGTT |
| | | AAATCAATTAGCAGTAAATGGTATAATGTTGATGAATT |
| | | GGATAAATTGCCAATGTCAATAAAATCAACGGAGGAAC |
| | | TGATTGAAATGAAGAATTCTGGAACTTTAACTGAAGAA |
| | | TTTGAGCTACTGATCTCCAACTCAGAAGATGACAATGA |
| | | GGGCTCCGGCGAGGGCAGGGGAAGTCTTCTAACATGCG |
| | | GGGACGTGGAGGAAAATCCCGGCCCAATGGAACTTTTG |
| | | ATACTGAAAGCCAATGCAATTACAACAATCCTCACGGC |
| | | CGTCACTTTCTGTTTCGCGTCTGGGCAAAATATCACAGA |
| | | AGAGTTCTACCAATCTACATGTTCAGCTGTGTCTAAAGG |
| | | TTACCTTAGCGCCCTCCGCACTGGCTGGTATACATCCGT |
| | | CATAACTATCGAGCTCTCTAATATAAAAGAAAATAAGT |
| | | GTAACGGAACAGACGCTAAAGTAAAGCTTATTAAACAA |
| | | GAGTTGGATAAGTATAAAAACGCGGTGACAGAATTGCA |
| | | GTTGCTGATGGGTGGGGGTTCCGGGGGTGGGTCCGGGG |
| | | GCGGATCTGGAAGCGCCATAGCTTCCGGAGTAGCAGTT |
| | | TGTAAGGTTTTGCACTTGGAGGGTGAAGTTAATAAAAT |
| | | TAAGTCAGCTCTGCTCAGTACTAATAAAGCTGTTGTCAG |
| | | CCTCAGCAACGGTGTCAGCGTCCTCACGTTTAAAGTACT |
| | | CGATCTTAAAAACTATATAGATAAACAACTGCTTCCCAT |
| | | ACTTAACAAACAGTCATGCAGCATCTCTAACATTGAAA |
| | | CAGTTATCGAGTTCCAGCAAAAGAATAACCGCCTTCTC |
| | | GAAATAACGCGGGAGTTTTCTGTAAATGCCGGAGTTAC |
| | | GACGCCAGTAAGCACATATATGCTCACAAACTCCGAAT |
| | | TGCTGAGTCTTATAAATGACATGCCAATAACAAACGAT |
| | | CAGAAGAAGCTGATGTCTAATAACGTCCAGATTGTCCG |
| | | ACAACAGTCTTACTCTATTATGTGTATAATAAAAGAGG |
| | | AAGTGCTTGCATACGTGGTGCAACTCCCTCTGTACGGA |
| | | GTTATAGACACCCCCTGCTGGAAGCTGCACACATCTCC |
| | | ACTCTGTACTACGAACACTAAAGAAGGTTCCAATATAT |
| | | GCCTTACCCGAACAGACCGCGGCTGGTATTGTGACAAT |
| | | GCAGGGAGTGTATCATTTTTTCCCCAAGCGGAGACGTG |
| | | TAAGGTTCAATCTAATCGCGTCTTCTGTGACACAATGAA |
| | | TAGCTTGACACTTCCATCCGAGGTGAATCTTTGTAACGT |
| | | CGACATCTTTAATCCCAAATACGATTGCAAGATCATGA |
| | | CCAGTAAAACAGATGTTTCATCTAGCGTAATAACTTCAC |
| | | TCGGCGCGATTGTCTCATGCTACGGAAAGACCAAATGC |
| | | ACGGCATCCAATAAGAACAGAGGAATTATCAAGACTTT |
| | | CTCCAATGGGTGCGACTACGTGAGTAATAAAGGCGTCG |
| | | ATACAGTGAGTGTAGGGAATACGCTGTATTACGTGAAC |
| | | AAACAGGAGGGAAAAAGTTTGTATGTGAAGGGAGAAC |
| | | CCATAATAAACTTTTACGATCCCCTCGTCTTTCCCTCTTG |
| | | TGAATTCTGTGCCTCAATATCTCAGGTAAATGAAAAAA |
| | | TAAATCAATCTTTGGCCTTTATACGCAAAAGCGACGAA |
| | | CTCCTGAGTGCGATTGGAGGATATATTCCCGAGGCCCC |
| | | CCGCGACGGACAGGCCTATGTAAGAAAGGACGGGGAA |
| | | TGGGTACTCTTGAGTACGTTCCTCGGAGGCCTTGTCCCC |
| | | AGGGGATCCCATCATCACCACCACCATTCCGCTTGGTCA |
| | | CATCCTCAATTTGAGAAATAATGAAATTATGTCACTATC |
| | | TAATTATACAGTATTTAGCCATCACAAGACCGTCCAGA |
| | | CTAGAGTAGCGCCTAGCTGGCAAAATACTGTGAACCGG |
| | | GTCGGCATGGCATCTCCACCTCCTCGCGGTCCGACCTGG |
| | | GCATCCGAAGGAGGACGCACGTCCACTCGGATGGCTAA |
| | | GGGAGAGCCTGCAGTAGCATAACCCCTTGGGGCCTCTA |
| | | AACGGGTCTTGAGGGGTTTTTTGGGTACC |
| 46 | RSV-T4scPreF | MELLILKANAITTILTAVTFCFASGQNITEEFYQSTCSAVSK |
| | | GYLSALRTGWYTSVITIELSNIKENKCNGTDAKVKLIKQEL |
| | | DKYKNAVTELQLLMGGGSGGGSGGGSGSAIASGVAVCK |
| | | VLHLEGEVNKIKSALLSTNKAVVSLSNGVSVLTFKVLDLK |
| | | NYIDKQLLPILNKQSCSISNIETVIEFQQKNNRLLEITREFSV |
| | | NAGVTTPVSTYMLTNSELLSLINDMPITNDQKKLMSNNVQ |
| | | IVRQQSYSIMCIIKEEVLAYVVQLPLYGVIDTPCWKLHTSP |
| | | LCTTNTKEGSNICLTRTDRGWYCDNAGSVSFFPQAETCKV |
| | | QSNRVFCDTMNSLTLPSEVNLCNVDIFNPKYDCKIMTSKT |
| | | DVSSSVITSLGAIVSCYGKTKCTASNKNRGIIKTFSNGCDY |
| | | VSNKGVDTVSVGNTLYYVNKQEGKSLYVKGEPIINFYDPL |
| | | VFPSCEFCASISQVNEKINQSLAFIRKSDELLSAIGGYIPEAP |
| | | RDGQAYVRKDGEWVLLSTFLGGLVPRGSHHHHHHSAWS |
| | | HPQFEK |
| 47 | pT7/RSV- | ACTAGTTAATACGACTCACTATAGGCTTTTTTTTGAAAA |
| | A2PreF | GTCTTGTGTTAGCCATGGCTACTTTTAAAGATGCATGCT |
| | T7 promoter: nt | TTCATTATCGTAGATTAACTGCTTTAAATCGGAGATTAT |
| | 7-24 | GCAACATTGGTGCAAATTCTATTTGGATGCCAGTTCCTG |
| | 2A peptide | ATGCGAAAATTAAGGGGTGGTGTTTAGAATGTTGTCAA |
| | CDS: nt 1,542- | ATAGCTGATTTAACCCATTGTTATGGTTGCTCATTGCCG |
| | 1,604 | CATGTTTGCAAATGGTGTGTTCAGAACAGAAGATGCTT |
| | RSV-A2PreF | CCTTGACAATGAACCTCATTTGCTTAAGCTTAGAACTGT |
| | CDS: nt 1,605- | GAAACATCCAATTACCAAAGACAAATTACAGTGTATCA |
| | 3,326 | TAGACTTGTACAATATAATATTTCCAATTAATGATAAAG |
| | HDV Ribozyme: | TAATTAGAAAATTTGAAAGAATGATAAAGCAAAGAGA |
| | nt 3,422-3,510 | ATGTAGGAATCAATATAAAATTGAATGGTATAATCATT |
| | T7 terminator: | TGCTGCTCCCAATTACATTAAATGCTGCTGCATTTAAGT |
| | nt 3,519-3,561 | TTGATGAAAATAATCTTTATTATGTTTTTGGGTTATATG |
| | | AGAAATCAGTCAGTGATATATATGCTCCATATAGAATT |
| | | GTTAACTTTATAAATGAATTTGATAAATTATTGCTTGAT |
| | | CATATTAACTTTACAAGAATGTCCAATCTACCAATAGA |
| | | GTTGAGAAACCATTACGCAAAGAAATACTTCCAATTAT |
| | | CAAGACTGCCATCATCAAAACTAAAGCAAATTTACTTTT |
| | | CAGATTTTACTAAAGAAACTGTGATTTTTAATACTTATA |
| | | CAAAAACGCCAGGAAGATCAATATACAGAAATGTAACT |
| | | GAATTTAATTGGAGAGATGAATTGGAGCTTTATTCTGAT |
| | | TTAAAAAATGATAAGAATAAATTAATTGCTGCAATGAT |
| | | GACGAGTAAGTATACTCGGTTCTATGCTCATGATAATA |
| | | ATTTTGGAAGGTTGAAAATGACAATATTTGAGTTGGGA |
| | | CATCATTGTCAGCCTAACTACGTGGCATCTAATCACCCA |
| | | GGCAATGCTTCCGATATCCAGTACTGTAAATGGTGTAAT |
| | | ATAAAATATTTTCTTAGTAAAATTGATTGGCGGATTCGT |
| | | GATATGTATAATTTATTGATGGAATTTATTAAGGATTGT |
| | | TATAAAAGTAATGTTAACGTTGGACATTGTAGTTCTGTT |
| | | GAAAACATATATCCTTTAATTAAAAGATTAATTTGGAGT |
| | | TTGTTTACTAATCACATGGATCAAACAATTGAAGAAGT |
| | | GTTTAATCACATGTCGCCAGTGTCAGTTGAAGGTACGA |
| | | ATGTCATCATGTTGATTCTTGGATTGAATATTAGTTTGT |
| | | ATAATGAAATTAAGCGCACTTTGAATGTAGATAGCATA |
| | | CCAATGGTACTTAATTTAAATGAATTCAGTAGTATAGTT |
| | | AAATCAATTAGCAGTAAATGGTATAATGTTGATGAATT |
| | | GGATAAATTGCCAATGTCAATAAAATCAACGGAGGAAC |
| | | TGATTGAAATGAAGAATTCTGGAACTTTAACTGAAGAA |
| | | TTTGAGCTACTGATCTCCAACTCAGAAGATGACAATGA |
| | | GGGCTCCGGCGAGGGCAGGGGAAGTCTTCTAACATGCG |
| | | GGGACGTGGAGGAAAATCCCGGCCCAATGGAGTTGCTA |
| | | ATCCTCAAAGCAAATGCAATTACCACAATCCTCACTGC |
| | | AGTCACATTTTGTTTTGCTTCTGGTCAAAACATCACTGA |
| | | AGAATTTTATCAATCAACATGCAGTGCAGTTAGCAAAG |
| | | GCTATCTTAGTGCTCTGAGAACTGGTTGGTATACCAGTG |
| | | TTATAACTATAGAATTAAGTAATATCAAGGAAAATAAG |
| | | TGTAATGGAACAGATGCTAAGGTAAAATTGATAAAACA |
| | | AGAATTAGATAAATATAAAAATGCTGTAACAGAATTGC |
| | | AGTTGCTCATGCAAAGCACACCACCAACAAACAATCGA |
| | | GCCAGAAGAGAACTACCAAGGTTTATGAATTATACACT |
| | | CAACAATGCCAAAAAAACCAATGTAACATTAAGCAAGA |
| | | AAAGGAAAAGAAGATTTCTTGTTTTTTTGTTAGGTGTTG |
| | | GATCTGCAATCGCCAGTGGCGTTGCTGTATGTAAGGTCC |
| | | TGCACCTAGAAGGGGAAGTGAACAAGATCAAAAGTGCT |
| | | CTACTATCCACAAACAAGGCTCTAGTCAGCTTATCAAAT |
| | | GGAGTTAGTGTCTTAACCTTCAAAGTGTTAGACCTCAAA |
| | | AACTATATAGATAAACAATTGTTACCTATTCTGAACAA |
| | | GCAAAGCTGCAGCATATCAAATATAGAAACTGTGATAG |
| | | AGTTCCAACAAAAGAACAACAGACTACTAGAGATTACC |
| | | AGGGAATTTAGTGTTAATGCAGGTGTAACTACACCTGT |
| | | AAGCACTTACATGTTAACTAATAGTGAATTATTGTCATT |
| | | AATCAATGATATGCCTATAACAAATGATCAGAAAAAGT |
| | | TAATGTCCAACAATGTTCAAATAGTTAGACAGCAAAGT |
| | | TACTCTATCATGTGCATAATAAAAGAGGAAGTCTTAGC |
| | | ATATGTAGTACAATTACCACTATATGGTGTTATAGATAC |
| | | ACCCTGTTGGAAACTACACACATCCCCTCTATGTACAAC |
| | | CAACACAAAAGAAGGGTCCAACATCTGTTTAACAAGAA |
| | | CTGACAGAGGATGGTACTGTGACAATGCAGGATCAGTA |
| | | TCTTTCTTCCCACAAGCTGAAACATGTAAAGTTCAATCA |
| | | AATCGAGTATTTTGTGACACAATGAACAGTTTAACATTA |
| | | CCAAGTGAAATAAATCTCTGCAATGTTGACATATTCAA |
| | | CCCCAAATATGATTGTAAAATTATGACTTCAAAAACAG |
| | | ATGTAAGCAGCTCCGTTATCACATCTCTAGGAGCCATTG |
| | | TGTCATGCTATGGCAAAACTAAATGTACAGCATCCAAT |
| | | AAAAATCGTGGAATCATAAAGACATTTTCTAACGGGTG |
| | | CGATTATGTATCAAATAAAGGGATGGACACTGTGTCTG |
| | | TAGGTAACACATTATATTATGTAAATAAGCAAGAAGGT |
| | | AAAAGTCTCTATGTAAAAGGTGAACCAATAATAAATTT |
| | | CTATGACCCATTAGTATTCCCCTCTGATGAATTTGATGC |
| | | ATCAATATCTCAAGTCAACGAGAAGATTAACCAGAGCC |
| | | TAGCATTTATTCGTAAATCCGATGAATTATTACATAATG |
| | | TAAATGCTGGTAAATCCACCACAAATATCATGATAACT |
| | | ACTATAATTATAGTGATTATAGTAATATTGTTATCATTA |
| | | ATTGCTGTTGGACTGCTCTTATACTGTAAGGCCAGAAGC |
| | | ACACCAGTCACACTAAGCAAAGATCAACTGAGTGGTAT |
| | | AAATAATATTGCATTTAGTAACTAATGAAATTATGTCAC |
| | | TATCTAATTATACAGTATTTAGCCATCACAAGACCGTCC |
| | | AGACTAGAGTAGCGCCTAGCTGGCAAAATACTGTGAAC |
| | | CGGGTCGGCATGGCATCTCCACCTCCTCGCGGTCCGACC |
| | | TGGGCATCCGAAGGAGGACGCACGTCCACTCGGATGGC |
| | | TAAGGGAGAGCCTGCAGTAGCATAACCCCTTGGGGCCT |
| | | CTAAACGGGTCTTGAGGGGTTTTTTGGGTACC |
| 48 | RSV-A2PreF | MELLILKANAITTILTAVTFCFASGQNITEEFYQSTCSAVSK |
| | | GYLSALRTGWYTSVITIELSNIKENKCNGTDAKVKLIKQEL |
| | | DKYKNAVTELQLLMQSTPPTNNRARRELPRFMNYTLNNA |
| | | KKTNVTLSKKRKRRFLVFLLGVGSAIASGVAVCKVLHLEG |
| | | EVNKIKSALLSTNKALVSLSNGVSVLTFKVLDLKNYIDKQ |
| | | LLPILNKQSCSISNIETVIEFQQKNNRLLEITREFSVNAGVTT |
| | | PVSTYMLTNSELLSLINDMPITNDQKKLMSNNVQIVRQQS |
| | | YSIMCIIKEEVLAYVVQLPLYGVIDTPCWKLHTSPLCTTNT |
| | | KEGSNICLTRTDRGWYCDNAGSVSFFPQAETCKVQSNRVF |
| | | CDTMNSLTLPSEINLCNVDIFNPKYDCKIMTSKTDVSSSVIT |
| | | SLGAIVSCYGKTKCTASNKNRGIIKTFSNGCDYVSNKGMD |
| | | TVSVGNTLYYVNKQEGKSLYVKGEPIINFYDPLVFPSDEFD |
| | | ASISQVNEKINQSLAFIRKSDELLHNVNAGKSTTNIMITTIII |
| | | VIIVILLSLIAVGLLLYCKARSTPVTLSKDQLSGINNIAFSN |
| 49 | pT7/RSV- | ACTAGTTAATACGACTCACTATAGGCTTTTTTTTGAAAA |
| | A2scPreF | GTCTTGTGTTAGCCATGGCTACTTTTAAAGATGCATGCT |
| | T7 promoter: nt | TTCATTATCGTAGATTAACTGCTTTAAATCGGAGATTAT |
| | 7-24 | GCAACATTGGTGCAAATTCTATTTGGATGCCAGTTCCTG |
| | 2A peptide | ATGCGAAAATTAAGGGGTGGTGTTTAGAATGTTGTCAA |
| | CDS: nt 1,542- | ATAGCTGATTTAACCCATTGTTATGGTTGCTCATTGCCG |
| | 1,604 | CATGTTTGCAAATGGTGTGTTCAGAACAGAAGATGCTT |
| | A2scPreF CDS: | CCTTGACAATGAACCTCATTTGCTTAAGCTTAGAACTGT |
| | nt 1,605-3,218 | GAAACATCCAATTACCAAAGACAAATTACAGTGTATCA |
| | HDV Ribozyme: | TAGACTTGTACAATATAATATTTCCAATTAATGATAAAG |
| | nt 3,314-3,402 | TAATTAGAAAATTTGAAAGAATGATAAAGCAAAGAGA |
| | T7 terminator: | ATGTAGGAATCAATATAAAATTGAATGGTATAATCATT |
| | nt 3,411-3,453 | TGCTGCTCCCAATTACATTAAATGCTGCTGCATTTAAGT |
| | | TTGATGAAAATAATCTTTATTATGTTTTTGGGTTATATG |
| | | AGAAATCAGTCAGTGATATATATGCTCCATATAGAATT |
| | | GTTAACTTTATAAATGAATTTGATAAATTATTGCTTGAT |
| | | CATATTAACTTTACAAGAATGTCCAATCTACCAATAGA |
| | | GTTGAGAAACCATTACGCAAAGAAATACTTCCAATTAT |
| | | CAAGACTGCCATCATCAAAACTAAAGCAAATTTACTTTT |
| | | CAGATTTTACTAAAGAAACTGTGATTTTTAATACTTATA |
| | | CAAAAACGCCAGGAAGATCAATATACAGAAATGTAACT |
| | | GAATTTAATTGGAGAGATGAATTGGAGCTTTATTCTGAT |
| | | TTAAAAAATGATAAGAATAAATTAATTGCTGCAATGAT |
| | | GACGAGTAAGTATACTCGGTTCTATGCTCATGATAATA |
| | | ATTTTGGAAGGTTGAAAATGACAATATTTGAGTTGGGA |
| | | CATCATTGTCAGCCTAACTACGTGGCATCTAATCACCCA |
| | | GGCAATGCTTCCGATATCCAGTACTGTAAATGGTGTAAT |
| | | ATAAAATATTTTCTTAGTAAAATTGATTGGCGGATTCGT |
| | | GATATGTATAATTTATTGATGGAATTTATTAAGGATTGT |
| | | TATAAAAGTAATGTTAACGTTGGACATTGTAGTTCTGTT |
| | | GAAAACATATATCCTTTAATTAAAAGATTAATTTGGAGT |
| | | TTGTTTACTAATCACATGGATCAAACAATTGAAGAAGT |
| | | GTTTAATCACATGTCGCCAGTGTCAGTTGAAGGTACGA |
| | | ATGTCATCATGTTGATTCTTGGATTGAATATTAGTTTGT |
| | | ATAATGAAATTAAGCGCACTTTGAATGTAGATAGCATA |
| | | CCAATGGTACTTAATTTAAATGAATTCAGTAGTATAGTT |
| | | AAATCAATTAGCAGTAAATGGTATAATGTTGATGAATT |
| | | GGATAAATTGCCAATGTCAATAAAATCAACGGAGGAAC |
| | | TGATTGAAATGAAGAATTCTGGAACTTTAACTGAAGAA |
| | | TTTGAGCTACTGATCTCCAACTCAGAAGATGACAATGA |
| | | GGGCTCCGGCGAGGGCAGGGGAAGTCTTCTAACATGCG |
| | | GGGACGTGGAGGAAAATCCCGGCCCAATGGAGCTGCTC |
| | | ATCCTCAAGGCCAACGCCATCACCACCATTCTGACCGCT |
| | | GTGACCTTCTGTTTCGCTTCCGGCCAGAACATCACAGAA |
| | | GAGTTCTACCAGTCCACATGCAGCGCCGTCTCCAAGGG |
| | | ATATCTCTCCGCTCTGAGAACCGGCTGGTATACAAGCGT |
| | | GATCACAATCGAACTGAGCAATATTAAGGAGAATAAGT |
| | | GCAACGGCACCGACGCTAAGGTCAAGCTGATCAAGCAA |
| | | GAGCTCGACAAATACAAGAACGCTGTGACCGAACTCCA |
| | | GCTGCTGATGGGCGGCGGCAGCGGCGGCGGCAGCGGC |
| | | GGCGGCAGCAGCGCTATTGCTAGCGGCGTGGCCGTGTG |
| | | CAAGGTCCTCCATCTGGAGGGAGAGGTCAACAAGATCA |
| | | AGAGCGCTCTGCTGTCCACCAACAAGGCTCTGGTGTCC |
| | | CTCAGCAACGGAGTGAGCGTGCTCACCTTCAAAGTGCT |
| | | CGATCTGAAAAACTACATTGATAAGCAGCTGCTGCCCA |
| | | TTCTGAACAAGCAAAGCTGCAGCATCAGCAATATCGAG |
| | | ACCGTGATCGAATTTCAACAGAAAAACAATAGACTGCT |
| | | CGAGATCACAAGAGAATTTTCCGTGAATGCCGGAGTGA |
| | | CAACCCCCGTGAGCACCTACATGCTGACCAATTCCGAG |
| | | CTGCTGTCCCTCATCAACGACATGCCCATCACCAACGAC |
| | | CAGAAGAAGCTCATGTCCAACAACGTCCAGATTGTGAG |
| | | GCAGCAGAGCTATAGCATTATGTGTATTATTAAGGAGG |
| | | AGGTGCTGGCCTACGTGGTCCAACTGCCTCTGTATGGCG |
| | | TCATCGACACCCCTTGCTGGAAGCTCCATACAAGCCCTC |
| | | TCTGTACCACAAACACCAAAGAGGGCTCCAACATTTGT |
| | | CTGACCAGAACAGATAGAGGCTGGTATTGTGATAACGC |
| | | CGGAAGCGTCAGCTTCTTTCCCCAAGCCGAGACATGCA |
| | | AGGTGCAATCCAATAGAGTGTTCTGCGACACCATGAAC |
| | | TCTCTGACACTGCCCAGCGAAATCAATCTGTGCAACGTC |
| | | GACATCTTCAACCCCAAGTACGACTGCAAGATCATGAC |
| | | CTCCAAAACCGACGTCTCCAGCAGCGTCATCACATCTCT |
| | | GGGCGCCATCGTGAGCTGCTATGGCAAGACCAAATGCA |
| | | CCGCTAGCAACAAGAATAGAGGAATCATCAAAACCTTT |
| | | AGCAACGGCTGTGACTACGTCTCCAACAAGGGAATGGA |
| | | CACAGTGTCCGTGGGCAACACACTGTACTATGTGAACA |
| | | AGCAAGAGGGCAAGTCTCTGTACGTCAAAGGCGAGCCC |
| | | ATCATCAACTTCTATGACCCCCTCGTGTTCCCTTCCTGC |
| | | GAGTTTTGCGCTTCCATCAGCCAAGTGAACGAGAAAAT |
| | | CAACCAGTCTCTGGCCTTCATTAGGAAGAGCGACGAGC |
| | | TGCTCCACAACGTGAACGCCGGCAAGAGCACCACCAAC |
| | | ATCATGATCACCACAATTATCATCGTGATTATTGTCATT |
| | | CTGCTGTCTCTGATTGCCGTGGGACTGCTGCTCTATTGC |
| | | AAGGCTAGATCCACACCCGTGACACTGTCCAAGGATCA |
| | | GCTGAGCGGCATCAACAACATTGCCTTCAGCAACTAAT |
| | | GAAATTATGTCACTATCTAATTATACAGTATTTAGCCAT |
| | | CACAAGACCGTCCAGACTAGAGTAGCGCCTAGCTGGCA |
| | | AAATACTGTGAACCGGGTCGGCATGGCATCTCCACCTC |
| | | CTCGCGGTCCGACCTGGGCATCCGAAGGAGGACGCACG |
| | | TCCACTCGGATGGCTAAGGGAGAGCCTGCAGTAGCATA |
| | | ACCCCTTGGGGCCTCTAAACGGGTCTTGAGGGGTTTTTT |
| | | GGGTACC |
| 50 | RSV-A2scPreF | MELLILKANAITTILTAVTFCFASGQNITEEFYQSTCSAVSK |
| | | GYLSALRTGWYTSVITIELSNIKENKCNGTDAKVKLIKQEL |
| | | DKYKNAVTELQLLMGGGSGGGSGGGSSAIASGVAVCKVL |
| | | HLEGEVNKIKSALLSTNKALVSLSNGVSVLTFKVLDLKNY |
| | | IDKQLLPILNKQSCSISNIETVIEFQQKNNRLLEITREFSVNA |
| | | GVTTPVSTYMLTNSELLSLINDMPITNDQKKLMSNNVQIV |
| | | RQQSYSIMCIIKEEVLAYVVQLPLYGVIDTPCWKLHTSPLC |
| | | TTNTKEGSNICLTRTDRGWYCDNAGSVSFFPQAETCKVQS |
| | | NRVFCDTMNSLTLPSEINLCNVDIFNPKYDCKIMTSKTDVS |
| | | SSVITSLGAIVSCYGKTKCTASNKNRGIIKTFSNGCDYVSN |
| | | KGMDTVSVGNTLYYVNKQEGKSLYVKGEPIINFYDPLVFP |
| | | SCEFCASISQVNEKINQSLAFIRKSDELLHNVNAGKSTTNI |
| | | MITTIIIVIIVILLSLIAVGLLLYCKARSTPVTLSKDQLSGINN |
| | | IAFSN |
| 51 | RA-PCR | CAACGGAGGAACTGATTGAAATGAAGAA |
| | forward primer | |
| 52 | RA-PCR reverse | TTGCCAGCTAGGCGCTACT |
| | primer | |
| 53 | Sequencing | GCTACTGATCTCCAACTCAGAAGATG |
| | forward primer | |
| 54 | Sequencing | TAGTCTGGACGGTCTTGTGA |
| | reverse primer | |
| 55 | pT7/NSP1-GFP- | ACTAGTTAATACGACTCACTATAGGCTTTTTTTTGAAAA |
| | NSP 1repeat | GTCTTGTGTTAGCCATGGCTACTTTTAAAGATGCATGCT |
| | T7 promoter: nt | TTCATTATCGTAGATTAACTGCTTTAAATCGGAGATTAT |
| | 7-27 | GCAACATTGGTGCAAATTCTATTTGGATGCCAGTTCCTG |
| | NSP1 CDS: nt | ATGCGAAAATTAAGGGGTGGTGTTTAGAATGTTGTCAA |
| | 54-1,541 | ATAGCTGATTTAACCCATTGTTATGGTTGCTCATTGCCG |
| | 2A peptide | CATGTTTGCAAATGGTGTGTTCAGAACAGAAGATGCTT |
| | CDS: nt 1,542- | CCTTGACAATGAACCTCATTTGCTTAAGCTTAGAACTGT |
| | 1,604 | GAAACATCCAATTACCAAAGACAAATTACAGTGTATCA |
| | GFP CDS: nt | TAGACTTGTACAATATAATATTTCCAATTAATGATAAAG |
| | 1,605-2,321 | TAATTAGAAAATTTGAAAGAATGATAAAGCAAAGAGA |
| | NSP1 CDS (last | ATGTAGGAATCAATATAAAATTGAATGGTATAATCATT |
| | 450bp): nt | TGCTGCTCCCAATTACATTAAATGCTGCTGCATTTAAGT |
| | 2,322-2,771 | TTGATGAAAATAATCTTTATTATGTTTTTGGGTTATATG |
| | HDV Ribozyme: | AGAAATCAGTCAGTGATATATATGCTCCATATAGAATT |
| | nt 2,864-2,952 | GTTAACTTTATAAATGAATTTGATAAATTATTGCTTGAT |
| | T7 Terminator: | CATATTAACTTTACAAGAATGTCCAATCTACCAATAGA |
| | nt 2,961-3,003 | GTTGAGAAACCATTACGCAAAGAAATACTTCCAATTAT |
| | | CAAGACTGCCATCATCAAAACTAAAGCAAATTTACTTTT |
| | | CAGATTTTACTAAAGAAACTGTGATTTTTAATACTTATA |
| | | CAAAAACGCCAGGAAGATCAATATACAGAAATGTAACT |
| | | GAATTTAATTGGAGAGATGAATTGGAGCTTTATTCTGAT |
| | | TTAAAAAATGATAAGAATAAATTAATTGCTGCAATGAT |
| | | GACGAGTAAGTATACTCGGTTCTATGCTCATGATAATA |
| | | ATTTTGGAAGGTTGAAAATGACAATATTTGAGTTGGGA |
| | | CATCATTGTCAGCCTAACTACGTGGCATCTAATCACCCA |
| | | GGCAATGCTTCCGATATCCAGTACTGTAAATGGTGTAAT |
| | | ATAAAATATTTTCTTAGTAAAATTGATTGGCGGATTCGT |
| | | GATATGTATAATTTATTGATGGAATTTATTAAGGATTGT |
| | | TATAAAAGTAATGTTAACGTTGGACATTGTAGTTCTGTT |
| | | GAAAACATATATCCTTTAATTAAAAGATTAATTTGGAGT |
| | | TTGTTTACTAATCACATGGATCAAACAATTGAAGAAGT |
| | | GTTTAATCACATGTCGCCAGTGTCAGTTGAAGGTACGA |
| | | ATGTCATCATGTTGATTCTTGGATTGAATATTAGTTTGT |
| | | ATAATGAAATTAAGCGCACTTTGAATGTAGATAGCATA |
| | | CCAATGGTACTTAATTTAAATGAATTCAGTAGTATAGTT |
| | | AAATCAATTAGCAGTAAATGGTATAATGTTGATGAATT |
| | | GGATAAATTGCCAATGTCAATAAAATCAACGGAGGAAC |
| | | TGATTGAAATGAAGAATTCTGGAACTTTAACTGAAGAA |
| | | TTTGAGCTACTGATCTCCAACTCAGAAGATGACAATGA |
| | | GGGCTCCGGCGAGGGCAGGGGAAGTCTTCTAACATGCG |
| | | GGGACGTGGAGGAAAATCCCGGCCCAGTGAGCAAGGG |
| | | CGAGGAGCTGTTCACCGGGGTGGTGCCCATCCTGGTCG |
| | | AGCTGGACGGCGACGTAAACGGCCACAAGTTCAGCGTG |
| | | TCCGGCGAGGGCGAGGGCGATGCCACCTACGGCAAGCT |
| | | GACCCTGAAGTTCATCTGCACCACCGGCAAGCTGCCCG |
| | | TGCCCTGGCCCACCCTCGTGACCACCCTGACCTACGGCG |
| | | TGCAGTGCTTCAGCCGCTACCCCGACCACATGAAGCAG |
| | | CACGACTTCTTCAAGTCCGCCATGCCCGAAGGCTACGTC |
| | | CAGGAGCGCACCATCTTCTTCAAGGACGACGGCAACTA |
| | | CAAGACCCGCGCCGAGGTGAAGTTCGAGGGCGACACCC |
| | | TGGTGAACCGCATCGAGCTGAAGGGCATCGACTTCAAG |
| | | GAGGACGGCAACATCCTGGGGCACAAGCTGGAGTACA |
| | | ACTACAACAGCCACAACGTCTATATCATGGCCGACAAG |
| | | CAGAAGAACGGCATCAAGGTGAACTTCAAGATCCGCCA |
| | | CAACATCGAGGACGGCAGCGTGCAGCTCGCCGACCACT |
| | | ACCAGCAGAACACCCCCATCGGCGACGGCCCCGTGCTG |
| | | CTGCCCGACAACCACTACCTGAGCACCCAGTCCGCCCT |
| | | GAGCAAAGACCCCAACGAGAAGCGCGATCACATGGTCC |
| | | TGCTGGAGTTCGTGACCGCCGCCGGGATCACTCTCGGC |
| | | ATGGACGAGCTGTACAAGTAATTATTGATGGAATTTATT |
| | | AAGGATTGTTATAAAAGTAATGTTAACGTTGGACATTG |
| | | TAGTTCTGTTGAAAACATATATCCTTTAATTAAAAGATT |
| | | AATTTGGAGTTTGTTTACTAATCACATGGATCAAACAAT |
| | | TGAAGAAGTGTTTAATCACATGTCGCCAGTGTCAGTTG |
| | | AAGGTACGAATGTCATCATGTTGATTCTTGGATTGAATA |
| | | TTAGTTTGTATAATGAAATTAAGCGCACTTTGAATGTAG |
| | | ATAGCATACCAATGGTACTTAATTTAAATGAATTCAGTA |
| | | GTATAGTTAAATCAATTAGCAGTAAATGGTATAATGTT |
| | | GATGAATTGGATAAATTGCCAATGTCAATAAAATCAAC |
| | | GGAGGAACTGATTGAAATGAAGAATTCTGGAACTTTAA |
| | | CTGAAGAATTTGAGCTACTGATCTCCAACTCAGAAGAT |
| | | GACAATGAGTGAAATTATGTCACTATCTAATTATACAGT |
| | | ATTTAGCCATCACAAGACCGTCCAGACTAGAGTAGCGC |
| | | CTAGCTGGCAAAATACTGTGAACCGGGTCGGCATGGCA |
| | | TCTCCACCTCCTCGCGGTCCGACCTGGGCATCCGAAGG |
| | | AGGACGCACGTCCACTCGGATGGCTAAGGGAGAGCCTG |
| | | CAGTAGCATAACCCCTTGGGGCCTCTAAACGGGTCTTG |
| | | AGGGGTTTTTTGGGTACC |
| 56 | pT7/NSP2-GFP- | AAGCTTTAATACGACTCACTATAGGCTTTTAAAGCGTCT |
| | NSP2repeat | CAGTCGCCGTTTGAGCCTTGCGGTGTAGCCATGGCTGA |
| | T7 promoter: nt | GCTAGCTTGCTTTTGCTATCCTCATTTGGAGAATGATAG |
| | 7-27 | CTATAAATTTATTCCTTTTAATAATTTAGCTATTAAAGC |
| | NSP2 CDS: nt | TATGCTGACAGCTAAAGTAGACAAAAAGGACATGGATA |
| | 70-1,020 | AGTTTTATGATTCAATTATTTATGGAATAGCACCGCCTC |
| | 2A peptide | CTCAATTTAAGAAACGGTATAATACTAATGATAATTCA |
| | CDS: nt 1,021- | AGAGGCATGAATTTTGAAACAATTATGTTTACTAAGGT |
| | 1,083 | GGCTATGTTGATATGTGAAGCTCTAAATTCATTGAAAGT |
| | GFP CDS: nt | GACGCAAGCAAACGTCTCTAATGTATTATCACGAGTAG |
| | 1,084-1,800 | TATCAATAAGGCATTTAGAAAATTTGGTGATACGTAAA |
| | NSP2 CDS (last | GAAAATCCACAGGATATTCTATTTCATTCAAAAGATTTA |
| | 168bp): nt | CTTTTGAAATCAACACTGATTGCTATTGGACAGTCTAAA |
| | 1,801-1,968 | GAAATTGAAACTACAATAACTGCAGAAGGAGGAGAAA |
| | HDV Ribozyme: | TTGTATTTCAAAACGCTGCCTTCACCATGTGGAAACTAA |
| | nt 2,031-2,119 | CTTATTTAGAACATCAATTGATGCCAATTCTGGATCAGA |
| | T7 Terminator: | ATTTTATTGAATATAAAGTTACATTGAACGAAGATAAA |
| | nt 2,128-2,170 | CCAATTTCAGATGTTCATGTTAAAGAATTAGTCGCTGAA |
| | | CTTCGATGGCAATATAACAAGTTTGCTGTAATCACACAT |
| | | GGTAAGGGTCATTATAGAATTGTAAAGTATTCATCAGTT |
| | | GCTAATCACGCTGACAGAGTATATGCAACTTTCAAGAG |
| | | TAATGTTAAAACTGGAGTTAATAATGATTTTAACCTACT |
| | | TGATCAAAGAATTATTTGGCAAAACTGGTATGCATTTAC |
| | | ATCATCAATGAAACAGGGTAATACACTTGACGTGTGTA |
| | | AAAGGTTGCTTTTCCAAAAAATGAAACCAGAAAAAAAT |
| | | CCATTTAAAGGGCTGTCAACGGATAGAAAAATGGACGA |
| | | AGTTTCTCAAGTTGGCGTTGGCTCCGGCGAGGGCAGGG |
| | | GAAGTCTTCTAACATGCGGGGACGTGGAGGAAAATCCC |
| | | GGCCCAGTGAGCAAGGGCGAGGAGCTGTTCACCGGGGT |
| | | GGTGCCCATCCTGGTCGAGCTGGACGGCGACGTAAACG |
| | | GCCACAAGTTCAGCGTGTCCGGCGAGGGCGAGGGCGAT |
| | | GCCACCTACGGCAAGCTGACCCTGAAGTTCATCTGCAC |
| | | CACCGGCAAGCTGCCCGTGCCCTGGCCCACCCTCGTGA |
| | | CCACCCTGACCTACGGCGTGCAGTGCTTCAGCCGCTACC |
| | | CCGACCACATGAAGCAGCACGACTTCTTCAAGTCCGCC |
| | | ATGCCCGAAGGCTACGTCCAGGAGCGCACCATCTTCTT |
| | | CAAGGACGACGGCAACTACAAGACCCGCGCCGAGGTG |
| | | AAGTTCGAGGGCGACACCCTGGTGAACCGCATCGAGCT |
| | | GAAGGGCATCGACTTCAAGGAGGACGGCAACATCCTGG |
| | | GGCACAAGCTGGAGTACAACTACAACAGCCACAACGTC |
| | | TATATCATGGCCGACAAGCAGAAGAACGGCATCAAGGT |
| | | GAACTTCAAGATCCGCCACAACATCGAGGACGGCAGCG |
| | | TGCAGCTCGCCGACCACTACCAGCAGAACACCCCCATC |
| | | GGCGACGGCCCCGTGCTGCTGCCCGACAACCACTACCT |
| | | GAGCACCCAGTCCGCCCTGAGCAAAGACCCCAACGAGA |
| | | AGCGCGATCACATGGTCCTGCTGGAGTTCGTGACCGCC |
| | | GCCGGGATCACTCTCGGCATGGACGAGCTGTACAAGTA |
| | | ACAAAGAATTATTTGGCAAAACTGGTATGCATTTACAT |
| | | CATCAATGAAACAGGGTAATACACTTGACGTGTGTAAA |
| | | AGGTTGCTTTTCCAAAAAATGAAACCAGAAAAAAATCC |
| | | ATTTAAAGGGCTGTCAACGGATAGAAAAATGGACGAAG |
| | | TTTCTCAAGTTGGCGTTTAATTCGCTATCAATTTGAGGA |
| | | TGATGATGGCTTAGCAAGAATAGAAAGCGCTTATGTGA |
| | | CCGGGTCGGCATGGCATCTCCACCTCCTCGCGGTCCGAC |
| | | CTGGGCATCCGAAGGAGGACGCACGTCCACTCGGATGG |
| | | CTAAGGGAGAGCCTGCAGTAGCATAACCCCTTGGGGCC |
| | | TCTAAACGGGTCTTGAGGGGTTTTTTGGGTACC |
| 57 | pT7/NSP3-GFP- | ACTAGTTAATACGACTCACTATAGGCATTTAATGCTTTT |
| | NSP3repeat | CAGTGGTTGATGCTCAAGATGGAGTCTACGCAACAGAT |
| | T7 promoter: nt | GGCCGTCTCAATTATTAACTCTTCTTTTGAAGCTGCAGT |
| | 7-27 | TGTAGCTGCAACCTCAGCTCTTGAGAATATGGGAATAG |
| | NSP3 CDS: nt | AATATGATTATCAGGATATATATTCTAGAGTAAAGAAT |
| | 49-993 | AAATTTGATTTTGTGATGGACGATTCTGGTGTTAAAAAT |
| | 2A peptide | AATCTGATTGGTAAAGCAATAACTATTGATCAAGCTTTG |
| | CDS: nt 994- | AATAATAAATTTGGATCTGCTATAAGAAATAGAAACTG |
| | 1,056 | GCTTGCTGATACTTCTAGAGCAGCTAAATTAGATGAGG |
| | GFP CDS: nt | ATGTAAACAAACTAAGAATGATGTTATCATCAAAAGGA |
| | 1,057-1,773 | ATTGATCAAAAAATGAGAGTTTTAAACGCATGCTTCAG |
| | NSP3 CDS (last | TGTAAAAAGAATACCTGGAAAATCATCATCTATTATTA |
| | 168bp): nt | AATGCACAAAATTGATGCGTGATAAATTGGAACGTGGT |
| | 1,774-1,941 | GAAGTTGAAGTGGATGATTCATTTGTGGATGAAAAAAT |
| | HDV Ribozyme: | GGAAGTGGATACCATTGACTGGAAATCGCGCTATGAGC |
| | nt 2,074-2,162 | AATTGGAGCAAAGGTTTGAATCATTGAAATCCAGGGTA |
| | T7 Terminator: | AATGAAAAATATAATAATTGGGTGTTGAAAGCAAGAAA |
| | nt 2,171-2,213 | AATGAATGAAAATATGCATTCTCTTCAAAATGTCATCTC |
| | | TCAACAGCAAGCACATATAGCTGAGCTTCAAGTGTACA |
| | | ATAATAAACTAGAACGTGATTTGCAAAATAAAATTGGA |
| | | TCCCTTACTTCTTCGATTGAATGGTATTTAAGATCAATG |
| | | GAATTAGACCCTGAAATAAAGGCAGACATTGAACAGCA |
| | | AATTAACTCAATTGATGCGATAAATCCATTGCACGCTTT |
| | | TGATGACTTAGAATCAGTAATACGTAATTTGATATCTGA |
| | | TTATGACAAATTATTCCTTATGTTCAAAGGATTAATACA |
| | | GAGATGTAATTATCAATATTCATTTGGTTGCGAAGGCTC |
| | | CGGCGAGGGCAGGGGAAGTCTTCTAACATGCGGGGACG |
| | | TGGAGGAAAATCCCGGCCCAGTGAGCAAGGGCGAGGA |
| | | GCTGTTCACCGGGGTGGTGCCCATCCTGGTCGAGCTGG |
| | | ACGGCGACGTAAACGGCCACAAGTTCAGCGTGTCCGGC |
| | | GAGGGCGAGGGCGATGCCACCTACGGCAAGCTGACCCT |
| | | GAAGTTCATCTGCACCACCGGCAAGCTGCCCGTGCCCT |
| | | GGCCCACCCTCGTGACCACCCTGACCTACGGCGTGCAG |
| | | TGCTTCAGCCGCTACCCCGACCACATGAAGCAGCACGA |
| | | CTTCTTCAAGTCCGCCATGCCCGAAGGCTACGTCCAGG |
| | | AGCGCACCATCTTCTTCAAGGACGACGGCAACTACAAG |
| | | ACCCGCGCCGAGGTGAAGTTCGAGGGCGACACCCTGGT |
| | | GAACCGCATCGAGCTGAAGGGCATCGACTTCAAGGAGG |
| | | ACGGCAACATCCTGGGGCACAAGCTGGAGTACAACTAC |
| | | AACAGCCACAACGTCTATATCATGGCCGACAAGCAGAA |
| | | GAACGGCATCAAGGTGAACTTCAAGATCCGCCACAACA |
| | | TCGAGGACGGCAGCGTGCAGCTCGCCGACCACTACCAG |
| | | CAGAACACCCCCATCGGCGACGGCCCCGTGCTGCTGCC |
| | | CGACAACCACTACCTGAGCACCCAGTCCGCCCTGAGCA |
| | | AAGACCCCAACGAGAAGCGCGATCACATGGTCCTGCTG |
| | | GAGTTCGTGACCGCCGCCGGGATCACTCTCGGCATGGA |
| | | CGAGCTGTACAAGTAAGACATTGAACAGCAAATTAACT |
| | | CAATTGATGCGATAAATCCATTGCACGCTTTTGATGACT |
| | | TAGAATCAGTAATACGTAATTTGATATCTGATTATGACA |
| | | AATTATTCCTTATGTTCAAAGGATTAATACAGAGATGTA |
| | | ATTATCAATATTCATTTGGTTGCGAATAACCATTTTGAT |
| | | ACATGTTGAACAATCAAATACAGTGTTAGTATGTTGTCA |
| | | TCTATGCATAACCCTCTATGAGCACAATAGTTAAAAGCT |
| | | AACACTGTCAAAAACCTAAATGGCTATAGGGGCGTTAT |
| | | GTGGCCGGGTCGGCATGGCATCTCCACCTCCTCGCGGTC |
| | | CGACCTGGGCATCCGAAGGAGGACGCACGTCCACTCGG |
| | | ATGGCTAAGGGAGAGCCTGCAGTAGCATAACCCCTTGG |
| | | GGCCTCTAAACGGGTCTTGAGGGGTTTTTTGGGTACC |
| 58 | pT7/NSP4-GFP- | ACTAGTTAATACGACTCACTATAGGCTTTTAAAAGTTCT |
| | NSP 4repeat | GTTCCGAGAGAGCGCGTGCGGAAAGATGGAAAAGCTTA |
| | T7 promoter: nt | CCGACCTCAATTATACATTGAGTGTAATCACTCTAATGA |
| | 7-27 | ACAATACATTGCACACAATACTTGAGGATCCAGGAATG |
| | NSP4 CDS: nt | GCGTATTTTCCTTATATAGCATCTGTCTTAACAGTTTTGT |
| | 65-589 | TTGCGCTACATAAAGCATCCATTCCAACAATGAAAATT |
| | 2A peptide | GCATTGAAAACGTCAAAATGTTCATATAAAGTGGTGAA |
| | CDS: nt 590- | ATATTGTATTGTAACAATTTTTAATACGTTGTTAAAATT |
| | 652 | GGCAGGTTATAAAGAGCAGATAACTACTAAAGATGAGA |
| | GFP CDS: nt | TAGAAAAGCAAATGGACAGAGTAGTCAAAGAAATGAG |
| | 653-1,369 | ACGCCAGCTAGAAATGATTGACAAATTGACTACACGTG |
| | NSP4 CDS (last | AAATTGAACAAGTAGAGTTGCTTAAACGCATTTACGAT |
| | 168bp): nt | AAATTGACGGTGCAAACGACAGGTGAAATAGATATGAC |
| | 1,370-1,537 | AAAAGAGATCAATCAAAAAAACGTGAGAACGCTAGAA |
| | HDV Ribozyme: | GAATGGGAAAGTGGAAAAAATCCTTATGAACCAAGAG |
| | nt 1,720-1,808 | AAGTGACTGCAGCAATGGGCTCCGGCGAGGGCAGGGG |
| | T7 Terminator: | AAGTCTTCTAACATGCGGGGACGTGGAGGAAAATCCCG |
| | nt 1,817-1,859 | GCCCAGTGAGCAAGGGCGAGGAGCTGTTCACCGGGGTG |
| | | GTGCCCATCCTGGTCGAGCTGGACGGCGACGTAAACGG |
| | | CCACAAGTTCAGCGTGTCCGGCGAGGGCGAGGGCGATG |
| | | CCACCTACGGCAAGCTGACCCTGAAGTTCATCTGCACC |
| | | ACCGGCAAGCTGCCCGTGCCCTGGCCCACCCTCGTGAC |
| | | CACCCTGACCTACGGCGTGCAGTGCTTCAGCCGCTACCC |
| | | CGACCACATGAAGCAGCACGACTTCTTCAAGTCCGCCA |
| | | TGCCCGAAGGCTACGTCCAGGAGCGCACCATCTTCTTC |
| | | AAGGACGACGGCAACTACAAGACCCGCGCCGAGGTGA |
| | | AGTTCGAGGGCGACACCCTGGTGAACCGCATCGAGCTG |
| | | AAGGGCATCGACTTCAAGGAGGACGGCAACATCCTGGG |
| | | GCACAAGCTGGAGTACAACTACAACAGCCACAACGTCT |
| | | ATATCATGGCCGACAAGCAGAAGAACGGCATCAAGGTG |
| | | AACTTCAAGATCCGCCACAACATCGAGGACGGCAGCGT |
| | | GCAGCTCGCCGACCACTACCAGCAGAACACCCCCATCG |
| | | GCGACGGCCCCGTGCTGCTGCCCGACAACCACTACCTG |
| | | AGCACCCAGTCCGCCCTGAGCAAAGACCCCAACGAGAA |
| | | GCGCGATCACATGGTCCTGCTGGAGTTCGTGACCGCCG |
| | | CCGGGATCACTCTCGGCATGGACGAGCTGTACAAGTAA |
| | | ATTGAACAAGTAGAGTTGCTTAAACGCATTTACGATAA |
| | | ATTGACGGTGCAAACGACAGGTGAAATAGATATGACAA |
| | | AAGAGATCAATCAAAAAAACGTGAGAACGCTAGAAGA |
| | | ATGGGAAAGTGGAAAAAATCCTTATGAACCAAGAGAA |
| | | GTGACTGCAGCAATGTAAGAGGTTGAGCTGCCGTCGAC |
| | | TGTCCTCGGAAGCGGCGGAGTTCTTTACAGTAAGCACC |
| | | ATCGGACCTGATGGCTGACTGAGAAGCCACAGTCAGCC |
| | | ATATCGCGTGTGGCTCAAGCCTTAATCCCGTTTAACCAA |
| | | TCCGGTCAGCACCGGACGTTAATGGAAGGAACGGTCTT |
| | | AATGTGACCGGGTCGGCATGGCATCTCCACCTCCTCGC |
| | | GGTCCGACCTGGGCATCCGAAGGAGGACGCACGTCCAC |
| | | TCGGATGGCTAAGGGAGAGCCTGCAGTAGCATAACCCC |
| | | TTGGGGCCTCTAAACGGGTCTTGAGGGGTTTTTTGGGTA |
| | | CC |
| 59 | pT7/NSP5-GFP- | AAGCTTTAATACGACTCACTATAGGCTTTTAAAGCGCTA |
| | NSP5repeat | CAGTGATGTCTCTCAGTATTGACGTGACGAGTCTTCCTT |
| | T7 promoter: nt | CTATTCCTTCAACTATATATAAGAATGAATCGTCTTCAA |
| | 7-27 | CAACGTCAACTCTTTCTGGAAAATCTATTGGTAGGAGTG |
| | NSP5 CDS: nt | AACAGTACATTTCACCAGATGCAGAAGCATTCAATAAA |
| | 45-638 | TACATGCTGTCGAAGTCTCCAGAGGATATTGGACCATCT |
| | 2A peptide | GATTCTGCTTCAAACGATCCACTCACCAGTTTTTCGATT |
| | CDS: nt 639- | AGATCGAATGCAGTTAAGACAAACGCAGACGCTGGCGT |
| | 701 | GTCTATGGATTCATCAGCACAATCACGACCTTCAAGTA |
| | GFP CDS: nt | ATGTCGGATGCGATCAAGTGGATTTCTCCTTAAATAAA |
| | 702-1,418 | GGCTTAAAAGTAAAAGCTAATTTGGACTCATCAATATC |
| | NSP5 CDS (last | AATATCTACGGATACTAAAAAGGAGAAATCAAAACAA |
| | 168bp): nt | AACCATAAAAGTAGGAAGCACTACCCAAGAATTGAAGC |
| | 1,419-1,586 | AGAGTCTGATTCAGATGATTATGTACTGGATGATTCAG |
| | HDV Ribozyme: | ATAGTGATGATGGTAAATGTAAGAACTGTAAATATAAG |
| | nt 1,636-1,724 | AAGAAATACTTCGCATTAAGAATGAGAATGAAACAAGT |
| | T7 Terminator: | CGCAATGCAATTGATTGAAGATTTGGGCTCCGGCGAGG |
| | nt 1,733-1,775 | GCAGGGGAAGTCTTCTAACATGCGGGGACGTGGAGGAA |
| | | AATCCCGGCCCAGTGAGCAAGGGCGAGGAGCTGTTCAC |
| | | CGGGGTGGTGCCCATCCTGGTCGAGCTGGACGGCGACG |
| | | TAAACGGCCACAAGTTCAGCGTGTCCGGCGAGGGCGAG |
| | | GGCGATGCCACCTACGGCAAGCTGACCCTGAAGTTCAT |
| | | CTGCACCACCGGCAAGCTGCCCGTGCCCTGGCCCACCC |
| | | TCGTGACCACCCTGACCTACGGCGTGCAGTGCTTCAGCC |
| | | GCTACCCCGACCACATGAAGCAGCACGACTTCTTCAAG |
| | | TCCGCCATGCCCGAAGGCTACGTCCAGGAGCGCACCAT |
| | | CTTCTTCAAGGACGACGGCAACTACAAGACCCGCGCCG |
| | | AGGTGAAGTTCGAGGGCGACACCCTGGTGAACCGCATC |
| | | GAGCTGAAGGGCATCGACTTCAAGGAGGACGGCAACAT |
| | | CCTGGGGCACAAGCTGGAGTACAACTACAACAGCCACA |
| | | ACGTCTATATCATGGCCGACAAGCAGAAGAACGGCATC |
| | | AAGGTGAACTTCAAGATCCGCCACAACATCGAGGACGG |
| | | CAGCGTGCAGCTCGCCGACCACTACCAGCAGAACACCC |
| | | CCATCGGCGACGGCCCCGTGCTGCTGCCCGACAACCAC |
| | | TACCTGAGCACCCAGTCCGCCCTGAGCAAAGACCCCAA |
| | | CGAGAAGCGCGATCACATGGTCCTGCTGGAGTTCGTGA |
| | | CCGCCGCCGGGATCACTCTCGGCATGGACGAGCTGTAC |
| | | AAGTAAAGGAAGCACTACCCAAGAATTGAAGCAGAGT |
| | | CTGATTCAGATGATTATGTACTGGATGATTCAGATAGTG |
| | | ATGATGGTAAATGTAAGAACTGTAAATATAAGAAGAAA |
| | | TACTTCGCATTAAGAATGAGAATGAAACAAGTCGCAAT |
| | | GCAATTGATTGAAGATTTGTAAGTCTGACCTGGGAACA |
| | | CACTAGGGAGCTCCCCACTCCCGTTTTGTGACCGGGTCG |
| | | GCATGGCATCTCCACCTCCTCGCGGTCCGACCTGGGCAT |
| | | CCGAAGGAGGACGCACGTCCACTCGGATGGCTAAGGGA |
| | | GAGCCTGCAGTAGCATAACCCCTTGGGGCCTCTAAACG |
| | | GGTCTTGAGGGGTTTTTTGGGTACC |

The disclosed subject matter is not to be limited in scope by the specific embodiments and examples described herein. Indeed, various modifications of the disclosure in addition to those described will become apparent to those skilled in the art from the foregoing description and accompanying figures. Such modifications are intended to fall within the scope of the appended claims.

All references (e.g., publications or patents or patent applications) cited herein are incorporated herein by reference in their entirety and for all purposes to the same extent as if each individual reference (e.g., publication or patent or patent application) was specifically and individually indicated to be incorporated by reference in its entirety for all purposes. Other embodiments are within the following claims.

What is claimed is:

1. An isolated nucleic acid molecule comprising:
a promoter sequence; and
a cDNA molecule encoding: a rotavirus non-structural protein; a 2A peptide downstream of the rotavirus non-structural protein; and a heterologous protein downstream of the 2A peptide.

2. The isolated nucleic acid molecule of claim 1, wherein the rotavirus non-structural protein is NSP1, NSP3, or NSP5.

3. The isolated nucleic acid molecule of claim 2, wherein the rotavirus non-structural protein is NSP1.

4. The isolated nucleic acid molecule of claim 2, wherein the rotavirus non-structural protein is NSP3.

5. The isolated nucleic acid molecule of claim 2, wherein the rotavirus non-structural protein is NSP5.

6. The isolated nucleic acid molecule of any one of claims 1-5, comprising a nucleic acid encoding an antigenomic hepatitis delta ribozyme, and wherein the promoter is a T7 promoter.

7. The isolated nucleic acid molecule of any one of claims 1-6, wherein the heterologous protein is a viral protein or fragment thereof.

8. The isolated nucleic acid molecule of claim 7, wherein the viral protein or fragment thereof is a SARS-COV-2 spike protein or a fragment thereof.

9. The isolated nucleic acid molecule of claim 8, wherein the viral protein or fragment thereof is the S1 domain of SARS-COV-2 spike protein (SEQ ID NO: 36) or the receptor binding domain of SARS-COV-2 spike protein (SEQ ID NO: 37).

10. The isolated nucleic acid molecule of claim 7, wherein the viral protein or fragment thereof is an RSV F protein or fragment thereof.

11. The isolated nucleic acid molecule of claim 10, wherein the viral protein or fragment thereof is RSV-T4PreF (SEQ ID NO: 44), RSV-T4scPreF (SEQ ID NO: 46), RSV-A2PreF (SEQ ID NO: 48), or RSV-A2scPreF (SEQ ID NO: 50).

12. The isolated nucleic acid molecule of any one of claims 1-6, wherein the heterologous protein is a fluorescent protein.

13. The isolated nucleic acid molecule of claim 12, wherein the fluorescent protein is: green fluorescent protein (GFP); enhanced GFP (eGFP); superfolder GFP; AcGFPl; ZsGreenl; enhanced blue fluorescent protein (EBFP), EBFP2, Azurite, mKalama; cyan fluorescent protein (CFP); enhanced CFP (ECFP); Cerulean; mHoneydew; CyPet; yellow fluorescent protein (YFP); Citrine; Venus; mBanana; ZsYellow1; Ypet; mOrange; tdTomato; LSSmOrange, PSmOrange PSmOrange2; DsRed; DsRed-monomer; DsRed-Express2; mRFPi; mCherry; mStrawberry; mRaspberry; niPluni; E2-Crimson; iRFP670; iRFP682; iRFP702; or iRFP720.

14. The isolated nucleic acid molecule of claim 13, wherein the fluorescent protein is GFP.

15. A recombinant rotavirus comprising in its genome a cDNA sequence encoding a 2A peptide downstream of NSP1, NSP3, or NSP5, and a heterologous gene downstream of the 2A peptide.

16. The recombinant rotavirus of claim 15, wherein the heterologous gene is downstream of NSP1.

17. The recombinant rotavirus of claim 15, wherein the heterologous gene is downstream of NSP3.

18. The recombinant rotavirus of claim 15, wherein the heterologous gene is downstream of NSP5.

19. The recombinant rotavirus of any one of claims 15-18, wherein the heterologous gene encodes a viral protein or fragment thereof.

20. The recombinant rotavirus of claim 19, wherein the viral protein or fragment thereof is a SARS-COV-2 spike protein or a variant or fragment thereof.

21. The recombinant rotavirus of claim 20, wherein the viral protein or fragment thereof is the S1 domain of SARS-COV-2 spike protein (SEQ ID NO: 36) or the receptor binding domain of SARS-COV-2 spike protein (SEQ ID NO: 37).

22. The recombinant rotavirus of claim 21, wherein the viral protein or fragment thereof is an RSV F protein or a variant or fragment thereof.

23. The recombinant rotavirus of claim 22, wherein the viral protein or fragment thereof is RSV-T4PreF (SEQ ID NO: 44), RSV-T4scPreF (SEQ ID NO: 46), RSV-A2PreF (SEQ ID NO: 48), or RSV-A2scPreF (SEQ ID NO: 50).

24. The isolated nucleic acid molecule of any one of claims 15-18, wherein the heterologous gene encodes a fluorescent protein.

25. The isolated nucleic acid molecule of claim 24, wherein the fluorescent protein is: green fluorescent protein (GFP); enhanced GFP (eGFP); superfolder GFP; AcGFPl; ZsGreenl; enhanced blue fluorescent protein (EBFP), EBFP2, Azurite, mKalama; cyan fluorescent protein (CFP); enhanced CFP (ECFP); Cerulean; mHoneydew; CyPet; yellow fluorescent protein (YFP); Citrine; Venus; mBanana; ZsYellow1; Ypet; mOrange; tdTomato; LSSmOrange, PSmOrange PSmOrange2; DsRed; DsRed-monomer; DsRed-Express2; mRFPi; mCherry; mStrawberry; mRaspberry; niPluni; E2-Crimson; iRFP670; iRFP682; iRFP702; or iRFP720.

26. The isolated nucleic acid molecule of claim 25, wherein the fluorescent protein is GFP.

27. An immunogenic composition comprising (i) an effective amount of the recombinant rotavirus of any one of claims 15-23, and (ii) a pharmaceutically acceptable carrier.

28. A method for treating or preventing an infection in a subject, comprising administering an effective amount of the immunogenic composition according to claim 27 to the subject.

29. A method for inducing a protective immune response in a subject, comprising administering an effective amount of the immunogenic composition of claim 27 to the subject.

30. The method of claim 29, wherein the immunogenic composition is administered to a mucous membrane of the subject.

31. The method of claim 30, wherein administration of the immunogenic composition is oral.

32. The method of any one of claims 28-31, comprising a first administration of the immunogenic composition and a second administration of the immunogenic composition.

33. The method of any one of claims 28-32, wherein the protective immune response is a humoral immune response and/or a cellular immune response.

34. The method of claim 33, wherein the second administration is performed from one month to two months after the first administration.

35. The method of any one of claims 28-34, wherein the subject is a human.

36. Use of the recombinant rotavirus of any one of claims 15-23 or the immunogenic composition of claim 27 for preventing or treating an infection.

37. The recombinant rotavirus of any one of claims 15-23 or the immunogenic composition of claim 27, for use in preventing or treating an infection in a subject.

38. In vitro use of the recombinant rotavirus of any one of claims 15-23 or the immunogenic composition of claim 27 expressing the heterologous protein in eukaryotic cells.

39. A method for rescuing recombinant rotavirus, the method comprising:
a) transfecting cells with
i) eleven individual rotavirus genomic segment plasmids (RGSP), each RGSP having a promoter and encoding one of a single rotavirus protein VP1, VP2, VP3, VP4, NSP1, VP6, NSP3, NSP2, VP7, NSP4, or NSP5, wherein one or more of the plasmids encoding NSP1, NSP3, and NSP5 protein includes a sequence encoding a 2A protein that is downstream of the NSP protein and a sequence encoding a heterologous protein that is downstream of the sequence encoding the 2A protein, and
ii) five individual helper plasmids (HPs), each HP having a promoter and encoding one of a fusogenic Fusion-Associated Small Transmembrane (FAST) protein, RNA capping enzyme DIR, RNA capping enzyme D12L, NSP2 protein, or NSP5 protein;
b) maintaining the transfected cells in conditions suitable for the production of recombinant rotavirus; and
c) harvesting the resulting recombinant rotavirus.

40. The method of claim 39, wherein the RGSPs comprise a nucleic acid encoding an antigenomic hepatitis delta ribozyme, and wherein the promoter of the RGSPs is a T7 promoter.

41. The method of any one of claim 39 or 40, wherein the transfected cells are Vero cells.

42. The method of any one of claims 39-41, comprising co-culturing the transfected cells of step (b) with cells that amplify replication of the recombinant rotavirus from the transfected cells.

43. The method of claim 42, wherein the cells that amplify replication of the recombinant rotavirus from the transfected cells are MA104 cells.
